(19) United States
(12) Patent Application Publication
(10) Pub. No.: US 2013/0210625 A1
(43) Pub. Date: Aug. 15, 2013

(54) SPIROHETEROCYCLIC PYRROLIDINE DERIVATIVES BASED PESTICIDES

(71) Applicants: Michel Muehlebach, Stein (CH); Jurgen Harry Schaetzer, Stein (CH)

(72) Inventors: Michel Muehlebach, Stein (CH); Jurgen Harry Schaetzer, Stein (CH)

(73) Assignee: SYNGENTA CROP PROTECTION LLC, Greensboro, NC (US)

(21) Appl. No.: 13/701,464

(22) PCT Filed: May 19, 2011
(86) PCT No.: PCT/EP11/58209

§ 371 Date: Feb. 12, 2013

(30) Foreign Application Priority Data
May 31, 2010 (EP) 10164516.6
Dec. 13, 2010 (EP) 10194687.9

(51) Int. Cl. A01N 43/90; C07D 211/94; C07D 405/12; C07D 471/10

(52) U.S. Cl. CPC A01N 43/90; C07D 471/10; C07D 211/94; C07D 405/12
USPC 504/116.1; 546/16; 514/278; 546/224; 546/215; 546/214

(57) ABSTRACT

Compounds of the formula (I), wherein the substituents are as defined in claim 1, are useful as a pesticides.

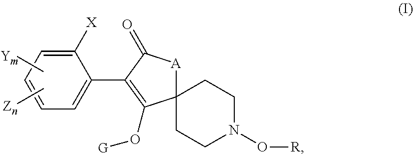

The present invention relates to new substituted spiroheterocyclic pyrrolidine dione derivatives, to processes for preparing them, to pesticidal, in particular insecticidal, acaricidal, molluscicidal and nematicidal compositions comprising them and to methods of using them to combat and control pests such as insect, acarine, mollusc and nematode pests.

Spiroheterocyclic pyrrolidine dione derivatives are disclosed for example in WO 09/049,851, WO 10/063,670 and WO 10/066,780.

It has now surprisingly been found that certain new substituted spiroheterocyclic pyrrolidine dione derivatives have good insecticidal properties.

The present invention therefore provides compounds of the formula I

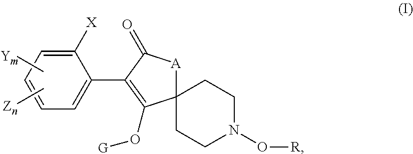

wherein
X, Y and Z independently of each other are C₁₋₄alkyl, C₃₋₆cycloalkyl, C₁₋₄haloalkyl C₁₋₄alkoxy, halogen, phenyl or phenyl substituted by C₁₋₄alkyl, C₁₋₄haloalkyl, halogen or cyano;
m and n, independently of each other, are 0, 1, 2 or 3 and m+n is 0, 1, 2 or 3;
G is hydrogen, a metal, ammonium, sulfonium or a latentiating group;
A is either NR¹ or NOR¹, wherein R¹ is hydrogen, C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cyclo-alkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄)alkyl, C₁₋₄alkylsulfinyl(C₁₋₄)alkyl or C₁₋₄alkylsulfonyl(C₁₋₄)alkyl;
R is hydrogen, C₁₋₆alkyl, C₁₋₆haloalkyl, C₁₋₆cyanoalkyl, C₂₋₆alkenyl, C₂₋₆haloalkenyl, C₃-C₆alkynyl, benzyl, C₁₋₄alkoxy(C₁₋₄)alkyl, or C₁₋₄alkoxy(C₁₋₄)alkoxy(C₁₋₄)alkyl; and
with the proviso that at least one of X, Y or Z is C₂₋₆alkenyl, C₂₋₆alkynyl, —CHO, C₁₋₆alkylcarbonyl or C₁₋₆alkoxycarbonyl;
or an agrochemically acceptable salt or an N-oxide thereof.

In the compounds of the formula I, each alkyl moiety either alone or as part of a larger group is a straight or branched chain and is, for example, methyl, ethyl, n-propyl, n-butyl, iso-propyl, sec-butyl, iso-butyl, tert-butyl, n-pentyl, iso-pentyl and n-hexyl.

Alkoxy groups preferably have a preferred chain length of from 1 to 6 carbon atoms. Alkoxy is, for example, methoxy, ethoxy, propoxy, i-propoxy, n-butoxy, isobutoxy, sec-butoxy or tert-butoxy. Such groups can be part of a larger group such as alkoxyalkyl and alkoxyalkoxyalkyl. Alkoxyalkyl and alkylthioalkyl groups preferably have a chain length of 1 to 4 carbon atoms. Alkoxyalkyl is, for example, methoxymethyl, methoxyethyl, ethoxymethyl, ethoxyethyl, n-propoxymethyl, n-propoxyethyl or isopropoxymethyl. In alkylthioalkyl groups, oxygen is replaced by sulphur.

Halogen is generally fluorine, chlorine, bromine or iodine. This also applies, correspondingly, to halogen in combination with other meanings, such as haloalkyl or haloalkenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl.

The preferred alkenyl and alkynyl radicals having 2 to 6 carbon atoms can be straight or branched and can contain more than 1 double or triple bond. Examples are vinyl, (E)- or (Z)-propenyl, 2-methyl-propenyl, allyl, 3-methyl-but-2-enyl, ethynyl, prop-1-ynyl, propargyl, butenyl, butynyl, pentenyl and pentynyl.

The cycloalkyl and cycloalkylalkyl groups preferably have from 3 to 6 ring carbon atoms, for example cyclopropyl, cyclobutyl, cyclopentyl and cyclohexyl. In these rings, a methylene group can be replaced by a heteroatom such as oxygen, sulphur, or nitrogen in form of a group NR₀, where R₀ is C₁₋₆alkyl or C₁₋₆alkoxy, which leads, for example, to oxetan-3-yl, tetrahydrofuran-2-yl, tetrahydropyran-2-yl, tetrahydrofuran-3-yl, tetrahydropyran-3-yl, tetrahydropyran-4-yl, tetrahydro-thiofuranyl, tetrahydro-thiopyranyl, N—(C₁₋₄)alkyl-piperidinyl or N—(C₁₋₄)alkoxy-piperidinyl rings. Cycloalkylalkyl and furanylalkyl groups preferably have a chain length of 1 to 4 carbon atoms. Cycloalkylalkyl is, for example, cyclopropylmethyl, cyclobutylmethyl, cyclopentylmethyl, or cyclohexylmethyl. Furanylalkyl is, for example, furan-2-ylmethyl or furan-3-ylmethyl. The same apply when a methylene group in the cycloalkyl moiety is replaced by O, S or NR₀, where R₀ is C₁₋₆alkyl or C₁₋₆alkoxy, to form groups such as, for example, oxetan-3-ylmethyl, tetrahydrofuran-2-ylmethyl, tetrahydropyran-2-ylmethyl, tetrahydrofuran-3-ylmethyl, tetrahydropyran-3-ylmethyl, tetrahydropyran-4-ylmethyl or tetrahydro-thiopyran-4-ylmethyl.

Phenyl, also as part of a substituent such as benzyl, may be substituted, preferably by alkyl, haloalkyl, halogen or cyano groups. In this case, the substituents can be in ortho, meta and/or para position. The preferred substituent positions are the ortho and para positions, especially the ortho position to the ring attachment point.

The latentiating groups G are selected to allow its removal by one or a combination of biochemical, chemical or physical processes to afford compounds of formula I where G is hydrogen before, during or following application to the treated area or plants. Examples of these processes include enzymatic cleavage, chemical hydrolysis and photoloysis. Compounds bearing such groups G may offer certain advantages, such as improved penetration of the cuticula of the plants treated, increased tolerance of crops, improved compatibility or stability in formulated mixtures containing other insecticides, herbicide safeners, plant growth regulators, herbicides or fungicides, or reduced leaching in soils.

The latentiating group G is preferably selected from the groups C₁-C₈alkyl, C₂-C₃haloalkyl, phenylC₁-C₈alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano or by nitro), heteroarylC₁-C₈alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₈alkenyl, C₃-C₈haloalkenyl, C₃-C₈alkynyl, C(X^{a})—R^{a}, C(X^{b})—X^{c}—R^{b}, C(X^{d})—N(R^{c})—R^{d}, —SO₂—R^{e}, —P(X^{e})(R^{f})—R^{g} or CH₂—X^{f}—R^{h} wherein X^{a}, X^{b}, X^{c}, X^{d}, X^{e} and X^{f} are independently of each other oxygen or sulfur;

R^{a} is H, C₁-C₁₈alkyl, C₂-C₁₈alkenyl, C₂-C₁₈alkynyl, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynylC₁-C₅oxyalkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or nitro,
R^{b} is C₁-C₁₈alkyl, C₃-C₁₈alkenyl, C₃-C₁₈alkynyl, C₂-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkyl-thio, C₁-C₃alkylsulfinyl, C₁-C₃alkylsulfonyl, halogen, cyano, or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃halo-alkoxy, halogen, cyano or nitro, heteroaryl or heteroaryl substituted by C₁-C₃ alkyl, C₁₋₃haloalkyl, C₁-C₃alkoxy, C₁-C₃haloalkoxy, halogen, cyano or nitro,
R^{c} and R^{d} are each independently of each other hydrogen, C₁-C₁₀alkyl, C₃-C₁₀alkenyl, C₃-C₁₀alkynyl, C₂-C₁₀haloalkyl, C₁-C₁₀cyanoalkyl, C₁-C₁₀nitroalkyl, C₁-C₁₀-aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₂-C₅alkylaminoalkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl, (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃alkyl, C₁-C₃haloalkyl, C₁-C₃alkoxy, C₁-C₃haloalkoxy, halogen, cyano or nitro, heteroarylamino or heteroarylamino substituted by C₁-C₃alkyl, C₁-C₃haloalkyl, C₁-C₃alkoxy, C₁-C₃haloalkoxy, halogen, cyano or nitro, diheteroarylamino or diheteroarylamino substituted by C₁-C₃alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or by nitro, diphenylamino or diphenylamino substituted by C₁-C₃alkyl, C₁-C₃haloalkyl, C₁-C₃alkoxy, C₁-C₃haloalkoxy, halogen, cyano or by nitro or C₃-C₇cycloalkylamino, di-C₃-C₇cycloalkylamino or C₃-C₇cycloalkoxy or R^{c} and R^{d} may join together to form a 3-7 membered ring, optionally containing one heteroatom selected from O or S,
R^{e} is C₁-C₁₀alkyl, C₂-C₁₀alkenyl, C₂-C₁₀alkynyl, C₁-C₁₀haloalkyl, C₁-C₁₀cyanoalkyl, C₁-C₁₀nitroalkyl, C₁-C₁₀-aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀alkoxy, C₁-C₁₀haloalkoxy, C₁-C₅alkylamino or C₂-C₈dialkylamino
R^{f} and R^{g} are each independently of each other C₁-C₁₀alkyl, C₂-C₁₀alkenyl, C₂-C₁₀alkynyl, C₁-C₁₀alkoxy, C₁-C₁₀haloalkyl, C₁-C₁₀cyanoalkyl, C₁-C₁₀nitroalkyl, C₁-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₂-C₅alkylaminoalkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃alkylsulfonyl, halogen, cyano, or by nitro), C₂-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen, cyano or nitro, heteroaryl or heteroaryl substituted by C₁-C₃ alkyl, C₁-C₃haloalkyl, C₁-C₃alkoxy, C₁-C₃haloalkoxy, halogen, cyano or by nitro, heteroarylamino or heteroarylamino substituted by C₁-C₃ alkyl, C₁-C₃haloalkyl, C₁-C₃alkoxy, C₁-C₃haloalkoxy, halogen, cyano or by nitro, diheteroarylamino or diheteroarylamino substituted by C₁-C₃ alkyl, C₁-C₃haloalkyl, C₁-C₃alkoxy, C₁-C₃haloalkoxy, halogen, cyano or nitro, phenylamino or phenylamino substituted by C₁-C₃alkyl, C₁-C₃haloalkyl, C₁-C₃alkoxy, C₁-C₃haloalkoxy, halogen, cyano or nitro, diphenylamino, or diphenylamino substituted by C₁-C₃alkyl, C₁-C₃haloalkyl, C₁-C₃alkoxy, C₁-C₃haloalkoxy, halogen, cyano or nitro, or C₃-C₇cycloalkylamino, diC₃-C₇cycloalkylamino or C₃-C₇cycloalkoxy, C₁-C₁₀haloalkoxy, C₁-C₅alkylamino or C₂-C₈dialkylamino, benzyloxy or phenoxy, wherein the benzyl and phenyl groups may in turn be substituted by C₁-C₃alkyl, C₁-C₃haloalkyl, C₁-C₃alkoxy, C₁-C₃haloalkoxy, halogen, cyano or nitro, and
R^{h} is C₁-C₁₀alkyl, C₃-C₁₀alkenyl, C₃-C₁₀alkynyl, C₁-C₁₀haloalkyl, C₁-C₁₀cyanoalkyl, C₁-C₁₀nitroalkyl, C₂-C₁₀aminoalkyl, C₁-C₅alkylaminoC₁-C₅alkyl, C₂-C₈dialkylaminoC₁-C₅alkyl, C₃-C₇cycloalkylC₁-C₅alkyl, C₁-C₅alkoxyC₁-C₅alkyl, C₃-C₅alkenyloxyC₁-C₅alkyl, C₃-C₅alkynyloxyC₁-C₅alkyl, C₁-C₅alkylthioC₁-C₅alkyl, C₁-C₅alkylsulfinylC₁-C₅alkyl, C₁-C₅alkylsulfonylC₁-C₅alkyl, C₂-C₈alkylideneaminoxyC₁-C₅alkyl, C₁-C₅alkylcarbonylC₁-C₅alkyl, C₁-C₅alkoxycarbonylC₁-C₅alkyl, aminocarbonylC₁-C₅alkyl, C₁-C₅alkylaminocarbonylC₁-C₅alkyl, C₂-C₈dialkylaminocarbonylC₁-C₅alkyl, C₁-C₅alkylcarbonylaminoC₁-C₅alkyl, N—C₁-C₅alkylcarbonyl-N—C₁-C₅alkylaminoC₁-C₅alkyl, C₃-C₆-trialkylsilylC₁-C₅alkyl, phenylC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroarylC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), phenoxyC₁-C₅alkyl (wherein the phenyl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), heteroaryloxyC₁-C₅alkyl (wherein the heteroaryl may optionally be substituted by C₁-C₃alkyl, C₁-C₃haloalkyl, C₁-C₃alkoxy, C₁-C₃haloalkoxy, C₁-C₃alkylthio, C₁-C₃alkylsulfinyl, C₁-C₃ alkylsulfonyl, halogen, cyano or by nitro), C₃-C₅haloalkenyl, C₃-C₈cycloalkyl, phenyl or phenyl substituted by C₁-C₃alkyl, C₁-C₃haloalkyl, C₁-C₃alkoxy, C₁-C₃haloalkoxy, halogen or by nitro, or heteroaryl, or heteroaryl substituted by C₁-C₃alkyl, C₁-C₃haloalkyl, C₁-C₃alkoxy, C₁-C₃haloalkoxy, halogen, cyano or by nitro.

In particular, the latentiating group G is a group —C(X^{a})—R^{a} or —C(X^{b})—X^{c}—R^{b}, and the meanings of X^{a}, R^{a}, X^{b}, X^{c} and R^{b} are as defined above.

It is also preferred that G is hydrogen, an alkali metal or alkaline earth metal, or an ammonium or sulfonium group, where hydrogen is especially preferred.

Depending on the nature of the substituents, compounds of formula I may exist in different isomeric forms. When G is hydrogen, for example, compounds of formula I may exist in different tautomeric forms:

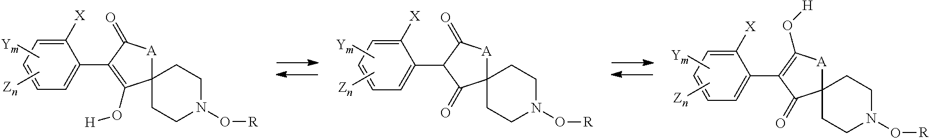

This invention covers all such isomers and tautomers and mixtures thereof in all proportions. Also, when substituents contain double bonds, cis- and trans-isomers can exist. These isomers, too, are within the scope of the claimed compounds of the formula I.

The invention relates also to the agriculturally acceptable salts which the compounds of formula I are able to form with transition metal, alkali metal and alkaline earth metal bases, amines, quaternary ammonium bases or tertiary sulfonium bases.

Among the transition metal, alkali metal and alkaline earth metal salt formers, special mention should be made of the hydroxides of copper, iron, lithium, sodium, potassium, magnesium and calcium, and preferably the hydroxides, bicarbonates and carbonates of sodium and potassium.

Examples of amines suitable for ammonium salt formation include ammonia as well as primary, secondary and tertiary C₁-C₁₈alkylamines, C₁-C₄hydroxyalkylamines and C₂-C₄alkoxyalkyl-amines, for example methylamine, ethylamine, n-propylamine, propylamine, the four butylamine isomers, n-amylamine, i-amylamine, hexylamine, heptylamine, octylamine, nonylamine, decylamine, pentadecylamine, hexadecylamine, heptadecylamine, octadecylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, di-i-propylamine, di-n-butylamine, di-n-amylamine, di-i-amylamine, dihexylamine, diheptylamine, dioctylamine, ethanolamine, n-propanolamine, i-propanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, trimethylamine, triethylamine, tri-n-propylamine, tri-i-opropylamine, tri-n-butylamine, tri-i-butylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, i-propylamine and di-i-propylamine.

Preferred quaternary ammonium bases suitable for salt formation correspond, for example, to the formula [N(Rₐ R_{b} R_{c} R_{d})]OH, wherein Rₐ, R_{b}, R_{c} and R_{d} are each independently of the others hydrogen or C₁-C₄alkyl. Further suitable tetraalkylammonium bases with other anions can be obtained, for example, by anion exchange reactions.

Preferred tertiary sulfonium bases suitable for salt formation correspond, for example, to the formula [SRₑR_{f}R_{g}]OH, wherein Rₑ, R_{f} and R_{g} are each independently of the others C₁-C₄ alkyl. Trimethylsulfonium hydroxide is especially preferred. Suitable sulfonium bases may be obtained from the reaction of thioethers, in particular dialkylsulfides, with alkylhalides, followed by conversion to a suitable base, for example a hydroxide, by anion exchange reactions.

It should be understood that in those compounds of formula I, where G is a metal, ammonium or sulfonium as mentioned above and as such represents a cation, the corresponding negative charge is largely delocalised across the O—C═C—C═O unit.

The compounds of formula I according to the invention also include hydrates which may be formed during the salt formation.

The preferred values of R, A, X, Y, Z, m and n in the compounds of formula I in any combination thereof are set out below, and can be combined with any values of G, in particular with any preferred values of G, as defined above.

Preferably R is hydrogen, C₁₋₆alkyl, C₁₋₆haloalkyl, C₁₋₆cyanoalkyl, C₂₋₆alkenyl, C₂₋₆haloalkenyl, C₃₋₆alkynyl, benzyl, C₁₋₄alkoxy(C₁₋₄)alkyl or C₁₋₄alkoxy(C₁₋₄)alkoxy(C₁₋₄)alkyl, in particular hydrogen, methyl, ethyl, n-propyl, isopropyl, cyanomethyl, trifluoromethyl, 2,2,2-trifluoroethyl, allyl, 3,3-dichloroallyl, propargyl, benzyl, methoxymethyl, ethoxymethyl, methoxyethyl or methoxyethoxymethyl.

Preferably A is either NR¹ or NOR¹, wherein R¹ is hydrogen, C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cycloalkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄)alkyl, C₁₋₄alkylsulfinyl(C₁₋₄)alkyl or C₁₋₄alkylsulfonyl(C₁₋₄)alkyl, in particular

hydrogen, methyl, ethyl, isopropyl, trifluoromethyl, 2,2,2-trifluoroethyl, 2,2-difluoroethyl, 2-fluoroethyl, cyclopropyl, cyclobutyl, cyclopentyl, cyclohexyl, cyclopropylmethyl, cyclobutylmethyl, cyclopentylmethyl, cyclohexylmethyl, allyl, propargyl, benzyl, methoxymethyl, ethoxymethyl, methoxyethyl, methoxypropyl, methoxyethoxymethyl, methoxymethoxyethyl, oxetan-3-yl, tetrahydrofuran-2-yl, tetrahydropyran-2-yl, tetrahydrofuran-3-yl, tetrahydropyran-4-yl, tetrahydrothiopyran-4-yl, 1-methoxy-piperidin-4-yl, oxetan-3-ylmethyl, tetrahydrofuran-2-ylmethyl, tetrahydropyran-2-ylmethyl, tetrahydrofuran-3-ylmethyl, tetrahydropyran-3-ylmethyl, tetrahydropyran-4-ylmethyl, methylthioethyl, 2-methanesulfinyl-ethyl, 2-methanesulfonyl-ethyl, furan-2-ylmethyl, furan-3-ylmethyl or tetrahydro-thiopyran-4-ylmethyl.

Preferably X is C₁-C₄alkyl, C₃-C₆cycloalkyl, C₁-C₄alkoxy or halogen.

More preferably X is methyl, ethyl, cyclopropyl, methoxy, fluoro, bromo or chloro.

Preferably Y and Z, independently of each other, are C₁-C₄alkyl, C₃-C₆cycloalkyl, C₁-C₄alkoxy, halogen, phenyl, phenyl substituted by C₁-C₄alkyl or halo-substituted phenyl and m+n is 1, 2 or 3 and in particular m+n is 1 or 2.

More preferably Y and Z, independently of each other, are methyl, ethyl, cyclopropyl, methoxy, fluoro, bromo or chloro, phenyl or halo-substituted phenyl (in particular fluorophenyl or chlorophenyl and especially 4-chlorophenyl or 4-fluorophenyl) and m+n is 1, 2 or 3 and in particular m+n is 1 or 2.

Yet more preferably Y and Z are each independently methyl, ethyl, cyclopropyl, methoxy, fluoro, bromo or chloro and m+n is 1, 2 or 3, in particular, m+n is 1 or 2.

At least one of X, Y or Z is C₂₋₆alkenyl, C₂₋₆alkynyl, —CHO, C₁₋₆alkylcarbonyl or C₁₋₆alkoxycarbonyl; in particular, at least one of X, Y or Z is C₂₋₆alkenyl or C₂₋₆alkynyl, preferably vinyl or ethynyl.

It is also preferred that at least one of X, Y or Z is vinyl, (E)-propenyl, (Z)-propenyl, 2-methyl-propenyl, allyl, 3-methyl-but-2-enyl, ethynyl, prop-1-ynyl, propargyl, —CHO, methylcarbonyl, ethylcarbonyl, isopropylcarbonyl, methoxycarbonyl, ethoxycarbonyl, isopropoxycarbonyl or tert-butyloxycarbonyl.

More preferably at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl.

In one preferred group of compounds of the formula (I), R is hydrogen, methyl, ethyl, n-propyl, isopropyl, cyanomethyl, trifluoromethyl, 2,2,2-trifluoroethyl, allyl, 3,3-dichloroallyl, propargyl, benzyl, methoxymethyl, ethoxymethyl, methoxyethyl or methoxyethoxymethyl, A is either NR¹ or NOR¹, wherein R¹ is hydrogen, methyl, ethyl, isopropyl, trifluoromethyl, 2,2,2-trifluoroethyl, 2,2-difluoroethyl, 2-fluoroethyl, cyclopropyl, cyclobutyl, cyclopentyl, cyclohexyl, cyclopropylmethyl, cyclobutylmethyl, cyclopentylmethyl, cyclohexylmethyl, allyl, propargyl, benzyl, methoxymethyl, ethoxymethyl, methoxyethyl, methoxypropyl, methoxyethoxymethyl, methoxymethoxyethyl, oxetan-3-yl, tetrahydrofuran-2-yl, tetrahydropyran-2-yl, tetrahydrofuran-3-yl, tetrahydropyran-4-yl, tetrahydrothiopyran-4-yl, 1-methoxy-piperidin-4-yl, oxetan-3-ylmethyl, tetrahydrofuran-2-ylmethyl, tetrahydropyran-2-ylmethyl, tetrahydrofuran-3-ylmethyl, tetrahydropyran-3-ylmethyl, tetrahydropyran-4-ylmethyl, methylthioethyl, 2-methanesulfinyl-ethyl, 2-methanesulfonyl-ethyl, furan-2-ylmethyl, furan-3-ylmethyl or tetrahydro-thiopyran-4-ylmethyl, X is methyl, ethyl, cyclopropyl, methoxy, fluoro, bromo or chloro, Y and Z, independently of each other, are methyl, ethyl, cyclopropyl, methoxy, fluoro, chloro, bromo, phenyl or phenyl substituted by halogen or C₁-C₂alkyl, with the proviso that at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl, G has the meanings assigned to it above and m+n is 1, 2 or 3.

In another preferred group of compounds of the formula (I), R is hydrogen, methyl, ethyl, isopropyl, cyanomethyl, 2,2,2-trifluoroethyl, allyl, propargyl, benzyl, methoxymethyl, ethoxymethyl or methoxyethyl, A is either NR¹ or NOR¹, wherein R¹ is hydrogen, methyl, ethyl, isopropyl, trifluoromethyl, 2,2,2-trifluoroethyl, 2,2-difluoroethyl, 2-fluoroethyl, cyclopropyl, cyclobutyl, cyclopentyl, cyclohexyl, cyclopropylmethyl, cyclobutylmethyl, cyclopentylmethyl, cyclohexylmethyl, allyl, propargyl, benzyl, methoxymethyl, ethoxymethyl, methoxyethyl, methoxypropyl, methoxyethoxymethyl, methoxymethoxyethyl, oxetan-3-yl, tetrahydrofuran-2-yl, tetrahydropyran-2-yl, tetrahydrofuran-3-yl, tetrahydropyran-4-yl, tetrahydrothiopyran-4-yl, 1-methoxy-piperidin-4-yl, oxetan-3-ylmethyl, tetrahydrofuran-2-ylmethyl, tetrahydropyran-2-ylmethyl, tetrahydrofuran-3-ylmethyl, tetrahydropyran-3-ylmethyl, tetrahydropyran-4-ylmethyl, methylthioethyl, 2-methanesulfinyl-ethyl, 2-methanesulfonyl-ethyl, furan-2-ylmethyl, furan-3-ylmethyl or tetrahydro-thiopyran-4-ylmethyl, X is methyl, ethyl, cyclopropyl, methoxy, fluoro, bromo or chloro, Y and Z, independently of each other, are methyl, ethyl, cyclopropyl, methoxy, fluoro, chloro, bromo, phenyl or phenyl substituted by halogen or C₁-C₂alkyl, with the proviso that at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl, G has the meanings assigned to it above and m+n is 1, 2 or 3.

In yet another preferred group of compounds of the formula (I), R is hydrogen, methyl, ethyl, isopropyl, cyanomethyl, 2,2,2-trifluoroethyl, allyl, propargyl, benzyl, methoxymethyl, ethoxymethyl or methoxyethyl, A is NH, X is methyl, ethyl, cyclopropyl, methoxy, fluoro, bromo or chloro, Y and Z, independently of each other, are methyl, ethyl, cyclopropyl, methoxy, fluoro, chloro, bromo, phenyl or phenyl substituted by halogen or C₁-C₂alkyl, with the proviso that at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl, G has the meanings assigned to it above and m+n is 1, 2 or 3.

In even another preferred group of compounds of the formula (I), R is hydrogen, methyl, ethyl, isopropyl, cyanomethyl, 2,2,2-trifluoroethyl, allyl, propargyl, benzyl, methoxymethyl, ethoxymethyl or methoxyethyl, A is NR¹, wherein R¹ is methyl, ethyl, isopropyl, 2,2,2-trifluoroethyl, cyclopropyl, cyclopentyl, cyclohexyl, cyclopropylmethyl, cyclopentylmethyl, cyclohexylmethyl, allyl, propargyl, benzyl, methoxymethyl, methoxyethyl, methoxypropyl, methoxyethoxymethyl, methoxymethoxyethyl, tetrahydrofuran-2-ylmethyl, tetrahydrofuran-3-ylmethyl, tetrahydropyran-4-ylmethyl, methylthioethyl or tetrahydro-thiopyran-4-ylmethyl, X is methyl, ethyl, cyclopropyl, methoxy, fluoro, bromo or chloro, Y and Z, independently of each other, are methyl, ethyl, cyclopropyl, methoxy, fluoro, chloro, bromo, phenyl or phenyl substituted by halogen or C₁-C₂alkyl, with the proviso that at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl, G has the meanings assigned to it above and m+n is 1, 2 or 3.

In still another preferred group of compounds of the formula (I), R is hydrogen, methyl, ethyl, isopropyl, cyanomethyl, 2,2,2-trifluoroethyl, allyl, propargyl, benzyl, methoxymethyl, ethoxymethyl or methoxyethyl, A is NOR¹, wherein R¹ is hydrogen, methyl, ethyl, isopropyl, 2,2,2-trifluoroethyl, cyclopropyl, cyclopentyl, cyclohexyl, cyclopropylmethyl, cyclopentylmethyl, cyclohexylmethyl, allyl, propargyl, benzyl, methoxymethyl, ethoxymethyl, methoxyethyl, methoxypropyl, methoxyethoxymethyl, methoxymethoxyethyl, oxetan-3-yl, tetrahydrofuran-2-yl, tetrahydropyran-2-yl, tetrahydrofuran-3-yl, tetrahydropyran-4-yl, tetrahydrothiopyran-4-yl, 1-methoxy-piperidin-4-yl, oxetan-3-ylmethyl, tetrahydrofuran-2-ylmethyl, tetrahydropyran-2-ylmethyl, tetrahydrofuran-3-ylmethyl, tetrahydropyran-3-ylmethyl, tetrahydropyran-4-ylmethyl, methylthioethyl, furan-2-ylmethyl, furan-3-ylmethyl or tetrahydro-thiopyran-4-ylmethyl, X is methyl, ethyl, cyclopropyl, methoxy, fluoro, bromo or chloro, Y and Z, independently of each other, are methyl, ethyl, cyclopropyl, methoxy, fluoro, chloro, bromo, phenyl or phenyl substituted by halogen or C₁-C₂alkyl, with the proviso that at least one of X, Y or Z is vinyl, (E)-propenyl, (Z)-propenyl, 2-methyl-propenyl, allyl, ethynyl, prop-1-ynyl, propargyl, —CHO, methylcarbonyl, ethylcarbonyl, methoxycarbonyl, ethoxycarbonyl or tert-butyloxycarbonyl, G has the meanings assigned to it above and m+n is 1, 2 or 3.

In still another preferred group of compounds of the formula (I), R is methyl, A is NH, NCH₃, NOCH₃ or NO-tetrahydrofuran-3-yl, X is methyl or ethynyl, Y and Z, independently of each other, are methyl, vinyl, ethynyl, prop-1-ynyl, methylcarbonyl or methoxycarbonyl, G is ethoxycarbonyl and m+n is 1 or 2.

The invention covers also salts of the compounds of the formula I with amines, alkali metal and alkaline earth metal bases or quaternary ammonium bases.

Among the alkali metal and alkaline earth metal hydroxides as salt formers, special mention should be made of the hydroxides of lithium, sodium, potassium, magnesium and calcium, but especially the hydroxides of sodium and potassium. The compounds of formula I according to the invention also include hydrates which may be formed during the salt formation.

Examples of amines suitable for ammonium salt formation include ammonia as well as primary, secondary and tertiary C₁-C₁₈alkylamines, C₁-C₄hydroxyalkylamines and C₂-C₄alkoxyalkylamines, for example methylamine, ethylamine, n-propylamine, isopropylamine, the four butylamine isomers, n-amylamine, isoamylamine, hexylamine, heptylamine, octylamine, nonylamine, decylamine, pentadecylamine, hexadecylamine, heptadecylamine, octadecylamine, methylethylamine, methylisopropylamine, methylhexylamine, methylnonylamine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diisoamylamine, dihexylamine, diheptylamine, dioctylamine, ethanolamine, n-propanolamine, isopropanolamine, N,N-diethanolamine, N-ethylpropanolamine, N-butylethanolamine, allylamine, n-but-2-enylamine, n-pent-2-enylamine, 2,3-dimethylbut-2-enylamine, dibut-2-enylamine, n-hex-2-enylamine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylenediamines, benzidines, naphthylamines and o-, m- and p-chloroanilines; but especially triethylamine, isopropylamine and diisopropylamine.

Preferred quaternary ammonium bases suitable for salt formation correspond, for example, to the formula [N(Rₐ R_{b} R_{c} R_{d})]OH wherein Rₐ, R_{b}, R_{c} and R_{d} are each independently of the others C₁-C₄alkyl. Further suitable tetraalkylammonium bases with other anions can be obtained, for example, by anion exchange reactions.

The compounds of the invention may be made by a variety of methods. For example, the compounds of formula I, wherein the substituents have the meanings assigned to them above, can be prepared by means of processes known per se, e.g. by treating compounds of formula II with an alkylating, acylating, phosphorylating or sulfonylating agent G-Q in the presence of at least one equivalent of a base, where G is the alkyl, acyl, phosphoryl or sulfonyl group to be incorporated and Q is a nucleofuge:

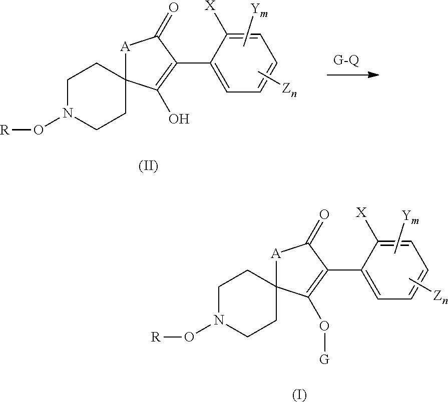

Compounds of formula I, in which X, Y, Z, m, n, A and R are as defined above and wherein G is a latentiating group of the formula —C(X^{a})—R^{a}, C(X^{b})—X^{c}—R^{b} or —C(X^{d})—NR^{c}R^{d} may be prepared by procedures known in the art, described for example in WO 09/049,851. Typically, compounds of formula II, in which X, Y, Z, m, n, A and R are as defined above, are treated with an acylating agent such as an acid halide (especially acid chloride), acid anhydride, haloformate (especially chloroformate), halothioformate (especially chlorothioformate), isocyanate, isothiocycanate, carbamoyl halide (especially carbamoyl chloride) or thiocarbamoyl halide (especially thiocarbamoyl chloride) in the presence of at least one equivalent of a suitable base, optionally in the presence of a suitable solvent. The base may be inorganic such as an alkali metal carbonate or hydroxide or a metal hydride, or an organic base such as a tertiary amine or metal alkoxide. Examples of suitable inorganic bases include sodium carbonate, sodium or potassium hydroxide, sodium hydride, and suitable organic bases include trialkylamines such as trimethylamine and triethylamine, pyridines or other amine bases such as 1,4-diazobicyclo[2.2.2]octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent. For cases, where the acylating agent is a carboxylic acid, acylation is preferably effected in the presence of a coupling agent such as 2-chloro-1-methylpyridinium iodide, N,N′-dicyclohexycarbodiimide, 1-(3-dimethylaminopropyl)-3-ethylcarbodiimide and N,N′-carbodiimidazole, and a base such as triethylamine or pyridine in a suitable solvent such as tetrahydrofuran, dichloromethane and acetonitrile.

Compounds of formula I, in which X, Y, Z, m, n, A and R are as defined above and wherein G is a latentiating group of the formula C(X^{b})—X^{c}—R^{b} or —C(X^{d})—NR^{c}R^{d}, may be also be prepared by treating compounds of formula II, in which X, Y, Z, m, n, A and R are as defined above, with phosgene or a phosgene equivalent, optionally in the presence of a solvent such as toluene or ethyl acetate, and a base and reacting the resultant chloroformate, or equivalent, with an alcohol, thiol or amine under known conditions, as described, for example, in U.S. Pat. No. 6,774,133, U.S. Pat. No. 6,555,567 and U.S. Pat. No. 6,479,489.

Compounds of formula I, in which X, Y, Z, m, n, A and R are as defined above and wherein G is a latentiating group of the formula —P(X^{e})R^{f}R^{g}, may be prepared from compounds of formula II, in which X, Y, Z, m, n, A and R are as defined above, using procedures described, for example, in U.S. Pat. No. 6,774,133, U.S. Pat. No. 6,555,567 and U.S. Pat. No. 6,479,489.

Compounds of formula I, in which X, Y, Z, m, n, A and R are as defined above and wherein G is a latentiating group of the formula —SO₂R^{e}, may be prepared by reaction of compounds of formula II, in which X, Y, Z, m, n, A and R are as defined above, with an alkyl or aryl sulfonyl halide, preferably in the presence of at least one equivalent of base.

Compounds of formula I, in which X, Y, Z, m, n, A and R are as defined above and wherein G is C₁-C₆alkyl, C₂-C₆alkenyl, C₃-C₆alkynyl or a latentiating group of the formula CH₂—X^{f}—R^{h}, may be prepared by treatment of a compound of formula II, in which X, Y, Z, m, n, A and R are as defined above, with a compound of formula G-Y″ wherein Y″ is a halogen (especially bromine or iodine), sulfonate (especially mesylate or tosylate) or a sulfate preferably in the presence of a base, under known conditions.

Compounds of formula III, in which X, Y, Z, m, n, A and G are as defined above,

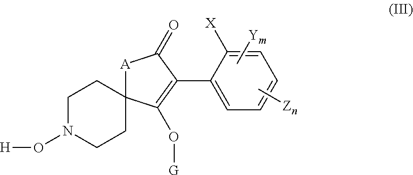

can be obtained by catalytic hydrogenation of compounds of formula I, in which X, Y, Z, m, n, A and G are as defined above and in which R is represented by a benzyl group.

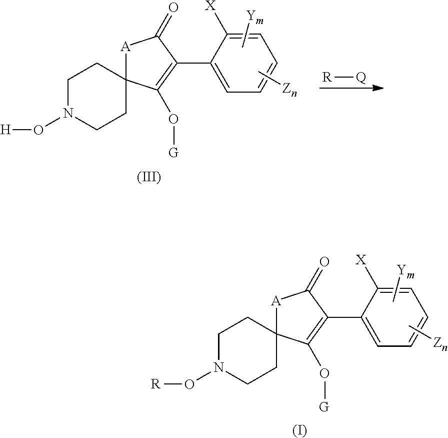

Compounds of formula I, in which X, Y, Z, m, n, R, A and G are as defined above, can be obtained by treating compounds of formula III, in which X, Y, Z, m, n, A and G are as defined above, with an alkylating agent R-Q, wherein R represents the alkyl group to be incorporated and Q represents a nucleofuge, in the presence of at least one equivalent of a base, and optionally in the presence of a suitable solvent.

Compounds of formula II may be prepared via the cyclisation of compounds of formula IV,

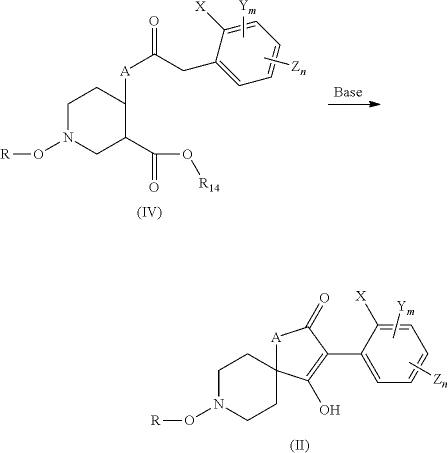

wherein R₁₄ is C₁₋₆alkyl, preferably in the presence of base, and optionally in the presence of a suitable solvent, by known methods described, for example, in WO 09/049,851. X, Y, Z, m, n, A and R are as defined above.

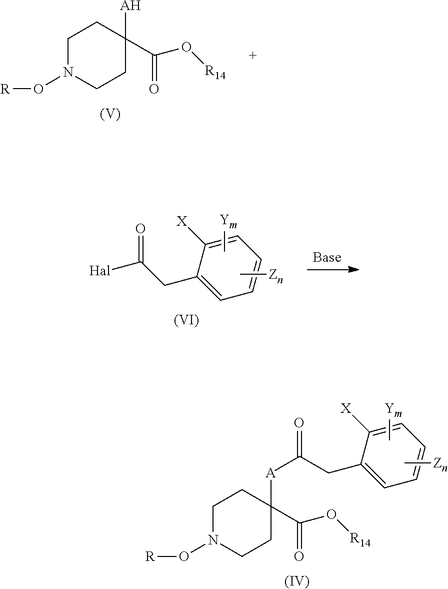

Compounds of formula IV, which are novel and thus constitute another subject of the invention, may be prepared by reacting piperidine-4-carboxylic acid derivatives of formula V with phenylacetyl halides of formula VI, preferably in the presence of base in a suitable solvent by known methods described, for example, in WO 09/049,851. X, Y, Z, m, n, R, A and R₁₄ are as defined above. The base may be inorganic such as an alkali metal carbonate or hydroxide or a metal hydride, or an organic base such as a tertiary amine or metal alkoxide. Examples of suitable inorganic bases include sodium carbonate, sodium or potassium hydroxide, sodium hydride, and suitable organic bases include trialkylamines such as trimethylamine and triethylamine, pyridines or other amine bases such as 1,4-diazobicyclo[2.2.2]octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent. In the situation wherein A is NOH, acylation methods of α-hydroxylamino acid derivatives of formula V are of extreme advantage where N-acylation selectivity can be achieved according, for example, to Vallée and Blandin, Organic & Biomolecular Chemistry, 4, 3125-3141, (2006) or to WO 1996/35714, and whereby the use of transition metal, alkali metal, and alkaline earth metal bases is preferred. The use of a mild base, especially bicarbonates and carbonates of lithium, sodium, potassium and cesium, and more particularly lithium, sodium, potassium and cesium hydrogen carbonate, and even more particularly sodium and potassium hydrogen carbonate in solvents like dichloromethane, tetrahydrofuran, dioxane or mixtures thereof are preferred reaction conditions. The solvent system for these mild basic acylation conditions may also be aqueous biphasic employing, for example, ethyl acetate (or dichloromethane, or any related organic solvent) and water, as described, for example, by Ito et al., Heterocycles, 57, 881-894, (2002).

Phenylacetyl halides of formula VI, wherein Hal is Cl or Br and in which X, Y, Z, m, n are as defined above, are known compounds or can be prepared by known methods, described for example in WO 09/049,851.

For the particular situation in which A is NOR¹, wherein R¹ is C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cycloalkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄)alkyl, C₁₋₄alkylsulfinyl(C₁₋₄)alkyl or C₁₋₄alkylsulfonyl(C₁₋₄)alkyl, compounds of formula IVa, wherein R₁₄ is as defined above, may be prepared by treatment of a compound of formula IVb (R¹═H) with an alkylating agent of formula R¹—Y″, wherein R¹ is the alkyl group to be incorporated and Y″ is a halogen (especially bromine or iodine), sulfonate (especially mesylate or tosylate) or a sulfate, preferably in the presence of a base, under known conditions.

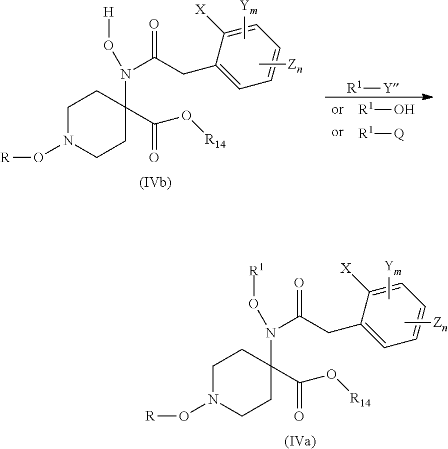

Alternatively, compounds of formula IVa, wherein R₁₄ is as defined above and wherein R¹ is C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cycloalkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄)alkyl, C₁₋₄alkylsulfinyl(C₁₋₄)alkyl or C₁₋₄alkylsulfonyl(C₁₋₄)alkyl may be prepared by treatment of a compound of formula IVb (R¹═H) with an alcohol of formula R¹—OH, wherein R¹ is the alkyl group to be incorporated, under the known conditions of the Mitsunobu reaction. Typically, the alcohol R¹—OH is reacted with the hydroxamic acid derivative IVb in the presence of a dialkyl azodicarboxylate (preferably diethyl azodicarboxylate DEAD, diisopropyl azodicarboxylate DIAD, 1,1′-(azodicarbonyl)dipiperidine ADDP, N,N,N′,N′-tetramethylazodicarboxamide TMAD or equivalents) and a trialkyl- or triaryl phosphine (preferably triphenyl phosphine, tributyl phosphine or equivalents) usually in solvents like tetrahydrofuran, dioxane, dichloromethane, chloroform, dimethylformamide, toluene or benzene at −20 to 80° C., preferably at 0° C. to 30° C. These known conditions are described for example by O. Mitsunobu, Synthesis 1981, 1-28; B. R. Castro, Org. React. 1983, 29, 1-162; D. L. Hughes, Org. React. 1992, 42, 335-656 or D. L. Hughes, Org. Prep. Preced. Int. 1996, 28, 127-164. Compounds of formula IVa, wherein R₁₄ is as defined above and wherein R¹ is tetrahydrofuran-2-yl or tetrahydropyran-2-yl may be prepared by treatment of a compound of formula IVb (R¹═H) with 2,3-dihydro-furan or 3,4-dihydro-2H-pyran preferably in the presence of an acid catalyst (especially p-toluene sulfonic acic or pyridinium p-toluenesulfonate), under known conditions described for example by Shanzer at al., J. Am. Chem. Soc. 129, 347-354, (2007).

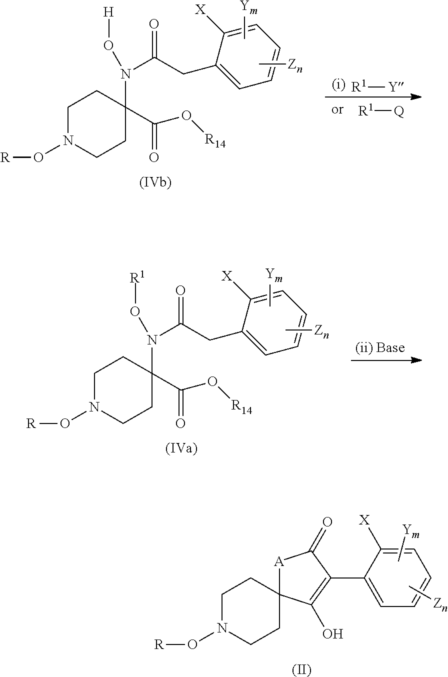

Compounds of formula II, in which A is NOR¹, may also be prepared in a two-step one-pot process involving

(i) O-alkylation of compounds of formula IVb with an agent R¹—Y″ or R¹-Q, where R¹ is the alkyl group to be incorporated and wherein Y″ and Q represents a nucleofuge as defined above, in the presence of at least one equivalent of a base, and
(ii) cyclisation of the intermediate compounds of formula IVa, preferably in the presence of additional base, at least one more equivalent, and optionally in the presence of a suitable solvent, by methods described above.
R₁₄ is typically C₁-C₆alkyl. The base for steps (i) and (ii) may be the same or different.

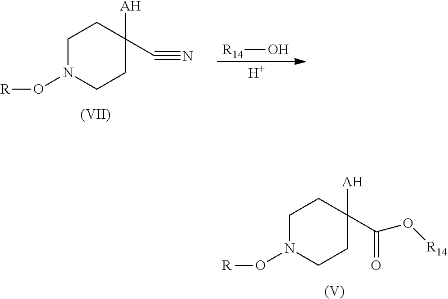

Piperidine-4-carboxylic acid derivatives of the formula V, wherein R₁₄ is C₁-C₆alkyl, may be prepared by reacting nitriles of the formula VII with an alcohol of the formula R₁₄OH, wherein R₁₄ is C₁-C₆alkyl, preferably in the presence of a strong acid (especially sulfuric acid or hydrochloric acid), under known conditions. For the particular situation where R₁₄ is methyl, a compound of the formula VII may also be treated with acetyl chloride in methanol. R and A are as defined above.

Piperidine-4-carboxylic acid derivatives of the formula V, wherein R₁₄ is C₁-C₆alkyl, can also be prepared by known methods from acids of formula VIII. Esterification of VIII with an alcohol of the formula R₁₄OH, wherein R₁₄ is C₁-C₆alkyl, under thionyl chloride activation is a typical example for the preparation of esters V, as described for example in WO09/049,851, but other known esterification methods may also be applied, like for example treatment of a compound of the formula VIII with an alcohol of the formula R₁₄OH under acidic conditions (typically H₂SO₄ or HCl). For the particular situation where R₁₄ is methyl, a compound of the formula VIII may also be treated with diazomethane or trimethylsilyldiazomethane, or with acetyl chloride in methanol. The compounds VIII, VII and V can be reacted and/or isolated as free amines or amine salts (eg a hydrohalide salt, more specifically a hydrochloride or hydrobromide salt, or any other equivalent salt).

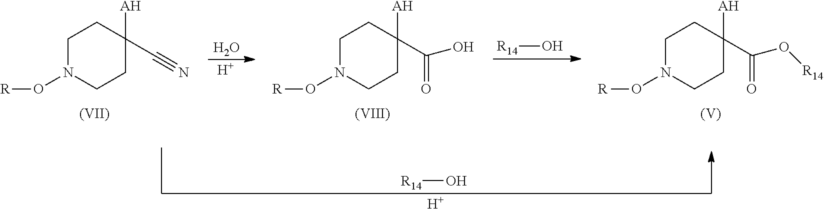

Hydrolysis of nitriles of the formula VII into acids of formula VIII is typically performed with water under acidic conditions, for example in presence of hydrochloric or sulfuric acid.

Nitriles of the formula VII, wherein R and A are as defined above, may be prepared from ketones of formula X,

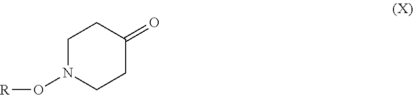

wherein R is as defined above, by means of Strecker-type chemistry utilizing known methods described, for example, in WO 10/63670 and in WO 10/66780.

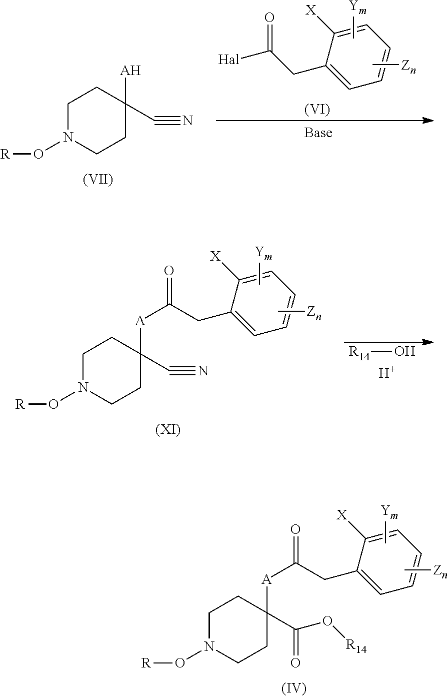

Alternatively, compounds of formula IV wherein R₁₄ is C₁-C₆alkyl, may be prepared by subjecting nitrile derivatives of formula XI to alcoholysis with R₁₄OH, preferably in acidic media (especially sulfuric acid or hydrochloric acid) by known methods described, for example, in WO 09/049,851. X, Y, Z, m, n, A and R are as defined above.

Nitrile compounds of formula XI may be themselves prepared by reacting compounds of formula VII with phenylacetyl halides of formula VI, preferably in the presence of base in a suitable solvent by known methods described, for example, in WO 09/049,851. X, Y, Z, m, n, A and R are as defined above. The base may be inorganic such as an alkali metal carbonate or hydroxide or a metal hydride, or an organic base such as a tertiary amine or metal alkoxide. Examples of suitable inorganic bases include sodium carbonate, sodium or potassium hydroxide, sodium hydride, and suitable organic bases include trialkylamines such as trimethylamine and triethylamine, pyridines or other amine bases such as 1,4-diazobicyclo[2.2.2]octane and 1,8-diazabicyclo[5.4.0]undec-7-ene. Preferred bases include triethylamine and pyridine. Suitable solvents for this reaction are selected to be compatible with the reagents and include ethers such as tetrahydrofuran and 1,2-dimethoxyethane and halogenated solvents such as dichloromethane and chloroform. Certain bases, such as pyridine and triethylamine, may be employed successfully as both base and solvent.

These reaction conditions do also apply for the particular situation where A is NOH, allowing to prepare compounds of the formula IVb from nitriles of the formula VII, in which A is NOH, via compounds of the formula XIb. Of extreme advantage are also acylation methods of nitrile derivatives of formula VII where N-acylation selectivity can be achieved according, for example, to Vallée and Blandin, Organic & Biomolecular Chemistry, 4, 3125-3141, (2006) or to WO 1996/35714, and whereby the use of transition metal, alkali metal, and alkaline earth metal bases is preferred. The use of a mild base, especially bicarbonates and carbonates of lithium, sodium, potassium and cesium, and more particularly lithium, sodium, potassium and cesium hydrogen carbonate, and even more particularly sodium and potassium hydrogen carbonate in solvents like dichloromethane, tetrahydrofuran, dioxane or mixtures thereof are preferred reaction conditions. The solvent system for these mild basic acylation conditions may also be aqueous biphasic employing, for example, ethyl acetate (or dichloromethane, or any related organic solvent) and water, as described, for example, by Ito et al., Heterocycles, 57, 881-894, (2002). The transformation of compounds of the formula XIb into compounds of the formula IVb under alcoholysis conditions with R₁₄OH uses same conditions as described above for the conversion of compounds of the formula XI into compounds of the formula IV.

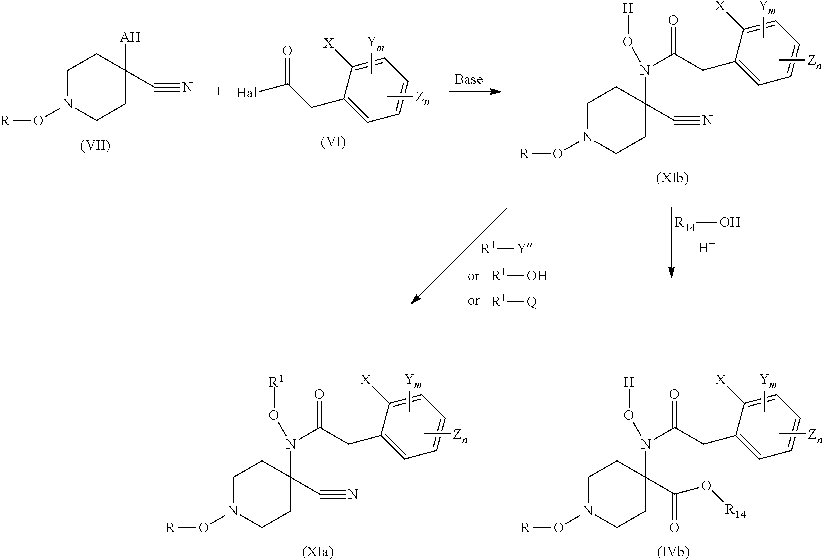

Compounds of formula XIa, in which A is NOR¹, wherein R¹ is C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cycloalkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄)alkyl, C₁₋₄alkylsulfinyl(C₁₋₄)alkyl or C₁₋₄alkylsulfonyl(C₁₋₄)alkyl can moreover be obtained by treating compounds of formula XIb with agents R¹—Y″, R¹—OH, R¹-Q or 2,3-dihydro-furan or 3,4-dihydro-2H-pyran, wherein R¹, Y and Q are as defined above, under the same conditions described above for the conversion of compounds of the formula IVb into compounds of the formula IVa.

The group of compounds D1 comprising compounds of the formula I, and compounds of the formula II, and intermediates of the formula IV or XI, and precursors of the compounds of the formula VI (the corresponding acids or esters), wherein at least one of X, Y or Z is C₂₋₆alkenyl, C₂₋₆alkynyl, —CHO, C₁₋₆alkylcarbonyl or C₁₋₆alkoxycarbonyl, may be prepared by reacting a corresponding halogen precursor forming a group of compounds D2,

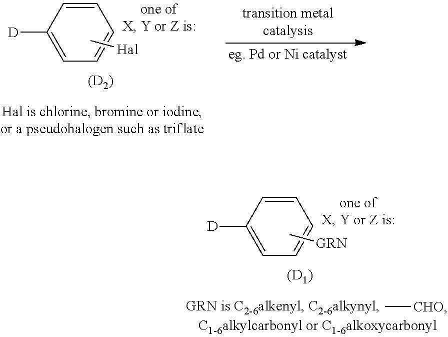

wherein Hal is chlorine, bromine, iodine or a pseudohalogen such as C₁₋₄haloalkyl-sulfonate, especially triflate, by means of a transition metal-catalyzed reaction with an appropriate reaction partner. D represents one of the following fragments D3-D8:

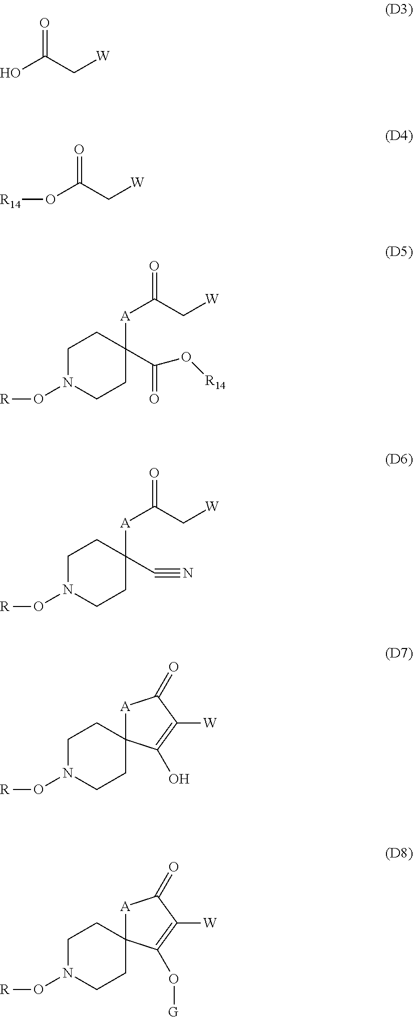

wherein W denotes the position of attachment to the phenyl-moiety in the group of compounds of the formula D1 and D2, and in which R₁₄ is C₁₋₆alkyl. A and R are as defined above. Transition metal-catalyzed reactions, in particular under palladium- or nickel-catalyzed conditions, may be defined as a Negishi coupling (involving an organozinc reagent such as HC≡CZnBr or H₂C═CHZnCl), a Suzuki coupling (involving an organoboron reagent such as 2-ethenyl-4,4,5,5-tetramethyl-1,3,2-dioxaborolane; potassium (ethenyl)trifluoroborate; or triethenylboroxin [optionally as 1:1-pyridine complex]), a Stille coupling (involving an organostannane reagent such as Bu₃SnCH═CH₂, Me₃SnCH═CH₂ [see for example J. Org. Chem. 52, 422-4 (1988)], or tributyl(1-ethoxyvinyl)tin), a Kumada coupling (involving an Grignard reagent such as H₂C═CHMgCl), a Hiyama coupling (involving an organosilicon reagent such as ethenyltrimethyl-silane or alkenylsilanolates), a Sonogashira reaction (involving a terminal alkyne reagent such as ethynyltrimethyl-silane or 2-methyl-but-3-yn-2-ol), a Heck reaction (involving an olefin reagent such as ethylene under pressure, see for example C. R. Smith et al., Tetrahedron 66, 1102-1110 (2010), a carbonylation reaction (in particular an alkoxycarbonylation involving carbon monoxide (or another source of CO, such as for example molybdenum hexacarbonyl) and an alcohol R₁₄OH) or other related and known reactions from the literature. Further details on metal-catalyzed cross-coupling conditions may be found, for example in Metal-catalyzed Cross-coupling Reactions, ed. by Diederich F, Stang P J, Wiley-VCH, Weinheim, (1998) or Modern Arylation Methods, ed. by Ackermann L, Wiley-VCH, Weinheim, (2009). An additional hydrolysis or deprotection, in particular desilylation, step may be necessary to convert the direct product of the transition metal-catalyzed reaction into a compound of the formula D₁, wherein at least one of X, Y or Z is C₂₋₆alkenyl, C₂₋₆alkynyl, —CHO, C₁₋₆alkylcarbonyl or C₁₋₆alkoxycarbonyl.

Worthwhile mentioning are the following particular example:

(a) Sonogashira reaction with ethynyltrimethyl-silane, followed by desilylation:

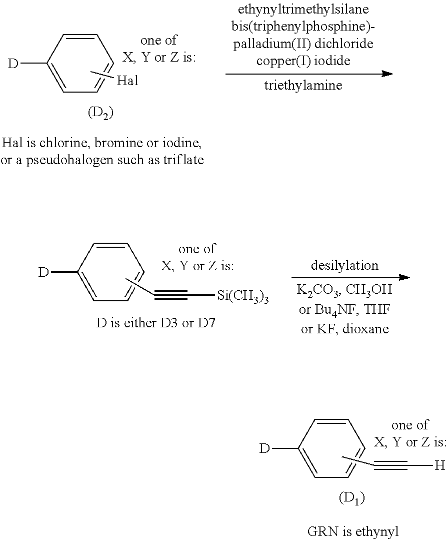

Typical desilylation conditions may be found, for example, in N. Fujii et al., J. Org. Chem. 74, 7052-58 (2009); B. Wen et al., Org. Lett. 13, 168-171 (2011); or P. Wessig et al., J. Org. Chem. 69, 7582-7591 (2004).

(b) Stille reaction with tributyl(1-ethoxyvinyl)tin, followed by hydrolysis of the enol-ether:

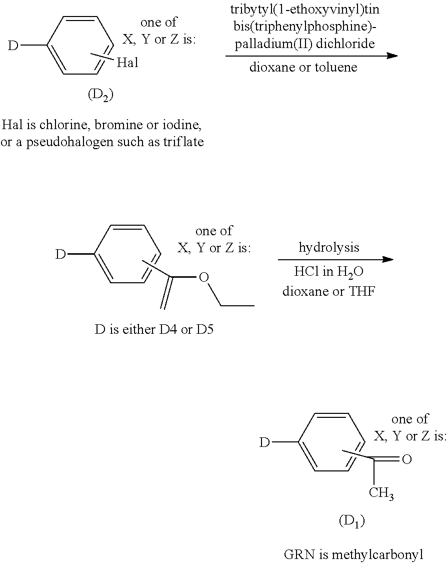

Typical hydrolysis conditions may be found, for example, in J. K. Stille et al., J. Org. Chem. 55, 3114-8 (1990).

(c) Carbonylation reaction involving molybdenum hexacarbonyl as a solid source of carbon monoxide:

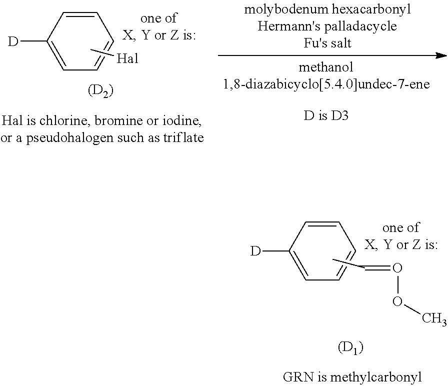

Typical conditions for such a reaction may be found, for example, in M. Larhed et al., J. Comb. Chem. 7, 574-83, (2005). Conventional carbonylation reaction conditions involving carbon monoxide and an alcohol R₁₄OH may be found, for example, in W. R. Moser et al., J. Am. Chem. Soc. 110, 2816-20 (1988).

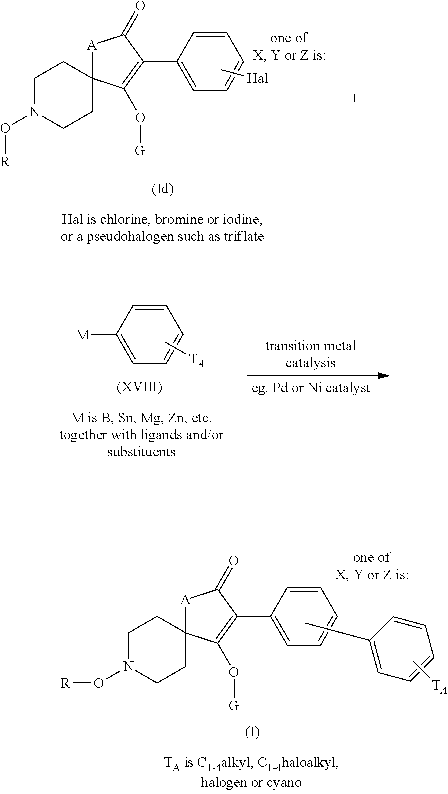

Compounds of the formula I, wherein X, Y or Z is phenyl or phenyl substituted by C₁₋₄alkyl, C₁₋₄haloalkyl, halogen or cyano, may be prepared by reacting a corresponding halogen precursor of the formula Id, wherein Hal is chlorine, bromine, iodine or a pseudohalogen such as C₁₋₄haloalkylsulfonate, especially triflate, with an appropriate organometallic phenyl species of the formula XVIII, wherein T_{A} is C₁₋₄alkyl, C₁₋₄haloalkyl, halogen or cyano and M is for example B, Sn, Si, Mg or Zn holding further ligands and/or substituents, by means of a transition metal-catalyzed reaction. The organometallic species of the formula XVIII is for example an aryl boronic acid T_{A}-Phenyl-B(OH)₂, or a suitable salt or ester thereof, which will react with a compound of the formula Id under palladium- or nickel-catalyzed conditions, such as for example the Suzuki-Miyaura conditions. A variety of metals, catalysts and ligands may be used in this reaction type. Reaction conditions and catalytic systems for such a transformation have been described, for example, in WO08/071,405. A, G and R are as defined above.

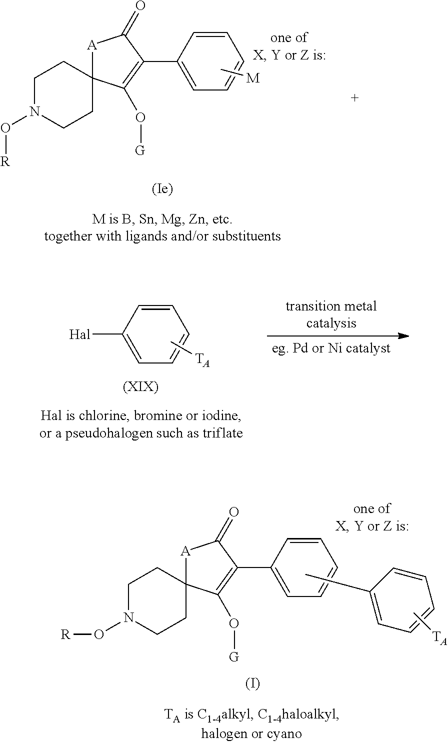

One person skilled in the art will recognize that the polarity at the two reacting centers in this cross-coupling process may be reversed. Compounds of the formula I, wherein X, Y or Z is phenyl or phenyl substituted by C₁₋₄alkyl, C₁₋₄haloalkyl, halogen or cyano, may be also prepared by reacting a corresponding organometallic species of the formula Ie, wherein M is for example B, Sn, Si, Mg or Zn holding further ligands and/or substituents, with an aryl halide of the formula XIX, wherein Hal is chlorine, bromine, iodine or a pseudohalogen such as C₁₋₄haloalkylsulfonate, especially triflate, by means of a transition metal-catalyzed reaction and under similar conditions as described above. A, G and R are as defined above.

The sulfur oxidation state of compounds of the formula I, II, Ill, IV and XI, and of intermediates of the formula V, VII and VIII, wherein A is incorporating such a S atom, like for example when A is either NR¹ or NOR¹ wherein R¹ is C₁₋₄alkylthio(C₁₋₄)alkyl, may be easily adapted from the sulfide oxidation state into the sulfoxide or sulfone level by means of an oxidation reaction involving reagents such as, for example, m-chloroperbenzoic acid (MCPBA), oxone, sodium periodate, sodium hypochlorite or tert-butyl hypochlorite amongst many others.

Compounds of the formula IV and XI, and salts thereof, are novel, have been specifically designed for the synthesis of the compounds of the formula I and as such form a further aspect of the invention. Thus compounds of the formula IV

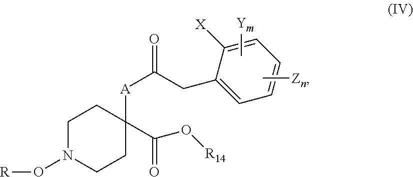

and salts thereof, wherein X, Y, Z, m, n, A and R have the meanings assigned to them above and R₁₄ is C₁₋₆alkyl; and compounds of the formula XI

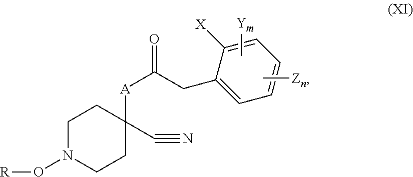

and salts thereof, wherein X, Y, Z, m, n, A and R have the meanings assigned to them above are novel.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or dilu-ent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reaction is advantageously carried out in a temperature range from approximately −80° C. to approximately +140° C., preferably from approximately −30° C. to approximately+100° C., in many cases in the range between ambient temperature and approximately +80° C.

A compound I can be converted in a manner known per se into another compound I by replacing one or more substituents of the starting compound I in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds I can be prepared in a manner known per se. Thus, for example, acid addition salts of compounds I are obtained by treatment with a suitable acid or a suitable ion exchanger reagent and salts with bases are obtained by treatment with a suitable base or with a suitable ion exchanger reagent.

Salts of compounds I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds I can be converted in a manner known per se into other salts of compounds I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds I, which have salt-forming properties can be obtained in free form or in the form of salts.

The compounds I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl celulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereose-lective or enantioselective synthesis, for example by carrying out the process according to the invention with starting materials of a suitable stereochemistry.

It is advantageous to isolate or synthesize in each case the biologically more effective iso-mer, for example enantiomer or diastereomer, or isomer mixture, for example enantiomer mixture or diastereomer mixture, if the individual components have a different biological activity.

The compounds I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables 1 to 333 below can be prepared according to the methods described above. The examples which follow are intended to illustrate the invention and show preferred compounds of formula I.

TABLE 1
This table discloses the 220 compounds T1.001 to T1.220 of the formula Ia:

| | |
|---|---|
| | (Ia) |
| 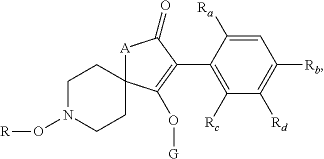 | |

wherein R is CH₃, A is NH, G is hydrogen and Rₐ,
R_{b}, R_{c} and R_{d} are as defined below:

| No. | Rₐ | R_{b} | R_{c} | R_{d} |
|---|---|---|---|---|
| T1.001 | Br | CH═CH₂ | H | H |
| T1.002 | Br | C≡CH | H | H |
| T1.003 | Cl | CH═CH₂ | H | H |
| T1.004 | Cl | C≡CH | H | H |
| T1.005 | CH₃ | CHO | H | H |
| T1.006 | CH₃ | COCH₃ | H | H |
| T1.007 | CH₃ | COOCH₃ | H | H |
| T1.008 | CH₃ | CH═CH₂ | H | H |
| T1.009 | CH₃ | C≡CH | H | H |
| T1.010 | CH₃ | C≡CCH₃ | H | H |
| T1.011 | CH₃CH₂ | CH═CH₂ | H | H |
| T1.012 | CH₃CH₂ | C≡CH | H | H |
| T1.013 | CH₃O | CH═CH₂ | H | H |
| T1.014 | CH₃O | C≡CH | H | H |
| T1.015 | CH═CH₂ | Br | H | H |
| T1.016 | CH═CH₂ | Cl | H | H |
| T1.017 | CH═CH₂ | CH₃ | H | H |
| T1.018 | CH═CH₂ | CH═CH₂ | H | H |
| T1.019 | CH═CH₂ | C≡CH | H | H |
| T1.020 | CH═CH₂ | 4-Cl—C₆H₄ | H | H |
| T1.021 | C≡CH | Br | H | H |
| T1.022 | C≡CH | Cl | H | H |
| T1.023 | C≡CH | CH₃ | H | H |
| T1.024 | C≡CH | CH═CH₂ | H | H |
| T1.025 | C≡CH | C≡CH | H | H |
| T1.026 | C≡CH | 4-Cl—C₆H₄ | H | H |
| T1.027 | Br | H | CH═CH₂ | H |
| T1.028 | Br | H | C≡CH | H |
| T1.029 | Cl | H | CH═CH₂ | H |
| T1.030 | Cl | H | C≡CH | H |
| T1.031 | CH₃ | H | CHO | H |
| T1.032 | CH₃ | H | COCH₃ | H |
| T1.033 | CH₃ | H | COOCH₃ | H |
| T1.034 | CH₃ | H | CH═CH₂ | H |
| T1.035 | CH₃ | H | C≡CH | H |
| T1.036 | CH₃ | H | C≡CCH₃ | H |
| T1.037 | CH₃CH₂ | H | CH═CH₂ | H |
| T1.038 | CH₃CH₂ | H | C≡CH | H |
| T1.039 | CH₃O | H | CH═CH₂ | H |
| T1.040 | CH₃O | H | C≡CH | H |
| T1.041 | CH═CH₂ | H | CH═CH₂ | H |
| T1.042 | CH═CH₂ | H | C≡CH | H |
| T1.043 | C≡CH | H | C≡CH | H |
| T1.044 | Br | H | H | CH═CH₂ |
| T1.045 | Br | H | H | C≡CH |
| T1.046 | Cl | H | H | CH═CH₂ |
| T1.047 | Cl | H | H | C≡CH |
| T1.048 | CH₃ | H | H | CHO |
| T1.049 | CH₃ | H | H | COCH₃ |
| T1.050 | CH₃ | H | H | COOCH₃ |
| T1.051 | CH₃ | H | H | CH═CH₂ |
| T1.052 | CH₃ | H | H | C≡CH |
| T1.053 | CH₃ | H | H | C≡CCH₃ |
| T1.054 | CH₃CH₂ | H | H | CH═CH₂ |
| T1.055 | CH₃CH₂ | H | H | C≡CH |
| T1.056 | CH₃O | H | H | CH═CH₂ |
| T1.057 | CH₃O | H | H | C≡CH |
| T1.058 | CH═CH₂ | H | H | Br |
| T1.059 | CH═CH₂ | H | H | Cl |
| T1.060 | CH═CH₂ | H | H | CH₃ |
| T1.061 | CH═CH₂ | H | H | CH═CH₂ |
| T1.062 | CH═CH₂ | H | H | C≡CH |
| T1.063 | CH═CH₂ | H | H | 4-Cl—C₆H₄ |
| T1.064 | C≡CH | H | H | Br |
| T1.065 | C≡CH | H | H | Cl |
| T1.066 | C≡CH | H | H | CH₃ |
| T1.067 | C≡CH | H | H | CH═CH₂ |
| T1.068 | C≡CH | H | H | C≡CH |
| T1.069 | C≡CH | H | H | 4-Cl—C₆H₄ |
| T1.070 | Br | CH═CH₂ | CH₃ | H |
| T1.071 | Br | C≡CH | CH₃ | H |
| T1.072 | Cl | CH═CH₂ | CH₃ | H |
| T1.073 | Cl | C≡CH | CH₃ | H |
| T1.074 | CH₃ | CHO | CH₃ | H |
| T1.075 | CH₃ | COCH₃ | CH₃ | H |
| T1.076 | CH₃ | COOCH₃ | CH₃ | H |
| T1.077 | CH₃ | CH═CH₂ | CH₃ | H |
| T1.078 | CH₃ | C≡CH | CH₃ | H |
| T1.079 | CH₃ | C≡CCH₃ | CH₃ | H |
| T1.080 | CH₃CH₂ | CH═CH₂ | CH₃ | H |
| T1.081 | CH₃CH₂ | C≡CH | CH₃ | H |
| T1.082 | CH₃O | CH═CH₂ | CH₃ | H |
| T1.083 | CH₃O | C≡CH | CH₃ | H |
| T1.084 | CH═CH₂ | Br | CH₃ | H |
| T1.085 | CH═CH₂ | Cl | CH₃ | H |
| T1.086 | CH═CH₂ | CH₃ | CH₃ | H |
| T1.087 | CH═CH₂ | CH═CH₂ | CH₃ | H |
| T1.088 | CH═CH₂ | C≡CH | CH₃ | H |
| T1.089 | CH═CH₂ | 4-Cl—C₆H₄ | CH₃ | H |
| T1.090 | C≡CH | Br | CH₃ | H |
| T1.091 | C≡CH | Cl | CH₃ | H |
| T1.092 | C≡CH | CH₃ | CH₃ | H |
| T1.093 | C≡CH | CH═CH₂ | CH₃ | H |
| T1.094 | C≡CH | C≡CH | CH₃ | H |
| T1.095 | C≡CH | 4-Cl—C₆H₄ | CH₃ | H |
| T1.096 | Br | CH═CH₂ | H | CH₃ |
| T1.097 | Br | C≡CH | H | CH₃ |
| T1.098 | Cl | CH═CH₂ | H | CH₃ |
| T1.099 | Cl | C≡CH | H | CH₃ |
| T1.100 | CH₃ | CHO | H | CH₃ |
| T1.101 | CH₃ | COCH₃ | H | CH₃ |
| T1.102 | CH₃ | COOCH₃ | H | CH₃ |
| T1.103 | CH₃ | CH═CH₂ | H | CH₃ |
| T1.104 | CH₃ | C≡CH | H | CH₃ |
| T1.105 | CH₃ | C≡CCH₃ | H | CH₃ |
| T1.106 | CH₃CH₂ | CH═CH₂ | H | CH₃ |
| T1.107 | CH₃CH₂ | C≡CH | H | CH₃ |
| T1.108 | CH₃O | CH═CH₂ | H | CH₃ |
| T1.109 | CH₃O | C≡CH | H | CH₃ |
| T1.110 | CH═CH₂ | Br | H | CH₃ |
| T1.111 | CH═CH₂ | Cl | H | CH₃ |
| T1.112 | CH═CH₂ | CH₃ | H | CH₃ |
| T1.113 | CH═CH₂ | CH═CH₂ | H | CH₃ |
| T1.114 | CH═CH₂ | C≡CH | H | CH₃ |
| T1.115 | CH═CH₂ | 4-Cl—C₆H₄ | H | CH₃ |
| T1.116 | C≡CH | Br | H | CH₃ |
| T1.117 | C≡CH | Cl | H | CH₃ |
| T1.118 | C≡CH | CH₃ | H | CH₃ |
| T1.119 | C≡CH | CH═CH₂ | H | CH₃ |
| T1.120 | C≡CH | C≡CH | H | CH₃ |
| T1.121 | C≡CH | 4-Cl—C₆H₄ | H | CH₃ |
| T1.122 | Br | CH₃ | CH═CH₂ | H |
| T1.123 | Br | CH₃ | C≡CH | H |
| T1.124 | Cl | CH₃ | CH═CH₂ | H |
| T1.125 | Cl | CH₃ | C≡CH | H |
| T1.126 | CH₃ | CH₃ | CHO | H |
| T1.127 | CH₃ | CH₃ | COCH₃ | H |
| T1.128 | CH₃ | CH₃ | COOCH₃ | H |
| T1.129 | CH₃ | CH₃ | C≡CCH₃ | H |
| T1.130 | CH₃CH₂ | CH₃ | CH═CH₂ | H |
| T1.131 | CH₃CH₂ | CH₃ | C≡CH | H |
| T1.132 | CH₃O | CH₃ | CH═CH₂ | H |
| T1.133 | CH₃O | CH₃ | C≡CH | H |
| T1.134 | CH═CH₂ | CH₃ | CH═CH₂ | H |
| T1.135 | CH═CH₂ | CH₃ | C≡CH | H |
| T1.136 | C≡CH | CH₃ | C≡CH | H |
| T1.137 | Br | H | CH═CH₂ | CH₃ |
| T1.138 | Br | H | C≡CH | CH₃ |
| T1.139 | Cl | H | CH═CH₂ | CH₃ |
| T1.140 | Cl | H | C≡CH | CH₃ |
| T1.141 | CH₃ | H | CHO | CH₃ |
| T1.142 | CH₃ | H | COCH₃ | CH₃ |
| T1.143 | CH₃ | H | COOCH₃ | CH₃ |
| T1.144 | CH₃ | H | CH═CH₂ | CH₃ |
| T1.145 | CH₃ | H | C≡CH | CH₃ |
| T1.146 | CH₃ | H | C≡CCH₃ | CH₃ |
| T1.147 | CH₃CH₂ | H | CH═CH₂ | CH₃ |
| T1.148 | CH₃CH₂ | H | C≡CH | CH₃ |
| T1.149 | CH₃O | H | CH═CH₂ | CH₃ |
| T1.150 | CH₃O | H | C≡CH | CH₃ |
| T1.151 | CH═CH₂ | H | Br | CH₃ |
| T1.152 | CH═CH₂ | H | Cl | CH₃ |
| T1.153 | CH═CH₂ | H | CH₃ | CH₃ |
| T1.154 | CH═CH₂ | H | CH═CH₂ | CH₃ |
| T1.155 | CH═CH₂ | H | C≡CH | CH₃ |
| T1.156 | C≡CH | H | Br | CH₃ |
| T1.157 | C≡CH | H | Cl | CH₃ |
| T1.158 | C≡CH | H | CH₃ | CH₃ |
| T1.159 | C≡CH | H | CH═CH₂ | CH₃ |
| T1.160 | C≡CH | H | C≡CH | CH₃ |
| T1.161 | Br | CH₃ | H | CH═CH₂ |
| T1.162 | Br | CH₃ | H | C≡CH |
| T1.163 | Cl | CH₃ | H | CH═CH₂ |
| T1.164 | Cl | CH₃ | H | C≡CH |
| T1.165 | CH₃ | CH₃ | H | CHO |
| T1.166 | CH₃ | CH₃ | H | COCH₃ |
| T1.167 | CH₃ | CH₃ | H | COOCH₃ |
| T1.168 | CH₃ | CH₃ | H | CH═CH₂ |
| T1.169 | CH₃ | CH₃ | H | C≡CH |
| T1.170 | CH₃ | CH₃ | H | C≡CCH₃ |
| T1.171 | CH₃CH₂ | CH₃ | H | CH═CH₂ |
| T1.172 | CH₃CH₂ | CH₃ | H | C≡CH |
| T1.173 | CH₃O | CH₃ | H | CH═CH₂ |
| T1.174 | CH₃O | CH₃ | H | C≡CH |
| T1.175 | CH═CH₂ | CH₃ | H | Br |
| T1.176 | CH═CH₂ | CH₃ | H | Cl |
| T1.177 | CH═CH₂ | CH₃ | H | CH═CH₂ |
| T1.178 | CH═CH₂ | CH₃ | H | C≡CH |
| T1.179 | CH═CH₂ | CH₃ | H | 4-Cl—C₆H₄ |
| T1.180 | C≡CH | CH₃ | H | Br |
| T1.181 | C≡CH | CH₃ | H | Cl |
| T1.182 | C≡CH | CH₃ | H | CH═CH₂ |
| T1.183 | C≡CH | CH₃ | H | C≡CH |
| T1.184 | C≡CH | CH₃ | H | 4-Cl—C₆H₄ |
| T1.185 | Br | H | CH₃ | CH═CH₂ |
| T1.186 | Br | H | CH₃ | C≡CH |
| T1.187 | Cl | H | CH₃ | CH═CH₂ |
| T1.188 | Cl | H | CH₃ | C≡CH |
| T1.189 | CH₃ | H | CH₃ | CHO |
| T1.190 | CH₃ | H | CH₃ | COCH₃ |
| T1.191 | CH₃ | H | CH₃ | COOCH₃ |
| T1.192 | CH₃ | H | CH₃ | CH═CH₂ |
| T1.193 | CH₃ | H | CH₃ | C≡CH |
| T1.194 | CH₃ | H | CH₃ | C≡CCH₃ |
| T1.195 | CH₃CH₂ | H | CH₃ | CH═CH₂ |
| T1.196 | CH₃CH₂ | H | CH₃ | C≡CH |
| T1.197 | CH₃O | H | CH₃ | CH═CH₂ |
| T1.198 | CH₃O | H | CH₃ | C≡CH |
| T1.199 | CH═CH₂ | H | CH₃ | Br |
| T1.200 | CH═CH₂ | H | CH₃ | Cl |
| T1.201 | CH═CH₂ | H | CH₃ | CH═CH₂ |
| T1.202 | CH═CH₂ | H | CH₃ | C≡CH |
| T1.203 | CH═CH₂ | H | CH₃ | 4-Cl—C₆H₄ |
| T1.204 | C≡CH | H | CH₃ | Br |
| T1.205 | C≡CH | H | CH₃ | Cl |
| T1.206 | C≡CH | H | CH₃ | CH═CH₂ |
| T1.207 | C≡CH | H | CH₃ | C≡CH |
| T1.208 | C≡CH | H | CH₃ | 4-Cl—C₆H₄ |
| T1.209 | Cyclo-C3 | CH₃ | CH═CH₂ | H |
| T1.210 | Cyclo-C3 | CH₃ | C≡CH | H |
| T1.211 | Cyclo-C3 | C≡CH | CH₃ | H |
| T1.212 | Cyclo-C3 | C≡CCH₃ | CH₃ | H |
| T1.213 | CH═CH₂ | CH₃ | CH₃ | CH₃ |
| T1.214 | CH═CH₂ | CH₃ | CH₃ | Cl |
| T1.215 | C≡CH | CH₃ | CH₃ | CH₃ |
| T1.216 | C≡CH | CH₃ | CH₃ | Cl |
| T1.217 | C≡CCH₃ | CH₃ | CH₃ | CH₃ |
| T1.218 | C≡CCH₃ | CH₃ | CH₃ | Cl |
| T1.219 | CH₃ | C≡CH | CH₃ | Cl |
| T1.220 | CH₃ | C≡CCH₃ | CH₃ | Cl |

Cyclo-C3 means cyclopropyl.

Table 2: This table discloses the 220 compounds T2.001 to T2.220 of the formula Ia, wherein R is hydrogen, A is NH, G is hydrogen and Rₐ, R_{b}, R_{c} and R_{d} are as defined in Table 1.
Table 3: This table discloses the 220 compounds T3.001 to T3.220 of the formula Ia, wherein R is CH₂CH₃, A is NH, G is hydrogen and Rₐ, R_{b}, R_{c} and R_{d} are as defined in Table 1.
Table 4: This table discloses the 220 compounds T4.001 to T4.220 of the formula Ia, wherein R is CH₂CF₃, A is NH, G is hydrogen and Rₐ, R_{b}, R_{c} and R_{d} are as defined in Table 1.
Table 5: This table discloses the 220 compounds T5.001 to T5.220 of the formula Ia, wherein R is CH₂OCH₃, A is NH, G is hydrogen and Rₐ, R_{b}, R_{c} and R_{d} are as defined in Table 1.
Table 6: This table discloses the 220 compounds T6.001 to T6.220 of the formula Ia, wherein R is CH₂CH₂OCH₃, A is NH, G is hydrogen and Rₐ, R_{b}, R_{c} and R_{d} are as defined in Table 1.
Table 7: This table discloses the 220 compounds T7.001 to T7.220 of the formula Ia, wherein R is allyl, A is NH, G is hydrogen and Rₐ, R_{b}, R_{c} and R_{d} are as defined in Table 1.
Table 8: This table discloses the 220 compounds T8.001 to T8.220 of the formula Ia, wherein R is propargyl, A is NH, G is hydrogen and Rₐ, R_{b}, R_{c} and R_{d} are as defined in Table 1.
Table 9: This table discloses the 220 compounds T9.001 to T9.220 of the formula Ia, wherein R is benzyl, A is NH, G is hydrogen and Rₐ, R_{b}, R_{c} and R_{d} are as defined in Table 1.
Table 10: This table discloses the 220 compounds T10.001 to T10.220 of the formula Ia, wherein R is CH₃, A is NCH₃, G is hydrogen and Rₐ, R_{b}, R_{c} and R_{d} are as defined in Table 1.
Table 11: This table discloses the 220 compounds T11.001 to T11.220 of the formula Ia, wherein R is hydrogen, A is NCH₃, G is hydrogen and Rₐ, R_{b}, R_{c} and R_{d} are as defined in Table 1.
Table 12: This table discloses the 220 compounds T12.001 to T12.220 of the formula Ia, wherein R is CH₂CH₃, A is NCH₃, G is hydrogen and Rₐ, R_{b}, R_{c} and R_{d} are as defined in Table 1.
Table 13: This table discloses the 220 compounds T13.001 to T13.220 of the formula Ia, wherein R is CH₂CF₃, A is NCH₃, G is hydrogen and Rₐ, R_{b}, R_{c} and R_{d} are as defined in Table 1.
Table 14: This table discloses the 220 compounds T14.001 to T14.220 of the formula Ia, wherein R is CH₂OCH₃, A is NCH₃, G is hydrogen and Rₐ, R_{b}, R_{c} and R_{d} are as defined in Table 1.
Table 15: This table discloses the 220 compounds T15.001 to T15.220 of the formula Ia, wherein R is CH₂CH₂OCH₃, A is NCH₃, G is hydrogen and Rₐ, R_{b}, R_{c} and R_{d} are as defined in Table 1.
Table 16: This table discloses the 220 compounds T16.001 to T16.220 of the formula Ia, wherein R is allyl, A is NCH₃, G is hydrogen and Rₐ, R_{b}, R_{c} and R_{d} are as defined in Table 1.
Table 17: This table discloses the 220 compounds T17.001 to T17.220 of the formula Ia, wherein R is propargyl, A is NCH₃, G is hydrogen and Rₐ, R_{b}, R_{c} and R_{d} are as defined in Table 1.
Table 18: This table discloses the 220 compounds T18.001 to T18.220 of the formula Ia, wherein R is benzyl, A is NCH₃, G is hydrogen and Rₐ, R_{b}, R_{c} and R_{d} are as defined in Table 1.
Table 19: This table discloses the 220 compounds T19.001 to T19.220 of the formula Ia, wherein R is CH₃, A is NCH₂CH₃, G is hydrogen and Rₐ, R_{b}, R_{c} and R_{d} are as defined in Table 1.
Table 20: This table discloses the 220 compounds T20.001 to T20.220 of the formula Ia, wherein R is hydrogen, A is NCH₂CH₃, G is hydrogen and Rₐ, R_{b}, R_{c} and R_{d} are as defined in Table 1.
Table 21: This table discloses the 220 compounds T21.001 to T21.220 of the formula Ia, wherein R is CH₂CH₃, A is NCH₂CH₃, G is hydrogen and Rₐ, R_{b}, R_{c} and R_{d} are as defined in Table 1.
Table 22: This table discloses the 220 compounds T22.001 to T22.220 of the formula Ia, wherein R is CH₂CF₃, A is NCH₂CH₃, G is hydrogen and Rₐ, R_{b}, R_{c} and R_{d} are as defined in Table 1.
Table 23: This table discloses the 220 compounds T23.001 to T23.220 of the formula Ia, wherein R is CH₂OCH₃, A is NCH₂CH₃, G is hydrogen and Rₐ, R_{b}, R_{c} and R_{d} are as defined in Table 1.
Table 24: This table discloses the 220 compounds T24.001 to T24.220 of the formula Ia, wherein R is CH₂CH₂OCH₃, A is NCH₂CH₃, G is hydrogen and Rₐ, R_{b}, R_{c} and R_{d} are as defined in Table 1.
Table 25: This table discloses the 220 compounds T25.001 to T25.220 of the formula Ia, wherein R is allyl, A is NCH₂CH₃, G is hydrogen and Rₐ, R_{b}, R_{c} and R_{d} are as defined in Table 1.
Table 26: This table discloses the 220 compounds T26.001 to T26.220 of the formula Ia, wherein R is propargyl, A is NCH₂CH₃, G is hydrogen and Rₐ, R_{b}, R_{c} and R_{d} are as defined in Table 1.
Table 27: This table discloses the 220 compounds T27.001 to T27.220 of the formula Ia, wherein R is benzyl, A is NCH₂CH₃, G is hydrogen and Rₐ, R_{b}, R_{c} and R_{d} are as defined in Table 1.
Table 28: This table discloses the 220 compounds T28.001 to T28.220 of the formula Ia, wherein R is CH₃, A is NCH(CH₃)₂, G is hydrogen and Rₐ, R_{b}, R_{c} and R_{d} are as defined in Table 1.
Table 29: This table discloses the 220 compounds T29.001 to T29.220 of the formula Ia, wherein R is hydrogen, A is NCH(CH₃)₂, G is hydrogen and Rₐ, R_{b}, R_{c} and R_{d} are as defined in Table 1.
Table 30: This table discloses the 220 compounds T30.001 to T30.220 of the formula Ia, wherein R is CH₂CH₃, A is NCH(CH₃)₂, G is hydrogen and Rₐ, R_{b}, R_{c} and R_{d} are as defined in Table 1.
Table 31: This table discloses the 220 compounds T31.001 to T31.220 of the formula Ia, wherein R is CH₂CF₃, A is NCH(CH₃)₂, G is hydrogen and Rₐ, R_{b}, R_{c} and R_{d} are as defined in Table 1.
Table 32: This table discloses the 220 compounds T32.001 to T32.220 of the formula Ia, wherein R is CH₂OCH₃, A is NCH(CH₃)₂, G is hydrogen and Rₐ, R_{b}, R_{c} and R_{d} are as defined in Table 1.
Table 33: This table discloses the 220 compounds T33.001 to T33.220 of the formula Ia, wherein R is CH₂CH₂OCH₃, A is NCH(CH₃)₂, G is hydrogen and Rₐ, R_{b}, R_{c} and R_{d} are as defined in Table 1.
Table 34: This table discloses the 220 compounds T34.001 to T34.220 of the formula Ia, wherein R is allyl, A is NCH(CH₃)₂, G is hydrogen and Rₐ, R_{b}, R_{c} and R_{d} are as defined in Table 1.
Table 35: This table discloses the 220 compounds T35.001 to T35.220 of the formula Ia, wherein R is propargyl, A is NCH(CH₃)₂, G is hydrogen and Rₐ, R_{b}, R_{c} and R_{d} are as defined in Table 1.
Table 36: This table discloses the 220 compounds T36.001 to T36.220 of the formula Ia, wherein R is benzyl, A is NCH(CH₃)₂, G is hydrogen and Rₐ, R_{b}, R_{c} and R_{d} are as defined in Table 1.
Table 37: This table discloses the 220 compounds T37.001 to T37.220 of the formula Ia, wherein R is CH₃, A is N-cyclopropyl, G is hydrogen and Rₐ, R_{b}, R_{c} and R_{d} are as defined in Table 1.
Table 38: This table discloses the 220 compounds T38.001 to T38.220 of the formula Ia, wherein R is hydrogen, A is N-cyclopropyl, G is hydrogen and Rₐ, R_{b}, R_{c} and R_{d} are as defined in Table 1.
Table 39: This table discloses the 220 compounds T39.001 to T39.220 of the formula Ia, wherein R is CH₂CH₃, A is N-cyclopropyl, G is hydrogen and Rₐ, R_{b}, R_{c} and R_{d} are as defined in Table 1.
Table 40: This table discloses the 220 compounds T40.001 to T40.220 of the formula Ia, wherein R is CH₂CF₃, A is N-cyclopropyl, G is hydrogen and Rₐ, R_{b}, R_{c} and R_{d} are as defined in Table 1.
Table 41: This table discloses the 220 compounds T41.001 to T41.220 of the formula Ia, wherein R is CH₂OCH₃, A is N-cyclopropyl, G is hydrogen and Rₐ, R_{b}, R_{c} and R_{d} are as defined in Table 1.
Table 42: This table discloses the 220 compounds T42.001 to T42.220 of the formula Ia, wherein R is CH₂CH₂OCH₃, A is N-cyclopropyl, G is hydrogen and Rₐ, R_{b}, R_{c} and R_{d} are as defined in Table 1.
Table 43: This table discloses the 220 compounds T43.001 to T43.220 of the formula Ia, wherein R is allyl, A is N-cyclopropyl, G is hydrogen and Rₐ, R_{b}, R_{c} and R_{d} are as defined in Table 1.
Table 44: This table discloses the 220 compounds T44.001 to T44.220 of the formula Ia, wherein R is propargyl, A is N-cyclopropyl, G is hydrogen and Rₐ, R_{b}, R_{c} and R_{d} are as defined in Table 1.
Table 45: This table discloses the 220 compounds T45.001 to T45.220 of the formula Ia, wherein R is benzyl, A is N-cyclopropyl, G is hydrogen and Rₐ, R_{b}, R_{c} and R_{d} are as defined in Table 1.
Table 46: This table discloses the 220 compounds T46.001 to T46.220 of the formula Ia, wherein R is CH₃, A is NCH₂CH═CH₂, G is hydrogen and Rₐ, R_{b}, R_{c} and R_{d} are as defined in Table 1.
Table 47: This table discloses the 220 compounds T47.001 to T47.220 of the formula Ia, wherein R is hydrogen, A is NCH₂CH═CH₂, G is hydrogen and Rₐ, R_{b}, R_{c} and R_{d} are as defined in Table 1.
Table 48: This table discloses the 220 compounds T48.001 to T48.220 of the formula Ia, wherein R is CH₂CH₃, A is NCH₂CH═CH₂, G is hydrogen and Rₐ, R_{b}, R_{c} and R_{d} are as defined in Table 1.
Table 49: This table discloses the 220 compounds T49.001 to T49.220 of the formula Ia, wherein R is CH₂CF₃, A is NCH₂CH═CH₂, G is hydrogen and Rₐ, R_{b}, R_{c} and R_{d} are as defined in Table 1.
Table 50: This table discloses the 220 compounds T50.001 to T50.220 of the formula Ia, wherein R is CH₂OCH₃, A is NCH₂CH═CH₂, G is hydrogen and Rₐ, R_{b}, R_{c} and R_{d} are as defined in Table 1.
Table 51: This table discloses the 220 compounds T51.001 to T51.220 of the formula Ia, wherein R is CH₂CH₂OCH₃, A is NCH₂CH═CH₂, G is hydrogen and Rₐ, R_{b}, R_{c} and R_{d} are as defined in Table 1.
Table 52: This table discloses the 220 compounds T52.001 to T52.220 of the formula Ia, wherein R is allyl, A is NCH₂CH═CH₂, G is hydrogen and Rₐ, R_{b}, R_{c} and R_{d} are as defined in Table 1.
Table 53: This table discloses the 220 compounds T53.001 to T53.220 of the formula Ia, wherein R is propargyl, A is NCH₂CH═CH₂, G is hydrogen and Rₐ, R_{b}, R_{c} and R_{d} are as defined in Table 1.
Table 54: This table discloses the 220 compounds T54.001 to T54.220 of the formula Ia, wherein R is benzyl, A is NCH₂CH═CH₂, G is hydrogen and Rₐ, R_{b}, R_{c} and R_{d} are as defined in Table 1.
Table 55: This table discloses the 220 compounds T55.001 to T55.220 of the formula Ia, wherein R is CH₃, A is NCH₂C≡CH, G is hydrogen and Rₐ, R_{b}, R_{c} and R_{d} are as defined in Table 1.
Table 56: This table discloses the 220 compounds T56.001 to T56.220 of the formula Ia, wherein R is hydrogen, A is NCH₂C≡CH, G is hydrogen and Rₐ, R_{b}, R_{c} and R_{d} are as defined in Table 1.
Table 57: This table discloses the 220 compounds T57.001 to T57.220 of the formula Ia, wherein R is CH₂CH₃, A is NCH₂C≡CH, G is hydrogen and Rₐ, R_{b}, R_{c} and R_{d} are as defined in Table 1.
Table 58: This table discloses the 220 compounds T58.001 to T58.220 of the formula Ia, wherein R is CH₂CF₃, A is NCH₂C≡CH, G is hydrogen and Rₐ, R_{b}, R_{c} and R_{d} are as defined in Table 1.
Table 59: This table discloses the 220 compounds T59.001 to T59.220 of the formula Ia, wherein R is CH₂OCH₃, A is NCH₂C≡CH, G is hydrogen and Rₐ, R_{b}, R_{c} and R_{d} are as defined in Table 1.
Table 60: This table discloses the 220 compounds T60.001 to T60.220 of the formula Ia, wherein R is CH₂CH₂OCH₃, A is NCH₂C≡CH, G is hydrogen and Rₐ, R_{b}, R_{c} and R_{d} are as defined in Table 1.
Table 61: This table discloses the 220 compounds T61.001 to T61.220 of the formula Ia, wherein R is allyl, A is NCH₂C≡CH, G is hydrogen and Rₐ, R_{b}, R_{c} and R_{d} are as defined in Table 1.
Table 62: This table discloses the 220 compounds T62.001 to T62.220 of the formula Ia, wherein R is propargyl, A is NCH₂C≡CH, G is hydrogen and Rₐ, R_{b}, R_{c} and R_{d} are as defined in Table 1.
Table 63: This table discloses the 220 compounds T63.001 to T63.220 of the formula Ia, wherein R is benzyl, A is NCH₂C≡CH, G is hydrogen and Rₐ, R_{b}, R_{c} and R_{d} are as defined in Table 1.
Table 64: This table discloses the 220 compounds T64.001 to T64.220 of the formula Ia, wherein R is CH₃, A is NCH₂cyclo-C3, G is hydrogen and Rₐ, R_{b}, R_{c} and R_{d} are as defined in Table 1.
Table 65: This table discloses the 220 compounds T65.001 to T65.220 of the formula Ia, wherein R is hydrogen, A is NCH₂cyclo-C3, G is hydrogen and Rₐ, R_{b}, R_{c} and R_{d} are as defined in Table 1.
Table 66: This table discloses the 220 compounds T66.001 to T66.220 of the formula Ia, wherein R is CH₂CH₃, A is NCH₂cyclo-C3, G is hydrogen and Rₐ, R_{b}, R_{c} and R_{d} are as defined in Table 1.
Table 67: This table discloses the 220 compounds T67.001 to T67.220 of the formula Ia, wherein R is CH₂CF₃, A is NCH₂cyclo-C3, G is hydrogen and Rₐ, R_{b}, R_{c} and R_{d} are as defined in Table 1.
Table 68: This table discloses the 220 compounds T68.001 to T68.220 of the formula Ia, wherein R is CH₂OCH₃, A is NCH₂cyclo-C3, G is hydrogen and Rₐ, R_{b}, R_{c} and R_{d} are as defined in Table 1.
Table 69: This table discloses the 220 compounds T69.001 to T69.220 of the formula Ia, wherein R is CH₂CH₂OCH₃, A is NCH₂cyclo-C3, G is hydrogen and Rₐ, R_{b}, R_{c} and R_{d} are as defined in Table 1.
Table 70: This table discloses the 220 compounds T70.001 to T70.220 of the formula Ia, wherein R is allyl, A is NCH₂cyclo-C3, G is hydrogen and Rₐ, R_{b}, R_{c} and R_{d} are as defined in Table 1.
Table 71: This table discloses the 220 compounds T71.001 to T71.220 of the formula Ia, wherein R is propargyl, A is NCH₂cyclo-C3, G is hydrogen and Rₐ, R_{b}, R_{c} and R_{d} are as defined in Table 1.
Table 72: This table discloses the 220 compounds T72.001 to T72.220 of the formula Ia, wherein R is benzyl, A is NCH₂cyclo-C3, G is hydrogen and Rₐ, R_{b}, R_{c} and R_{d} are as defined in Table 1.
Table 73: This table discloses the 220 compounds T73.001 to T73.220 of the formula Ia, wherein R is CH₃, A is NCH₂OCH₃, G is hydrogen and Rₐ, R_{b}, R_{c} and R_{d} are as defined in Table 1.
Table 74: This table discloses the 220 compounds T74.001 to T74.220 of the formula Ia, wherein R is hydrogen, A is NCH₂OCH₃, G is hydrogen and Rₐ, R_{b}, R_{c} and R_{d} are as defined in Table 1.
Table 75: This table discloses the 220 compounds T75.001 to T75.220 of the formula Ia, wherein R is CH₂CH₃, A is NCH₂OCH₃, G is hydrogen and Rₐ, R_{b}, R_{c} and R_{d} are as defined in Table 1.
Table 76: This table discloses the 220 compounds T76.001 to T76.220 of the formula Ia, wherein R is CH₂CF₃, A is NCH₂OCH₃, G is hydrogen and Rₐ, R_{b}, R_{c} and R_{d} are as defined in Table 1.
Table 77: This table discloses the 220 compounds T77.001 to T77.220 of the formula Ia, wherein R is CH₂OCH₃, A is NCH₂OCH₃, G is hydrogen and Rₐ, R_{b}, R_{c} and R_{d} are as defined in Table 1.
Table 78: This table discloses the 220 compounds T78.001 to T78.220 of the formula Ia, wherein R is CH₂CH₂OCH₃, A is NCH₂OCH₃, G is hydrogen and Rₐ, R_{b}, R_{c} and R_{d} are as defined in Table 1.
Table 79: This table discloses the 220 compounds T79.001 to T79.220 of the formula Ia, wherein R is allyl, A is NCH₂OCH₃, G is hydrogen and Rₐ, R_{b}, R_{c} and R_{d} are as defined in Table 1.
Table 80: This table discloses the 220 compounds T80.001 to T80.220 of the formula Ia, wherein R is propargyl, A is NCH₂OCH₃, G is hydrogen and Rₐ, R_{b}, R_{c} and R_{d} are as defined in Table 1.
Table 81: This table discloses the 220 compounds T81.001 to T81.220 of the formula Ia, wherein R is benzyl, A is NCH₂OCH₃, G is hydrogen and Rₐ, R_{b}, R_{c} and R_{d} are as defined in Table 1.
Table 82: This table discloses the 220 compounds T82.001 to T82.220 of the formula Ia, wherein R is CH₃, A is NCH₂CH₂OCH₃, G is hydrogen and Rₐ, R_{b}, R_{c} and R_{d} are as defined in Table 1.
Table 83: This table discloses the 220 compounds T83.001 to T83.220 of the formula Ia, wherein R is hydrogen, A is NCH₂CH₂OCH₃, G is hydrogen and Rₐ, R_{b}, R_{c} and R_{d} are as defined in Table 1.
Table 84: This table discloses the 220 compounds T84.001 to T84.220 of the formula Ia, wherein R is CH₂CH₃, A is NCH₂CH₂OCH₃, G is hydrogen and Rₐ, R_{b}, R_{c} and R_{d} are as defined in Table 1.
Table 85: This table discloses the 220 compounds T85.001 to T85.220 of the formula Ia, wherein R is CH₂CF₃, A is NCH₂CH₂OCH₃, G is hydrogen and Rₐ, R_{b}, R_{c} and R_{d} are as defined in Table 1.
Table 86: This table discloses the 220 compounds T86.001 to T86.220 of the formula Ia, wherein R is CH₂OCH₃, A is NCH₂CH₂OCH₃, G is hydrogen and Rₐ, R_{b}, R_{c} and R_{d} are as defined in Table 1.
Table 87: This table discloses the 220 compounds T87.001 to T87.220 of the formula Ia, wherein R is CH₂CH₂OCH₃, A is NCH₂CH₂OCH₃, G is hydrogen and Rₐ, R_{b}, R_{c} and R_{d} are as defined in Table 1.
Table 88: This table discloses the 220 compounds T88.001 to T88.220 of the formula Ia, wherein R is allyl, A is NCH₂CH₂OCH₃, G is hydrogen and Rₐ, R_{b}, R_{c} and R_{d} are as defined in Table 1.
Table 89: This table discloses the 220 compounds T89.001 to T89.220 of the formula Ia, wherein R is propargyl, A is NCH₂CH₂OCH₃, G is hydrogen and Rₐ, R_{b}, R_{c} and R_{d} are as defined in Table 1.
Table 90: This table discloses the 220 compounds T90.001 to T90.220 of the formula Ia, wherein R is benzyl, A is NCH₂CH₂OCH₃, G is hydrogen and Rₐ, R_{b}, R_{c} and R_{d} are as defined in Table 1.
Table 91: This table discloses the 220 compounds T91.001 to T91.220 of the formula Ia, wherein R is CH₃, A is NCH₂CH₂CH₂OCH₃, G is hydrogen and Rₐ, R_{b}, R_{c} and R_{d} are as defined in Table 1.
Table 92: This table discloses the 220 compounds T92.001 to T92.220 of the formula Ia, wherein R is hydrogen, A is NCH₂CH₂CH₂OCH₃, G is hydrogen and Rₐ, R_{b}, R_{c} and R_{d} are as defined in Table 1.
Table 93: This table discloses the 220 compounds T93.001 to T93.220 of the formula Ia, wherein R is CH₂CH₃, A is NCH₂CH₂CH₂OCH₃, G is hydrogen and Rₐ, R_{b}, R_{c} and R_{d} are as defined in Table 1.
Table 94: This table discloses the 220 compounds T94.001 to T94.220 of the formula Ia, wherein R is CH₂CF₃, A is NCH₂CH₂CH₂OCH₃, G is hydrogen and Rₐ, R_{b}, R_{c} and R_{d} are as defined in Table 1.
Table 95: This table discloses the 220 compounds T95.001 to T95.220 of the formula Ia, wherein R is CH₂OCH₃, A is NCH₂CH₂CH₂OCH₃, G is hydrogen and Rₐ, R_{b}, R_{c} and R_{d} are as defined in Table 1.
Table 96: This table discloses the 220 compounds T96.001 to T96.220 of the formula Ia, wherein R is CH₂CH₂OCH₃, A is NCH₂CH₂CH₂OCH₃, G is hydrogen and Rₐ, R_{b}, R_{c} and R_{d} are as defined in Table 1.
Table 97: This table discloses the 220 compounds T97.001 to T97.220 of the formula Ia, wherein R is allyl, A is NCH₂CH₂CH₂OCH₃, G is hydrogen and Rₐ, R_{b}, R_{c} and R_{d} are as defined in Table 1.
Table 98: This table discloses the 220 compounds T98.001 to T98.220 of the formula Ia, wherein R is propargyl, A is NCH₂CH₂CH₂OCH₃, G is hydrogen and Rₐ, R_{b}, R_{c} and R_{d} are as defined in Table 1.
Table 99: This table discloses the 220 compounds T99.001 to T99.220 of the formula Ia, wherein R is benzyl, A is NCH₂CH₂CH₂OCH₃, G is hydrogen and Rₐ, R_{b}, R_{c} and R_{d} are as defined in Table 1.
Table 100: This table discloses the 220 compounds T100.001 to T100.220 of the formula Ia, wherein R is CH₃, A is NCH₂OCH₂CH₂OCH₃, G is hydrogen and Rₐ, R_{b}, R_{c} and R_{d} are as defined in Table 1.
Table 101: This table discloses the 220 compounds T101.001 to T101.220 of the formula Ia, wherein R is hydrogen, A is NCH₂OCH₂CH₂OCH₃, G is hydrogen and Rₐ, R_{b}, R_{c} and R_{d} are as defined in Table 1.
Table 102: This table discloses the 220 compounds T102.001 to T102.220 of the formula Ia, wherein R is CH₂CH₃, A is NCH₂OCH₂CH₂OCH₃, G is hydrogen and Rₐ, R_{b}, R_{c} and R_{d} are as defined in Table 1.
Table 103: This table discloses the 220 compounds T103.001 to T103.220 of the formula Ia, wherein R is CH₂CF₃, A is NCH₂OCH₂CH₂OCH₃, G is hydrogen and Rₐ, R_{b}, R_{c} and R_{d} are as defined in Table 1.
Table 104: This table discloses the 220 compounds T104.001 to T104.220 of the formula Ia, wherein R is CH₂OCH₃, A is NCH₂OCH₂CH₂OCH₃, G is hydrogen and Rₐ, R_{b}, R_{c} and R_{d} are as defined in Table 1.
Table 105: This table discloses the 220 compounds T105.001 to T105.220 of the formula Ia, wherein R is CH₂CH₂OCH₃, A is NCH₂OCH₂CH₂OCH₃, G is hydrogen and Rₐ, R_{b}, R_{c} and R_{d} are as defined in Table 1.
Table 106: This table discloses the 220 compounds T106.001 to T106.220 of the formula Ia, wherein R is allyl, A is NCH₂OCH₂CH₂OCH₃, G is hydrogen and Rₐ, R_{b}, R_{c} and R_{d} are as defined in Table 1.
Table 107: This table discloses the 220 compounds T107.001 to T107.220 of the formula Ia, wherein R is propargyl, A is NCH₂OCH₂CH₂OCH₃, G is hydrogen and Rₐ, R_{b}, R_{c} and R_{d} are as defined in Table 1.
Table 108: This table discloses the 220 compounds T108.001 to T108.220 of the formula Ia, wherein R is benzyl, A is NCH₂OCH₂CH₂OCH₃, G is hydrogen and Rₐ, R_{b}, R_{c} and R_{d} are as defined in Table 1.
Table 109: This table discloses the 220 compounds T109.001 to T109.220 of the formula Ia, wherein R is CH₃, A is NCH₂CH₂OCH₂OCH₃, G is hydrogen and Rₐ, R_{b}, R_{c} and R_{d} are as defined in Table 1.
Table 110: This table discloses the 220 compounds T110.001 to T110.220 of the formula Ia, wherein R is hydrogen, A is NCH₂CH₂OCH₂OCH₃, G is hydrogen and Rₐ, R_{b}, R_{c} and R_{d} are as defined in Table 1.
Table 111: This table discloses the 220 compounds T111.001 to T111.220 of the formula Ia, wherein R is CH₂CH₃, A is NCH₂CH₂OCH₂OCH₃, G is hydrogen and Rₐ, R_{b}, R_{c} and R_{d} are as defined in Table 1.
Table 112: This table discloses the 220 compounds T112.001 to T112.220 of the formula Ia, wherein R is CH₂CF₃, A is NCH₂CH₂OCH₂OCH₃, G is hydrogen and Rₐ, R_{b}, R_{c} and R_{d} are as defined in Table 1.
Table 113: This table discloses the 220 compounds T113.001 to T113.220 of the formula Ia, wherein R is CH₂OCH₃, A is NCH₂CH₂OCH₂OCH₃, G is hydrogen and Rₐ, R_{b}, R_{c} and R_{d} are as defined in Table 1.
Table 114: This table discloses the 220 compounds T114.001 to T114.220 of the formula Ia, wherein R is CH₂CH₂OCH₃, A is NCH₂CH₂OCH₂OCH₃, G is hydrogen and Rₐ, R_{b}, R_{c} and R_{d} are as defined in Table 1.
Table 115: This table discloses the 220 compounds T115.001 to T115.220 of the formula Ia, wherein R is allyl, A is NCH₂CH₂OCH₂OCH₃, G is hydrogen and Rₐ, R_{b}, R_{c} and R_{d} are as defined in Table 1.
Table 116: This table discloses the 220 compounds T116.001 to T116.220 of the formula Ia, wherein R is propargyl, A is NCH₂CH₂OCH₂OCH₃, G is hydrogen and Rₐ, R_{b}, R_{c} and R_{d} are as defined in Table 1.
Table 117: This table discloses the 220 compounds T117.001 to T117.220 of the formula Ia, wherein R is benzyl, A is NCH₂CH₂OCH₂OCH₃, G is hydrogen and Rₐ, R_{b}, R_{c} and R_{d} are as defined in Table 1.
Table 118: This table discloses the 220 compounds T118.001 to T118.220 of the formula Ia, wherein R is CH₃, A is NOH, G is hydrogen and Rₐ, R_{b}, R_{c} and R_{d} are as defined in Table 1.
Table 119: This table discloses the 220 compounds T119.001 to T119.220 of the formula Ia, wherein R is hydrogen, A is NOH, G is hydrogen and Rₐ, R_{b}, R_{c} and R_{d} are as defined in Table 1.
Table 120: This table discloses the 220 compounds T120.001 to T120.220 of the formula Ia, wherein R is CH₂CH₃, A is NOH, G is hydrogen and Rₐ, R_{b}, R_{c} and R_{d} are as defined in Table 1.
Table 121: This table discloses the 220 compounds T121.001 to T121.220 of the formula Ia, wherein R is CH₂CF₃, A is NOH, G is hydrogen and Rₐ, R_{b}, R_{c} and R_{d} are as defined in Table 1.
Table 122: This table discloses the 220 compounds T122.001 to T122.220 of the formula Ia, wherein R is CH₂OCH₃, A is NOH, G is hydrogen and Rₐ, R_{b}, R_{c} and R_{d} are as defined in Table 1.
Table 123: This table discloses the 220 compounds T123.001 to T123.220 of the formula Ia, wherein R is CH₂CH₂OCH₃, A is NOH, G is hydrogen and Rₐ, R_{b}, R_{c} and R_{d} are as defined in Table 1.
Table 124: This table discloses the 220 compounds T124.001 to T124.220 of the formula Ia, wherein R is allyl, A is NOH, G is hydrogen and Rₐ, R_{b}, R_{c} and R_{d} are as defined in Table 1.
Table 125: This table discloses the 220 compounds T125.001 to T125.220 of the formula Ia, wherein R is propargyl, A is NOH, G is hydrogen and Rₐ, R_{b}, R_{c} and R_{d} are as defined in Table 1.
Table 126: This table discloses the 220 compounds T126.001 to T126.220 of the formula Ia, wherein R is benzyl, A is NOH, G is hydrogen and Rₐ, R_{b}, R_{c} and R_{d} are as defined in Table 1.
Table 127: This table discloses the 220 compounds T127.001 to T127.220 of the formula Ia, wherein R is CH₃, A is NOCH₃, G is hydrogen and Rₐ, R_{b}, R_{c} and R_{d} are as defined in Table 1.
Table 128: This table discloses the 220 compounds T128.001 to T128.220 of the formula Ia, wherein R is hydrogen, A is NOCH₃, G is hydrogen and Rₐ, R_{b}, R_{c} and R_{d} are as defined in Table 1.
Table 129: This table discloses the 220 compounds T129.001 to T129.220 of the formula Ia, wherein R is CH₂CH₃, A is NOCH₃, G is hydrogen and Rₐ, R_{b}, R_{c} and R_{d} are as defined in Table 1.
Table 130: This table discloses the 220 compounds T130.001 to T130.220 of the formula Ia, wherein R is CH₂CF₃, A is NOCH₃, G is hydrogen and Rₐ, R_{b}, R_{c} and R_{d} are as defined in Table 1.
Table 131: This table discloses the 220 compounds T131.001 to T131.220 of the formula Ia, wherein R is CH₂OCH₃, A is NOCH₃, G is hydrogen and Rₐ, R_{b}, R_{c} and R_{d} are as defined in Table 1.
Table 132: This table discloses the 220 compounds T132.001 to T132.220 of the formula Ia, wherein R is CH₂CH₂OCH₃, A is NOCH₃, G is hydrogen and Rₐ, R_{b}, R_{c} and R_{d} are as defined in Table 1.
Table 133: This table discloses the 220 compounds T133.001 to T133.220 of the formula Ia, wherein R is allyl, A is NOCH₃, G is hydrogen and Rₐ, R_{b}, R_{c} and R_{d} are as defined in Table 1.
Table 134: This table discloses the 220 compounds T134.001 to T134.220 of the formula Ia, wherein R is propargyl, A is NOCH₃, G is hydrogen and Rₐ, R_{b}, R_{c} and R_{d} are as defined in Table 1.
Table 135: This table discloses the 220 compounds T135.001 to T135.220 of the formula Ia, wherein R is benzyl, A is NOCH₃, G is hydrogen and Rₐ, R_{b}, R_{c} and R_{d} are as defined in Table 1.
Table 136: This table discloses the 220 compounds T136.001 to T136.220 of the formula Ia, wherein R is CH₃, A is NOCH₂CH₃, G is hydrogen and Rₐ, R_{b}, R_{c} and R_{d} are as defined in Table 1.
Table 137: This table discloses the 220 compounds T137.001 to T137.220 of the formula Ia, wherein R is hydrogen, A is NOCH₂CH₃, G is hydrogen and Rₐ, R_{b}, R_{c} and R_{d} are as defined in Table 1.
Table 138: This table discloses the 220 compounds T138.001 to T138.220 of the formula Ia, wherein R is CH₂CH₃, A is NOCH₂CH₃, G is hydrogen and Rₐ, R_{b}, R_{c} and R_{d} are as defined in Table 1.
Table 139: This table discloses the 220 compounds T139.001 to T139.220 of the formula Ia, wherein R is CH₂CF₃, A is NOCH₂CH₃, G is hydrogen and Rₐ, R_{b}, R_{c} and R_{d} are as defined in Table 1.
Table 140: This table discloses the 220 compounds T140.001 to T140.220 of the formula Ia, wherein R is CH₂OCH₃, A is NOCH₂CH₃, G is hydrogen and Rₐ, R_{b}, R_{c} and R_{d} are as defined in Table 1.
Table 141: This table discloses the 220 compounds T141.001 to T141.220 of the formula Ia, wherein R is CH₂CH₂OCH₃, A is NOCH₂CH₃, G is hydrogen and Rₐ, R_{b}, R_{c} and R_{d} are as defined in Table 1.
Table 142: This table discloses the 220 compounds T142.001 to T142.220 of the formula Ia, wherein R is allyl, A is NOCH₂CH₃, G is hydrogen and Rₐ, R_{b}, R_{c} and R_{d} are as defined in Table 1.
Table 143: This table discloses the 220 compounds T143.001 to T143.220 of the formula Ia, wherein R is propargyl, A is NOCH₂CH₃, G is hydrogen and Rₐ, R_{b}, R_{c} and R_{d} are as defined in Table 1.
Table 144: This table discloses the 220 compounds T144.001 to T144.220 of the formula Ia, wherein R is benzyl, A is NOCH₂CH₃, G is hydrogen and Rₐ, R_{b}, R_{c} and R_{d} are as defined in Table 1.
Table 145: This table discloses the 220 compounds T145.001 to T145.220 of the formula Ia, wherein R is CH₃, A is NOCH(CH₃)₂, G is hydrogen and Rₐ, R_{b}, R_{c} and R_{d} are as defined in Table 1.
Table 146: This table discloses the 220 compounds T146.001 to T146.220 of the formula Ia, wherein R is hydrogen, A is NOCH(CH₃)₂, G is hydrogen and Rₐ, R_{b}, R_{c} and R_{d} are as defined in Table 1.
Table 147: This table discloses the 220 compounds T147.001 to T147.220 of the formula Ia, wherein R is CH₂CH₃, A is NOCH(CH₃)₂, G is hydrogen and Rₐ, R_{b}, R_{c} and R_{d} are as defined in Table 1.
Table 148: This table discloses the 220 compounds T148.001 to T148.220 of the formula Ia, wherein R is CH₂CF₃, A is NOCH(CH₃)₂, G is hydrogen and Rₐ, R_{b}, R_{c} and R_{d} are as defined in Table 1.
Table 149: This table discloses the 220 compounds T149.001 to T149.220 of the formula Ia, wherein R is CH₂OCH₃, A is NOCH(CH₃)₂, G is hydrogen and Rₐ, R_{b}, R_{c} and R_{d} are as defined in Table 1.
Table 150: This table discloses the 220 compounds T150.001 to T150.220 of the formula Ia, wherein R is CH₂CH₂OCH₃, A is NOCH(CH₃)₂, G is hydrogen and Rₐ, R_{b}, R_{c} and R_{d} are as defined in Table 1.
Table 151: This table discloses the 220 compounds T151.001 to T151.220 of the formula Ia, wherein R is allyl, A is NOCH(CH₃)₂, G is hydrogen and Rₐ, R_{b}, R_{c} and R_{d} are as defined in Table 1.
Table 152: This table discloses the 220 compounds T152.001 to T152.220 of the formula Ia, wherein R is propargyl, A is NOCH(CH₃)₂, G is hydrogen and Rₐ, R_{b}, R_{c} and R_{d} are as defined in Table 1.
Table 153: This table discloses the 220 compounds T153.001 to T153.220 of the formula Ia, wherein R is benzyl, A is NOCH(CH₃)₂, G is hydrogen and Rₐ, R_{b}, R_{c} and R_{d} are as defined in Table 1.
Table 154: This table discloses the 220 compounds T154.001 to T154.220 of the formula Ia, wherein R is CH₃, A is NO-cyclopropyl, G is hydrogen and Rₐ, R_{b}, R_{c} and R_{d} are as defined in Table 1.
Table 155: This table discloses the 220 compounds T155.001 to T155.220 of the formula Ia, wherein R is hydrogen, A is NO-cyclopropyl, G is hydrogen and Rₐ, R_{b}, R_{c} and R_{d} are as defined in Table 1.
Table 156: This table discloses the 220 compounds T156.001 to T156.220 of the formula Ia, wherein R is CH₂CH₃, A is NO-cyclopropyl, G is hydrogen and Rₐ, R_{b}, R_{c} and R_{d} are as defined in Table 1.
Table 157: This table discloses the 220 compounds T157.001 to T157.220 of the formula Ia, wherein R is CH₂CF₃, A is NO-cyclopropyl, G is hydrogen and Rₐ, R_{b}, R_{c} and R_{d} are as defined in Table 1.
Table 158: This table discloses the 220 compounds T158.001 to T158.220 of the formula Ia, wherein R is CH₂OCH₃, A is NO-cyclopropyl, G is hydrogen and Rₐ, R_{b}, R_{c} and R_{d} are as defined in Table 1.
Table 159: This table discloses the 220 compounds T159.001 to T159.220 of the formula Ia, wherein R is CH₂CH₂OCH₃, A is NO-cyclopropyl, G is hydrogen and Rₐ, R_{b}, R_{c} and R_{d} are as defined in Table 1.
Table 160: This table discloses the 220 compounds T160.001 to T160.220 of the formula Ia, wherein R is allyl, A is NO-cyclopropyl, G is hydrogen and Rₐ, R_{b}, R_{c} and R_{d} are as defined in Table 1.
Table 161: This table discloses the 220 compounds T161.001 to T161.220 of the formula Ia, wherein R is propargyl, A is NO-cyclopropyl, G is hydrogen and Rₐ, R_{b}, R_{c} and R_{d} are as defined in Table 1.
Table 162: This table discloses the 220 compounds T162.001 to T162.220 of the formula Ia, wherein R is benzyl, A is NO-cyclopropyl, G is hydrogen and Rₐ, R_{b}, R_{c} and R_{d} are as defined in Table 1.
Table 163: This table discloses the 220 compounds T163.001 to T163.220 of the formula Ia, wherein R is CH₃, A is NOCH₂CH═CH₂, G is hydrogen and Rₐ, R_{b}, R_{c} and R_{d} are as defined in Table 1.
Table 164: This table discloses the 220 compounds T164.001 to T164.220 of the formula Ia, wherein R is hydrogen, A is NOCH₂CH═CH₂, G is hydrogen and Rₐ, R_{b}, R_{c} and R_{d} are as defined in Table 1.
Table 165: This table discloses the 220 compounds T165.001 to T165.220 of the formula Ia, wherein R is CH₂CH₃, A is NOCH₂CH═CH₂, G is hydrogen and Rₐ, R_{b}, R_{c} and R_{d} are as defined in Table 1.
Table 166: This table discloses the 220 compounds T166.001 to T166.220 of the formula Ia, wherein R is CH₂CF₃, A is NOCH₂CH═CH₂, G is hydrogen and Rₐ, R_{b}, R_{c} and R_{d} are as defined in Table 1.
Table 167: This table discloses the 220 compounds T167.001 to T167.220 of the formula Ia, wherein R is CH₂OCH₃, A is NOCH₂CH═CH₂, G is hydrogen and Rₐ, R_{b}, R_{c} and R_{d} are as defined in Table 1.
Table 168: This table discloses the 220 compounds T168.001 to T168.220 of the formula Ia, wherein R is CH₂CH₂OCH₃, A is NOCH₂CH═CH₂, G is hydrogen and Rₐ, R_{b}, R_{c} and R_{d} are as defined in Table 1.
Table 169: This table discloses the 220 compounds T169.001 to T169.220 of the formula Ia, wherein R is allyl, A is NOCH₂CH═CH₂, G is hydrogen and Rₐ, R_{b}, R_{c} and R_{d} are as defined in Table 1.
Table 170: This table discloses the 220 compounds T170.001 to T170.220 of the formula Ia, wherein R is propargyl, A is NOCH₂CH═CH₂, G is hydrogen and Rₐ, R_{b}, R_{c} and R_{d} are as defined in Table 1.
Table 171: This table discloses the 220 compounds T171.001 to T171.220 of the formula Ia, wherein R is benzyl, A is NOCH₂CH═CH₂, G is hydrogen and Rₐ, R_{b}, R_{c} and R_{d} are as defined in Table 1.
Table 172: This table discloses the 220 compounds T172.001 to T172.220 of the formula Ia, wherein R is CH₃, A is NOCH₂C≡CH, G is hydrogen and Rₐ, R_{b}, R_{c} and R_{d} are as defined in Table 1.
Table 173: This table discloses the 220 compounds T173.001 to T173.220 of the formula Ia, wherein R is hydrogen, A is NOCH₂C≡CH, G is hydrogen and Rₐ, R_{b}, R_{c} and R_{d} are as defined in Table 1.
Table 174: This table discloses the 220 compounds T174.001 to T174.220 of the formula Ia, wherein R is CH₂CH₃, A is NOCH₂C≡CH, G is hydrogen and Rₐ, R_{b}, R_{c} and R_{d} are as defined in Table 1.
Table 175: This table discloses the 220 compounds T175.001 to T175.220 of the formula Ia, wherein R is CH₂CF₃, A is NOCH₂C≡CH, G is hydrogen and Rₐ, R_{b}, R_{c} and R_{d} are as defined in Table 1.
Table 176: This table discloses the 220 compounds T176.001 to T176.220 of the formula Ia, wherein R is CH₂OCH₃, A is NOCH₂C≡CH, G is hydrogen and Rₐ, R_{b}, R_{c} and R_{d} are as defined in Table 1.
Table 177: This table discloses the 220 compounds T177.001 to T177.220 of the formula Ia, wherein R is CH₂CH₂OCH₃, A is NOCH₂C≡CH, G is hydrogen and Rₐ, R_{b}, R_{c} and R_{d} are as defined in Table 1.
Table 178: This table discloses the 220 compounds T178.001 to T178.220 of the formula Ia, wherein R is allyl, A is NOCH₂C≡CH, G is hydrogen and Rₐ, R_{b}, R_{c} and R_{d} are as defined in Table 1.
Table 179: This table discloses the 220 compounds T179.001 to T179.220 of the formula Ia, wherein R is propargyl, A is NOCH₂C≡CH, G is hydrogen and Rₐ, R_{b}, R_{c} and R_{d} are as defined in Table 1.
Table 180: This table discloses the 220 compounds T180.001 to T180.220 of the formula Ia, wherein R is benzyl, A is NOCH₂C≡CH, G is hydrogen and Rₐ, R_{b}, R_{c} and R_{d} are as defined in Table 1.
Table 181: This table discloses the 220 compounds T181.001 to T181.220 of the formula Ia, wherein R is CH₃, A is NOCH₂cyclo-C3, G is hydrogen and Rₐ, R_{b}, R_{c} and R_{d} are as defined in Table 1.
Table 182: This table discloses the 220 compounds T182.001 to T182.220 of the formula Ia, wherein R is hydrogen, A is NOCH₂cyclo-C3, G is hydrogen and Rₐ, R_{b}, R_{c} and R_{d} are as defined in Table 1.
Table 183: This table discloses the 220 compounds T183.001 to T183.220 of the formula Ia, wherein R is CH₂CH₃, A is NOCH₂cyclo-C3, G is hydrogen and Rₐ, R_{b}, R_{c} and R_{d} are as defined in Table 1.
Table 184: This table discloses the 220 compounds T184.001 to T184.220 of the formula Ia, wherein R is CH₂CF₃, A is NOCH₂cyclo-C3, G is hydrogen and Rₐ, R_{b}, R_{c} and R_{d} are as defined in Table 1.
Table 185: This table discloses the 220 compounds T185.001 to T185.220 of the formula Ia, wherein R is CH₂OCH₃, A is NOCH₂cyclo-C3, G is hydrogen and Rₐ, R_{b}, R_{c} and R_{d} are as defined in Table 1.
Table 186: This table discloses the 220 compounds T186.001 to T186.220 of the formula Ia, wherein R is CH₂CH₂OCH₃, A is NOCH₂cyclo-C3, G is hydrogen and Rₐ, R_{b}, R_{c} and R_{d} are as defined in Table 1.
Table 187: This table discloses the 220 compounds T187.001 to T187.220 of the formula Ia, wherein R is allyl, A is NOCH₂cyclo-C3, G is hydrogen and Rₐ, R_{b}, R_{c} and R_{d} are as defined in Table 1.
Table 188: This table discloses the 220 compounds T188.001 to T188.220 of the formula Ia, wherein R is propargyl, A is NOCH₂cyclo-C3, G is hydrogen and Rₐ, R_{b}, R_{c} and R_{d} are as defined in Table 1.
Table 189: This table discloses the 220 compounds T189.001 to T189.220 of the formula Ia, wherein R is benzyl, A is NOCH₂cyclo-C3, G is hydrogen and Rₐ, R_{b}, R_{c} and R_{d} are as defined in Table 1.
Table 190: This table discloses the 220 compounds T190.001 to T190.220 of the formula Ia, wherein R is CH₃, A is NOCH₂OCH₃, G is hydrogen and Rₐ, R_{b}, R_{c} and R_{d} are as defined in Table 1.
Table 191: This table discloses the 220 compounds T191.001 to T191.220 of the formula Ia, wherein R is hydrogen, A is NOCH₂OCH₃, G is hydrogen and Rₐ, R_{b}, R_{c} and R_{d} are as defined in Table 1.
Table 192: This table discloses the 220 compounds T192.001 to T192.220 of the formula Ia, wherein R is CH₂CH₃, A is NOCH₂OCH₃, G is hydrogen and Rₐ, R_{b}, R_{c} and R_{d} are as defined in Table 1.
Table 193: This table discloses the 220 compounds T193.001 to T193.220 of the formula Ia, wherein R is CH₂CF₃, A is NOCH₂OCH₃, G is hydrogen and Rₐ, R_{b}, R_{c} and R_{d} are as defined in Table 1.
Table 194: This table discloses the 220 compounds T194.001 to T194.220 of the formula Ia, wherein R is CH₂OCH₃, A is NOCH₂OCH₃, G is hydrogen and Rₐ, R_{b}, R_{c} and R_{d} are as defined in Table 1.
Table 195: This table discloses the 220 compounds T195.001 to T195.220 of the formula Ia, wherein R is CH₂CH₂OCH₃, A is NOCH₂OCH₃, G is hydrogen and Rₐ, R_{b}, R_{c} and R_{d} are as defined in Table 1.
Table 196: This table discloses the 220 compounds T196.001 to T196.220 of the formula Ia, wherein R is allyl, A is NOCH₂OCH₃, G is hydrogen and Rₐ, R_{b}, R_{c} and R_{d} are as defined in Table 1.
Table 197: This table discloses the 220 compounds T197.001 to T197.220 of the formula Ia, wherein R is propargyl, A is NOCH₂OCH₃, G is hydrogen and Rₐ, R_{b}, R_{c} and R_{d} are as defined in Table 1.
Table 198: This table discloses the 220 compounds T198.001 to T198.220 of the formula Ia, wherein R is benzyl, A is NOCH₂OCH₃, G is hydrogen and Rₐ, R_{b}, R_{c} and R_{d} are as defined in Table 1.
Table 199: This table discloses the 220 compounds T199.001 to T199.220 of the formula Ia, wherein R is CH₃, A is NOCH₂CH₂OCH₃, G is hydrogen and Rₐ, R_{b}, R_{c} and R_{d} are as defined in Table 1.
Table 200: This table discloses the 220 compounds T200.001 to T200.220 of the formula Ia, wherein R is hydrogen, A is NOCH₂CH₂OCH₃, G is hydrogen and Rₐ, R_{b}, R_{c} and R_{d} are as defined in Table 1.
Table 201: This table discloses the 220 compounds T201.001 to T201.220 of the formula Ia, wherein R is CH₂CH₃, A is NOCH₂CH₂OCH₃, G is hydrogen and Rₐ, R_{b}, R_{c} and R_{d} are as defined in Table 1.
Table 202: This table discloses the 220 compounds T202.001 to T202.220 of the formula Ia, wherein R is CH₂CF₃, A is NOCH₂CH₂OCH₃, G is hydrogen and Rₐ, R_{b}, R_{c} and R_{d} are as defined in Table 1.
Table 203: This table discloses the 220 compounds T203.001 to T203.220 of the formula Ia, wherein R is CH₂OCH₃, A is NOCH₂CH₂OCH₃, G is hydrogen and Rₐ, R_{b}, R_{c} and R_{d} are as defined in Table 1.
Table 204: This table discloses the 220 compounds T204.001 to T204.220 of the formula Ia, wherein R is CH₂CH₂OCH₃, A is NOCH₂CH₂OCH₃, G is hydrogen and Rₐ, R_{b}, R_{c} and R_{d} are as defined in Table 1.
Table 205: This table discloses the 220 compounds T205.001 to T205.220 of the formula Ia, wherein R is allyl, A is NOCH₂CH₂OCH₃, G is hydrogen and Rₐ, R_{b}, R_{c} and R_{d} are as defined in Table 1.
Table 206: This table discloses the 220 compounds T206.001 to T206.220 of the formula Ia, wherein R is propargyl, A is NOCH₂CH₂OCH₃, G is hydrogen and Rₐ, R_{b}, R_{c} and R_{d} are as defined in Table 1.
Table 207: This table discloses the 220 compounds T207.001 to T207.220 of the formula Ia, wherein R is benzyl, A is NOCH₂CH₂OCH₃, G is hydrogen and Rₐ, R_{b}, R_{c} and R_{d} are as defined in Table 1.
Table 208: This table discloses the 220 compounds T208.001 to T208.220 of the formula Ia, wherein R is CH₃, A is NOCH₂CH₂CH₂OCH₃, G is hydrogen and Rₐ, R_{b}, R_{c} and R_{d} are as defined in Table 1.
Table 209: This table discloses the 220 compounds T209.001 to T209.220 of the formula Ia, wherein R is hydrogen, A is NOCH₂CH₂CH₂OCH₃, G is hydrogen and Rₐ, R_{b}, R_{c} and R_{d} are as defined in Table 1.
Table 210: This table discloses the 220 compounds T210.001 to T210.220 of the formula Ia, wherein R is CH₂CH₃, A is NOCH₂CH₂CH₂OCH₃, G is hydrogen and Rₐ, R_{b}, R_{c} and R_{d} are as defined in Table 1.
Table 211: This table discloses the 220 compounds T211.001 to T211.220 of the formula Ia, wherein R is CH₂CF₃, A is NOCH₂CH₂CH₂OCH₃, G is hydrogen and Rₐ, R_{b}, R_{c} and R_{d} are as defined in Table 1.
Table 212: This table discloses the 220 compounds T212.001 to T212.220 of the formula Ia, wherein R is CH₂OCH₃, A is NOCH₂CH₂CH₂OCH₃, G is hydrogen and Rₐ, R_{b}, R_{c} and R_{d} are as defined in Table 1.
Table 213: This table discloses the 220 compounds T213.001 to T213.220 of the formula Ia, wherein R is CH₂CH₂OCH₃, A is NOCH₂CH₂CH₂OCH₃, G is hydrogen and Rₐ, R_{b}, R_{c} and R_{d} are as defined in Table 1.
Table 214: This table discloses the 220 compounds T214.001 to T214.220 of the formula Ia, wherein R is allyl, A is NOCH₂CH₂CH₂OCH₃, G is hydrogen and Rₐ, R_{b}, R_{c} and R_{d} are as defined in Table 1.
Table 215: This table discloses the 220 compounds T215.001 to T215.220 of the formula Ia, wherein R is propargyl, A is NOCH₂CH₂CH₂OCH₃, G is hydrogen and Rₐ, R_{b}, R_{c} and R_{d} are as defined in Table 1.
Table 216: This table discloses the 220 compounds T216.001 to T216.220 of the formula Ia, wherein R is benzyl, A is NOCH₂CH₂CH₂OCH₃, G is hydrogen and Rₐ, R_{b}, R_{c} and R_{d} are as defined in Table 1.
Table 217: This table discloses the 220 compounds T217.001 to T217.220 of the formula Ia, wherein R is CH₃, A is NOCH₂OCH₂CH₂OCH₃, G is hydrogen and Rₐ, R_{b}, R_{c} and R_{d} are as defined in Table 1.
Table 218: This table discloses the 220 compounds T218.001 to T218.220 of the formula Ia, wherein R is hydrogen, A is NOCH₂OCH₂CH₂OCH₃, G is hydrogen and Rₐ, R_{b}, R_{c} and R_{d} are as defined in Table 1.
Table 219: This table discloses the 220 compounds T219.001 to T219.220 of the formula Ia, wherein R is CH₂CH₃, A is NOCH₂OCH₂CH₂OCH₃, G is hydrogen and Rₐ, R_{b}, R_{c} and R_{d} are as defined in Table 1.
Table 220: This table discloses the 220 compounds T220.001 to T220.220 of the formula Ia, wherein R is CH₂CF₃, A is NOCH₂OCH₂CH₂OCH₃, G is hydrogen and Rₐ, R_{b}, R_{c} and R_{d} are as defined in Table 1.
Table 221: This table discloses the 220 compounds T221.001 to T221.220 of the formula Ia, wherein R is CH₂OCH₃, A is NOCH₂OCH₂CH₂OCH₃, G is hydrogen and Rₐ, R_{b}, R_{c} and R_{d} are as defined in Table 1.
Table 222: This table discloses the 220 compounds T222.001 to T222.220 of the formula Ia, wherein R is CH₂CH₂OCH₃, A is NOCH₂OCH₂CH₂OCH₃, G is hydrogen and Rₐ, R_{b}, R_{c} and R_{d} are as defined in Table 1.
Table 223: This table discloses the 220 compounds T223.001 to T223.220 of the formula Ia, wherein R is allyl, A is NOCH₂OCH₂CH₂OCH₃, G is hydrogen and Rₐ, R_{b}, R_{c} and R_{d} are as defined in Table 1.
Table 224: This table discloses the 220 compounds T224.001 to T224.220 of the formula Ia, wherein R is propargyl, A is NOCH₂OCH₂CH₂OCH₃, G is hydrogen and Rₐ, R_{b}, R_{c} and R_{d} are as defined in Table 1.
Table 225: This table discloses the 220 compounds T225.001 to T225.220 of the formula Ia, wherein R is benzyl, A is NOCH₂OCH₂CH₂OCH₃, G is hydrogen and Rₐ, R_{b}, R_{c} and R_{d} are as defined in Table 1.
Table 226: This table discloses the 220 compounds T226.001 to T226.220 of the formula Ia, wherein R is CH₃, A is NOCH₂CH₂OCH₂OCH₃, G is hydrogen and Rₐ, R_{b}, R_{c} and R_{d} are as defined in Table 1.
Table 227: This table discloses the 220 compounds T227.001 to T227.220 of the formula Ia, wherein R is hydrogen, A is NOCH₂CH₂OCH₂OCH₃, G is hydrogen and Rₐ, R_{b}, R_{c} and R_{d} are as defined in Table 1.
Table 228: This table discloses the 220 compounds T228.001 to T228.220 of the formula Ia, wherein R is CH₂CH₃, A is NOCH₂CH₂OCH₂OCH₃, G is hydrogen and Rₐ, R_{b}, R_{c} and R_{d} are as defined in Table 1.
Table 229: This table discloses the 220 compounds T229.001 to T229.220 of the formula Ia, wherein R is CH₂CF₃, A is NOCH₂CH₂OCH₂OCH₃, G is hydrogen and Rₐ, R_{b}, R_{c} and R_{d} are as defined in Table 1.
Table 230: This table discloses the 220 compounds T230.001 to T230.220 of the formula Ia, wherein R is CH₂OCH₃, A is NOCH₂CH₂OCH₂OCH₃, G is hydrogen and Rₐ, R_{b}, R_{c} and R_{d} are as defined in Table 1.
Table 231: This table discloses the 220 compounds T231.001 to T231.220 of the formula Ia, wherein R is CH₂CH₂OCH₃, A is NOCH₂CH₂OCH₂OCH₃, G is hydrogen and Rₐ, R_{b}, R_{c} and R_{d} are as defined in Table 1.
Table 232: This table discloses the 220 compounds T232.001 to T232.220 of the formula Ia, wherein R is allyl, A is NOCH₂CH₂OCH₂OCH₃, G is hydrogen and Rₐ, R_{b}, R_{c} and R_{d} are as defined in Table 1.
Table 233: This table discloses the 220 compounds T233.001 to T233.220 of the formula Ia, wherein R is propargyl, A is NOCH₂CH₂OCH₂OCH₃, G is hydrogen and Rₐ, R_{b}, R_{c} and R_{d} are as defined in Table 1.
Table 234: This table discloses the 220 compounds T234.001 to T234.220 of the formula Ia, wherein R is benzyl, A is NOCH₂CH₂OCH₂OCH₃, G is hydrogen and Rₐ, R_{b}, R_{c} and R_{d} are as defined in Table 1.
Table 235: This table discloses the 220 compounds T235.001 to T235.220 of the formula Ia, wherein R is CH₃, A is NOCH₂-cyclohexyl, G is hydrogen and Rₐ, R_{b}, R_{c} and R_{d} are as defined in Table 1.
Table 236: This table discloses the 220 compounds T236.001 to T236.220 of the formula Ia, wherein R is hydrogen, A is NOCH₂-cyclohexyl, G is hydrogen and Rₐ, R_{b}, R_{c} and R_{d} are as defined in Table 1.
Table 237: This table discloses the 220 compounds T237.001 to T237.220 of the formula Ia, wherein R is CH₂CH₃, A is NOCH₂-cyclohexyl, G is hydrogen and Rₐ, R_{b}, R_{c} and R_{d} are as defined in Table 1.
Table 238: This table discloses the 220 compounds T238.001 to T238.220 of the formula Ia, wherein R is CH₂CF₃, A is NOCH₂-cyclohexyl, G is hydrogen and Rₐ, R_{b}, R_{c} and R_{d} are as defined in Table 1.
Table 239: This table discloses the 220 compounds T239.001 to T239.220 of the formula Ia, wherein R is CH₂OCH₃, A is NOCH₂-cyclohexyl, G is hydrogen and Rₐ, R_{b}, R_{c} and R_{d} are as defined in Table 1.
Table 240: This table discloses the 220 compounds T240.001 to T240.220 of the formula Ia, wherein R is CH₂CH₂OCH₃, A is NOCH₂-cyclohexyl, G is hydrogen and Rₐ, R_{b}, R_{c} and R_{d} are as defined in Table 1.
Table 241: This table discloses the 220 compounds T241.001 to T241.220 of the formula Ia, wherein R is allyl, A is NOCH₂-cyclohexyl, G is hydrogen and Rₐ, R_{b}, R_{c} and R_{d} are as defined in Table 1.
Table 242: This table discloses the 220 compounds T242.001 to T242.220 of the formula Ia, wherein R is propargyl, A is NOCH₂-cyclohexyl, G is hydrogen and Rₐ, R_{b}, R_{c} and R_{d} are as defined in Table 1.
Table 243: This table discloses the 220 compounds T243.001 to T243.220 of the formula Ia, wherein R is benzyl, A is NOCH₂-cyclohexyl, G is hydrogen and Rₐ, R_{b}, R_{c} and R_{d} are as defined in Table 1.
Table 244: This table discloses the 220 compounds T244.001 to T244.220 of the formula Ia, wherein R is CH₃, A is NO-(tetrahydrofuran-2-yl), G is hydrogen and Rₐ, R_{b}, R_{c} and R_{d} are as defined in Table 1.
Table 245: This table discloses the 220 compounds T245.001 to T245.220 of the formula Ia, wherein R is hydrogen, A is NO-(tetrahydrofuran-2-yl), G is hydrogen and Rₐ, R_{b}, R_{c} and R_{d} are as defined in Table 1.
Table 246: This table discloses the 220 compounds T246.001 to T246.220 of the formula Ia, wherein R is CH₂CH₃, A is NO-(tetrahydrofuran-2-yl), G is hydrogen and Rₐ, R_{b}, R_{c} and R_{d} are as defined in Table 1.
Table 247: This table discloses the 220 compounds T247.001 to T247.220 of the formula Ia, wherein R is CH₂CF₃, A is NO-(tetrahydrofuran-2-yl), G is hydrogen and Rₐ, R_{b}, R_{c} and R_{d} are as defined in Table 1.
Table 248: This table discloses the 220 compounds T248.001 to T248.220 of the formula Ia, wherein R is CH₂OCH₃, A is NO-(tetrahydrofuran-2-yl), G is hydrogen and Rₐ, R_{b}, R_{c} and R_{d} are as defined in Table 1.
Table 249: This table discloses the 220 compounds T249.001 to T249.220 of the formula Ia, wherein R is CH₂CH₂OCH₃, A is NO-(tetrahydrofuran-2-yl), G is hydrogen and Rₐ, R_{b}, R_{c} and R_{d} are as defined in Table 1.
Table 250: This table discloses the 220 compounds T250.001 to T250.220 of the formula Ia, wherein R is allyl, A is NO-(tetrahydrofuran-2-yl), G is hydrogen and Rₐ, R_{b}, R_{c} and R_{d} are as defined in Table 1.
Table 251: This table discloses the 220 compounds T251.001 to T251.220 of the formula Ia, wherein R is propargyl, A is NO-(tetrahydrofuran-2-yl), G is hydrogen and Rₐ, R_{b}, R_{c} and R_{d} are as defined in Table 1.
Table 252: This table discloses the 220 compounds T252.001 to T252.220 of the formula Ia, wherein R is benzyl, A is NO-(tetrahydrofuran-2-yl), G is hydrogen and Rₐ, R_{b}, R_{c} and R_{d} are as defined in Table 1.
Table 253: This table discloses the 220 compounds T253.001 to T253.220 of the formula Ia, wherein R is CH₃, A is NO-(tetrahydropyran-2-yl), G is hydrogen and Rₐ, R_{b}, R_{c} and R_{d} are as defined in Table 1.
Table 254: This table discloses the 220 compounds T254.001 to T254.220 of the formula Ia, wherein R is hydrogen, A is NO-(tetrahydropyran-2-yl), G is hydrogen and Rₐ, R_{b}, R_{c} and R_{d} are as defined in Table 1.
Table 255: This table discloses the 220 compounds T255.001 to T255.220 of the formula Ia, wherein R is CH₂CH₃, A is NO-(tetrahydropyran-2-yl), G is hydrogen and Rₐ, R_{b}, R_{c} and R_{d} are as defined in Table 1.
Table 256: This table discloses the 220 compounds T256.001 to T256.220 of the formula Ia, wherein R is CH₂CF₃, A is NO-(tetrahydropyran-2-yl), G is hydrogen and Rₐ, R_{b}, R_{c} and R_{d} are as defined in Table 1.
Table 257: This table discloses the 220 compounds T257.001 to T257.220 of the formula Ia, wherein R is CH₂OCH₃, A is NO-(tetrahydropyran-2-yl), G is hydrogen and Rₐ, R_{b}, R_{c} and R_{d} are as defined in Table 1.
Table 258: This table discloses the 220 compounds T258.001 to T258.220 of the formula Ia, wherein R is CH₂CH₂OCH₃, A is NO-(tetrahydropyran-2-yl), G is hydrogen and Rₐ, R_{b}, and R_{d} are as defined in Table 1.
Table 259: This table discloses the 220 compounds T259.001 to T259.220 of the formula Ia, wherein R is allyl, A is NO-(tetrahydropyran-2-yl), G is hydrogen and Rₐ, R_{b}, R_{c} and R_{d} are as defined in Table 1.
Table 260: This table discloses the 220 compounds T260.001 to T260.220 of the formula Ia, wherein R is propargyl, A is NO-(tetrahydropyran-2-yl), G is hydrogen and Rₐ, R_{b}, R_{c} and R_{d} are as defined in Table 1.
Table 261: This table discloses the 220 compounds T261.001 to T261.220 of the formula Ia, wherein R is benzyl, A is NO-(tetrahydropyran-2-yl), G is hydrogen and Rₐ, R_{b}, R_{c} and R_{d} are as defined in Table 1.
Table 262: This table discloses the 220 compounds T262.001 to T262.220 of the formula Ia, wherein R is CH₃, A is NO-(tetrahydropyran-4-yl), G is hydrogen and Rₐ, R_{b}, R_{c} and R_{d} are as defined in Table 1.
Table 263: This table discloses the 220 compounds T263.001 to T263.220 of the formula Ia, wherein R is hydrogen, A is NO-(tetrahydropyran-4-yl), G is hydrogen and Rₐ, R_{b}, R_{c} and R_{d} are as defined in Table 1.
Table 264: This table discloses the 220 compounds T264.001 to T264.220 of the formula Ia, wherein R is CH₂CH₃, A is NO-(tetrahydropyran-4-yl), G is hydrogen and Rₐ, R_{b}, R_{c} and R_{d} are as defined in Table 1.
Table 265: This table discloses the 220 compounds T265.001 to T265.220 of the formula Ia, wherein R is CH₂CF₃, A is NO-(tetrahydropyran-4-yl), G is hydrogen and Rₐ, R_{b}, R_{c} and R_{d} are as defined in Table 1.
Table 266: This table discloses the 220 compounds T266.001 to T266.220 of the formula Ia, wherein R is CH₂OCH₃, A is NO-(tetrahydropyran-4-yl), G is hydrogen and Rₐ, R_{b}, R_{c} and R_{d} are as defined in Table 1.
Table 267: This table discloses the 220 compounds T267.001 to T267.220 of the formula Ia, wherein R is CH₂CH₂OCH₃, A is NO-(tetrahydropyran-4-yl), G is hydrogen and Rₐ, R_{b}, R_{c} and R_{d} are as defined in Table 1.
Table 268: This table discloses the 220 compounds T268.001 to T268.220 of the formula Ia, wherein R is allyl, A is NO-(tetrahydropyran-4-yl), G is hydrogen and Rₐ, R_{b}, R_{c} and R_{d} are as defined in Table 1.
Table 269: This table discloses the 220 compounds T269.001 to T269.220 of the formula Ia, wherein R is propargyl, A is NO-(tetrahydropyran-4-yl), G is hydrogen and Rₐ, R_{b}, R_{c} and R_{d} are as defined in Table 1.
Table 270: This table discloses the 220 compounds T270.001 to T270.220 of the formula Ia, wherein R is benzyl, A is NO-(tetrahydropyran-4-yl), G is hydrogen and Rₐ, R_{b}, R_{c} and R_{d} are as defined in Table 1.
Table 271: This table discloses the 220 compounds T271.001 to T271.220 of the formula Ia, wherein R is CH₃, A is NO-(tetrahydrofuran-2-ylmethyl), G is hydrogen and Rₐ, R_{b}, R_{c} and R_{d} are as defined in Table 1.
Table 272: This table discloses the 220 compounds T272.001 to T272.220 of the formula Ia, wherein R is hydrogen, A is NO-(tetrahydrofuran-2-ylmethyl), G is hydrogen and Rₐ, R_{b}, R_{c} and R_{d} are as defined in Table 1.
Table 273: This table discloses the 220 compounds T273.001 to T273.220 of the formula Ia, wherein R is CH₂CH₃, A is NO-(tetrahydrofuran-2-ylmethyl), G is hydrogen and Rₐ, R_{b}, R_{c} and R_{d} are as defined in Table 1.
Table 274: This table discloses the 220 compounds T274.001 to T274.220 of the formula Ia, wherein R is CH₂CF₃, A is NO-(tetrahydrofuran-2-ylmethyl), G is hydrogen and Rₐ, R_{b}, R_{c} and R_{d} are as defined in Table 1.
Table 275: This table discloses the 220 compounds T275.001 to T275.220 of the formula Ia, wherein R is CH₂OCH₃, A is NO-(tetrahydrofuran-2-ylmethyl), G is hydrogen and Rₐ, R_{b}, R_{c} and R_{d} are as defined in Table 1.
Table 276: This table discloses the 220 compounds T276.001 to T276.220 of the formula Ia, wherein R is CH₂CH₂OCH₃, A is NO-(tetrahydrofuran-2-ylmethyl), G is hydrogen and Rₐ, R_{b}, R_{c} and R_{d} are as defined in Table 1.
Table 277: This table discloses the 220 compounds T277.001 to T277.220 of the formula Ia, wherein R is allyl, A is NO-(tetrahydrofuran-2-ylmethyl), G is hydrogen and Rₐ, R_{b}, R_{c} and R_{d} are as defined in Table 1.
Table 278: This table discloses the 220 compounds T278.001 to T278.220 of the formula Ia, wherein R is propargyl, A is NO-(tetrahydrofuran-2-ylmethyl), G is hydrogen and Rₐ, R_{b}, R_{c} and R_{d} are as defined in Table 1.
Table 279: This table discloses the 220 compounds T279.001 to T279.220 of the formula Ia, wherein R is benzyl, A is NO-(tetrahydrofuran-2-ylmethyl), G is hydrogen and Rₐ, R_{b}, R_{c} and R_{d} are as defined in Table 1.
Table 280: This table discloses the 220 compounds T280.001 to T280.220 of the formula Ia, wherein R is CH₃, A is NO-(tetrahydrofuran-3-ylmethyl), G is hydrogen and Rₐ, R_{b}, R_{c} and R_{d} are as defined in Table 1.
Table 281: This table discloses the 220 compounds T281.001 to T281.220 of the formula Ia, wherein R is hydrogen, A is NO-(tetrahydrofuran-3-ylmethyl), G is hydrogen and Rₐ, R_{b}, R_{c} and R_{d} are as defined in Table 1.
Table 282: This table discloses the 220 compounds T282.001 to T282.220 of the formula Ia, wherein R is CH₂CH₃, A is NO-(tetrahydrofuran-3-ylmethyl), G is hydrogen and Rₐ, R_{b}, R_{c} and R_{d} are as defined in Table 1.
Table 283: This table discloses the 220 compounds T283.001 to T283.220 of the formula Ia, wherein R is CH₂CF₃, A is NO-(tetrahydrofuran-3-ylmethyl), G is hydrogen and Rₐ, R_{b}, R_{c} and R_{d} are as defined in Table 1.
Table 284: This table discloses the 220 compounds T284.001 to T284.220 of the formula Ia, wherein R is CH₂OCH₃, A is NO-(tetrahydrofuran-3-ylmethyl), G is hydrogen and Rₐ, R_{b}, R_{c} and R_{d} are as defined in Table 1.
Table 285: This table discloses the 220 compounds T285.001 to T285.220 of the formula Ia, wherein R is CH₂CH₂OCH₃, A is NO-(tetrahydrofuran-3-ylmethyl), G is hydrogen and Rₐ, R_{b}, R_{c} and R_{d} are as defined in Table 1.
Table 286: This table discloses the 220 compounds T286.001 to T286.220 of the formula Ia, wherein R is allyl, A is NO-(tetrahydrofuran-3-ylmethyl), G is hydrogen and Rₐ, R_{b}, R_{c} and R_{d} are as defined in Table 1.
Table 287: This table discloses the 220 compounds T287.001 to T287.220 of the formula Ia, wherein R is propargyl, A is NO-(tetrahydrofuran-3-ylmethyl), G is hydrogen and Rₐ, R_{b}, R_{c} and R_{d} are as defined in Table 1.
Table 288: This table discloses the 220 compounds T288.001 to T288.220 of the formula Ia, wherein R is benzyl, A is NO-(tetrahydrofuran-3-ylmethyl), G is hydrogen and Rₐ, R_{b}, R_{c} and R_{d} are as defined in Table 1.
Table 289: This table discloses the 220 compounds T289.001 to T289.220 of the formula Ia, wherein R is CH₃, A is NO-(tetrahydropyran-4-ylmethyl), G is hydrogen and Rₐ, R_{b}, R_{c} and R_{d} are as defined in Table 1.
Table 290: This table discloses the 220 compounds T290.001 to T290.220 of the formula Ia, wherein R is hydrogen, A is NO-(tetrahydropyran-4-ylmethyl), G is hydrogen and Rₐ, R_{b}, R_{c} and R_{d} are as defined in Table 1.
Table 291: This table discloses the 220 compounds T291.001 to T291.220 of the formula Ia, wherein R is CH₂CH₃, A is NO-(tetrahydropyran-4-ylmethyl), G is hydrogen and Rₐ, R_{b}, R_{c} and R_{d} are as defined in Table 1.
Table 292: This table discloses the 220 compounds T292.001 to T292.220 of the formula Ia, wherein R is CH₂CF₃, A is NO-(tetrahydropyran-4-ylmethyl), G is hydrogen and Rₐ, R_{b}, R_{c} and R_{d} are as defined in Table 1.
Table 293: This table discloses the 220 compounds T293.001 to T293.220 of the formula Ia, wherein R is CH₂OCH₃, A is NO-(tetrahydropyran-4-ylmethyl), G is hydrogen and Rₐ, R_{b}, R_{c} and R_{d} are as defined in Table 1.
Table 294: This table discloses the 220 compounds T294.001 to T294.220 of the formula Ia, wherein R is CH₂CH₂OCH₃, A is NO-(tetrahydropyran-4-ylmethyl), G is hydrogen and Rₐ, R_{b}, R_{c} and R_{d} are as defined in Table 1.
Table 295: This table discloses the 220 compounds T295.001 to T295.220 of the formula Ia, wherein R is allyl, A is NO-(tetrahydropyran-4-ylmethyl), G is hydrogen and Rₐ, R_{b}, R_{c} and R_{d} are as defined in Table 1.
Table 296: This table discloses the 220 compounds T296.001 to T296.220 of the formula Ia, wherein R is propargyl, A is NO-(tetrahydropyran-4-ylmethyl), G is hydrogen and Rₐ, R_{b}, R_{c} and R_{d} are as defined in Table 1.
Table 297: This table discloses the 220 compounds T297.001 to T297.220 of the formula Ia, wherein R is benzyl, A is NO-(tetrahydropyran-4-ylmethyl), G is hydrogen and Rₐ, R_{b}, R_{c} and R_{d} are as defined in Table 1.
Table 298: This table discloses the 220 compounds T298.001 to T298.220 of the formula Ia, wherein R is CH₃, A is NO-cyclohexyl, G is hydrogen and Rₐ, R_{b}, R_{c} and R_{d} are as defined in Table 1.
Table 299: This table discloses the 220 compounds T299.001 to T299.220 of the formula Ia, wherein R is hydrogen, A is NO-cyclohexyl, G is hydrogen and Rₐ, R_{b}, R_{c} and R_{d} are as defined in Table 1.
Table 300: This table discloses the 220 compounds T300.001 to T300.220 of the formula Ia, wherein R is CH₂CH₃, A is NO-cyclohexyl, G is hydrogen and Rₐ, R_{b}, R_{c} and R_{d} are as defined in Table 1.
Table 301: This table discloses the 220 compounds T301.001 to T301.220 of the formula Ia, wherein R is CH₂CF₃, A is NO-cyclohexyl, G is hydrogen and Rₐ, R_{b}, R_{c} and R_{d} are as defined in Table 1.
Table 302: This table discloses the 220 compounds T302.001 to T302.220 of the formula Ia, wherein R is CH₂OCH₃, A is NO-cyclohexyl, G is hydrogen and Rₐ, R_{b}, R_{c} and R_{d} are as defined in Table 1.
Table 303: This table discloses the 220 compounds T303.001 to T303.220 of the formula Ia, wherein R is CH₂CH₂OCH₃, A is NO-cyclohexyl, G is hydrogen and Rₐ, R_{b}, R_{c} and R_{d} are as defined in Table 1.
Table 304: This table discloses the 220 compounds T304.001 to T304.220 of the formula Ia, wherein R is allyl, A is NO-cyclohexyl, G is hydrogen and Rₐ, R_{b}, R_{c} and R_{d} are as defined in Table 1.
Table 305: This table discloses the 220 compounds T305.001 to T305.220 of the formula Ia, wherein R is propargyl, A is NO-cyclohexyl, G is hydrogen and Rₐ, R_{b}, R_{c} and R_{d} are as defined in Table 1.
Table 306: This table discloses the 220 compounds T306.001 to T306.220 of the formula Ia, wherein R is benzyl, A is NO-cyclohexyl, G is hydrogen and Rₐ, R_{b}, R_{c} and R_{d} are as defined in Table 1.
Table 307: This table discloses the 220 compounds T307.001 to T307.220 of the formula Ia, wherein R is CH₃, A is NO-(furan-2-ylmethyl), G is hydrogen and Rₐ, R_{b}, R_{c} and R_{d} are as defined in Table 1.
Table 308: This table discloses the 220 compounds T308.001 to T308.220 of the formula Ia, wherein R is hydrogen, A is NO-(furan-2-ylmethyl), G is hydrogen and Rₐ, R_{b}, R_{c} and R_{d} are as defined in Table 1.
Table 309: This table discloses the 220 compounds T309.001 to T309.220 of the formula Ia, wherein R is CH₂CH₃, A is NO-(furan-2-ylmethyl), G is hydrogen and Rₐ, R_{b}, R_{c} and R_{d} are as defined in Table 1.
Table 310: This table discloses the 220 compounds T310.001 to T310.220 of the formula Ia, wherein R is CH₂CF₃, A is NO-(furan-2-ylmethyl), G is hydrogen and Rₐ, R_{b}, R_{c} and R_{d} are as defined in Table 1.
Table 311: This table discloses the 220 compounds T311.001 to T311.220 of the formula Ia, wherein R is CH₂OCH₃, A is NO-(furan-2-ylmethyl), G is hydrogen and Rₐ, R_{b}, R_{c} and R_{d} are as defined in Table 1.
Table 312: This table discloses the 220 compounds T312.001 to T312.220 of the formula Ia, wherein R is CH₂CH₂OCH₃, A is NO-(furan-2-ylmethyl), G is hydrogen and Rₐ, R_{b}, R_{c} and R_{d} are as defined in Table 1.
Table 313: This table discloses the 220 compounds T313.001 to T313.220 of the formula Ia, wherein R is allyl, A is NO-(furan-2-ylmethyl), G is hydrogen and Rₐ, R_{b}, R_{c} and R_{d} are as defined in Table 1.
Table 314: This table discloses the 220 compounds T314.001 to T314.220 of the formula Ia, wherein R is propargyl, A is NO-(furan-2-ylmethyl), G is hydrogen and Rₐ, R_{b}, R_{c} and R_{d} are as defined in Table 1.
Table 315: This table discloses the 220 compounds T315.001 to T315.220 of the formula Ia, wherein R is benzyl, A is NO-(furan-2-ylmethyl), G is hydrogen and Rₐ, R_{b}, R_{c} and R_{d} are as defined in Table 1.
Table 316: This table discloses the 220 compounds T316.001 to T316.220 of the formula Ia, wherein R is CH₃, A is NO-(furan-3-ylmethyl), G is hydrogen and Rₐ, R_{b}, R_{c} and R_{d} are as defined in Table 1.
Table 317: This table discloses the 220 compounds T317.001 to T317.220 of the formula Ia, wherein R is hydrogen, A is NO-(furan-3-ylmethyl), G is hydrogen and Rₐ, R_{b}, R_{c} and R_{d} are as defined in Table 1.
Table 318: This table discloses the 220 compounds T318.001 to T318.220 of the formula Ia, wherein R is CH₂CH₃, A is NO-(furan-3-ylmethyl), G is hydrogen and Rₐ, R_{b}, R_{c} and R_{d} are as defined in Table 1.
Table 319: This table discloses the 220 compounds T319.001 to T319.220 of the formula Ia, wherein R is CH₂CF₃, A is NO-(furan-3-ylmethyl), G is hydrogen and Rₐ, R_{b}, R_{c} and R_{d} are as defined in Table 1.
Table 320: This table discloses the 220 compounds T320.001 to T320.220 of the formula Ia, wherein R is CH₂OCH₃, A is NO-(furan-3-ylmethyl), G is hydrogen and Rₐ, R_{b}, R_{c} and R_{d} are as defined in Table 1.
Table 321: This table discloses the 220 compounds T321.001 to T321.220 of the formula Ia, wherein R is CH₂CH₂OCH₃, A is NO-(furan-3-ylmethyl), G is hydrogen and Rₐ, R_{b}, R_{c} and R_{d} are as defined in Table 1.
Table 322: This table discloses the 220 compounds T322.001 to T322.220 of the formula Ia, wherein R is allyl, A is NO-(furan-3-ylmethyl), G is hydrogen and Rₐ, R_{b}, R_{c} and R_{d} are as defined in Table 1.
Table 323: This table discloses the 220 compounds T323.001 to T323.220 of the formula Ia, wherein R is propargyl, A is NO-(furan-3-ylmethyl), G is hydrogen and Rₐ, R_{b}, R_{c} and R_{d} are as defined in Table 1.
Table 324: This table discloses the 220 compounds T324.001 to T324.220 of the formula Ia, wherein R is benzyl, A is NO-(furan-3-ylmethyl), G is hydrogen and Rₐ, R_{b}, R_{c} and R_{d} are as defined in Table 1.
Table 325: This table discloses the 220 compounds T325.001 to T325.220 of the formula Ia, wherein R is CH₃, A is NOCH₂CH₂SCH₃, G is hydrogen and Rₐ, R_{b}, R_{c} and R_{d} are as defined in Table 1.
Table 326: This table discloses the 220 compounds T326.001 to T326.220 of the formula Ia, wherein R is hydrogen, A is NOCH₂CH₂SCH₃, G is hydrogen and Rₐ, R_{b}, R_{c} and R_{d} are as defined in Table 1.
Table 327: This table discloses the 220 compounds T327.001 to T327.220 of the formula Ia, wherein R is CH₂CH₃, A is NOCH₂CH₂SCH₃, G is hydrogen and Rₐ, R_{b}, R_{c} and R_{d} are as defined in Table 1.
Table 328: This table discloses the 220 compounds T328.001 to T328.220 of the formula Ia, wherein R is CH₂CF₃, A is NOCH₂CH₂SCH₃, G is hydrogen and Rₐ, R_{b}, R_{c} and R_{d} are as defined in Table 1.
Table 329: This table discloses the 220 compounds T329.001 to T329.220 of the formula Ia, wherein R is CH₂OCH₃, A is NOCH₂CH₂SCH₃, G is hydrogen and Rₐ, R_{b}, R_{c} and R_{d} are as defined in Table 1.
Table 330: This table discloses the 220 compounds T330.001 to T330.220 of the formula Ia, wherein R is CH₂CH₂OCH₃, A is NOCH₂CH₂SCH₃, G is hydrogen and Rₐ, R_{b}, R_{c} and R_{d} are as defined in Table 1.
Table 331: This table discloses the 220 compounds T331.001 to T331.220 of the formula Ia, wherein R is allyl, A is NOCH₂CH₂SCH₃, G is hydrogen and Rₐ, R_{b}, R_{c} and R_{d} are as defined in Table 1.
Table 332: This table discloses the 220 compounds T332.001 to T332.220 of the formula Ia, wherein R is propargyl, A is NOCH₂CH₂SCH₃, G is hydrogen and Rₐ, R_{b}, R_{c} and R_{d} are as defined in Table 1.
Table 333: This table discloses the 220 compounds T333.001 to T333.220 of the formula Ia, wherein R is benzyl, A is NOCH₂CH₂SCH₃, G is hydrogen and Rₐ, R_{b}, R_{c} and R_{d} are as defined in Table 1.

The compounds according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i.e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

The compounds of formula I can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for

example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula I include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera littoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locusta migratoria (locust), Chortiocetes terminifera (locust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blattella germanica (cockroach), Periplaneta americana (cockroach), Blatta orientalis (cockroach), termites of the Mastotermitidae (for example Mastotermes spp.), the Kalotermitidae (for example Neotermes spp.), the Rhinotermitidae (for example Coptotermes formosanus, Reticulitermes flavipes, R. speratu, R. virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp. (citrus nematodes), Haemonchus contortus (barber pole worm), Caenorhabditis elegans Jvinegar eelworm), Trichostrongylus spp. (gastro intestinal nematodes) and Deroceras reticulatum (slug).

Further examples of the above mentioned pests are:

from the order Acarina, for example,
Acarus siro, Aceria sheldoni, Aculus schlechtendali, Amblyomma spp., Argas spp., Boophilus spp., Brevipalpus spp., Bryobia praetiosa, Calipitrimerus spp., Chorioptes spp., Dermanyssus gallinae, Eotetranychus carpini, Eriophyes spp., Hyalomma spp., Ixodes spp., Olygonychus pratensis, Ornithodoros spp., Panonychus spp., Phyllocoptruta oleivora, Polyphagotarsonemus latus, Psoroptes spp., Rhipicephalus spp., Rhizoglyphus spp., Sarcoptes spp., Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Anthonomus spp., Atomaria linearis, Chaetocnema tibialis, Cosmopolites spp., Curculio spp., Dermestes spp., Diabrotica spp., Epilachna spp., Eremnus spp., Leptinotarsa decemLineata, Lissorhoptrus spp., Melolontha spp., Orycaephilus spp., Otiorhynchus spp., Phlyctinus spp., Popillia spp., Psylliodes spp., Rhizopertha spp., Scarabeidae, Sitophilus spp., Sitotroga spp., Tenebrio spp., Tribolium spp. and Trogoderma spp.;
from the order Diptera, for example,
Aedes spp., Antherigona soccata, Bibio hortulanus, Calliphora erythrocephala, Ceratitis spp., Chrysomyia spp., Culex spp., Cuterebra spp., Dacus spp., Drosophila melanogaster, Fannia spp., Gastrophilus spp., Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis pomonella, Sciara spp., Stomoxys spp., Tabanus spp., Tannia spp. and Tipula spp.;
from the order Heteroptera, for example,
Cimex spp., Distantiella theobroma, Dysdercus spp., Euchistus spp., Eurygaster spp., Leptocorisa spp., Nezara spp., Piesma spp., Rhodnius spp., Sahlbergella singularis, Scotinophara spp. and Triatoma spp.;
from the order Homoptera, for example,
Aleurothrixus floccosus, Aleyrodes brassicae, Aonidiella spp., Aphididae, Aphis spp., Aspidiotus spp., Bemisia tabaci, Ceroplaster spp., Chrysomphalus aonidium, Chrysomphalus dictyospermi, Coccus hesperidum, Empoasca spp., Eriosoma larigerum, Erythroneura spp., Gascardia spp., Laodelphax spp., Lecanium corni, Lepidosaphes spp., Macrosiphus spp., Myzus spp., Nephotettix spp., Nilaparvata spp., Parlatoria spp., Pemphigus spp., Planococcus spp., Pseudaulacaspis spp., Pseudococcus spp., Psylla spp., Pulvinaria aethiopica, Quadraspidiotus spp., Rhopalosiphum spp., Saissetia spp., Scaphoideus spp., Schizaphis spp., Sitobion spp., Trialeurodes vaporariorum, Trioza erytreae and Unaspis citri;
from the order Hymenoptera, for example,
Acromyrmex, Atta spp., Cephus spp., Diprion spp., Diprionidae, Gilpinia polytoma, Hoplocampa spp., Lasius spp., Monomorium pharaonis, Neodiprion spp., Solenopsis spp. and Vespa spp.;
from the order lsoptera, for example,

### Reticulitermes spp.;

from the order Lepidoptera, for example,
Acleris spp., Adoxophyes spp., Aegeria spp., Agrotis spp., Alabama argillaceae, Amylois spp., Anticarsia gemmatalis, Archips spp., Argyrotaenia spp., Autographa spp., Busseola fusca, Cadra cautella, Carposina nipponensis, Chilo spp., Choristoneura spp., Clysia ambiguella, Cnaphalocrocis spp., Cnephasia spp., Cochylis spp., Coleophora spp., Crocidolomia binotalis, Cryptophlebia leucotreta, Cydia spp., Diatraea spp., Diparopsis castanea, Earias spp., Ephestia spp., Eucosma spp., Eupoecilia ambiguella, Euproctis spp., Euxoa spp., Grapholita spp., Hedya nubiferana, Heliothis spp., Hellula undalis, Hyphantria cunea, Keiferia lycopersicella, Leucoptera scitella, Lithocollethis spp., Lobesia botrana, Lymantria spp., Lyonetia spp., Malacosoma spp., Mamestra brassicae, Manduca sexta, Operophtera spp., Ostrinia nubilalis, Pammene spp., Pandemis spp., Panolis flammea, Pectinophora gossypiela, Phthorimaea operculella, Pieris rapae, Pieris spp., Plutella xylostella, Prays spp., Scirpophaga spp., Sesamia spp., Sparganothis spp., Spodoptera spp., Synanthedon spp., Thaumetopoea spp., Tortrix spp., Trichoplusia ni and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Periplaneta spp. and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Frankliniella spp., Hercinothrips spp., Scirtothrips aurantii, Taeniothrips spp., Thrips palmi and Thrips tabaci; and
from the order Thysanura, for example,
Lepisma saccharina.

The active ingredients according to the invention can be used for controlling, i.e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants and ornamentals.

The term “crops” is to be understood as including also crops that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady® and LibertyLink®.

The term “crops” is also to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by δ-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). Truncated toxins, for example a truncated CryIA(b), are known. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 03/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Transgenic plants containing one or more genes that code for an insecticidal resistance and express one or more toxins are known and some of them are commercially available. Examples of such plants are: YieldGard® (maize variety that expresses a CryIA(b) toxin); YieldGard Rootworm® (maize variety that expresses a CryIIIB(b1) toxin); YieldGard Plus® (maize variety that expresses a CryIA(b) and a CryIIIB(b1) toxin); Starlink® (maize variety that expresses a Cry9(c) toxin); Herculex I® (maize variety that expresses a CryIF(a2) toxin and the enzyme phosphinothricine N-acetyltransferase (PAT) to achieve tolerance to the herbicide glufosinate ammonium); NuCOTN 33B® (cotton variety that expresses a CryIA(c) toxin); Bollgard I® (cotton variety that expresses a CryIA(c) toxin); Bollgard II® (cotton variety that expresses a CryIA(c) and a CryIIA(b) toxin); VIPCOT® (cotton variety that expresses a VIP toxin); NewLeaf® (potato variety that expresses a CryIIIA toxin); Nature-Gard® Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated CryIA(b) toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a CryIA(b) toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified CryIIIA toxin. This toxin is Cry3A055 modified by insertion of a cathepsin-D-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a CryIIIB(b1) toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein CryIF for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a CryIA(b) toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit and Nachhaltigkeit, Zentrum BATS, Clarastrasse 13, 4058 Basel, Switzerland) Report 2003.

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Crops that exhibit enhanced yield or quality include those with improved flowering or fruit ripening properties (such as delayed ripening); modified oil, starch, amino acid, fatty acid, vitamin, phenolic or other content (such as Vistive™ soybean variety); enhanced nutrient utilisation (such as improved nitrogen assimilation); and enhanced quality plant product (such as higher quality cotton fibre).

Further areas of use of the compounds and compositions according to the invention are the protection of stored goods and storerooms and the protection of raw materials, such as wood, textiles, floor coverings or buildings, and also in the hygiene sector, especially the protection of humans, domestic animals and productive livestock against pests of the mentioned type.

In the hygiene sector, the compounds and compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compounds and compositions according to the invention are also suitable for protecting against insect infestation in the case of materials such as wood, textiles, plastics, adhesives, glues, paints, paper and card, leather, floor coverings and buildings. The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The invention therefore provides a method of combating and controlling insects, acarines, nematodes or molluscs which comprises applying an insecticidally, acaricidally, nematicidally or molluscicidally effective amount of a compound of formula I, or a composition containing a compound of formula I, to a pest, a locus of pest, or to a plant susceptible to attack by a pest, The compounds of formula I are preferably used against insects or acarines.

The term “plant” as used herein includes seedlings, bushes and trees.

The invention therefore also relates to pesticidal compositions such as emulsifiable concentrates, suspension concentrates, directly sprayable or dilutable solutions, spreadable pastes, dilute emulsions, soluble powders, dispersible powders, wettable powders, dusts, granules or encapsulations in polymeric substances, which comprise—at least—one of the active ingredients according to the invention and which are to be selected to suit the intended aims and the prevailing circumstances.

In these compositions, the active ingredient is employed in pure form, a solid active ingredient for example in a specific particle size, or, preferably, together with—at least—one of the auxiliaries conventionally used in the art of formulation, such as extenders, for example solvents or solid carriers, or such as surface-active compounds (surfactants).

Examples of suitable solvents are: unhydrogenated or partially hydrogenated aromatic hydrocarbons, preferably the fractions C8 to C12 of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorbtive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues.

Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature.

Suitable non-ionic surfactants are, especially, polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopo [symbol] lypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene [symbol] glycol unit. Examples which may be mentioned are nonylphenoxypolyethoxyethanol, castor oil polyglycol ether, polypropylene glycol/polyethylene oxide adducts, tributylpheno [symbol] ixypolyethoxyethanol, polyethylene glycol or octylphenoxypolyethoxyethanol. Also suitable are fatty acid esters of polyoxyethylene sorbitan, such as polyoxyethylene sorbitan trioleate.

The cationic surfactants are, especially, quarternary ammonium salts which generally have at least one alkyl radical of approximately 8 to approximately 22 C atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethyhammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutyl [symbol] inaphthalenesulfonic acid or of a naphthalenesulfonic acid/formaldehyde condensate. Also possible are, furthermore, suitable phosphates, such as salts of the phosphoric ester of a p-nonylphenol/(4-14)ethylene oxide adduct, or phospholipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl) phosphate, tris-n-octyl phosphate and tris-butoxyethyl phosphate, where tris-(2-ethylhexyl) phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₃ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037373.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Syngenta AG, CH) and Actipron® (BP Oil UK Limited, GB).

The said surface-active substances may also be used in the formulations alone, that is to say without oil additives.

Furthermore, the addition of an organic solvent to the oil additive/surfactant mixture can contribute to a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

As a rule, the compositions comprise 0.1 to 99%, especially 0.1 to 95%, of active ingredient of thre formula land 1 to 99.9%, especially 5 to 99.9%, of at least one solid or liquid adjuvant, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient. Preferred compositions are composed in particular as follows (%=percent by weight):

### Emulsifiable Concentrates:

active ingredient: 1 to 95%, preferably 5 to 50%, more preferably 5 to 20%
surfactant: 1 to 30%, preferably 10 to 20%
solvent: 5 to 98%, preferably 70 to 85%

### Dusts:

active ingredient: 0.1 to 10%, preferably 2 to 5%,
solid carrier: 99.9 to 90%, preferably 99.9 to 99%

### Suspension Concentrates:

active ingredient: 5 to 75%, preferably 10 to 50%, more preferably 10 to 40%
water: 94 to 24%, preferably 88 to 30%
surfactant: 1 to 40%, preferably 2 to 30%

### Oil-Based Suspension Concentrates:

active ingredient: 2 to 75%, preferably 5 to 50%, more preferably 10 to 25%
oil: 94 to 24%, preferably 88 to 30%
surfactant: 1 to 40%, preferably 2 to 30%

### Wettable Powders:

active ingredient: 0.5 to 90%, preferably 1 to 80%, more preferably 25 to 75%
surfactant: 0.5 to 20%, preferably 1 to 15%
solid carrier: 5 to 99%, preferably 15 to 98%

### Granulates:

active ingredient: 0.5 to 30%, preferably 3 to 25%, more preferably 3 to 15%
solid carrier: 99.5 to 70%, preferably 97 to 85%

Preferably, the term “active ingredient” refers to one of the compounds selected from Tables 1 to 333 shown above. It also refers to mixtures of the compound of formula I, in particular a compound selected from said Tables 1 to 333, with other insecticides, fungicides, herbicides, safeners, adjuvants and the like, which mixtures are specifically disclosed below.

The compositions can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers; fertilizers, in particular nitrogen containing fertilizers such as ammonium nitrates and urea as described in WO08/017388, which can enhance the efficacy of the inventive compounds; or other active ingredients for achieving specific effects, for example ammonium or phosphonium salts, in particular halides, (hydrogen)sulphates, nitrates, (hydrogen)carbonates, citrates, tartrates, formiates and acetates, as described in WO07/068427 and WO07/068428, which also can enhance the efficacy of the inventive compounds and which can be used in combination with penetration enhancers such as alkoxalated fatty acids; bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compositions according to the invention are also suitable for the protection of plant propagation material, for example seeds, such as fruit, tubers or kernels, or nursery plants, against pests of the abovementioned type. The propagation material can be treated with the compositions prior to planting, for example seed can be treated prior to sowing. Alternatively, the compositions can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention.

Further methods of application of the compositions according to the invention comprise drip application onto the soil, dipping of parts of plants such as roots bulbs or tubers, drenching the soil, as well as soil injection. These methods are known in the art.

In order to apply a compound of formula I as an insecticide, acaricide, nematicide or molluscicide to a pest, a locus of pest, or to a plant susceptible to attack by a pest, a compound of formula I is usually formulated into a composition which includes, in addition to the compound of formula I, a suitable inert diluent or carrier and, optionally, a formulation adjuvant in form of a surface active agent (SFA) as described herein or, for example, in EP-B-1062217. SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula I. The composition is generally used for the control of pests such that a compound of formula I is applied at a rate of from 0.1 g to 10 kg per hectare, preferably from 1 g to 6 kg per hectare, more preferably from 1 g to 1 kg per hectare.

When used in a seed dressing, a compound of formula I is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal or molluscicidal composition comprising an insecticidally, acaricidally, nematicidally or molluscicidally effective amount of a compound of formula I and a suitable carrier or diluent therefor.

In a still further aspect the invention provides a method of combating and controlling pests at a locus which comprises treating the pests or the locus of the pests with an insecticidally, acaricidally, nematicidally or molluscicidally effective amount of a composition comprising a compound of formula I.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed treatment formulations. The formulation type chosen in any instance will depend upon the particular purpose en-visaged and the physical, chemical and biological properties of the compound of formula I.

Dustable powders (DP) may be prepared by mixing a compound of formula I with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula I with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula I with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula I and one or more powdered solid diluents or carriers, or from pre-formed blank granules by absorbing a compound of formula I (or a solution thereof, in a suitable agent) in a porous granular material (such as pumice, attapulgite clays, fuller's earth, kieselguhr, diatomaceous earths or ground corn cobs) or by adsorbing a compound of formula I (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula I in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula I in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula I either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula I is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula I. SCs may be prepared by ball or bead milling the solid compound of formula I in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula I may be dry milled and added to water, containing agents hereinbefore described, to produce the desired end product.

Oil-based suspension concentrate (OD) may be prepared similarly by suspending finely divided insoluble solid particles of a compound of formula I in an organic fluid (for example at least one mineral oil or vegetable oil). ODs may further comprise at least one penetration promoter (for example an alcohol ethoxylate or a related compound), at least one non-ionic surfactants and/or at least one anionic surfactant, and optionally at least one additive from the group of emulsifiers, foam-inhibiting agents, preservatives, anti-oxidants, dyestuffs, and/or inert filler materials. An OD is intended and suitable for dilution with water before use to produce a spray solution with sufficient stability to allow spray application through appropriate equipment.

Aerosol formulations comprise a compound of formula I and a suitable propellant (for example n-butane). A compound of formula I may also be dissolved or dispersed in a suitable medium (for example water or a water miscible liquid, such as n-propanol) to provide compositions for use in non-pressurised, hand-actuated spray pumps.

A compound of formula I may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula I and, optionally, a carrier or diluent therefor. The polymeric shell may be produced by either an interfacial polycondensation reaction or by a coacervation procedure. The compositions may provide for controlled release of the compound of formula I and they may be used for seed treatment. A compound of formula I may also be formulated in a biodegradable polymeric matrix to provide a slow, controlled release of the compound.

A compound of formula I may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC, OD and DC compositions described above. Compositions for treating seed may include an agent for assisting the adhesion of the composition to the seed (for example a mineral oil or a film-forming barrier).

A composition of the present invention may include one or more additives to improve the biological performance of the composition (for example by improving wetting, retention or distribution on surfaces; resistance to rain on treated surfaces; or uptake or mobility of a compound of formula I). Such additives include surface active agents (SFAs), spray additives based on oils, for example certain mineral oils, vegetable oils or natural plant oils (such as soy bean and rape seed oil), and blends of these with other bio-enhancing adjuvants (ingredients which may aid or modify the action of a compound of formula I). Increasing the effect of a compound of formula I may for example be achieved by adding ammonium and/or phosphonium salts, and/or optionally at least one penetration promotor such as fatty alcohol alkoxylates (for example rape oil methyl ester) or vegetable oil esters.

Wetting agents, dispersing agents and emulsifying agents may be surface active agents (SFAs) of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oeyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula I may be applied by any of the known means of applying pesticidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula I may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ODs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula I (for example 0.0001 to 10%, by weight) depending upon the purpose for which they are to be used.

A compound of formula I may be used in mixtures with fertilisers (for example nitrogen-, potassium- or phosphorus-containing fertilisers, and more particularly ammonium nitrate and/or urea fertilizers). Suitable formulation types include granules of fertiliser. The mixtures suitably contain up to 25% by weight of the compound of formula I.

The invention therefore also provides a fertiliser composition comprising a fertiliser and a compound of formula I.

The compositions of this invention may contain other compounds having biological activity, for example micronutrients or compounds having fungicidal activity or which possess plant growth regulating, herbicidal, safening, insecticidal, nematicidal or acaricidal activity.

The compound of formula I may be the sole active ingredient of the composition or it may be admixed with one or more additional active ingredients such as a pesticide (insect, acarine, mollusc and nematode pesticide), fungicide, synergist, herbicide, safener or plant growth regulator where appropriate. The activity of the compositions according to the invention may thereby be broadened considerably and may have surprising advantages which can also be described, in a wider sense, as synergistic activity. An additional active ingredient may: provide a composition having a broader spectrum of activity or increased persistence at a locus; provide a composition demonstrating better plant/crop tolerance by reducing phytotoxicity; provide a composition controlling insects in their different development stages; synergise the activity or complement the activity (for example by increasing the speed of effect or overcoming repellency) of the compound of formula I; or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)-(1R,3S)-2,2-dimethyl-3-(2-oxothiolan-3-ylidenemethyl)cyclopropane carboxylate;
b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;
c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;
d) Benzoyl ureas, such as diflubenzuron, triflumuron, hexaflumuron, flufenoxuron or chlorfluazuron;
e) Organic tin compounds, such as cyhexatin, fenbutatin oxide or azocyclotin;
f) Pyrazoles, such as tebufenpyrad and fenpyroximate;
g) Macrolides, such as avermectins or milbemycins, for example abamectin, emamectin benzoate, ivermectin, milbemycin, or spinosad, spinetoram or azadirachtin;
h) Hormones or pheromones;
i) Organochlorine compounds such as endosulfan, benzene hexachloride, DDT, chlordane or dieldrin;
j) Amidines, such as chlordimeform or amitraz;
k) Fumigant agents, such as chloropicrin, dichloropropane, methyl bromide or metam;
l) Neonicotinoid compounds such as imidacloprid, thiacloprid, acetamiprid, clothianidin, nitenpyram, dinotefuran or thiamethoxam;
m) Diacylhydrazines, such as tebufenozide, chromafenozide or methoxyfenozide;
n) Diphenyl ethers, such as diofenolan or pyriproxifen;
o) lndoxacarb;

### p) Chlorfenapyr;

q) Pymetrozine or pyrifluquinazon;
r) Spirotetramat, spirodiclofen or spiromesifen;
s) Flubendiamide, chloranthraliniprole, or cyanthraniliprole;
t) Cyenopyrafen or cyflumetofen; or

### u) Sulfoxaflor.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

The following mixtures of the compounds of formula I with active ingredients are preferred, wherein, preferably, the term “COMPOUND OF FORMULA I” refers to a compound selected from the Tables 1 to 333:
an adjuvant selected from the group of substances consisting of an oil of vegetable or animal origin, a mineral oil, alkyl esters of such oils or mixtures of such oils, and petroleum oils (alternative name) (628)+COMPOUND OF FORMULA I,
an acaricide selected from the group of substances consisting of 1,1-bis(4-chloro-phenyl)-2-ethoxyethanol (IUPAC name) (910)+COMPOUND OF FORMULA I, 2,4-dichlorophenyl benzenesulfonate (IUPAC/Chemical Abstracts name) (1059)+COMPOUND OF FORMULA I, 2-fluoro-N-methyl-N-1-naphthylacetamide (IUPAC name) (1295)+COMPOUND OF FORMULA I, 4-chlorophenyl phenyl sulfone (IUPAC name) (981)+COMPOUND OF FORMULA I, abamectin (1)+COMPOUND OF FORMULA I, acequinocyl (3)+COMPOUND OF FORMULA I, acetoprole [CCN]+COMPOUND OF FORMULA I, acrinathrin (9)+COMPOUND OF FORMULA I, aldicarb (16)+COMPOUND OF FORMULA I, aldoxycarb (863)+COMPOUND OF FORMULA I, alpha-cypermethrin (202)+COMPOUND OF FORMULA I, amidithion (870)+COMPOUND OF FORMULA I, amidoflumet [CCN]+COMPOUND OF FORMULA I, amidothioate (872)+COMPOUND OF FORMULA I, amiton (875)+COMPOUND OF FORMULA I, amiton hydrogen oxalate (875)+COMPOUND OF FORMULA I, amitraz (24)+COMPOUND OF FORMULA I, aramite (881)+COMPOUND OF FORMULA I, arsenous oxide (882)+COMPOUND OF FORMULA I, AVI 382 (compound code)+COMPOUND OF FORMULA I, AZ 60541 (compound code)+COMPOUND OF FORMULA I, azinphos-ethyl (44)+COMPOUND OF FORMULA I, azinphos-methyl (45)+COMPOUND OF FORMULA I, azobenzene (IUPAC name) (888)+COMPOUND OF FORMULA I, azocyclotin (46)+COMPOUND OF FORMULA I, azothoate (889)+COMPOUND OF FORMULA I, benomyl (62)+COMPOUND OF FORMULA I, benoxafos (alternative name) [CCN]+COMPOUND OF FORMULA I, benzoximate (71)+COMPOUND OF FORMULA I, benzyl benzoate (IUPAC name) [CCN]+COMPOUND OF FORMULA I, bifenazate (74)+COMPOUND OF FORMULA I, bifenthrin (76)+COMPOUND OF FORMULA I, binapacryl (907)+COMPOUND OF FORMULA I, brofenvalerate (alternative name)+COMPOUND OF FORMULA I, bromocyclen (918)+COMPOUND OF FORMULA I, bromophos (920)+COMPOUND OF FORMULA I, bromophos-ethyl (921)+COMPOUND OF FORMULA I, bromopropylate (94)+COMPOUND OF FORMULA I, buprofezin (99)+COMPOUND OF FORMULA I, butocarboxim (103)+COMPOUND OF FORMULA I, butoxycarboxim (104)+COMPOUND OF FORMULA I, butylpyridaben (alternative name)+COMPOUND OF FORMULA I, calcium polysulfide (IUPAC name) (111)+COMPOUND OF FORMULA I, camphechlor (941)+COMPOUND OF FORMULA I, carbanolate (943)+COMPOUND OF FORMULA I, carbaryl (115)+COMPOUND OF FORMULA I, carbofuran (118)+COMPOUND OF FORMULA I, carbophenothion (947)+COMPOUND OF FORMULA I, CGA 50′439 (development code) (125)+COMPOUND OF FORMULA I, chinomethionat (126)+COMPOUND OF FORMULA I, chlorbenside (959)+COMPOUND OF FORMULA I, chlordimeform (964)+COMPOUND OF FORMULA I, chlordimeform hydrochloride (964)+COMPOUND OF FORMULA I, chlorfenapyr (130)+COMPOUND OF FORMULA I, chlorfenethol (968)+COMPOUND OF FORMULA I, chlorfenson (970)+COMPOUND OF FORMULA I, chlorfensulphide (971)+COMPOUND OF FORMULA I, chlorfenvinphos (131)+COMPOUND OF FORMULA I, chlorobenzilate (975)+COMPOUND OF FORMULA I, chloromebuform (977)+COMPOUND OF FORMULA I, chloromethiuron (978)+COMPOUND OF FORMULA I, chloropropylate (983)+COMPOUND OF FORMULA I, chlorpyrifos (145)+COMPOUND OF FORMULA I, chlorpyrifos-methyl (146)+COMPOUND OF FORMULA I, chlorthiophos (994)+COMPOUND OF FORMULA I, cinerin I (696)+COMPOUND OF FORMULA I, cinerin II (696)+COMPOUND OF FORMULA I, cinerins (696)+COMPOUND OF FORMULA I, clofentezine (158)+COMPOUND OF FORMULA I, closantel (alternative name) [CCN]+COMPOUND OF FORMULA I, coumaphos (174)+COMPOUND OF FORMULA I, crotamiton (alternative name) [CCN]+COMPOUND OF FORMULA I, crotoxyphos (1010)+COMPOUND OF FORMULA I, cufraneb (1013)+COMPOUND OF FORMULA I, cyanthoate (1020)+COMPOUND OF FORMULA I, cyenopyrafen [CCN]+COMPOUND OF FORMULA I, cyflumetofen (CAS Reg. No.: 400882-07-7)+COMPOUND OF FORMULA I, cyhalothrin (196)+COMPOUND OF FORMULA I, cyhexatin (199)+COMPOUND OF FORMULA I, cypermethrin (201)+COMPOUND OF FORMULA I, DCPM (1032)+COMPOUND OF FORMULA I, DDT (219)+COMPOUND OF FORMULA I, demephion (1037)+COMPOUND OF FORMULA I, demephion-O (1037)+COMPOUND OF FORMULA I, demephion-S (1037)+COMPOUND OF FORMULA I, demeton (1038)+COMPOUND OF FORMULA I, demeton-methyl (224)+COMPOUND OF FORMULA I, demeton-O (1038)+COMPOUND OF FORMULA I, demeton-O-methyl (224)+COMPOUND OF FORMULA I, demeton-S (1038)+COMPOUND OF FORMULA I, demeton-5-methyl (224)+COMPOUND OF FORMULA I, demeton-5-methylsulphon (1039)+COMPOUND OF FORMULA I, diafenthiuron (226)+COMPOUND OF FORMULA I, dialifos (1042)+COMPOUND OF FORMULA I, diazinon (227)+COMPOUND OF FORMULA I, dichlofluanid (230)+COMPOUND OF FORMULA I, dichlorvos (236)+COMPOUND OF FORMULA I, dicliphos (alternative name)+COMPOUND OF FORMULA I, dicofol (242)+COMPOUND OF FORMULA I, dicrotophos (243)+COMPOUND OF FORMULA I, dienochlor (1071)+COMPOUND OF FORMULA I, diflovidazin [CCN]+COMPOUND OF FORMULA I, dimefox (1081)+COMPOUND OF FORMULA I, dimethoate (262)+COMPOUND OF FORMULA I, dinactin (alternative name) (653)+COMPOUND OF FORMULA I, dinex (1089)+COMPOUND OF FORMULA I, dinex-diclexine (1089)+COMPOUND OF FORMULA I, dinobuton (269)+COMPOUND OF FORMULA I, dinocap (270)+COMPOUND OF FORMULA I, dinocap-4 [CCN]+COMPOUND OF FORMULA I, dinocap-6 [CCN]+COMPOUND OF FORMULA I, dinocton (1090)+COMPOUND OF FORMULA I, dino-penton (1092)+COMPOUND OF FORMULA I, dinosulfon (1097)+COMPOUND OF FORMULA I, dinoterbon (1098)+COMPOUND OF FORMULA I, dioxathion (1102)+COMPOUND OF FORMULA I, diphenyl sulfone (IUPAC name) (1103)+COMPOUND OF FORMULA I, disulfuram (alternative name) [CCN]+COMPOUND OF FORMULA I, disulfoton (278)+COMPOUND OF FORMULA I, DNOC (282)+COMPOUND OF FORMULA I, dofenapyn (1113)+COMPOUND OF FORMULA I, doramectin (alternative name) [CCN]+COMPOUND OF FORMULA I, endosulfan (294)+COMPOUND OF FORMULA I, endothion (1121)+COMPOUND OF FORMULA I, EPN (297)+COMPOUND OF FORMULA I, eprinomectin (alternative name) [CCN]+COMPOUND OF FORMULA I, ethion (309)+COMPOUND OF FORMULA I, ethoate-methyl (1134)+COMPOUND OF FORMULA I, etoxazole (320)+COMPOUND OF FORMULA I, etrimfos (1142)+COMPOUND OF FORMULA I, fenazaflor (1147)+COMPOUND OF FORMULA I, fenazaquin (328)+COMPOUND OF FORMULA I, fenbutatin oxide (330)+COMPOUND OF FORMULA I, fenothiocarb (337)+COMPOUND OF FORMULA I, fenpropathrin (342)+COMPOUND OF FORMULA I, fenpyrad (alternative name)+COMPOUND OF FORMULA I, fenpyroximate (345)+COMPOUND OF FORMULA I, fenson (1157)+COMPOUND OF FORMULA I, fentrifanil (1161)+COMPOUND OF FORMULA I, fenvalerate (349)+COMPOUND OF FORMULA I, fipronil (354)+COMPOUND OF FORMULA I, fluacrypyrim (360)+COMPOUND OF FORMULA I, fluazuron (1166)+COMPOUND OF FORMULA I, flubenzimine (1167)+COMPOUND OF FORMULA I, flucycloxuron (366)+COMPOUND OF FORMULA I, flucythrinate (367)+COMPOUND OF FORMULA I, fluenetil (1169)+COMPOUND OF FORMULA I, flufenoxuron (370)+COMPOUND OF FORMULA I, flumethrin (372)+COMPOUND OF FORMULA I, fluorbenside (1174)+COMPOUND OF FORMULA I, fluvalinate (1184)+COMPOUND OF FORMULA I, FMC 1137 (development code) (1185)+COMPOUND OF FORMULA I, formetanate (405)+COMPOUND OF FORMULA I, formetanate hydrochloride (405)+COMPOUND OF FORMULA I, formothion (1192)+COMPOUND OF FORMULA I, formparanate (1193)+COMPOUND OF FORMULA I, gamma-HCH (430)+COMPOUND OF FORMULA I, glyodin (1205)+COMPOUND OF FORMULA I, halfenprox (424)+COMPOUND OF FORMULA I, heptenophos (432)+COMPOUND OF FORMULA I, hexadecyl cyclopropanecarboxylate (IUPAC/Chemical Abstracts name) (1216)+COMPOUND OF FORMULA I, hexythiazox (441)+COMPOUND OF FORMULA I, IKA 2002 (CAS Reg. No.: 211923-74-9)+COMPOUND OF FORMULA I, iodomethane (IUPAC name) (542)+COMPOUND OF FORMULA I, isocarbophos (alternative name) (473)+COMPOUND OF FORMULA I, isopropyl O-(methoxyaminothiophosphoryl)salicylate (IUPAC name) (473)+COMPOUND OF FORMULA I, ivermectin (alternative name) [CCN]+COMPOUND OF FORMULA I, jasmolin I (696)+COMPOUND OF FORMULA I, jasmolin II (696)+COMPOUND OF FORMULA I, jodfenphos (1248)+COMPOUND OF FORMULA I, lindane (430)+COMPOUND OF FORMULA I, lufenuron (490)+COMPOUND OF FORMULA I, malathion (492)+COMPOUND OF FORMULA I, malonoben (1254)+COMPOUND OF FORMULA I, mecarbam (502)+COMPOUND OF FORMULA I, mephosfolan (1261)+COMPOUND OF FORMULA I, mesulfen (alternative name) [CCN]+COMPOUND OF FORMULA I, methacrifos (1266)+COMPOUND OF FORMULA I, methamidophos (527)+COMPOUND OF FORMULA I, methidathion (529)+COMPOUND OF FORMULA I, methiocarb (530)+COMPOUND OF FORMULA I, methomyl (531)+COMPOUND OF FORMULA I, methyl bromide (537)+COMPOUND OF FORMULA I, metolcarb (550)+COMPOUND OF FORMULA I, mevinphos (556)+COMPOUND OF FORMULA I, mexacarbate (1290)+COMPOUND OF FORMULA I, milbemectin (557)+COMPOUND OF FORMULA I, milbemycin oxime (alternative name) [CCN]+COMPOUND OF FORMULA I, mipafox (1293)+COMPOUND OF FORMULA I, monocrotophos (561)+COMPOUND OF FORMULA I, morphothion (1300)+COMPOUND OF FORMULA I, moxidectin (alternative name) [CCN]+COMPOUND OF FORMULA I, naled (567)+COMPOUND OF FORMULA I, NC-184 (compound code)+COMPOUND OF FORMULA I, NC-512 (compound code)+COMPOUND OF FORMULA I, nifluridide (1309)+COMPOUND OF FORMULA I, nikkomycins (alternative name) [CCN]+COMPOUND OF FORMULA I, nitrilacarb (1313)+COMPOUND OF FORMULA I, nitrilacarb 1:1 zinc chloride complex (1313)+COMPOUND OF FORMULA I, NNI-0101 (compound code)+COMPOUND OF FORMULA I, NNI-0250 (compound code)+COMPOUND OF FORMULA I, omethoate (594)+COMPOUND OF FORMULA I, oxamyl (602)+COMPOUND OF FORMULA I, oxydeprofos (1324)+COMPOUND OF FORMULA I, oxydisulfoton (1325)+COMPOUND OF FORMULA I, pp'-DDT (219)+COMPOUND OF FORMULA I, parathion (615)+COMPOUND OF FORMULA I, permethrin (626)+COMPOUND OF FORMULA I, petroleum oils (alternative name) (628)+COMPOUND OF FORMULA I, phenkapton (1330)+COMPOUND OF FORMULA I, phenthoate (631)+COMPOUND OF FORMULA I, phorate (636)+COMPOUND OF FORMULA I, phosalone (637)+COMPOUND OF FORMULA I, phosfolan (1338)+COMPOUND OF FORMULA I, phosmet (638)+COMPOUND OF FORMULA I, phosphamidon (639)+COMPOUND OF FORMULA I, phoxim (642)+COMPOUND OF FORMULA I, pirimiphos-methyl (652)+COMPOUND OF FORMULA I, polychloroterpenes (traditional name) (1347)+COMPOUND OF FORMULA I, polynactins (alternative name) (653)+COMPOUND OF FORMULA I, proclonol (1350)+COMPOUND OF FORMULA I, profenofos (662)+COMPOUND OF FORMULA I, promacyl (1354)+COMPOUND OF FORMULA I, propargite (671)+COMPOUND OF FORMULA I, propetamphos (673)+COMPOUND OF FORMULA I, propoxur (678)+COMPOUND OF FORMULA I, prothidathion (1360)+COMPOUND OF FORMULA I, prothoate (1362)+COMPOUND OF FORMULA I, pyrethrin I (696)+COMPOUND OF FORMULA I, pyrethrin II (696)+COMPOUND OF FORMULA I, pyrethrins (696)+COMPOUND OF FORMULA I, pyridaben (699)+COMPOUND OF FORMULA I, pyridaphenthion (701)+COMPOUND OF FORMULA I, pyrimidifen (706)+COMPOUND OF FORMULA I, pyrimitate (1370)+COMPOUND OF FORMULA I, quinalphos (711)+COMPOUND OF FORMULA I, quintiofos (1381)+COMPOUND OF FORMULA I, R-1492 (development code) (1382)+COMPOUND OF FORMULA I, RA-17 (development code) (1383)+COMPOUND OF FORMULA I, rotenone (722)+COMPOUND OF FORMULA I, schradan (1389)+COMPOUND OF FORMULA I, sebufos (alternative name)+COMPOUND OF FORMULA I, selamectin (alternative name) [CCN]+COMPOUND OF FORMULA I, SI-0009 (compound code)+COMPOUND OF FORMULA I, sophamide (1402)+COMPOUND OF FORMULA I, spirodiclofen (738)+COMPOUND OF FORMULA I, spiromesifen (739)+COMPOUND OF FORMULA I, SSI-121 (development code) (1404)+COMPOUND OF FORMULA I, sulfuram (alternative name) [CCN]+COMPOUND OF FORMULA I, sulfluramid (750)+COMPOUND OF FORMULA I, sulfotep (753)+COMPOUND OF FORMULA I, sulfur (754)+COMPOUND OF FORMULA I, SZI-121 (development code) (757)+COMPOUND OF FORMULA I, tau-fluvalinate (398)+COMPOUND OF FORMULA I, tebufenpyrad (763)+COMPOUND OF FORMULA I, TEPP (1417)+COMPOUND OF FORMULA I, terbam (alternative name)+COMPOUND OF FORMULA I, tetrachlorvinphos (777)+COMPOUND OF FORMULA I, tetradifon (786)+COMPOUND OF FORMULA I, tetranactin (alternative name) (653)+COMPOUND OF FORMULA I, tetrasul (1425)+COMPOUND OF FORMULA I, thiafenox (alternative name)+COMPOUND OF FORMULA I, thiocarboxime (1431)+COMPOUND OF FORMULA I, thiofanox (800)+COMPOUND OF FORMULA I, thiometon (801)+COMPOUND OF FORMULA I, thioquinox (1436)+COMPOUND OF FORMULA I, thuringiensin (alternative name) [CCN]+COMPOUND OF FORMULA I, triamiphos (1441)+COMPOUND OF FORMULA I, triarathene (1443)+COMPOUND OF FORMULA I, triazophos (820)+COMPOUND OF FORMULA I, triazuron (alternative name)+COMPOUND OF FORMULA I, trichlorfon (824)+COMPOUND OF FORMULA I, trifenofos (1455)+COMPOUND OF FORMULA I, trinactin (alternative name) (653)+COMPOUND OF FORMULA I, vamidothion (847)+COMPOUND OF FORMULA I, vaniliprole [CCN] and YI-5302 (compound code)+COMPOUND OF FORMULA I,
an algicide selected from the group of substances consisting of bethoxazin [CCN]+COMPOUND OF FORMULA I, copper dioctanoate (IUPAC name) (170)+COMPOUND OF FORMULA I, copper sulfate (172)+COMPOUND OF FORMULA I, cybutryne [CCN]+COMPOUND OF FORMULA I, dichlone (1052)+COMPOUND OF FORMULA I, dichlorophen (232)+COMPOUND OF FORMULA I, endothal (295)+COMPOUND OF FORMULA I, fentin (347)+COMPOUND OF FORMULA I, hydrated lime [CCN]+COMPOUND OF FORMULA I, nabam (566)+COMPOUND OF FORMULA I, quinoclamine (714)+COMPOUND OF FORMULA I, quinonamid (1379)+COMPOUND OF FORMULA I, simazine (730)+COMPOUND OF FORMULA I, triphenyltin acetate (IUPAC name) (347) and triphenyltin hydroxide (IUPAC name) (347)+COMPOUND OF FORMULA I,
an anthelmintic selected from the group of substances consisting of abamectin (1)+COMPOUND OF FORMULA I, crufomate (1011)+COMPOUND OF FORMULA I, doramectin (alternative name) [CCN]+COMPOUND OF FORMULA I, emamectin (291)+COMPOUND OF FORMULA I, emamectin benzoate (291)+COMPOUND OF FORMULA I, eprinomectin (alternative name) [CCN]+COMPOUND OF FORMULA I, ivermectin (alternative name) [CCN]+COMPOUND OF FORMULA I, milbemycin oxime (alternative name) [CCN]+COMPOUND OF FORMULA I, moxidectin (alternative name) [CCN]+COMPOUND OF FORMULA I, piperazine [CCN]+COMPOUND OF FORMULA I, selamectin (alternative name) [CCN]+COMPOUND OF FORMULA I, spinosad (737) and thiophanate (1435)+COMPOUND OF FORMULA I,
an avicide selected from the group of substances consisting of chloralose (127)+COMPOUND OF FORMULA I, endrin (1122)+COMPOUND OF FORMULA I, fenthion (346)+COMPOUND OF FORMULA I, pyridin-4-amine (IUPAC name) (23) and strychnine (745)+COMPOUND OF FORMULA I,
a bactericide selected from the group of substances consisting of 1-hydroxy-1H-pyridine-2-thione (IUPAC name) (1222)+COMPOUND OF FORMULA I, 4-(quinoxalin-2-ylamino)benzenesulfonamide (IUPAC name) (748)+COMPOUND OF FORMULA I, 8-hydroxyquinoline sulfate (446)+COMPOUND OF FORMULA I, bronopol (97)+COMPOUND OF FORMULA I, copper dioctanoate (IUPAC name) (170)+COMPOUND OF FORMULA I, copper hydroxide (IUPAC name) (169)+COMPOUND OF FORMULA I, cresol [CCN]+COMPOUND OF FORMULA I, dichlorophen (232)+COMPOUND OF FORMULA I, dipyrithione (1105)+COMPOUND OF FORMULA I, dodicin (1112)+COMPOUND OF FORMULA I, fenaminosulf (1144)+COMPOUND OF FORMULA I, formaldehyde (404)+COMPOUND OF FORMULA I, hydrargaphen (alternative name) [CCN]+COMPOUND OF FORMULA I, kasugamycin (483)+COMPOUND OF FORMULA I, kasugamycin hydrochloride hydrate (483)+COMPOUND OF FORMULA I, nickel bis(dimethyldithiocarbamate) (IUPAC name) (1308)+COMPOUND OF FORMULA I, nitrapyrin (580)+COMPOUND OF FORMULA I, octhilinone (590)+COMPOUND OF FORMULA I, oxolinic acid (606)+COMPOUND OF FORMULA I, oxytetracycline (611)+COMPOUND OF FORMULA I, potassium hydroxyquinoline sulfate (446)+COMPOUND OF FORMULA I, probenazole (658)+COMPOUND OF FORMULA I, streptomycin (744)+COMPOUND OF FORMULA I, streptomycin sesquisulfate (744)+COMPOUND OF FORMULA I, tecloftalam (766)+COMPOUND OF FORMULA I, and thiomersal (alternative name) [CCN]+COMPOUND OF FORMULA I,
a biological agent selected from the group of substances consisting of Adoxophyes orana GV (alternative name) (12)+COMPOUND OF FORMULA I, Agrobacterium radiobacter (alternative name) (13)+COMPOUND OF FORMULA I, Amblyseius spp. (alternative name) (19)+COMPOUND OF FORMULA I, Anagrapha falcifera NPV (alternative name) (28)+COMPOUND OF FORMULA I, Anagrus atomus (alternative name) (29)+COMPOUND OF FORMULA I, Aphelinus abdominalis (alternative name) (33)+COMPOUND OF FORMULA I, Aphidius colemani (alternative name) (34)+COMPOUND OF FORMULA I, Aphidoletes aphidimyza (alternative name) (35)+COMPOUND OF FORMULA I, Autographa californica NPV (alternative name) (38)+COMPOUND OF FORMULA I, Bacillus firmus (alternative name) (48)+COMPOUND OF FORMULA I, Bacillus sphaericus Neide (scientific name) (49)+COMPOUND OF FORMULA I, Bacillus thuringiensis Berliner (scientific name) (51)+COMPOUND OF FORMULA I, Bacillus thuringiensis subsp. aizawai (scientific name) (51)+COMPOUND OF FORMULA I, Bacillus thuringiensis subsp. israelensis (scientific name) (51)+COMPOUND OF FORMULA I, Bacillus thuringiensis subsp. japonensis (scientific name) (51)+COMPOUND OF FORMULA I, Bacillus thuringiensis subsp. kurstaki (scientific name) (51)+COMPOUND OF FORMULA I, Bacillus thuringiensis subsp. tenebrionis (scientific name) (51)+COMPOUND OF FORMULA I, Beauveria bassiana (alternative name) (53)+COMPOUND OF FORMULA I, Beauveria brongniartii (alternative name) (54)+COMPOUND OF FORMULA I, Chrysoperla carnea (alternative name) (151)+COMPOUND OF FORMULA I, Cryptolaemus montrouzieri (alternative name) (178)+COMPOUND OF FORMULA I, Cydia pomonella GV (alternative name) (191)+COMPOUND OF FORMULA I, Dacnusa sibirica (alternative name) (212)+COMPOUND OF FORMULA I, Diglyphus isaea (alternative name) (254)+COMPOUND OF FORMULA I, Encarsia formosa (scientific name) (293)+COMPOUND OF FORMULA I, Eretmocerus eremicus (alternative name) (300)+COMPOUND OF FORMULA I, Helicoverpa zea NPV (alternative name) (431)+COMPOUND OF FORMULA I, Heterorhabditis bacteriophora and H. megidis (alternative name) (433)+COMPOUND OF FORMULA I, Hippodamia convergens (alternative name) (442)+COMPOUND OF FORMULA I, Leptomastix dactylopii (alternative name) (488)+COMPOUND OF FORMULA I, Macrolophus caliginosus (alternative name) (491)+COMPOUND OF FORMULA I, Mamestra brassicae NPV (alternative name) (494)+COMPOUND OF FORMULA I, Metaphycus helvolus (alternative name) (522)+COMPOUND OF FORMULA I, Metarhizium anisopliae var. acridum (scientific name) (523)+COMPOUND OF FORMULA I, Metarhizium anisopliae var. anisopliae (scientific name) (523)+COMPOUND OF FORMULA I, Neodiprion sertifer NPV and N. lecontei NPV (alternative name) (575)+COMPOUND OF FORMULA I, Orius spp. (alternative name) (596)+COMPOUND OF FORMULA I, Pasteuria usgae (alternative name)+COMPOUND OF FORMULA I, Paecilomyces fumosoroseus (alternative name) (613)+COMPOUND OF FORMULA I, Phytoseiulus persimilis (alternative name) (644)+COMPOUND OF FORMULA I, Spodoptera exigua multicapsid nuclear polyhedrosis virus (scientific name) (741)+COMPOUND OF FORMULA I, Steinernema bibionis (alternative name) (742)+COMPOUND OF FORMULA I, Steinernema carpocapsae (alternative name) (742)+COMPOUND OF FORMULA I, Steinernema feltiae (alternative name) (742)+COMPOUND OF FORMULA I, Steinernema glaseri (alternative name) (742)+COMPOUND OF FORMULA I, Steinernema riobrave (alternative name) (742)+COMPOUND OF FORMULA I, Steinernema riobravis (alternative name) (742)+COMPOUND OF FORMULA I, Steinernema scapterisci (alternative name) (742)+COMPOUND OF FORMULA I, Steinernema spp. (alternative name) (742)+COMPOUND OF FORMULA I, Trichoderma spp. (alternative name)+COMPOUND OF FORMULA I, Trichogramma spp. (alternative name) (826)+COMPOUND OF FORMULA I, Typhlodromus occidentalis (alternative name) (844) and Verticillium lecanii (alternative name) (848)+COMPOUND OF FORMULA I,
a soil sterilant selected from the group of substances consisting of dimethyl disulfide (IUPAC name)+COMPOUND OF FORMULA I, iodomethane (IUPAC name) (542) and methyl bromide (537)+COMPOUND OF FORMULA I,
a chemosterilant selected from the group of substances consisting of apholate [CCN]+COMPOUND OF FORMULA I, bisazir (alternative name) [CCN]+COMPOUND OF FORMULA I, busulfan (alternative name) [CCN]+COMPOUND OF FORMULA I, diflubenzuron (250)+COMPOUND OF FORMULA I, dimatif (alternative name) [CCN]+COMPOUND OF FORMULA I, hemel [CCN]+COMPOUND OF FORMULA I, hempa [CCN]+COMPOUND OF FORMULA I, metepa [CCN]+COMPOUND OF FORMULA I, methiotepa [CCN]+COMPOUND OF FORMULA I, methyl apholate [CCN]+COMPOUND OF FORMULA I, morzid [CCN]+COMPOUND OF FORMULA I, penfluoron (alternative name) [CCN]+COMPOUND OF FORMULA I, tepa [CCN]+COMPOUND OF FORMULA I, thiohempa (alternative name) [CCN]+COMPOUND OF FORMULA I, thiotepa (alternative name) [CCN]+COMPOUND OF FORMULA I, tretamine (alternative name) [CCN] and uredepa (alternative name) [CCN]+COMPOUND OF FORMULA I,
an insect pheromone selected from the group of substances consisting of (E)-dec-5-en-1-yl acetate with (E)-dec-5-en-1-ol (IUPAC name) (222)+COMPOUND OF FORMULA I, (E)-tridec-4-en-1-yl acetate (IUPAC name) (829)+COMPOUND OF FORMULA I, (E)-6-methylhept-2-en-4-ol (IUPAC name) (541)+COMPOUND OF FORMULA I, (E,Z)-tetradeca-4,10-dien-1-yl acetate (IUPAC name) (779)+COMPOUND OF FORMULA I, (Z)-dodec-7-en-1-yl acetate (IUPAC name) (285)+COMPOUND OF FORMULA I, (Z)-hexadec-11-enal (IUPAC name) (436)+COMPOUND OF FORMULA I, (Z)-hexadec-11-en-1-yl acetate (IUPAC name) (437)+COMPOUND OF FORMULA I, (Z)-hexadec-13-en-11-yn-1-yl acetate (IUPAC name) (438)+COMPOUND OF FORMULA I, (Z)-icos-13-en-10-one (IUPAC name) (448)+COMPOUND OF FORMULA I, (Z)-tetradec-7-en-1-al (IUPAC name) (782)+COMPOUND OF FORMULA I, (Z)-tetradec-9-en-1-ol (IUPAC name) (783)+COMPOUND OF FORMULA I, (Z)-tetradec-9-en-1-yl acetate (IUPAC name) (784)+COMPOUND OF FORMULA I, (7E,9Z)-dodeca-7,9-dien-1-yl acetate (IUPAC name) (283)+COMPOUND OF FORMULA I, (9Z,11E)-tetradeca-9,11-dien-1-yl acetate (IUPAC name) (780)+COMPOUND OF FORMULA I, (9Z,12E)-tetradeca-9,12-dien-1-yl acetate (IUPAC name) (781)+COMPOUND OF FORMULA I, 14-methyloctadec-1-ene (IUPAC name) (545)+COMPOUND OF FORMULA I, 4-methylnonan-5-ol with 4-methylnonan-5-one (IUPAC name) (544)+COMPOUND OF FORMULA I, alpha-multistriatin (alternative name) [CCN]+COMPOUND OF FORMULA I, brevicomin (alternative name) [CCN]+COMPOUND OF FORMULA I, codlelure (alternative name) [CCN]+COMPOUND OF FORMULA I, codlemone (alternative name) (167)+COMPOUND OF FORMULA I, cuelure (alternative name) (179)+COMPOUND OF FORMULA I, disparlure (277)+COMPOUND OF FORMULA I, (E,Z)-7,9-dodecadien-1-yl acetate (IUPAC name)+COMPOUND OF FORMULA I, dodec-8-en-1-yl acetate (IUPAC name) (286)+COMPOUND OF FORMULA I, dodec-9-en-1-yl acetate (IUPAC name) (287)+COMPOUND OF FORMULA I, dodeca-8+COMPOUND OF FORMULA I, 10-dien-1-yl acetate (IUPAC name) (284)+COMPOUND OF FORMULA I, dominicalure (alternative name) [CCN]+COMPOUND OF FORMULA I, ethyl 4-methyloctanoate (IUPAC name) (317)+COMPOUND OF FORMULA I, eugenol (alternative name) [CCN]+COMPOUND OF FORMULA I, exosex SPTab (alternative name)+COMPOUND OF FORMULA I, frontalin (alternative name) [CCN]+COMPOUND OF FORMULA I, gossyplure (alternative name) (420)+COMPOUND OF FORMULA I, grandlure (421)+COMPOUND OF FORMULA I, grandlure I (alternative name) (421)+COMPOUND OF FORMULA I, grandlure II (alternative name) (421)+COMPOUND OF FORMULA I, grandlure III (alternative name) (421)+COMPOUND OF FORMULA I, grandlure IV (alternative name) (421)+COMPOUND OF FORMULA I, hexylure [CCN]+COMPOUND OF FORMULA I, imicyafos (alternative name) [CCN]+COMPOUND OF FORMULA I, ipsdienol (alternative name) [CCN]+COMPOUND OF FORMULA I, ipsenol (alternative name) [CCN]+COMPOUND OF FORMULA I, japonilure (alternative name) (481)+COMPOUND OF FORMULA I, lineatin (alternative name) [CCN]+COMPOUND OF FORMULA I, litlure (alternative name) [CCN]+COMPOUND OF FORMULA I, looplure (alternative name) [CCN]+COMPOUND OF FORMULA I, medlure [CCN]+COMPOUND OF FORMULA I, megatomoic acid (alternative name) [CCN]+COMPOUND OF FORMULA I, methyl eugenol (alternative name) (540)+COMPOUND OF FORMULA I, muscalure (563)+COMPOUND OF FORMULA I, octadeca-2,13-dien-1-yl acetate (IUPAC name) (588)+COMPOUND OF FORMULA I, octadeca-3,13-dien-1-yl acetate (IUPAC name) (589)+COMPOUND OF FORMULA I, orfralure (alternative name) [CCN]+COMPOUND OF FORMULA I, oryctalure (alternative name) (317)+COMPOUND OF FORMULA I, ostramone (alternative name) [CCN]+COMPOUND OF FORMULA I, siglure [CCN]+COMPOUND OF FORMULA I, sordidin (alternative name) (736)+COMPOUND OF FORMULA I, sulcatol (alternative name) [CCN]+COMPOUND OF FORMULA I, tetradec-11-en-1-yl acetate (IUPAC name) (785)+COMPOUND OF FORMULA I, trimedlure (839)+COMPOUND OF FORMULA I, trimedlure A (alternative name) (839)+COMPOUND OF FORMULA I, trimedlure B₁ (alternative name) (839)+COMPOUND OF FORMULA I, trimedlure B₂ (alternative name) (839)+COMPOUND OF FORMULA I, trimedlure C (alternative name) (839) and trunc-call (alternative name) [CCN]+COMPOUND OF FORMULA I,
an insect repellent selected from the group of substances consisting of 2-(octylthio)-ethanol (IUPAC name) (591)+COMPOUND OF FORMULA I, butopyronoxyl (933)+COMPOUND OF FORMULA I, butoxy(polypropylene glycol) (936)+COMPOUND OF FORMULA I, dibutyl adipate (IUPAC name) (1046)+COMPOUND OF FORMULA I, dibutyl phthalate (1047)+COMPOUND OF FORMULA I, dibutyl succinate (IUPAC name) (1048)+COMPOUND OF FORMULA I, diethyltoluamide [CCN]+COMPOUND OF FORMULA I, dimethyl carbate [CCN]+COMPOUND OF FORMULA I, dimethyl phthalate [CCN]+COMPOUND OF FORMULA I, ethyl hexanediol (1137)+COMPOUND OF FORMULA I, hexamide [CCN]+COMPOUND OF FORMULA I, methoquin-butyl (1276)+COMPOUND OF FORMULA I, methylneodecanamide [CCN]+COMPOUND OF FORMULA I, oxamate [CCN] and picaridin [CCN]+COMPOUND OF FORMULA I,
an insecticide selected from the group of substances consisting of 1-dichloro-1-nitroethane (IUPAC/Chemical Abstracts name) (1058)+COMPOUND OF FORMULA I, 1,1-dichloro-2,2-bis(4-ethylphenyl)ethane (IUPAC name) (1056), +COMPOUND OF FORMULA I, 1,2-dichloropropane (IUPAC/Chemical Abstracts name) (1062)+COMPOUND OF FORMULA I, 1,2-dichloropropane with 1,3-dichloropropene (IUPAC name) (1063)+COMPOUND OF FORMULA I, 1-bromo-2-chloroethane (IUPAC/Chemical Abstracts name) (916)+COMPOUND OF FORMULA I, 2,2,2-trichloro-1-(3,4-dichloro-phenyl)ethyl acetate (IUPAC name) (1451)+COMPOUND OF FORMULA I, 2,2-dichlorovinyl 2-ethylsulfinylethyl methyl phosphate (IUPAC name) (1066)+COMPOUND OF FORMULA I, 2-(1,3-dithiolan-2-yl)phenyl dimethylcarbamate (IUPAC/Chemical Abstracts name) (1109)+COMPOUND OF FORMULA I, 2-(2-butoxyethoxy)ethyl thiocyanate (IUPAC/Chemical Abstracts name) (935)+COMPOUND OF FORMULA I, 2-(4,5-dimethyl-1,3-dioxolan-2-yl)phenyl methylcarbamate (IUPAC/Chemical Abstracts name) (1084)+COMPOUND OF FORMULA I, 2-(4-chloro-3,5-xylyloxy)ethanol (IUPAC name) (986)+COMPOUND OF FORMULA I, 2-chlorovinyl diethyl phosphate (IUPAC name) (984)+COMPOUND OF FORMULA I, 2-imidazolidone (IUPAC name) (1225)+COMPOUND OF FORMULA I, 2-isovalerylindan-1,3-dione (IUPAC name) (1246)+COMPOUND OF FORMULA I, 2-methyl(prop-2-ynyl)aminophenyl methylcarbamate (IUPAC name) (1284)+COMPOUND OF FORMULA I, 2-thiocyanatoethyl laurate (IUPAC name) (1433)+COMPOUND OF FORMULA I, 3-bromo-1-chloroprop-1-ene (IUPAC name) (917)+COMPOUND OF FORMULA I, 3-methyl-1-phenylpyrazol-5-yl dimethyl-carbamate (IUPAC name) (1283)+COMPOUND OF FORMULA I, 4-methyl(prop-2-ynyl)amino-3,5-xylyl methylcarbamate (IUPAC name) (1285)+COMPOUND OF FORMULA I, 5,5-dimethyl-3-oxocyclohex-1-enyl dimethylcarbamate (IUPAC name) (1085)+COMPOUND OF FORMULA I, abamectin (1)+COMPOUND OF FORMULA I, acephate (2)+COMPOUND OF FORMULA I, acetamiprid (4)+COMPOUND OF FORMULA I, acethion (alternative name) [CCN]+COMPOUND OF FORMULA I, acetoprole [CCN]+COMPOUND OF FORMULA I, acrinathrin (9)+COMPOUND OF FORMULA I, acrylonitrile (IUPAC name) (861)+COMPOUND OF FORMULA I, alanycarb (15)+COMPOUND OF FORMULA I, aldicarb (16)+COMPOUND OF FORMULA I, aldoxycarb (863)+COMPOUND OF FORMULA I, aldrin (864)+COMPOUND OF FORMULA I, allethrin (17)+COMPOUND OF FORMULA I, allosamidin (alternative name) [CCN]+COMPOUND OF FORMULA I, allyxycarb (866)+COMPOUND OF FORMULA I, alpha-cypermethrin (202)+COMPOUND OF FORMULA I, alpha-ecdysone (alternative name) [CCN]+COMPOUND OF FORMULA I, alpha-endosulfan [CCN]+COMPOUND OF FORMULA I, aluminium phosphide (640)+COMPOUND OF FORMULA I, amidithion (870)+COMPOUND OF FORMULA I, amidothioate (872)+COMPOUND OF FORMULA I, aminocarb (873)+COMPOUND OF FORMULA I, amiton (875)+COMPOUND OF FORMULA I, amiton hydrogen oxalate (875)+COMPOUND OF FORMULA I, amitraz (24)+COMPOUND OF FORMULA I, anabasine (877)+COMPOUND OF FORMULA I, athidathion (883)+COMPOUND OF FORMULA I, AVI 382 (compound code)+COMPOUND OF FORMULA I, AZ 60541 (compound code)+COMPOUND OF FORMULA I, azadirachtin (alternative name) (41)+COMPOUND OF FORMULA I, azamethiphos (42)+COMPOUND OF FORMULA I, azinphos-ethyl (44)+COMPOUND OF FORMULA I, azinphos-methyl (45)+COMPOUND OF FORMULA I, azothoate (889)+COMPOUND OF FORMULA I, Bacillus thuringiensis delta endotoxins (alternative name) (52)+COMPOUND OF FORMULA I, barium hexafluorosilicate (alternative name) [CCN]+COMPOUND OF FORMULA I, barium polysulfide (IUPAC/Chemical Abstracts name) (892)+COMPOUND OF FORMULA I, barthrin [CCN]+COMPOUND OF FORMULA I, Bayer 22/190 (development code) (893)+COMPOUND OF FORMULA I, Bayer 22408 (development code) (894)+COMPOUND OF FORMULA I, bendiocarb (58)+COMPOUND OF FORMULA I, benfuracarb (60)+COMPOUND OF FORMULA I, bensultap (66)+COMPOUND OF FORMULA I, beta-cyfluthrin (194)+COMPOUND OF FORMULA I, beta-cypermethrin (203)+COMPOUND OF FORMULA I, bifenthrin (76)+COMPOUND OF FORMULA I, bioallethrin (78)+COMPOUND OF FORMULA I, bioallethrin S-cyclopentenyl isomer (alternative name) (79)+COMPOUND OF FORMULA I, bioethanomethrin [CCN]+COMPOUND OF FORMULA I, biopermethrin (908)+COMPOUND OF FORMULA I, bioresmethrin (80)+COMPOUND OF FORMULA I, bis(2-chloroethyl)ether (IUPAC name) (909)+COMPOUND OF FORMULA I, bistrifluoron (83)+COMPOUND OF FORMULA I, borax (86)+COMPOUND OF FORMULA I, brofenvalerate (alternative name)+COMPOUND OF FORMULA I, bromfenvinfos (914)+COMPOUND OF FORMULA I, bromocyclen (918)+COMPOUND OF FORMULA I, bromo-DDT (alternative name) [CCN]+COMPOUND OF FORMULA I, bromophos (920)+COMPOUND OF FORMULA I, bromophos-ethyl (921)+COMPOUND OF FORMULA I, bufencarb (924)+COMPOUND OF FORMULA I, buprofezin (99)+COMPOUND OF FORMULA I, butacarb (926)+COMPOUND OF FORMULA I, butathiofos (927)+COMPOUND OF FORMULA I, butocarboxim (103)+COMPOUND OF FORMULA I, butonate (932)+COMPOUND OF FORMULA I, butoxycarboxim (104)+COMPOUND OF FORMULA I, butylpyridaben (alternative name)+COMPOUND OF FORMULA I, cadusafos (109)+COMPOUND OF FORMULA I, calcium arsenate [CCN]+COMPOUND OF FORMULA I, calcium cyanide (444)+COMPOUND OF FORMULA I, calcium polysulfide (IUPAC name) (111)+COMPOUND OF FORMULA I, camphechlor (941)+COMPOUND OF FORMULA I, carbanolate (943)+COMPOUND OF FORMULA I, carbaryl (115)+COMPOUND OF FORMULA I, carbofuran (118)+COMPOUND OF FORMULA I, carbon disulfide (IUPAC/Chemical Abstracts name) (945)+COMPOUND OF FORMULA I, carbon tetrachloride (IUPAC name) (946)+COMPOUND OF FORMULA I, carbophenothion (947)+COMPOUND OF FORMULA I, carbosulfan (119)+COMPOUND OF FORMULA I, cartap (123)+COMPOUND OF FORMULA I, cartap hydrochloride (123)+COMPOUND OF FORMULA I, celangulin (alternative name)+COMPOUND OF FORMULA I, cevadine (alternative name) (725)+COMPOUND OF FORMULA I, chlorantraniliprole [CCN]+COMPOUND OF FORMULA I, chlorbicyclen (960)+COMPOUND OF FORMULA I, chlordane (128)+COMPOUND OF FORMULA I, chlordecone (963)+COMPOUND OF FORMULA I, chlordimeform (964)+COMPOUND OF FORMULA I, chlordimeform hydrochloride (964)+COMPOUND OF FORMULA I, chlorethoxyfos (129)+COMPOUND OF FORMULA I, chlorfenapyr (130)+COMPOUND OF FORMULA I, chlorfenvinphos (131)+COMPOUND OF FORMULA I, chlorfluazuron (132)+COMPOUND OF FORMULA I, chlormephos (136)+COMPOUND OF FORMULA I, chloroform [CCN]+COMPOUND OF FORMULA I, chloropicrin (141)+COMPOUND OF FORMULA I, chlorphoxim (989)+COMPOUND OF FORMULA I, chlorprazophos (990)+COMPOUND OF FORMULA I, chlorpyrifos (145)+COMPOUND OF FORMULA I, chlorpyrifos-methyl (146)+COMPOUND OF FORMULA I, chlorthiophos (994)+COMPOUND OF FORMULA I, chromafenozide (150)+COMPOUND OF FORMULA I, cinerin I (696)+COMPOUND OF FORMULA I, cinerin II (696)+COMPOUND OF FORMULA I, cinerins (696)+COMPOUND OF FORMULA I, cis-resmethrin (alternative name)+COMPOUND OF FORMULA I, cismethrin (80)+COMPOUND OF FORMULA I, clocythrin (alternative name)+COMPOUND OF FORMULA I, cloethocarb (999)+COMPOUND OF FORMULA I, closantel (alternative name) [CCN]+COMPOUND OF FORMULA I, clothianidin (165)+COMPOUND OF FORMULA I, copper acetoarsenite [CCN]+COMPOUND OF FORMULA I, copper arsenate [CCN]+COMPOUND OF FORMULA I, copper oleate [CCN]+COMPOUND OF FORMULA I, coumaphos (174)+COMPOUND OF FORMULA I, coumithoate (1006)+COMPOUND OF FORMULA I, crotamiton (alternative name) [CCN]+COMPOUND OF FORMULA I, crotoxyphos (1010)+COMPOUND OF FORMULA I, crufomate (1011)+COMPOUND OF FORMULA I, cryolite (alternative name) (177)+COMPOUND OF FORMULA I, CS 708 (development code) (1012)+COMPOUND OF FORMULA I, cyanofenphos (1019)+COMPOUND OF FORMULA I, cyanophos (184)+COMPOUND OF FORMULA I, cyanthoate (1020)+COMPOUND OF FORMULA I, cyantraniliprole [CCN]+COMPOUND OF FORMULA I, cyclethrin [CCN]+COMPOUND OF FORMULA I, cycloprothrin (188)+COMPOUND OF FORMULA I, cyfluthrin (193)+COMPOUND OF FORMULA I, cyhalothrin (196)+COMPOUND OF FORMULA I, cypermethrin (201)+COMPOUND OF FORMULA I, cyphenothrin (206)+COMPOUND OF FORMULA I, cyromazine (209)+COMPOUND OF FORMULA I, cythioate (alternative name) [CCN]+COMPOUND OF FORMULA I, d-limonene (alternative name) [CCN]+COMPOUND OF FORMULA I, d-tetramethrin (alternative name) (788)+COMPOUND OF FORMULA I, DAEP (1031)+COMPOUND OF FORMULA I, dazomet (216)+COMPOUND OF FORMULA I, DDT (219)+COMPOUND OF FORMULA I, decarbofuran (1034)+COMPOUND OF FORMULA I, deltamethrin (223)+COMPOUND OF FORMULA I, demephion (1037)+COMPOUND OF FORMULA I, demephion-O (1037)+COMPOUND OF FORMULA I, demephion-S (1037)+COMPOUND OF FORMULA I, demeton (1038)+COMPOUND OF FORMULA I, demeton-methyl (224)+COMPOUND OF FORMULA I, demeton-O (1038)+COMPOUND OF FORMULA I, demeton-O-methyl (224)+COMPOUND OF FORMULA I, demeton-S (1038)+COMPOUND OF FORMULA I, demeton-5-methyl (224)+COMPOUND OF FORMULA I, demeton-5-methylsulphon (1039)+COMPOUND OF FORMULA I, diafenthiuron (226)+COMPOUND OF FORMULA I, dialifos (1042)+COMPOUND OF FORMULA I, diamidafos (1044)+COMPOUND OF FORMULA I, diazinon (227)+COMPOUND OF FORMULA I, dicapthon (1050)+COMPOUND OF FORMULA I, dichlofenthion (1051)+COMPOUND OF FORMULA I, dichlorvos (236)+COMPOUND OF FORMULA I, dicliphos (alternative name)+COMPOUND OF FORMULA I, dicresyl (alternative name) [CCN]+COMPOUND OF FORMULA I, dicrotophos (243)+COMPOUND OF FORMULA I, dicyclanil (244)+COMPOUND OF FORMULA I, dieldrin (1070)+COMPOUND OF FORMULA I, diethyl 5-methylpyrazol-3-yl phosphate (IUPAC name) (1076)+COMPOUND OF FORMULA I, diflubenzuron (250)+COMPOUND OF FORMULA I, dilor (alternative name) [CCN]+COMPOUND OF FORMULA I, dimefluthrin [CCN]+COMPOUND OF FORMULA I, dimefox (1081)+COMPOUND OF FORMULA I, dimetan (1085)+COMPOUND OF FORMULA I, dimethoate (262)+COMPOUND OF FORMULA I, dimethrin (1083)+COMPOUND OF FORMULA I, dimethylvinphos (265)+COMPOUND OF FORMULA I, dimetilan (1086)+COMPOUND OF FORMULA I, dinex (1089)+COMPOUND OF FORMULA I, dinex-diclexine (1089)+COMPOUND OF FORMULA I, dinoprop (1093)+COMPOUND OF FORMULA I, dinosam (1094)+COMPOUND OF FORMULA I, dinoseb (1095)+COMPOUND OF FORMULA I, dinotefuran (271)+COMPOUND OF FORMULA I, diofenolan (1099)+COMPOUND OF FORMULA I, dioxabenzofos (1100)+COMPOUND OF FORMULA I, dioxacarb (1101)+COMPOUND OF FORMULA I, dioxathion (1102)+COMPOUND OF FORMULA I, disulfoton (278)+COMPOUND OF FORMULA I, dithicrofos (1108)+COMPOUND OF FORMULA I, DNOC (282)+COMPOUND OF FORMULA I, doramectin (alternative name) [CCN]+COMPOUND OF FORMULA I, DSP (1115)+COMPOUND OF FORMULA I, ecdysterone (alternative name) [CCN]+COMPOUND OF FORMULA I, EI 1642 (development code) (1118)+COMPOUND OF FORMULA I, emamectin (291)+COMPOUND OF FORMULA I, emamectin benzoate (291)+COMPOUND OF FORMULA I, EMPC (1120)+COMPOUND OF FORMULA I, empenthrin (292)+COMPOUND OF FORMULA I, endosulfan (294)+COMPOUND OF FORMULA I, endothion (1121)+COMPOUND OF FORMULA I, endrin (1122)+COMPOUND OF FORMULA I, EPBP (1123)+COMPOUND OF FORMULA I, EPN (297)+COMPOUND OF FORMULA I, epofenonane (1124)+COMPOUND OF FORMULA I, eprinomectin (alternative name) [CCN]+COMPOUND OF FORMULA I, eremophilone oil+COMPOUND OF FORMULA I, esfenvalerate (302)+COMPOUND OF FORMULA I, etaphos (alternative name) [CCN]+COMPOUND OF FORMULA I, ethiofencarb (308)+COMPOUND OF FORMULA I, ethion (309)+COMPOUND OF FORMULA I, ethiprole (310)+COMPOUND OF FORMULA I, ethoate-methyl (1134)+COMPOUND OF FORMULA I, ethoprophos (312)+COMPOUND OF FORMULA I, ethyl formate (IUPAC name) [CCN]+COMPOUND OF FORMULA I, ethyl-DDD (alternative name) (1056)+COMPOUND OF FORMULA I, ethylene dibromide (316)+COMPOUND OF FORMULA I, ethylene dichloride (chemical name) (1136)+COMPOUND OF FORMULA I, ethylene oxide [CCN]+COMPOUND OF FORMULA I, etofenprox (319)+COMPOUND OF FORMULA I, etrimfos (1142)+COMPOUND OF FORMULA I, EXD (1143)+COMPOUND OF FORMULA I, famphur (323)+COMPOUND OF FORMULA I, fenamiphos (326)+COMPOUND OF FORMULA I, fenazaflor (1147)+COMPOUND OF FORMULA I, fenchlorphos (1148)+COMPOUND OF FORMULA I, fenethacarb (1149)+COMPOUND OF FORMULA I, fenfluthrin (1150)+COMPOUND OF FORMULA I, fenitrothion (335)+COMPOUND OF FORMULA I, fenobucarb (336)+COMPOUND OF FORMULA I, fenoxacrim (1153)+COMPOUND OF FORMULA I, fenoxycarb (340)+COMPOUND OF FORMULA I, fenpirithrin (1155)+COMPOUND OF FORMULA I, fenpropathrin (342)+COMPOUND OF FORMULA I, fenpyrad (alternative name)+COMPOUND OF FORMULA I, fensulfothion (1158)+COMPOUND OF FORMULA I, fenthion (346)+COMPOUND OF FORMULA I, fenthion-ethyl [CCN]+COMPOUND OF FORMULA I, fenvalerate (349)+COMPOUND OF FORMULA I, fipronil (354)+COMPOUND OF FORMULA I, flometoquin [CCN]+COMPOUND OF FORMULA I, flonicamid (358)+COMPOUND OF FORMULA I, flubendiamide (CAS. Reg. No.: 272451-65-7)+COMPOUND OF FORMULA I, flucofuron (1168)+COMPOUND OF FORMULA I, flucycloxuron (366)+COMPOUND OF FORMULA I, flucythrinate (367)+COMPOUND OF FORMULA I, fluenetil (1169)+COMPOUND OF FORMULA I, fluensulfon [CCN]+COMPOUND OF FORMULA I, flufenerim [CCN]+COMPOUND OF FORMULA I, flufenoxuron (370)+COMPOUND OF FORMULA I, flufenprox (1171)+COMPOUND OF FORMULA I, flufiprole [CCN]+COMPOUND OF FORMULA I, flumethrin (372)+COMPOUND OF FORMULA I, flupyradifurone [CCN]+COMPOUND OF FORMULA I, fluvalinate (1184)+COMPOUND OF FORMULA I, FMC 1137 (development code) (1185)+COMPOUND OF FORMULA I, fonofos (1191)+COMPOUND OF FORMULA I, formetanate (405)+COMPOUND OF FORMULA I, formetanate hydrochloride (405)+COMPOUND OF FORMULA I, formothion (1192)+COMPOUND OF FORMULA I, formparanate (1193)+COMPOUND OF FORMULA I, fosmethilan (1194)+COMPOUND OF FORMULA I, fospirate (1195)+COMPOUND OF FORMULA I, fosthiazate (408)+COMPOUND OF FORMULA I, fosthietan (1196)+COMPOUND OF FORMULA I, furathiocarb (412)+COMPOUND OF FORMULA I, furethrin (1200)+COMPOUND OF FORMULA I, gamma-cyhalothrin (197)+COMPOUND OF FORMULA I, gamma-HCH (430)+COMPOUND OF FORMULA I, guazatine (422)+COMPOUND OF FORMULA I, guazatine acetates (422)+COMPOUND OF FORMULA I, GY-81 (development code) (423)+COMPOUND OF FORMULA I, halfenprox (424)+COMPOUND OF FORMULA I, halofenozide (425)+COMPOUND OF FORMULA I, HCH (430)+COMPOUND OF FORMULA I, HEOD (1070)+COMPOUND OF FORMULA I, heptachlor (1211)+COMPOUND OF FORMULA I, heptenophos (432)+COMPOUND OF FORMULA I, heterophos [CCN]+COMPOUND OF FORMULA I, hexaflumuron (439)+COMPOUND OF FORMULA I, HHDN (864)+COMPOUND OF FORMULA I, hydramethylnon (443)+COMPOUND OF FORMULA I, hydrogen cyanide (444)+COMPOUND OF FORMULA I, hydroprene (445)+COMPOUND OF FORMULA I, hyquincarb (1223)+COMPOUND OF FORMULA I, imidacloprid (458)+COMPOUND OF FORMULA I, imiprothrin (460)+COMPOUND OF FORMULA I, indoxacarb (465)+COMPOUND OF FORMULA I, iodomethane (IUPAC name) (542)+COMPOUND OF FORMULA I, IPPA-152004 (compound code)+COMPOUND OF FORMULA I, IPSP (1229)+COMPOUND OF FORMULA I, isazofos (1231)+COMPOUND OF FORMULA I, isobenzan (1232)+COMPOUND OF FORMULA I, isocarbophos (alternative name) (473)+COMPOUND OF FORMULA I, isodrin (1235)+COMPOUND OF FORMULA I, isofenphos (1236)+COMPOUND OF FORMULA I, isolane (1237)+COMPOUND OF FORMULA I, isoprocarb (472)+COMPOUND OF FORMULA I, isopropyl O-(methoxyaminothiophosphoryl)salicylate (IUPAC name) (473)+COMPOUND OF FORMULA I, isoprothiolane (474)+COMPOUND OF FORMULA I, isothioate (1244)+COMPOUND OF FORMULA I, isoxathion (480)+COMPOUND OF FORMULA I, ivermectin (alternative name) [CCN]+COMPOUND OF FORMULA I, jasmolin I (696)+COMPOUND OF FORMULA I, jasmolin II (696)+COMPOUND OF FORMULA I, jodfenphos (1248)+COMPOUND OF FORMULA I, juvenile hormone I (alternative name) [CCN]+COMPOUND OF FORMULA I, juvenile hormone II (alternative name) [CCN]+COMPOUND OF FORMULA I, juvenile hormone III (alternative name) [CCN]+COMPOUND OF FORMULA I, kelevan (1249)+COMPOUND OF FORMULA I, kinoprene (484)+COMPOUND OF FORMULA I, lambda-cyhalothrin (198)+COMPOUND OF FORMULA I, lead arsenate [CCN]+COMPOUND OF FORMULA I, lepimectin (CCN)+COMPOUND OF FORMULA I, leptophos (1250)+COMPOUND OF FORMULA I, lindane (430)+COMPOUND OF FORMULA I, lirimfos (1251)+COMPOUND OF FORMULA I, lufenuron (490)+COMPOUND OF FORMULA I, lythidathion (1253)+COMPOUND OF FORMULA I, m-cumenyl methylcarbamate (IUPAC name) (1014)+COMPOUND OF FORMULA I, magnesium phosphide (IUPAC name) (640)+COMPOUND OF FORMULA I, malathion (492)+COMPOUND OF FORMULA I, malonoben (1254)+COMPOUND OF FORMULA I, mazidox (1255)+COMPOUND OF FORMULA I, mecarbam (502)+COMPOUND OF FORMULA I, mecarphon (1258)+COMPOUND OF FORMULA I, menazon (1260)+COMPOUND OF FORMULA I, mephosfolan (1261)+COMPOUND OF FORMULA I, mercurous chloride (513)+COMPOUND OF FORMULA I, mesulfenfos (1263)+COMPOUND OF FORMULA I, metaflumizone (CCN)+COMPOUND OF FORMULA I, metam (519)+COMPOUND OF FORMULA I, metam-potassium (alternative name) (519)+COMPOUND OF FORMULA I, metam-sodium (519)+COMPOUND OF FORMULA I, methacrifos (1266)+COMPOUND OF FORMULA I, methamidophos (527)+COMPOUND OF FORMULA I, methanesulfonyl fluoride (IUPAC/Chemical Abstracts name) (1268)+COMPOUND OF FORMULA I, methidathion (529)+COMPOUND OF FORMULA I, methiocarb (530)+COMPOUND OF FORMULA I, methocrotophos (1273)+COMPOUND OF FORMULA I, methomyl (531)+COMPOUND OF FORMULA I, methoprene (532)+COMPOUND OF FORMULA I, methoquin-butyl (1276)+COMPOUND OF FORMULA I, methothrin (alternative name) (533)+COMPOUND OF FORMULA I, methoxychlor (534)+COMPOUND OF FORMULA I, methoxyfenozide (535)+COMPOUND OF FORMULA I, methyl bromide (537)+COMPOUND OF FORMULA I, methyl isothiocyanate (543)+COMPOUND OF FORMULA I, methylchloroform (alternative name) [CCN]+COMPOUND OF FORMULA I, methylene chloride [CCN]+COMPOUND OF FORMULA I, metofluthrin [CCN]+COMPOUND OF FORMULA I, metolcarb (550)+COMPOUND OF FORMULA I, metoxadiazone (1288)+COMPOUND OF FORMULA I, mevinphos (556)+COMPOUND OF FORMULA I, mexacarbate (1290)+COMPOUND OF FORMULA I, milbemectin (557)+COMPOUND OF FORMULA I, milbemycin oxime (alternative name) [CCN]+COMPOUND OF FORMULA I, mipafox (1293)+COMPOUND OF FORMULA I, mirex (1294)+COMPOUND OF FORMULA I, monocrotophos (561)+COMPOUND OF FORMULA I, morphothion (1300)+COMPOUND OF FORMULA I, moxidectin (alternative name) [CCN]+COMPOUND OF FORMULA I, naftalofos (alternative name) [CCN]+COMPOUND OF FORMULA I, naled (567)+COMPOUND OF FORMULA I, naphthalene (IUPAC/Chemical Abstracts name) (1303)+COMPOUND OF FORMULA I, NC-170 (development code) (1306)+COMPOUND OF FORMULA I, NC-184 (compound code)+COMPOUND OF FORMULA I, nicotine (578)+COMPOUND OF FORMULA I, nicotine sulfate (578)+COMPOUND OF FORMULA I, nifluridide (1309)+COMPOUND OF FORMULA I, nitenpyram (579)+COMPOUND OF FORMULA I, nithiazine (1311)+COMPOUND OF FORMULA I, nitrilacarb (1313)+COMPOUND OF FORMULA I, nitrilacarb 1:1 zinc chloride complex (1313)+COMPOUND OF FORMULA I, NNI-0101 (compound code)+COMPOUND OF FORMULA I, NNI-0250 (compound code)+COMPOUND OF FORMULA I, nornicotine (traditional name) (1319)+COMPOUND OF FORMULA I, novaluron (585)+COMPOUND OF FORMULA I, noviflumuron (586)+COMPOUND OF FORMULA I, O-5-dichloro-4-iodophenyl O-ethyl ethylphosphonothioate (IUPAC name) (1057)+COMPOUND OF FORMULA I, O,O-diethyl O-4-methyl-2-oxo-2H-chromen-7-yl phosphorothioate (IUPAC name) (1074)+COMPOUND OF FORMULA I, O,O-diethyl O-6-methyl-2-propylpyrimidin-4-yl phosphorothioate (IUPAC name) (1075)+COMPOUND OF FORMULA I, O,O,O′,O′-tetrapropyl dithiopyrophosphate (IUPAC name) (1424)+COMPOUND OF FORMULA I, oleic acid (IUPAC name) (593)+COMPOUND OF FORMULA I, omethoate (594)+COMPOUND OF FORMULA I, oxamyl (602)+COMPOUND OF FORMULA I, oxydemeton-methyl (609)+COMPOUND OF FORMULA I, oxydeprofos (1324)+COMPOUND OF FORMULA I, oxydisulfoton (1325)+COMPOUND OF FORMULA I, pp'-DDT (219)+COMPOUND OF FORMULA I, para-dichlorobenzene [CCN]+COMPOUND OF FORMULA I, parathion (615)+COMPOUND OF FORMULA I, parathion-methyl (616)+COMPOUND OF FORMULA I, penfluoron (alternative name) [CCN]+COMPOUND OF FORMULA I, pentachlorophenol (623)+COMPOUND OF FORMULA I, pentachlorophenyl laurate (IUPAC name) (623)+COMPOUND OF FORMULA I, permethrin (626)+COMPOUND OF FORMULA I, petroleum oils (alternative name) (628)+COMPOUND OF FORMULA I, PH 60-38 (development code) (1328)+COMPOUND OF FORMULA I, phenkapton (1330)+COMPOUND OF FORMULA I, phenothrin (630)+COMPOUND OF FORMULA I, phenthoate (631)+COMPOUND OF FORMULA I, phorate (636)+COMPOUND OF FORMULA I, phosalone (637)+COMPOUND OF FORMULA I, phosfolan (1338)+COMPOUND OF FORMULA I, phosmet (638)+COMPOUND OF FORMULA I, phosnichlor (1339)+COMPOUND OF FORMULA I, phosphamidon (639)+COMPOUND OF FORMULA I, phosphine (IUPAC name) (640)+COMPOUND OF FORMULA I, phoxim (642)+COMPOUND OF FORMULA I, phoxim-methyl (1340)+COMPOUND OF FORMULA I, pirimetaphos (1344)+COMPOUND OF FORMULA I, pirimicarb (651)+COMPOUND OF FORMULA I, pirimiphos-ethyl (1345)+COMPOUND OF FORMULA I, pirimiphos-methyl (652)+COMPOUND OF FORMULA I, polychlorodicyclopentadiene isomers (IUPAC name) (1346)+COMPOUND OF FORMULA I, polychloroterpenes (traditional name) (1347)+COMPOUND OF FORMULA I, potassium arsenite [CCN]+COMPOUND OF FORMULA I, potassium thiocyanate [CCN]+COMPOUND OF FORMULA I, prallethrin (655)+COMPOUND OF FORMULA I, precocene I (alternative name) [CCN]+COMPOUND OF FORMULA I, precocene II (alternative name) [CCN]+COMPOUND OF FORMULA I, precocene III (alternative name) [CCN]+COMPOUND OF FORMULA I, primidophos (1349)+COMPOUND OF FORMULA I, profenofos (662)+COMPOUND OF FORMULA I, profluthrin [CCN]+COMPOUND OF FORMULA I, promacyl (1354)+COMPOUND OF FORMULA I, promecarb (1355)+COMPOUND OF FORMULA I, propaphos (1356)+COMPOUND OF FORMULA I, propetamphos (673)+COMPOUND OF FORMULA I, propoxur (678)+COMPOUND OF FORMULA I, prothidathion (1360)+COMPOUND OF FORMULA I, prothiofos (686)+COMPOUND OF FORMULA I, prothoate (1362)+COMPOUND OF FORMULA I, protrifenbute [CCN]+COMPOUND OF FORMULA I, pymetrozine (688)+COMPOUND OF FORMULA I, pyraclofos (689)+COMPOUND OF FORMULA I, pyrafluprole [CCN]+COMPOUND OF FORMULA I, pyrazophos (693)+COMPOUND OF FORMULA I, pyresmethrin (1367)+COMPOUND OF FORMULA I, pyrethrin I (696)+COMPOUND OF FORMULA I, pyrethrin II (696)+COMPOUND OF FORMULA I, pyrethrins (696)+COMPOUND OF FORMULA I, pyridaben (699)+COMPOUND OF FORMULA I, pyridalyl (700)+COMPOUND OF FORMULA I, pyridaphenthion (701)+COMPOUND OF FORMULA I, pyrifluquinazon [CCN]+COMPOUND OF FORMULA I, pyrimidifen (706)+COMPOUND OF FORMULA I, pyrimitate (1370)+COMPOUND OF FORMULA I, pyriprole [CCN]+COMPOUND OF FORMULA I, pyriproxyfen (708)+COMPOUND OF FORMULA I, quassia (alternative name) [CCN]+COMPOUND OF FORMULA I, quinalphos (711)+COMPOUND OF FORMULA I, quinalphos-methyl (1376)+COMPOUND OF FORMULA I, quinothion (1380)+COMPOUND OF FORMULA I, quintiofos (1381)+COMPOUND OF FORMULA I, R-1492 (development code) (1382)+COMPOUND OF FORMULA I, rafoxanide (alternative name) [CCN]+COMPOUND OF FORMULA I, resmethrin (719)+COMPOUND OF FORMULA I, rotenone (722)+COMPOUND OF FORMULA I, RU 15525 (development code) (723)+COMPOUND OF FORMULA I, RU 25475 (development code) (1386)+COMPOUND OF FORMULA I, ryania (alternative name) (1387)+COMPOUND OF FORMULA I, ryanodine (traditional name) (1387)+COMPOUND OF FORMULA I, sabadilla (alternative name) (725)+COMPOUND OF FORMULA I, schradan (1389)+COMPOUND OF FORMULA I, sebufos (alternative name)+COMPOUND OF FORMULA I, selamectin (alternative name) [CCN]+COMPOUND OF FORMULA I, SI-0009 (compound code)+COMPOUND OF FORMULA I, SI-0205 (compound code)+COMPOUND OF FORMULA I, SI-0404 (compound code)+COMPOUND OF FORMULA I, SI-0405 (compound code)+COMPOUND OF FORMULA I, silafluofen (728)+COMPOUND OF FORMULA I, SN 72129 (development code) (1397)+COMPOUND OF FORMULA I, sodium arsenite [CCN]+COMPOUND OF FORMULA I, sodium cyanide (444)+COMPOUND OF FORMULA I, sodium fluoride (IUPAC/Chemical Abstracts name) (1399)+COMPOUND OF FORMULA I, sodium hexafluorosilicate (1400)+COMPOUND OF FORMULA I, sodium pentachlorophenoxide (623)+COMPOUND OF FORMULA I, sodium selenate (IUPAC name) (1401)+COMPOUND OF FORMULA I, sodium thiocyanate [CCN]+COMPOUND OF FORMULA I, sophamide (1402)+COMPOUND OF FORMULA I, spinetoram [CCN]+COMPOUND OF FORMULA I, spinosad (737)+COMPOUND OF FORMULA I, spiromesifen (739)+COMPOUND OF FORMULA I, spirotetramat [CCN]+COMPOUND OF FORMULA I, sulcofuron (746)+COMPOUND OF FORMULA I, sulcofuron-sodium (746)+COMPOUND OF FORMULA I, sulfluramid (750)+COMPOUND OF FORMULA I, sulfotep (753)+COMPOUND OF FORMULA I, sulfoxaflor [CCN]+COMPOUND OF FORMULA I, sulfuryl fluoride (756)+COMPOUND OF FORMULA I, sulprofos (1408)+COMPOUND OF FORMULA I, tar oils (alternative name) (758)+COMPOUND OF FORMULA I, tau-fluvalinate (398)+COMPOUND OF FORMULA I, tazimcarb (1412)+COMPOUND OF FORMULA I, TDE (1414)+COMPOUND OF FORMULA I, tebufenozide (762)+COMPOUND OF FORMULA I, tebufenpyrad (763)+COMPOUND OF FORMULA I, tebupirimfos (764)+COMPOUND OF FORMULA I, teflubenzuron (768)+COMPOUND OF FORMULA I, tefluthrin (769)+COMPOUND OF FORMULA I, temephos (770)+COMPOUND OF FORMULA I, TEPP (1417)+COMPOUND OF FORMULA I, terallethrin (1418)+COMPOUND OF FORMULA I, terbam (alternative name)+COMPOUND OF FORMULA I, terbufos (773)+COMPOUND OF FORMULA I, tetrachloroethane [CCN]+COMPOUND OF FORMULA I, tetrachlorvinphos (777)+COMPOUND OF FORMULA I, tetramethrin (787)+COMPOUND OF FORMULA I, tetramethylfluthrin (CAS. Reg. No.: 84937-88-2)+COMPOUND OF FORMULA I, theta-cypermethrin (204)+COMPOUND OF FORMULA I, thiacloprid (791)+COMPOUND OF FORMULA I, thiafenox (alternative name)+COMPOUND OF FORMULA I, thiamethoxam (792)+COMPOUND OF FORMULA I, thicrofos (1428)+COMPOUND OF FORMULA I, thiocarboxime (1431)+COMPOUND OF FORMULA I, thiocyclam (798)+COMPOUND OF FORMULA I, thiocyclam hydrogen oxalate (798)+COMPOUND OF FORMULA I, thiodicarb (799)+COMPOUND OF FORMULA I, thiofanox (800)+COMPOUND OF FORMULA I, thiometon (801)+COMPOUND OF FORMULA I, thionazin (1434)+COMPOUND OF FORMULA I, thiosultap (803)+COMPOUND OF FORMULA I, thiosultap-sodium (803)+COMPOUND OF FORMULA I, thuringiensin (alternative name) [CCN]+COMPOUND OF FORMULA I, tolfenpyrad (809)+COMPOUND OF FORMULA I, tralomethrin (812)+COMPOUND OF FORMULA I, transfluthrin (813)+COMPOUND OF FORMULA I, transpermethrin (1440)+COMPOUND OF FORMULA I, triamiphos (1441)+COMPOUND OF FORMULA I, triazamate (818)+COMPOUND OF FORMULA I, triazophos (820)+COMPOUND OF FORMULA I, triazuron (alternative name)+COMPOUND OF FORMULA I, trichlorfon (824)+COMPOUND OF FORMULA I, trichlormetaphos-3 (alternative name) [CCN]+COMPOUND OF FORMULA I, trichloronat (1452)+COMPOUND OF FORMULA I, trifenofos (1455)+COMPOUND OF FORMULA I, triflumuron (835)+COMPOUND OF FORMULA I, trimethacarb (840)+COMPOUND OF FORMULA I, triprene (1459)+COMPOUND OF FORMULA I, vamidothion (847)+COMPOUND OF FORMULA I, vaniliprole [CCN]+COMPOUND OF FORMULA I, veratridine (alternative name) (725)+COMPOUND OF FORMULA I, veratrine (alternative name) (725)+COMPOUND OF FORMULA I, XMC (853)+COMPOUND OF FORMULA I, xylylcarb (854)+COMPOUND OF FORMULA I, YI-5302 (compound code)+COMPOUND OF FORMULA I, zeta-cypermethrin (205)+COMPOUND OF FORMULA I, zetamethrin (alternative name)+COMPOUND OF FORMULA I, zinc phosphide (640)+COMPOUND OF FORMULA I, zolaprofos (1469), ZJ0967 (development code)+COMPOUND OF FORMULA I, ZJ3757 (development code)+COMPOUND OF FORMULA I, and ZXI 8901 (development code) (858)+COMPOUND OF FORMULA I,
a molluscicide selected from the group of substances consisting of bis(tributyltin) oxide (IUPAC name) (913)+COMPOUND OF FORMULA I, bromoacetamide [CCN]+COMPOUND OF FORMULA I, calcium arsenate [CCN]+COMPOUND OF FORMULA I, cloethocarb (999)+COMPOUND OF FORMULA I, copper acetoarsenite [CCN]+COMPOUND OF FORMULA I, copper sulfate (172)+COMPOUND OF FORMULA I, fentin (347)+COMPOUND OF FORMULA I, ferric phosphate (IUPAC name) (352)+COMPOUND OF FORMULA I, metaldehyde (518)+COMPOUND OF FORMULA I, methiocarb (530)+COMPOUND OF FORMULA I, niclosamide (576)+COMPOUND OF FORMULA I, niclosamide-olamine (576)+COMPOUND OF FORMULA I, pentachlorophenol (623)+COMPOUND OF FORMULA I, sodium pentachlorophenoxide (623)+COMPOUND OF FORMULA I, tazimcarb (1412)+COMPOUND OF FORMULA I, thiodicarb (799)+COMPOUND OF FORMULA I, tralopyril [CCN]+COMPOUND OF FORMULA I, tributyltin oxide (913)+COMPOUND OF FORMULA I, trifenmorph (1454)+COMPOUND OF FORMULA I, trimethacarb (840)+COMPOUND OF FORMULA I, triphenyltin acetate (IUPAC name) (347) and triphenyltin hydroxide (IUPAC name) (347)+COMPOUND OF FORMULA I,
a nematicide selected from the group of substances consisting of AKD-3088 (compound code)+COMPOUND OF FORMULA I, 1,2-dibromo-3-chloropropane (IUPAC/Chemical Abstracts name) (1045)+COMPOUND OF FORMULA I, 1,2-dichloropropane (IUPAC/Chemical Abstracts name) (1062)+COMPOUND OF FORMULA 1,1,2-dichloropropane with 1,3-dichloropropene (IUPAC name) (1063)+COMPOUND OF FORMULA I, 1,3-dichloropropene (233)+COMPOUND OF FORMULA I, 3,4-dichlorotetrahydrothiophene 1,1-dioxide (IUPAC/Chemical Abstracts name) (1065)+COMPOUND OF FORMULA I, 3-(4-chlorophenyl)-5-methylrhodanine (IUPAC name) (980)+COMPOUND OF FORMULA I, 5-methyl-6-thioxo-1,3,5-thiadiazinan-3-ylacetic acid (IUPAC name) (1286)+COMPOUND OF FORMULA I, 6-isopentenylaminopurine (alternative name) (210)+COMPOUND OF FORMULA I, abamectin (1)+COMPOUND OF FORMULA I, acetoprole [CCN]+COMPOUND OF FORMULA I, alanycarb (15)+COMPOUND OF FORMULA I, aldicarb (16)+COMPOUND OF FORMULA I, aldoxycarb (863)+COMPOUND OF FORMULA I, AZ 60541 (compound code)+COMPOUND OF FORMULA I, benclothiaz [CCN]+COMPOUND OF FORMULA I, benomyl (62)+COMPOUND OF FORMULA I, butylpyridaben (alternative name)+COMPOUND OF FORMULA I, cadusafos (109)+COMPOUND OF FORMULA I, carbofuran (118)+COMPOUND OF FORMULA I, carbon disulfide (945)+COMPOUND OF FORMULA I, carbosulfan (119)+COMPOUND OF FORMULA I, chloropicrin (141)+COMPOUND OF FORMULA I, chlorpyrifos (145)+COMPOUND OF FORMULA I, cloethocarb (999)+COMPOUND OF FORMULA I, cytokinins (alternative name) (210)+COMPOUND OF FORMULA I, dazomet (216)+COMPOUND OF FORMULA I, DBCP (1045)+COMPOUND OF FORMULA I, DCIP (218)+COMPOUND OF FORMULA I, diamidafos (1044)+COMPOUND OF FORMULA I, dichlofenthion (1051)+COMPOUND OF FORMULA I, dicliphos (alternative name)+COMPOUND OF FORMULA I, dimethoate (262)+COMPOUND OF FORMULA I, doramectin (alternative name) [CCN]+COMPOUND OF FORMULA I, emamectin (291)+COMPOUND OF FORMULA I, emamectin benzoate (291)+COMPOUND OF FORMULA I, eprinomectin (alternative name) [CCN]+COMPOUND OF FORMULA I, ethoprophos (312)+COMPOUND OF FORMULA I, ethylene dibromide (316)+COMPOUND OF FORMULA I, fenamiphos (326)+COMPOUND OF FORMULA I, fenpyrad (alternative name)+COMPOUND OF FORMULA I, fensulfothion (1158)+COMPOUND OF FORMULA I, fluensulfone (CAS. Reg. No.: 318290-98-1)+COMPOUND OF FORMULA I, fosthiazate (408)+COMPOUND OF FORMULA I, fosthietan (1196)+COMPOUND OF FORMULA I, furfural (alternative name) [CCN]+COMPOUND OF FORMULA I, GY-81 (development code) (423)+COMPOUND OF FORMULA I, heterophos [CCN]+COMPOUND OF FORMULA I, imicyafos [CCN]+COMPOUND OF FORMULA I, imicyafos (alternative name) [CCN]+COMPOUND OF FORMULA I, iodomethane (IUPAC name) (542)+COMPOUND OF FORMULA I, isamidofos (1230)+COMPOUND OF FORMULA I, isazofos (1231)+COMPOUND OF FORMULA I, ivermectin (alternative name) [CCN]+COMPOUND OF FORMULA I, kinetin (alternative name) (210)+COMPOUND OF FORMULA I, mecarphon (1258)+COMPOUND OF FORMULA I, metam (519)+COMPOUND OF FORMULA I, metam-potassium (alternative name) (519)+COMPOUND OF FORMULA I, metam-sodium (519)+COMPOUND OF FORMULA I, methyl bromide (537)+COMPOUND OF FORMULA I, methyl isothiocyanate (543)+COMPOUND OF FORMULA I, milbemycin oxime (alternative name) [CCN]+COMPOUND OF FORMULA I, moxidectin (alternative name) [CCN]+COMPOUND OF FORMULA I, Myrothecium verrucaria composition (alternative name) (565)+COMPOUND OF FORMULA I, NC-184 (compound code)+COMPOUND OF FORMULA I, oxamyl (602)+COMPOUND OF FORMULA I, phorate (636)+COMPOUND OF FORMULA I, phosphamidon (639)+COMPOUND OF FORMULA I, phosphocarb [CCN]+COMPOUND OF FORMULA I, sebufos (alternative name)+COMPOUND OF FORMULA I, selamectin (alternative name) [CCN]+COMPOUND OF FORMULA I, spinosad (737)+COMPOUND OF FORMULA I, terbam (alternative name)+COMPOUND OF FORMULA I, terbufos (773)+COMPOUND OF FORMULA I, tetrachlorothiophene (IUPAC/Chemical Abstracts name) (1422)+COMPOUND OF FORMULA I, thiafenox (alternative name)+COMPOUND OF FORMULA I, thionazin (1434)+COMPOUND OF FORMULA I, triazophos (820)+COMPOUND OF FORMULA I, triazuron (alternative name)+COMPOUND OF FORMULA I, xylenols [CCN]+COMPOUND OF FORMULA I, YI-5302 (compound code) and zeatin (alternative name) (210)+COMPOUND OF FORMULA I,
a nitrification inhibitor selected from the group of substances consisting of potassium ethylxanthate [CCN] and nitrapyrin (580)+COMPOUND OF FORMULA I,
a plant activator selected from the group of substances consisting of acibenzolar (6)+COMPOUND OF FORMULA I, acibenzolar-5-methyl (6)+COMPOUND OF FORMULA I, probenazole (658) and Reynoutria sachalinensis extract (alternative name) (720)+COMPOUND OF FORMULA I,
a rodenticide selected from the group of substances consisting of 2-isovalerylindan-1,3-dione (IUPAC name) (1246)+COMPOUND OF FORMULA I, 4-(quinoxalin-2-ylamino)benzenesulfonamide (IUPAC name) (748)+COMPOUND OF FORMULA I, alpha-chlorohydrin [CCN]+COMPOUND OF FORMULA I, aluminium phosphide (640)+COMPOUND OF FORMULA I, antu (880)+COMPOUND OF FORMULA I, arsenous oxide (882)+COMPOUND OF FORMULA I, barium carbonate (891)+COMPOUND OF FORMULA I, bisthiosemi (912)+COMPOUND OF FORMULA I, brodifacoum (89)+COMPOUND OF FORMULA I, bromadiolone (91)+COMPOUND OF FORMULA I, bromethalin (92)+COMPOUND OF FORMULA I, calcium cyanide (444)+COMPOUND OF FORMULA I, chloralose (127)+COMPOUND OF FORMULA I, chlorophacinone (140)+COMPOUND OF FORMULA I, cholecalciferol (alternative name) (850)+COMPOUND OF FORMULA I, coumachlor (1004)+COMPOUND OF FORMULA I, coumafuryl (1005)+COMPOUND OF FORMULA I, coumatetralyl (175)+COMPOUND OF FORMULA I, crimidine (1009)+COMPOUND OF FORMULA I, difenacoum (246)+COMPOUND OF FORMULA I, difethialone (249)+COMPOUND OF FORMULA I, diphacinone (273)+COMPOUND OF FORMULA I, ergocalciferol (301)+COMPOUND OF FORMULA I, flocoumafen (357)+COMPOUND OF FORMULA I, fluoroacetamide (379)+COMPOUND OF FORMULA I, flupropadine (1183)+COMPOUND OF FORMULA I, flupropadine hydrochloride (1183)+COMPOUND OF FORMULA I, gamma-HCH (430)+COMPOUND OF FORMULA I, HCH (430)+COMPOUND OF FORMULA I, hydrogen cyanide (444)+COMPOUND OF FORMULA I, iodomethane (IUPAC name) (542)+COMPOUND OF FORMULA I, lindane (430)+COMPOUND OF FORMULA I, magnesium phosphide (IUPAC name) (640)+COMPOUND OF FORMULA I, methyl bromide (537)+COMPOUND OF FORMULA I, norbormide (1318)+COMPOUND OF FORMULA I, phosacetim (1336)+COMPOUND OF FORMULA I, phosphine (IUPAC name) (640)+COMPOUND OF FORMULA I, phosphorus [CCN]+COMPOUND OF FORMULA I, pindone (1341)+COMPOUND OF FORMULA I, potassium arsenite [CCN]+COMPOUND OF FORMULA I, pyrinuron (1371)+COMPOUND OF FORMULA I, scilliroside (1390)+COMPOUND OF FORMULA I, sodium arsenite [CCN]+COMPOUND OF FORMULA I, sodium cyanide (444)+COMPOUND OF FORMULA I, sodium fluoro-acetate (735)+COMPOUND OF FORMULA I, strychnine (745)+COMPOUND OF FORMULA I, thallium sulfate [CCN]+COMPOUND OF FORMULA I, warfarin (851) and zinc phosphide (640)+COMPOUND OF FORMULA I,
a synergist selected from the group of substances consisting of 2-(2-butoxyethoxy)-ethyl piperonylate (IUPAC name) (934)+COMPOUND OF FORMULA I, 5-(1,3-benzodioxol-5-yl)-3-hexylcyclohex-2-enone (IUPAC name) (903)+COMPOUND OF FORMULA I, farnesol with nerolidol (alternative name) (324)+COMPOUND OF FORMULA I, MB-599 (development code) (498)+COMPOUND OF FORMULA I, MGK 264 (development code) (296)+COMPOUND OF FORMULA I, piperonyl butoxide (649)+COMPOUND OF FORMULA I, piprotal (1343)+COMPOUND OF FORMULA I, propyl isomer (1358)+COMPOUND OF FORMULA I, S421 (development code) (724)+COMPOUND OF FORMULA I, sesamex (1393)+COMPOUND OF FORMULA I, sesasmolin (1394) and sulfoxide (1406)+COMPOUND OF FORMULA I,
an animal repellent selected from the group of substances consisting of anthraquinone (32)+COMPOUND OF FORMULA I, chloralose (127)+COMPOUND OF FORMULA I, copper naphthenate [CCN]+COMPOUND OF FORMULA I, copper oxychloride (171)+COMPOUND OF FORMULA I, diazinon (227)+COMPOUND OF FORMULA I, dicyclopentadiene (chemical name) (1069)+COMPOUND OF FORMULA I, guazatine (422)+COMPOUND OF FORMULA I, guazatine acetates (422)+COMPOUND OF FORMULA I, methiocarb (530)+COMPOUND OF FORMULA I, pyridin-4-amine (IUPAC name) (23)+COMPOUND OF FORMULA I, thiram (804)+COMPOUND OF FORMULA I, trimethacarb (840)+COMPOUND OF FORMULA I, zinc naphthenate [CCN] and ziram (856)+COMPOUND OF FORMULA I,
a virucide selected from the group of substances consisting of imanin (alternative name) [CCN] and ribavirin (alternative name) [CCN]+COMPOUND OF FORMULA I,
a wound protectant selected from the group of substances consisting of mercuric oxide (512)+COMPOUND OF FORMULA I, octhilinone (590) and thiophanate-methyl (802)+COMPOUND OF FORMULA I,
an insecticide selected from the group consisting of the compound of the formula A-1

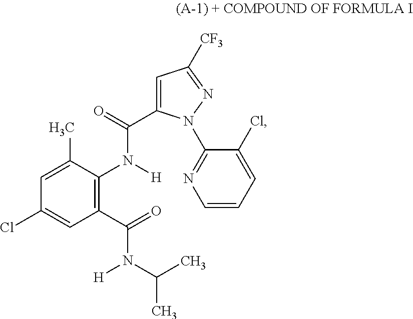 [SMILES: Cc1cc(Cl)cc(C(=O)NC(C)C)c1NC(=O)c1cc(C(F)(F)F)nn1-c1ncccc1Cl]

the formula A-2

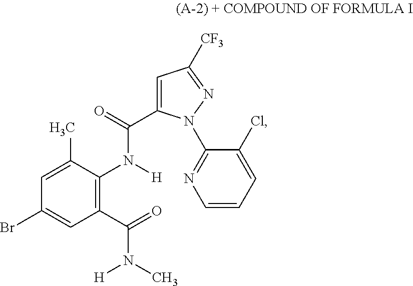 [SMILES: CNC(=O)c1cc(Br)cc(C)c1NC(=O)c1cc(C(F)(F)F)nn1-c1ncccc1Cl]

the formula A-3

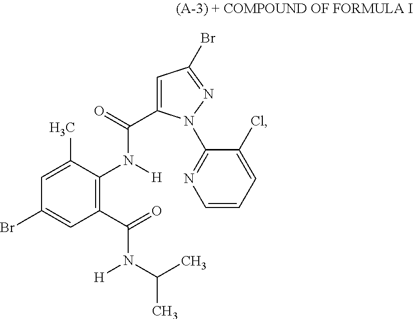 [SMILES: Cc1cc(Br)cc(C(=O)NC(C)C)c1NC(=O)c1cc(Br)nn1-c1ncccc1Cl]

the formula A-4

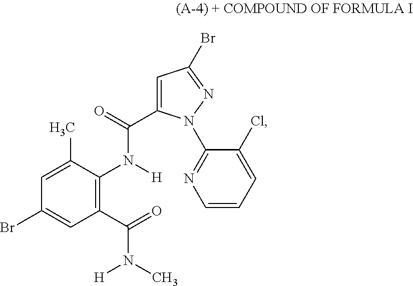 [SMILES: CNC(=O)c1cc(Br)cc(C)c1NC(=O)c1cc(Br)nn1-c1ncccc1Cl]

the formula A-5

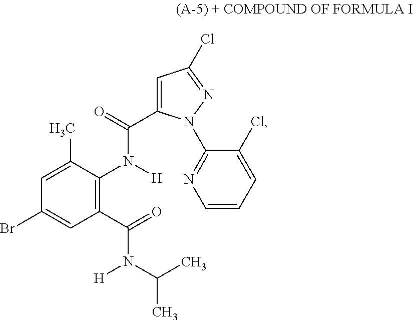 [SMILES: Cc1cc(Br)cc(C(=O)NC(C)C)c1NC(=O)c1cc(Cl)nn1-c1ncccc1Cl]

the formula A-6

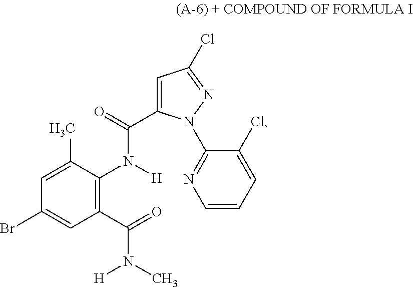 [SMILES: CNC(=O)c1cc(Br)cc(C)c1NC(=O)c1cc(Cl)nn1-c1ncccc1Cl]

the formula A-7

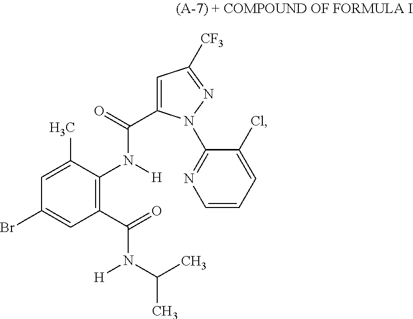 [SMILES: Cc1cc(Br)cc(C(=O)NC(C)C)c1NC(=O)c1cc(C(F)(F)F)nn1-c1ncccc1Cl]

the formula A-8

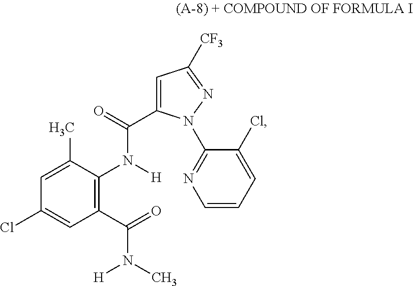 [SMILES: CNC(=O)c1cc(Cl)cc(C)c1NC(=O)c1cc(C(F)(F)F)nn1-c1ncccc1Cl]

the formula A-9

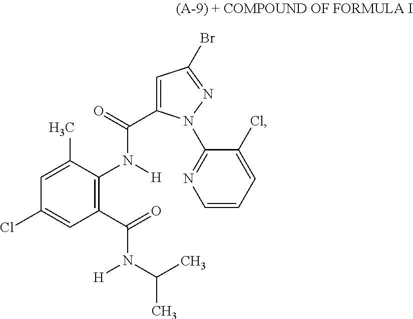 [SMILES: Cc1cc(Cl)cc(C(=O)NC(C)C)c1NC(=O)c1cc(Br)nn1-c1ncccc1Cl]

the formula A-10

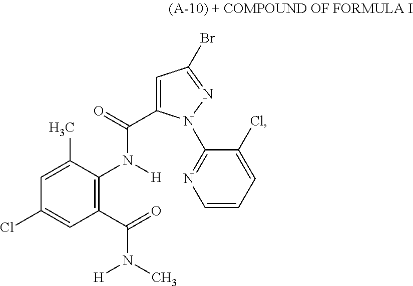 [SMILES: CNC(=O)c1cc(Cl)cc(C)c1NC(=O)c1cc(Br)nn1-c1ncccc1Cl]

the formula A-11

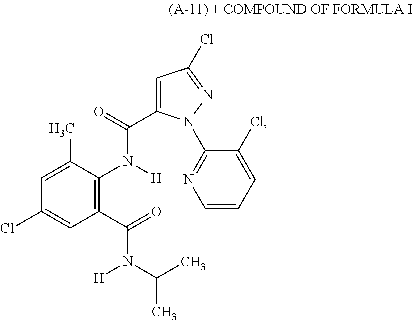 [SMILES: Cc1cc(Cl)cc(C(=O)NC(C)C)c1NC(=O)c1cc(Cl)nn1-c1ncccc1Cl]

the formula A-12

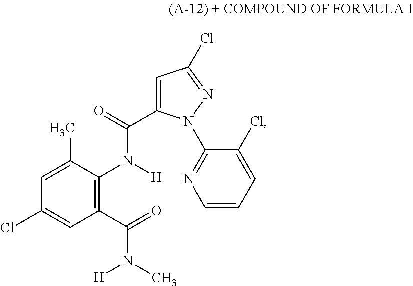 [SMILES: CNC(=O)c1cc(Cl)cc(C)c1NC(=O)c1cc(Cl)nn1-c1ncccc1Cl]

the formula A-13

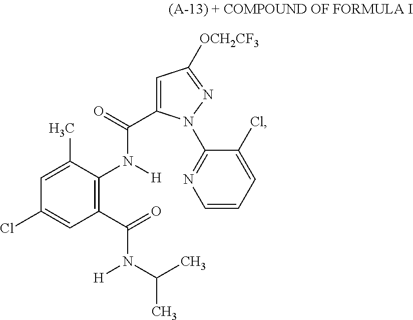

the formula A-14

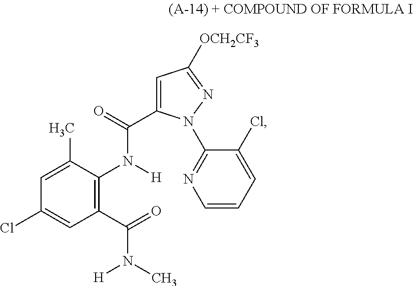

the formula A-15

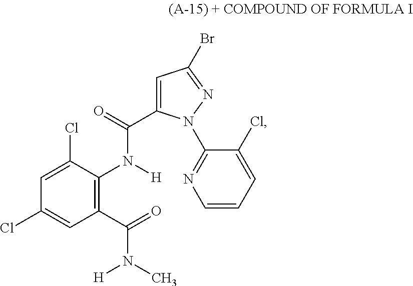 [SMILES: CNC(=O)c1cc(Cl)cc(Cl)c1NC(=O)c1cc(Br)nn1-c1ncccc1Cl]

the formula A-16

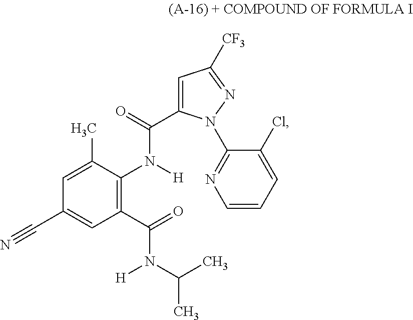 [SMILES: Cc1cc(C#N)cc(C(=O)NC(C)C)c1NC(=O)c1cc(C(F)(F)F)nn1-c1ncccc1Cl]

the formula A-17

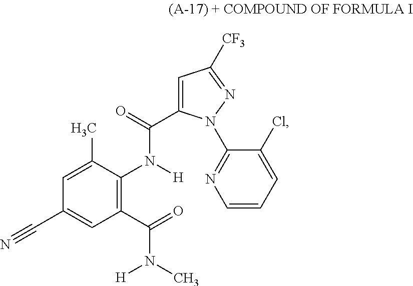 [SMILES: CNC(=O)c1cc(C#N)cc(C)c1NC(=O)c1cc(C(F)(F)F)nn1-c1ncccc1Cl]

the formula A-18

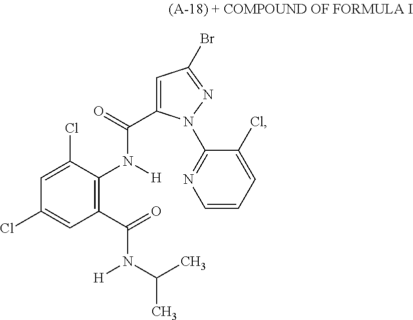 [SMILES: CC(C)NC(=O)c1cc(Cl)cc(Cl)c1NC(=O)c1cc(Br)nn1-c1ncccc1Cl]

the formula A-19

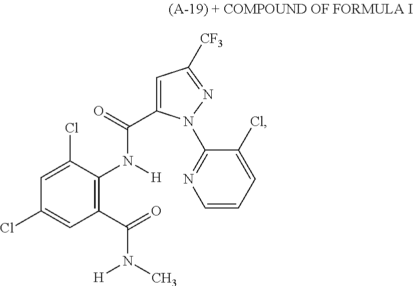 [SMILES: CNC(=O)c1cc(Cl)cc(Cl)c1NC(=O)c1cc(C(F)(F)F)nn1-c1ncccc1Cl]

the formula A-20

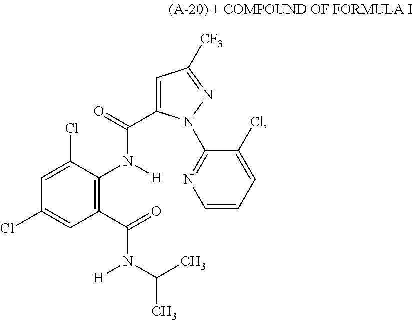 [SMILES: CC(C)NC(=O)c1cc(Cl)cc(Cl)c1NC(=O)c1cc(C(F)(F)F)nn1-c1ncccc1Cl]

the formula A-21

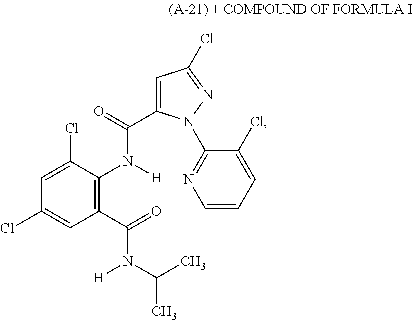 [SMILES: CC(C)NC(=O)c1cc(Cl)cc(Cl)c1NC(=O)c1cc(Cl)nn1-c1ncccc1Cl]

the formula A-22

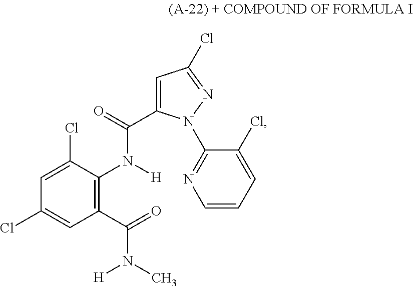 [SMILES: CNC(=O)c1cc(Cl)cc(Cl)c1NC(=O)c1cc(Cl)nn1-c1ncccc1Cl]

the formula A-23

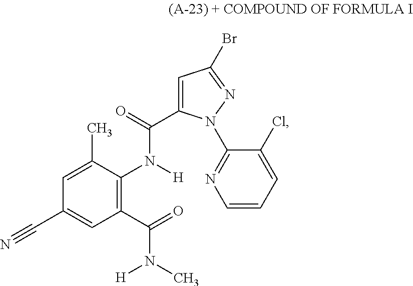 [SMILES: CNC(=O)c1cc(C#N)cc(C)c1NC(=O)c1cc(Br)nn1-c1ncccc1Cl]

the formula A-24

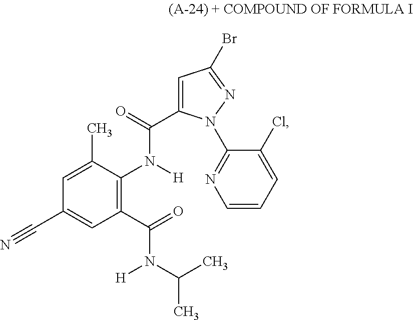 [SMILES: Cc1cc(C#N)cc(C(=O)NC(C)C)c1NC(=O)c1cc(Br)nn1-c1ncccc1Cl]

the formula A-25

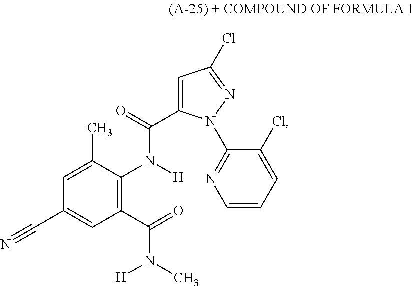 [SMILES: CNC(=O)c1cc(C#N)cc(C)c1NC(=O)c1cc(Cl)nn1-c1ncccc1Cl]

the formula A-26

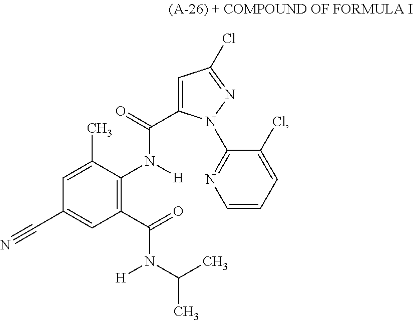 [SMILES: Cc1cc(C#N)cc(C(=O)NC(C)C)c1NC(=O)c1cc(Cl)nn1-c1ncccc1Cl]

and the formula A-27

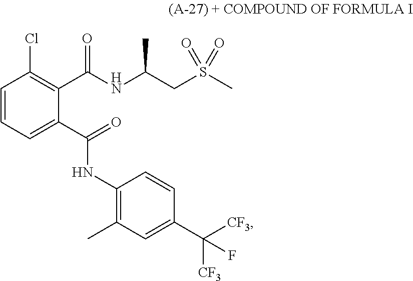 [SMILES: Cc1cc(C(F)(C(F)(F)F)C(F)(F)F)ccc1NC(=O)c1cccc(Cl)c1C(=O)N[C@@H](C)CS(C)(=O)=O]

an insecticide selected from the group consisting of the compound of the formula A-28

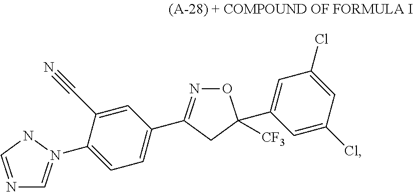

and the formula A-29

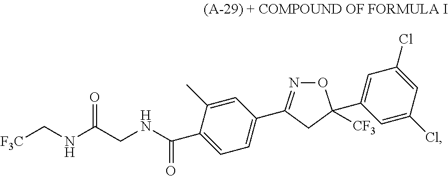 [SMILES: Cc1cc(C2=NOC(c3cc(Cl)cc(Cl)c3)(C(F)(F)F)C2)ccc1C(=O)NCC(=O)NCC(F)(F)F]

and the formula A-30

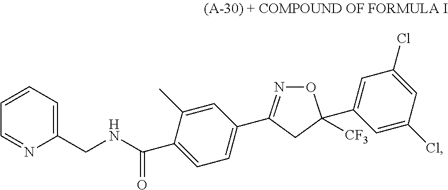 [SMILES: Cc1cc(C2=NOC(c3cc(Cl)cc(Cl)c3)(C(F)(F)F)C2)ccc1C(=O)NCc1ccccn1]

an insecticide selected from the group consisting of the compound of the formula A-31 [BYI2960]

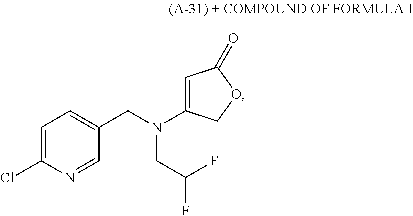 [SMILES: O=C1C=C(N(Cc2ccc(Cl)nc2)CC(F)F)CO1]

the formula A-32

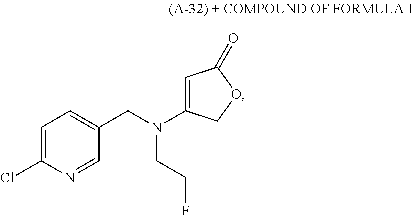 [SMILES: O=C1C=C(N(CCF)Cc2ccc(Cl)nc2)CO1]

the formula A-33

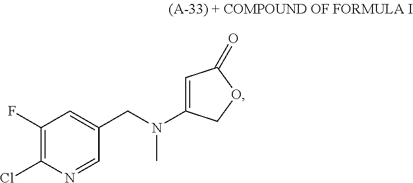 [SMILES: CN(Cc1cnc(Cl)c(F)c1)C1=CC(=O)OC1]

and an insecticide of the formula A-34

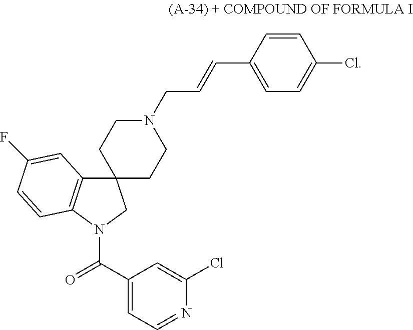 [SMILES: O=C(c1ccnc(Cl)c1)N1CC2(CCN(C/C=C/c3ccc(Cl)cc3)CC2)c2cc(F)ccc21]

The references in brackets behind the active ingredients, e.g. [3878-19-1] refer to the Chemical Abstracts Registry number. The compounds of the formula A-1 to A-26 are described in WO 03/015518 or in WO 04/067528. The compound of the formula A-27 is described in WO 06/022225 and in WO 07/112,844. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright® 1995-2004]; for example, the compound “acetoprole” is described under the internet address http://www.alanwood.net/pesticides/acetoprole.htmL.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The compounds of formula I according to the invention can also be used in combination with one or more fungicides. In particular, in the following mixtures of the compounds of formula I with fungicides, the term COMPOUND OF FORMULA I preferably refers to a compound selected from one of the Tables 1 to 333:

COMPOUND OF FORMULA I+(E)-N-methyl-2-[2-(2,5-dimethylphenoxymethyl)phenyl]-2-methoxy-iminoacetamide (SSF-129), COMPOUND OF FORMULA I+4-bromo-2-cyano-N,N-dimethyl-6-trifluoromethylbenzimidazole-1-sulphonamide, COMPOUND OF FORMULA I+α[N-(3-chloro-2,6-xylyl)-2-methoxyacetamido]-γ-butyrolactone, COMPOUND OF FORMULA I+4-chloro-2-cyano-N,N-dimethyl-5-p-tolylimidazole-1-sulfonamide (IKF-916, cyamidazosulfamid), COMPOUND OF FORMULA I+3-5-dichloro-N-(3-chloro-1-ethyl-1-methyl-2-oxopropyl)-4-methylbenzamide (RH-7281, zoxamide), COMPOUND OF FORMULA I+N-allyl-4,5-dimethyl-2-trimethylsilylthiophene-3-carboxamide (MON65500), COMPOUND OF FORMULA I+N-(1-cyano-1,2-dimethylpropyl)-2-(2,4-dichlorophenoxy)propionamide (AC382042), COMPOUND OF FORMULA I+N-(2-methoxy-5-pyridyl)-cyclopropane carboxamide, COMPOUND OF FORMULA I+acibenzolar, COMPOUND OF FORMULA I+alanycarb, COMPOUND OF FORMULA I+aldimorph, COMPOUND OF FORMULA I+ametoctradin, COMPOUND OF FORMULA I+amisulbrom, COMPOUND OF FORMULA I+anilazine, COMPOUND OF FORMULA I+azaconazole, COMPOUND OF FORMULA I+azoxystrobin, COMPOUND OF FORMULA I+benalaxyl, COMPOUND OF FORMULA I+benalaxyl-M, COMPOUND OF FORMULA I+benomyl, COMPOUND OF FORMULA I+benthiavalicarb, COMPOUND OF FORMULA I+benzodiflupyr, COMPOUND OF FORMULA I+benzovindiflupyr, COMPOUND OF FORMULA I+biloxazol, COMPOUND OF FORMULA I+bitertanol, COMPOUND OF FORMULA I+bixafen, COMPOUND OF FORMULA I+blasticidin S, COMPOUND OF FORMULA I+boscalid, COMPOUND OF FORMULA I+bromuconazole, COMPOUND OF FORMULA I+bupirimate, COMPOUND OF FORMULA I+captafol, COMPOUND OF FORMULA I+captan, COMPOUND OF FORMULA I+carbendazim, COMPOUND OF FORMULA I+carbendazim chlorhydrate, COMPOUND OF FORMULA I+carboxin, COMPOUND OF FORMULA I+carpropamid, carvone, COMPOUND OF FORMULA I+CGA41396, COMPOUND OF FORMULA I+CGA41397, COMPOUND OF FORMULA I+chinomethionate, COMPOUND OF FORMULA I+chlazafenone, COMPOUND OF FORMULA I+chlorodincarb, COMPOUND OF FORMULA I+chlorothalonil, COMPOUND OF FORMULA I+chlorozolinate, COMPOUND OF FORMULA I+clozylacon, COMPOUND OF FORMULA I+copper containing compounds such as copper oxychloride, copper oxyquinolate, copper sulphate, copper tallate and Bordeaux mixture, COMPOUND OF FORMULA I+coumoxystrobin, COMPOUND OF FORMULA I+cyazofamid, COMPOUND OF FORMULA I+cyflufenamid, COMPOUND OF FORMULA I+cymoxanil, COMPOUND OF FORMULA I+cyproconazole, COMPOUND OF FORMULA I+cyprodinil, COMPOUND OF FORMULA I+debacarb, COMPOUND OF FORMULA I+di-2-pyridyl disulphide 1,1′-dioxide, COMPOUND OF FORMULA I+dicloaminstrobin, COMPOUND OF FORMULA I+diclofenoxystrobin, COMPOUND OF FORMULA I+dichlofluanid, COMPOUND OF FORMULA I+diclomezine, COMPOUND OF FORMULA I+dicloran, COMPOUND OF FORMULA I+diethofencarb, COMPOUND OF FORMULA I+difenoconazole, COMPOUND OF FORMULA I+difenzoquat, COMPOUND OF FORMULA I+diflumetorim, COMPOUND OF FORMULA I+O,O-di-iso-propyl-5-benzyl thiophosphate, COMPOUND OF FORMULA I+dimefluazole, COMPOUND OF FORMULA I+dimetconazole, COMPOUND OF FORMULA I+dimethomorph, COMPOUND OF FORMULA I+dimethirimol, COMPOUND OF FORMULA I+dimoxystrobin, COMPOUND OF FORMULA I+diniconazole, COMPOUND OF FORMULA I+dinocap, COMPOUND OF FORMULA I+dithianon, COMPOUND OF FORMULA I+dodecyl dimethyl ammonium chloride, COMPOUND OF FORMULA I+dodemorph, COMPOUND OF FORMULA I+dodine, COMPOUND OF FORMULA I+doguadine, COMPOUND OF FORMULA I+edifenphos, COMPOUND OF FORMULA I+enoxastrobin, COMPOUND OF FORMULA I+epoxiconazole, COMPOUND OF FORMULA I+ethirimol, COMPOUND OF FORMULA I+ethyl(Z)-N-benzyl-N([methyl(methyl-thioethylideneaminooxycarbonyl)amino]thio)-β-alaninate, COMPOUND OF FORMULA I+etridiazole, COMPOUND OF FORMULA I+famoxadone, COMPOUND OF FORMULA I+fenamidone (RPA407213), COMPOUND OF FORMULA I+fenaminstrobin, COMPOUND OF FORMULA I+fenarimol, COMPOUND OF FORMULA I+fenbuconazole, COMPOUND OF FORMULA I+fenfuram, COMPOUND OF FORMULA I+fenhexamid (KBR2738), COMPOUND OF FORMULA I+fenoxanil, COMPOUND OF FORMULA I+fenoxystrobin, COMPOUND OF FORMULA I+fenpiclonil, COMPOUND OF FORMULA I+fenpropidin, COMPOUND OF FORMULA I+fenpropimorph, COMPOUND OF FORMULA I+fenpyrazamine, COMPOUND OF FORMULA I+fenpyrazamine/ipfenpyrazolone, COMPOUND OF FORMULA I+fentin acetate, COMPOUND OF FORMULA I+fentin hydroxide, COMPOUND OF FORMULA I+ferbam, COMPOUND OF FORMULA I+ferimzone, COMPOUND OF FORMULA I+fluazinam, COMPOUND OF FORMULA I+fludioxonil, COMPOUND OF FORMULA I+flufenoxystrobin, COMPOUND OF FORMULA I+flumetover, COMPOUND OF FORMULA I+flumorph, COMPOUND OF FORMULA I+fluopicolide, COMPOUND OF FORMULA I+fluopyram, COMPOUND OF FORMULA I+fluoxastrobin, COMPOUND OF FORMULA I+fluoroimide, COMPOUND OF FORMULA I+fluquinconazole, COMPOUND OF FORMULA I+flusilazole, COMPOUND OF FORMULA I+flutianil, COMPOUND OF FORMULA I+flutolanil, COMPOUND OF FORMULA I+flutriafol, COMPOUND OF FORMULA I+fluxapyroxad, COMPOUND OF FORMULA I+folpet, COMPOUND OF FORMULA I+fosetyl, COMPOUND OF FORMULA I+fosetyl-aluminium, COMPOUND OF FORMULA I+fuberidazole, COMPOUND OF FORMULA I+furalaxyl, COMPOUND OF FORMULA I+furametpyr, COMPOUND OF FORMULA I+guazatine, COMPOUND OF FORMULA I+hexaconazole, COMPOUND OF FORMULA I+hydroxyisoxazole, COMPOUND OF FORMULA I+hymexazole, COMPOUND OF FORMULA I+imazalil, COMPOUND OF FORMULA I+imibenconazole, COMPOUND OF FORMULA I+iminoctadine, COMPOUND OF FORMULA I+iminoctadine triacetate, COMPOUND OF FORMULA I+ipconazole, COMPOUND OF FORMULA I+iprobenfos, COMPOUND OF FORMULA I+iprodione, COMPOUND OF FORMULA I+iprovalicarb (SZX0722), COMPOUND OF FORMULA I+isopropanyl butyl carbamate, COMPOUND OF FORMULA I+isoprothiolane, COMPOUND OF FORMULA I+isopyrazam, COMPOUND OF FORMULA I+isotianil, COMPOUND OF FORMULA I+kasugamycin, COMPOUND OF FORMULA I+kresoxim-methyl, COMPOUND OF FORMULA I+LY186054, COMPOUND OF FORMULA I+LY211795, COMPOUND OF FORMULA I+LY248908, COMPOUND OF FORMULA I+mancozeb, COMPOUND OF FORMULA I+mandipropamid, COMPOUND OF FORMULA I+maneb, COMPOUND OF FORMULA I+mefenoxam, COMPOUND OF FORMULA I+mepanipyrim, COMPOUND OF FORMULA I+mepronil, COMPOUND OF FORMULA I+meptyldinocap, COMPOUND OF FORMULA I+metalaxyl, COMPOUND OF FORMULA I+metconazole, COMPOUND OF FORMULA I+metiram, COMPOUND OF FORMULA I+metiram-zinc, COMPOUND OF FORMULA I+metominostrobin, COMPOUND OF FORMULA I+metrafenone, COMPOUND OF FORMULA I+myclobutanil, COMPOUND OF FORMULA I+neoasozin, COMPOUND OF FORMULA I+nickel dimethyldithiocarbamate, COMPOUND OF FORMULA I+nicobifen, COMPOUND OF FORMULA I+nitrothal-isopropyl, COMPOUND OF FORMULA I+nuarimol, COMPOUND OF FORMULA I+ofurace, COMPOUND OF FORMULA I+organomercury compounds, COMPOUND OF FORMULA I+orysastrobin, COMPOUND OF FORMULA I+oxadixyl, COMPOUND OF FORMULA I+oxasulfuron, COMPOUND OF FORMULA I+oxolinic acid, COMPOUND OF FORMULA I+oxpoconazole, COMPOUND OF FORMULA I+oxycarboxin, COMPOUND OF FORMULA I+pefurazoate, COMPOUND OF FORMULA I+penconazole, COMPOUND OF FORMULA I+pencycuron, COMPOUND OF FORMULA I+penflufen, COMPOUND OF FORMULA I+penthiopyrad, COMPOUND OF FORMULA I+phenazin oxide, COMPOUND OF FORMULA I+phosetyl-Al, COMPOUND OF FORMULA I+phosphorus acids, COMPOUND OF FORMULA I+phthalide, COMPOUND OF FORMULA I+picoxystrobin (ZA1963), COMPOUND OF FORMULA I+polyoxin D, COMPOUND OF FORMULA I+polyram, COMPOUND OF FORMULA I+probenazole, COMPOUND OF FORMULA I+prochloraz, COMPOUND OF FORMULA I+procymidone, COMPOUND OF FORMULA I+propamocarb, COMPOUND OF FORMULA I+propiconazole, COMPOUND OF FORMULA I+propineb, COMPOUND OF FORMULA I+propionic acid, COMPOUND OF FORMULA I+proquinazid, COMPOUND OF FORMULA I+prothioconazole, COMPOUND OF FORMULA I+pyraclostrobin, COMPOUND OF FORMULA I+pyraoxystrobin, COMPOUND OF FORMULA I+pyrazophos, COMPOUND OF FORMULA I+pyribencarb, COMPOUND OF FORMULA I+pyrifenox, COMPOUND OF FORMULA I+pyrimethanil, COMPOUND OF FORMULA I+pyrisoxazole, COMPOUND OF FORMULA I+pyroquilon, COMPOUND OF FORMULA I+pyroxyfur, COMPOUND OF FORMULA I+pyrrolnitrin, COMPOUND OF FORMULA I+quaternary ammonium compounds, COMPOUND OF FORMULA I+quinomethionate, COMPOUND OF FORMULA I+quinoxyfen, COMPOUND OF FORMULA I+quintozene, COMPOUND OF FORMULA I+sedaxane, COMPOUND OF FORMULA I+sipconazole (F-155), COMPOUND OF FORMULA I+sodium pentachlorophenate, COMPOUND OF FORMULA I+spiroxamine, COMPOUND OF FORMULA I+streptomycin, COMPOUND OF FORMULA I+sulphur, COMPOUND OF FORMULA I+tebuconazole, COMPOUND OF FORMULA I+tecloftalam, COMPOUND OF FORMULA I+tecnazene, COMPOUND OF FORMULA I+terbufloquin, COMPOUND OF FORMULA I+tetraconazole, COMPOUND OF FORMULA I+thiabendazole, COMPOUND OF FORMULA I+thifluzamid, COMPOUND OF FORMULA I+2-(thiocyanomethylthio)benzothiazole, COMPOUND OF FORMULA I+thiophanate-methyl, COMPOUND OF FORMULA I+thiram, COMPOUND OF FORMULA I+tiadinil, COMPOUND OF FORMULA I+timibenconazole, COMPOUND OF FORMULA I+tolclofos-methyl, COMPOUND OF FORMULA I+tolylfluanid, COMPOUND OF FORMULA I+triadimefon, COMPOUND OF FORMULA I+triadimenol, COMPOUND OF FORMULA I+triazbutil, COMPOUND OF FORMULA I+triazoxide, COMPOUND OF FORMULA I+triclopyricarb, COMPOUND OF FORMULA I+tricyclazole, COMPOUND OF FORMULA I+tridemorph, COMPOUND OF FORMULA I+trifloxystrobin, COMPOUND OF FORMULA I+triforine, COMPOUND OF FORMULA I+triflumizole, COMPOUND OF FORMULA I+triticonazole, COMPOUND OF FORMULA I+validamycin A, COMPOUND OF FORMULA I+valiphenal, COMPOUND OF FORMULA I+vapam, COMPOUND OF FORMULA I+vinclozolin, COMPOUND OF FORMULA I+zineb and COMPOUND OF FORMULA I+ziram.

The compounds of formula I may be mixed with soil, peat or other rooting media for the protection of plants against seed-borne, soil-borne or foliar fungal diseases.

The compounds of formula I according to the invention can also be used in combination with one or more other synergists. In particular, the following mixtures of the COMPOUND OF FORMULA I, where this term preferably refers to a compound selected from one of the Tables 1 to 333, are important:

COMPOUND OF FORMULA I+piperonyl butoxide, COMPOUND OF FORMULA I+sesamex, COMPOUND OF FORMULA I+safroxan and COMPOUND OF FORMULA I+dodecyl imidazole.

The compounds of formula I according to the invention can also be used in combination with one or more other herbicides. In particular, the following mixtures of the COMPOUND OF FORMULA I, where this term preferably refers to a compound selected from one of the Tables 1 to 333, are important:

COMPOUND OF FORMULA I+acetochlor, COMPOUND OF FORMULA I+acifluorfen, COMPOUND OF FORMULA I+acifluorfen-sodium, COMPOUND OF FORMULA I+aclonifen, COMPOUND OF FORMULA I+acrolein, COMPOUND OF FORMULA I+alachlor, COMPOUND OF FORMULA I+alloxydim, COMPOUND OF FORMULA I+allyl alcohol, COMPOUND OF FORMULA I+ametryn, COMPOUND OF FORMULA I+amicarbazone, COMPOUND OF FORMULA I+amidosulfuron, COMPOUND OF FORMULA I+aminocyclopyrachlor, COMPOUND OF FORMULA I+aminopyralid, COMPOUND OF FORMULA I+amitrole, COMPOUND OF FORMULA I+ammonium sulfamate, COMPOUND OF FORMULA I+anilofos, COMPOUND OF FORMULA I+asulam, COMPOUND OF FORMULA I+atraton, COMPOUND OF FORMULA I+atrazine, COMPOUND OF FORMULA I+azimsulfuron, COMPOUND OF FORMULA I+BCPC, COMPOUND OF FORMULA I+beflubutamid, COMPOUND OF FORMULA I+benazolin, COMPOUND OF FORMULA I+bencarbazone, COMPOUND OF FORMULA I+benfluralin, COMPOUND OF FORMULA I+benfuresate, COMPOUND OF FORMULA I+bensulfuron, COMPOUND OF FORMULA I+bensulfuron-methyl, COMPOUND OF FORMULA I+bensulide, COMPOUND OF FORMULA I+bentazone, COMPOUND OF FORMULA I+benzfendizone, COMPOUND OF FORMULA I+benzobicyclon, COMPOUND OF FORMULA I+benzofenap, COMPOUND OF THE FORMULA I+bicyclopyrone, COMPOUND OF FORMULA I+bifenox, COMPOUND OF FORMULA I+bilanafos, COMPOUND OF FORMULA I+bispyribac, COMPOUND OF FORMULA I+bispyribac-sodium, COMPOUND OF FORMULA I+borax, COMPOUND OF FORMULA I+bromacil, COMPOUND OF FORMULA I+bromobutide, COMPOUND OF FORMULA I+bromoxynil, COMPOUND OF FORMULA I+butachlor, COMPOUND OF FORMULA I+butafenacil, COMPOUND OF FORMULA I+butamifos, COMPOUND OF FORMULA I+butralin, COMPOUND OF FORMULA I+butroxydim, COMPOUND OF FORMULA I+butylate, COMPOUND OF FORMULA I+cacodylic acid, COMPOUND OF FORMULA I+calcium chlorate, COMPOUND OF FORMULA I+cafenstrole, COMPOUND OF FORMULA I+carbetamide, COMPOUND OF FORMULA I+carfentrazone, COMPOUND OF FORMULA I+carfentrazone-ethyl, COMPOUND OF FORMULA I+CDEA, COMPOUND OF FORMULA I+CEPC, COMPOUND OF FORMULA I+chlorflurenol, COMPOUND OF FORMULA I+chlorflurenol-methyl, COMPOUND OF FORMULA I+chloridazon, COMPOUND OF FORMULA I+chlorimuron, COMPOUND OF FORMULA I+chlorimuron-ethyl, COMPOUND OF FORMULA I+chloroacetic acid, COMPOUND OF FORMULA I+chlorotoluron, COMPOUND OF FORMULA I+chlorpropham, COMPOUND OF FORMULA I+chlorsulfuron, COMPOUND OF FORMULA I+chlorthal, COMPOUND OF FORMULA I+chlorthal-dimethyl, COMPOUND OF FORMULA I+cinidon-ethyl, COMPOUND OF FORMULA I+cinmethylin, COMPOUND OF FORMULA I+cinosulfuron, COMPOUND OF FORMULA I+cisanilide, COMPOUND OF FORMULA I+clethodim, COMPOUND OF FORMULA I+clodinafop, COMPOUND OF FORMULA I+clodinafop-propargyl, COMPOUND OF FORMULA I+clomazone, COMPOUND OF FORMULA I+clomeprop, COMPOUND OF FORMULA I+clopyralid, COMPOUND OF FORMULA I+cloransulam, COMPOUND OF FORMULA I+cloransulam-methyl, COMPOUND OF FORMULA I+CMA, COMPOUND OF FORMULA I+4-CPB, COMPOUND OF FORMULA I+CPMF, COMPOUND OF FORMULA I+4-CPP, COMPOUND OF FORMULA I+CPPC, COMPOUND OF FORMULA I+cresol, COMPOUND OF FORMULA I+cumyluron, COMPOUND OF FORMULA I+cyanamide, COMPOUND OF FORMULA I+cyanazine, COMPOUND OF FORMULA I+cycloate, COMPOUND OF FORMULA I+cyclosulfamuron, COMPOUND OF FORMULA I+cycloxydim, COMPOUND OF FORMULA I+cyhalofop, COMPOUND OF FORMULA I+cyhalofop-butyl, COMPOUND OF FORMULA I+2,4-D, COMPOUND OF FORMULA I+3,4-DA, COMPOUND OF FORMULA I+daimuron, COMPOUND OF FORMULA I+dalapon, COMPOUND OF FORMULA I+dazomet, COMPOUND OF FORMULA I+2,4-DB, COMPOUND OF FORMULA I+3,4-DB, COMPOUND OF FORMULA I+2,4-DEB, COMPOUND OF FORMULA I+desmedipham, COMPOUND OF FORMULA I+dicamba, COMPOUND OF FORMULA I+dichlobenil, COMPOUND OF FORMULA I+ortho-dichlorobenzene, COMPOUND OF FORMULA I+para-dichlorobenzene, COMPOUND OF FORMULA I+dichlorprop, COMPOUND OF FORMULA I+dichlorprop-P, COMPOUND OF FORMULA I+diclofop, COMPOUND OF FORMULA I+diclofop-methyl, COMPOUND OF FORMULA I+diclosulam, COMPOUND OF FORMULA I+difenzoquat, COMPOUND OF FORMULA I+difenzoquat metilsulfate, COMPOUND OF FORMULA I+diflufenican, COMPOUND OF FORMULA I+diflufenzopyr, COMPOUND OF FORMULA I+dimefuron, COMPOUND OF FORMULA I+dimepiperate, COMPOUND OF FORMULA I+dimethachlor, COMPOUND OF FORMULA I+dimethametryn, COMPOUND OF FORMULA I+dimethenamid, COMPOUND OF FORMULA I+dimethenamid-P, COMPOUND OF FORMULA I+dimethipin, COMPOUND OF FORMULA I+dimethylarsinic acid, COMPOUND OF FORMULA I+dinitramine, COMPOUND OF FORMULA I+dinoterb, COMPOUND OF FORMULA I+diphenamid, COMPOUND OF FORMULA I+diquat, COMPOUND OF FORMULA I+diquat dibromide, COMPOUND OF FORMULA I+dithiopyr, COMPOUND OF FORMULA I+diuron, COMPOUND OF FORMULA I+DNOC, COMPOUND OF FORMULA I+3,4-DP, COMPOUND OF FORMULA I+DSMA, COMPOUND OF FORMULA I+EBEP, COMPOUND OF FORMULA I+endothal, COMPOUND OF FORMULA I+EPTC, COMPOUND OF FORMULA I+esprocarb, COMPOUND OF FORMULA I+ethalfluralin, COMPOUND OF FORMULA I+ethametsulfuron, COMPOUND OF FORMULA I+ethametsulfuron-methyl, COMPOUND OF FORMULA I+ethofumesate, COMPOUND OF FORMULA I+ethoxyfen, COMPOUND OF FORMULA I+ethoxysulfuron, COMPOUND OF FORMULA I+etobenzanid, COMPOUND OF FORMULA I+fenoxaprop-P, COMPOUND OF FORMULA I+fenoxaprop-P-ethyl, COMPOUND OF FORMULA I+fentrazamide, COMPOUND OF FORMULA I+ferrous sulfate, COMPOUND OF FORMULA I+flamprop-M, COMPOUND OF FORMULA I+flazasulfuron, COMPOUND OF FORMULA I+florasulam, COMPOUND OF FORMULA I+fluazifop, COMPOUND OF FORMULA I+fluazifop-butyl, COMPOUND OF FORMULA I+fluazifop-P, COMPOUND OF FORMULA I+fluazifop-P-butyl, COMPOUND OF FORMULA I+flucarbazone, COMPOUND OF FORMULA I+flucarbazone-sodium, COMPOUND OF FORMULA I+flucetosulfuron, COMPOUND OF FORMULA I+fluchloralin, COMPOUND OF FORMULA I+flufenacet, COMPOUND OF FORMULA I+flufenpyr, COMPOUND OF FORMULA I+flufenpyr-ethyl, COMPOUND OF FORMULA I+flumetsulam, COMPOUND OF FORMULA I+flumiclorac, COMPOUND OF FORMULA I+flumiclorac-pentyl, COMPOUND OF FORMULA I+flumioxazin, COMPOUND OF FORMULA I+fluometuron, COMPOUND OF FORMULA I+fluoroglycofen, COMPOUND OF FORMULA I+fluoroglycofen-ethyl, COMPOUND OF FORMULA I+flupropanate, COMPOUND OF FORMULA I+flupyrsulfuron, COMPOUND OF FORMULA I+flupyrsulfuron-methyl-sodium, COMPOUND OF FORMULA I+flurenol, COMPOUND OF FORMULA I+fluridone, COMPOUND OF FORMULA I+fluorochloridone, COMPOUND OF FORMULA I+fluoroxypyr, COMPOUND OF FORMULA I+flurtamone, COMPOUND OF FORMULA I+fluthiacet, COMPOUND OF FORMULA I+fluthiacet-methyl, COMPOUND OF FORMULA I+fomesafen, COMPOUND OF FORMULA I+foramsulfuron, COMPOUND OF FORMULA I+fosamine, COMPOUND OF FORMULA I+glufosinate, COMPOUND OF FORMULA I+glufosinate-ammonium, COMPOUND OF FORMULA I+glufosinate-P, COMPOUND OF FORMULA I+glyphosate, COMPOUND OF FORMULA I+glyphosate-trimesium, COMPOUND OF FORMULA I+halosulfuron, COMPOUND OF FORMULA I+halosulfuron-methyl, COMPOUND OF FORMULA I+haloxyfop, COMPOUND OF FORMULA I+haloxyfop-P, COMPOUND OF FORMULA I+HC-252, COMPOUND OF FORMULA I+hexazinone, COMPOUND OF FORMULA I+imazamethabenz, COMPOUND OF FORMULA I+imazamethabenz-methyl, COMPOUND OF FORMULA I+imazamox, COMPOUND OF FORMULA I+imazapic, COMPOUND OF FORMULA I+imazapyr, COMPOUND OF FORMULA I+imazaquin, COMPOUND OF FORMULA I+imazethapyr, COMPOUND OF FORMULA I+imazosulfuron, COMPOUND OF FORMULA I+indanofan, COMPOUND OF FORMULA I+indaziflam, COMPOUND OF FORMULA I+iodomethane, COMPOUND OF FORMULA I+iodosulfuron, COMPOUND OF FORMULA I+iodosulfuron-methyl-sodium, COMPOUND OF FORMULA I+iofensulfuron, COMPOUND OF FORMULA I+ioxynil, COMPOUND OF FORMULA I+ipfencarbazone, COMPOUND OF FORMULA I+isoproturon, COMPOUND OF FORMULA I+isouron, COMPOUND OF FORMULA I+isoxaben, COMPOUND OF FORMULA I+isoxachlortole, COMPOUND OF FORMULA I+isoxaflutole, COMPOUND OF FORMULA I+karbutilate, COMPOUND OF FORMULA I+lactofen, COMPOUND OF FORMULA I+lenacil, COMPOUND OF FORMULA I+linuron, COMPOUND OF FORMULA I+MAA, COMPOUND OF FORMULA I+MAMA, COMPOUND OF FORMULA I+MCPA, COMPOUND OF FORMULA I+MCPA-thioethyl, COMPOUND OF FORMULA I+MCPB, COMPOUND OF FORMULA I+mecoprop, COMPOUND OF FORMULA I+mecoprop-P, COMPOUND OF FORMULA I+mefenacet, COMPOUND OF FORMULA I+mefluidide, COMPOUND OF FORMULA I+mesosulfuron, COMPOUND OF FORMULA I+mesosulfuron-methyl, COMPOUND OF FORMULA I+mesotrione, COMPOUND OF FORMULA I+metam, COMPOUND OF FORMULA I+metamifop, COMPOUND OF FORMULA I+metamitron, COMPOUND OF FORMULA I+metazachlor, COMPOUND OF FORMULA I+methabenzthiazuron, COMPOUND OF FORMULA I+methylarsonic acid, COMPOUND OF FORMULA I+methyldymron, COMPOUND OF FORMULA I+methyl isothiocyanate, COMPOUND OF FORMULA I+metiozolin, COMPOUND OF FORMULA I+metobenzuron, COMPOUND OF FORMULA I+metolachlor, COMPOUND OF FORMULA I+S-metolachlor, COMPOUND OF FORMULA I+metosulam, COMPOUND OF FORMULA I+metoxuron, COMPOUND OF FORMULA I+metribuzin, COMPOUND OF FORMULA I+metsulfuron, COMPOUND OF FORMULA I+metsulfuron-methyl, COMPOUND OF FORMULA I+MK-616, COMPOUND OF FORMULA I+molinate, COMPOUND OF FORMULA I+monolinuron, COMPOUND OF FORMULA I+MSMA, COMPOUND OF FORMULA I+naproanilide, COMPOUND OF FORMULA I+napropamide, COMPOUND OF FORMULA I+naptalam, COMPOUND OF FORMULA I+neburon, COMPOUND OF FORMULA I+nicosulfuron, COMPOUND OF FORMULA I+nonanoic acid, COMPOUND OF FORMULA I+norflurazon, COMPOUND OF FORMULA I+oleic acid (fatty acids), COMPOUND OF FORMULA I+orbencarb, COMPOUND OF FORMULA I+orthosulfamuron, COMPOUND OF FORMULA I+oryzalin, COMPOUND OF FORMULA I+oxadiargyl, COMPOUND OF FORMULA I+oxadiazon, COMPOUND OF FORMULA I+oxasulfuron, COMPOUND OF FORMULA I+oxaziclomefone, COMPOUND OF FORMULA I+oxyfluorfen, COMPOUND OF FORMULA I+paraquat, COMPOUND OF FORMULA I+paraquat dichloride, COMPOUND OF FORMULA I+pebulate, COMPOUND OF FORMULA I+pendimethalin, COMPOUND OF FORMULA I+penoxsulam, COMPOUND OF FORMULA I+pentachlorophenol, COMPOUND OF FORMULA I+pentanochlor, COMPOUND OF FORMULA I+pentoxazone, COMPOUND OF FORMULA I+pethoxamid, COMPOUND OF FORMULA I+petrolium oils, COMPOUND OF FORMULA I+phenmedipham, COMPOUND OF FORMULA I+phenmedipham-ethyl, COMPOUND OF FORMULA I+picloram, COMPOUND OF FORMULA I+picolinafen, COMPOUND OF FORMULA I+pinoxaden, COMPOUND OF FORMULA I+piperophos, COMPOUND OF FORMULA I+potassium arsenite, COMPOUND OF FORMULA I+potassium azide, COMPOUND OF FORMULA I+pretilachlor, COMPOUND OF FORMULA I+primisulfuron, COMPOUND OF FORMULA I+primisulfuron-methyl, COMPOUND OF FORMULA I+prodiamine, COMPOUND OF FORMULA I+profluazol, COMPOUND OF FORMULA I+profoxydim, COMPOUND OF FORMULA I+prometon, COMPOUND OF FORMULA I+prometryn, COMPOUND OF FORMULA I+propachlor, COMPOUND OF FORMULA I+propanil, COMPOUND OF FORMULA I+propaquizafop, COMPOUND OF FORMULA I+propazine, COMPOUND OF FORMULA I+propham, COMPOUND OF FORMULA I+propisochlor, COMPOUND OF FORMULA I+propoxycarbazone, COMPOUND OF FORMULA I+propoxycarbazone-sodium, COMPOUND OF FORMULA I+propyrisulfuron, COMPOUND OF FORMULA I+propyzamide, COMPOUND OF FORMULA I+prosulfocarb, COMPOUND OF FORMULA I+prosulfuron, COMPOUND OF FORMULA I+pyraclonil, COMPOUND OF FORMULA I+pyraflufen, COMPOUND OF FORMULA I+pyraflufen-ethyl, COMPOUND OF FORMULA I+pyrasulfutole, COMPOUND OF FORMULA I+pyrazolynate, COMPOUND OF FORMULA I+pyrazosulfuron, COMPOUND OF FORMULA I+pyrazosulfuron-ethyl, COMPOUND OF FORMULA I+pyrazoxyfen, COMPOUND OF FORMULA I+pyribenzoxim, COMPOUND OF FORMULA I+pyributicarb, COMPOUND OF FORMULA I+pyridafol, COMPOUND OF FORMULA I+pyridate, COMPOUND OF FORMULA I+pyriftalid, COMPOUND OF FORMULA I+pyriminobac, COMPOUND OF FORMULA I+pyriminobac-methyl, COMPOUND OF FORMULA I+pyrimisulfan, COMPOUND OF FORMULA I+pyrithiobac, COMPOUND OF FORMULA I+pyrithiobac-sodium, COMPOUND OF FORMULA I+pyroxsulam, COMPOUND OF FORMULA I+pyroxasulfone, COMPOUND OF FORMULA I+quinclorac, COMPOUND OF FORMULA I+quinmerac, COMPOUND OF FORMULA I+quinoclamine, COMPOUND OF FORMULA I+quizalofop, COMPOUND OF FORMULA I+quizalofop-P, COMPOUND OF FORMULA I+rimsulfuron, COMPOUND OF FORMULA I+saflufenacil, COMPOUND OF FORMULA I+sethoxydim, COMPOUND OF FORMULA I+siduron, COMPOUND OF FORMULA I+simazine, COMPOUND OF FORMULA I+simetryn, COMPOUND OF FORMULA I+SMA, COMPOUND OF FORMULA I+sodium arsenite, COMPOUND OF FORMULA I+sodium azide, COMPOUND OF FORMULA I+sodium chlorate, COMPOUND OF FORMULA I+sulcotrione, COMPOUND OF FORMULA I+sulfentrazone, COMPOUND OF FORMULA I+sulfometuron, COMPOUND OF FORMULA I+sulfometuron-methyl, COMPOUND OF FORMULA I+sulfosate, COMPOUND OF FORMULA I+sulfosulfuron, COMPOUND OF FORMULA I+sulfuric acid, COMPOUND OF FORMULA I+tar oils, COMPOUND OF FORMULA I+2,3,6-TBA, COMPOUND OF FORMULA I+TCA, COMPOUND OF FORMULA I+TCA-sodium, COMPOUND OF FORMULA I+tebuthiuron, COMPOUND OF FORMULA I+tefuryltrione, COMPOUND OF FORMULA I+tembotrione, COMPOUND OF FORMULA I+tepraloxydim, COMPOUND OF FORMULA I+terbacil, COMPOUND OF FORMULA I+terbumeton, COMPOUND OF FORMULA I+terbuthylazine, COMPOUND OF FORMULA I+terbutryn, COMPOUND OF FORMULA I+thenylchlor, COMPOUND OF FORMULA I+thiazopyr, COMPOUND OF FORMULA I+thiencarbazone, COMPOUND OF FORMULA I+thiencarbazone-methyl, COMPOUND OF FORMULA I+thifensulfuron, COMPOUND OF FORMULA I+thifensulfuron-methyl, COMPOUND OF FORMULA I+thiobencarb, COMPOUND OF FORMULA I+tiocarbazil, COMPOUND OF FORMULA I+topramezone, COMPOUND OF FORMULA I+tralkoxydim, COMPOUND OF FORMULA I+triafamone, COMPOUND OF FORMULA I+tri-allate, COMPOUND OF FORMULA I+triasulfuron, COMPOUND OF FORMULA I+triaziflam, COMPOUND OF FORMULA I+tribenuron, COMPOUND OF FORMULA I+tribenuron-methyl, COMPOUND OF FORMULA I+tricamba, COMPOUND OF FORMULA I+triclopyr, COMPOUND OF FORMULA I+trietazine, COMPOUND OF FORMULA I+trifloxysulfuron, COMPOUND OF FORMULA I+trifloxysulfuron-sodium, COMPOUND OF FORMULA I+trifluralin, COMPOUND OF FORMULA I+triflusulfuron, COMPOUND OF FORMULA I+triflusulfuron-methyl, COMPOUND OF FORMULA I+trihydroxytriazine, COMPOUND OF FORMULA I+tritosulfuron, COMPOUND OF FORMULA I+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), COMPOUND OF FORMULA I+4-[(4,5-dihydro-3-methoxy-4-methyl-5-oxo)-1H-1,2,4-triazol-1-ylcarbonylsulfamoyl]-5-methylthiophene-3-carboxylic acid (BAY636), COMPOUND OF FORMULA I+BAY747 (CAS RN 335104-84-2), COMPOUND OF FORMULA I+topramezone (CAS RN 210631-68-8), COMPOUND OF FORMULA I+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), COMPOUND OF FORMULA I+4-hydroxy-3-[[2-(3-methoxypropyl)-6-(difluoromethyl)-3-pyridinyl]carbonyl]-bicyclo[3.2.1]oct-3-en-2-one and COMPOUND OF FORMULA I+ZJ0273.

The compounds of formula (I) according to the invention can also be used in combination with safeners. Preferably, in these mixtures, the compound of the formula (I) is one of those compounds listed in Tables 1 to 333 above. The following mixtures with safeners, especially, come into consideration:

compound of formula (I)+cloquintocet-mexyl, compound of formula (I)+cloquintocet acid and salts thereof, compound of formula (I)+cyprosulfamide, compound of formula (I)+fenchlorazole-ethyl, compound of formula (I)+fenchlorazole acid and salts thereof, compound of formula (I)+mefenpyr-diethyl, compound of formula (I)+mefenpyr diacid, compound of formula (I)+isoxadifen-ethyl, compound of formula (I)+isoxadifen acid, compound of formula (I)+furilazole, compound of formula (I)+furilazole R isomer, compound of formula (I)+benoxacor, compound of formula (I)+dichlormid, compound of formula (I)+AD-67, compound of formula (I)+oxabetrinil, compound of formula (I)+cyometrinil, compound of formula (I)+cyometrinil Z-isomer, compound of formula (I)+fenclorim, compound of formula (I)+cyprosulfamide, compound of formula (I)+naphthalic anhydride, compound of formula (I)+flurazole, compound of formula (I)+N-(2-methoxybenzoyl)-4-[(methylaminocarbonyl)amino]benzenesulfonamide, compound of formula (I)+CL 304,415, compound of formula (I)+dicyclonon, compound of formula (I)+fluxofenim, compound of formula (I)+DKA-24, compound of formula (I)+R-29148 and compound of formula (I)+PPG-1292. A safening effect can also be observed for the mixtures compound of the formula (I)+dymron, compound of the formula (I)+MCPA, compound of the formula (I)+mecopropand compound of the formula (I)+mecoprop-P.

The mixing partners of the compound of formula I may also be in the form of esters or salts, as mentioned e.g. in The Pesticide Manual, 12th Edition (BCPC), 2000.

In the above-mentioned mixtures of compounds of formula I, in particular a compound selected from said Tables 1 to 333, with other insecticides, fungicides, herbicides, safeners, adjuvants and the like, the mixing ratios can vary over a large range and are, preferably

100:1 to 1:6000, especially 50:1 to 1:50, more especially 20:1 to 1:20, even more especially 10:1 to 1:10. Those mixing ratios are understood to include, on the one hand, ratios by weight and also, on other hand, molar ratios.

The mixtures can advantageously be used in the above-mentioned formulations (in which case “active ingredient” relates to the respective mixture of compound of formula I with the mixing partner).

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The mixtures comprising a compound of formula I selected from Tables 1 to 333 and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables 1 to 333 and the active ingredients as described above is not essential for working the present invention.

The invention is illustrated by the following preparation examples. The H-NMR data of certain compounds of this invention show line broadening at room temperature, suggesting the existence of plural conformational isomers due to, for example keto-enol tautomerism, hindered rotation, ring inversion in the piperidine moeity or nitrogen inversion at the piperidine N—OR center. Broad signals have been labeled with ‘br’ accordingly.

### EXAMPLE 1

### Preparation of 3-Carboxymethyl-4-methyl-benzoic acid methyl ester

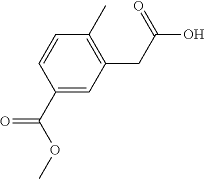

A 20-ml microwave vial was charged with (5-bromo-2-methyl-phenyl)-acetic acid (573 mg, 2.5 mmol), Herrmann's palladacycle (Pd₂(OAc)₂(P(o-tol)₃)₂, 117 mg, 0.12 mmol), Fu's salt ([(t-Bu)₃PH]BF₄, 145 mg, 0.50 mmol), molybdenum hexacarbonyl (Mo(CO)₆, 660 mg, 2.5 mmol) and methanol (17 ml). 1,8-Diazabicyclo[5.4.0]undec-7-ene (DBU, 1.12 ml, 1.14 g, 7.5 mmol) was added dropwise and the mixture stirred vigorously at room temperature for 5 minutes. The vial was then capped and irradiated with microwaves at 110° C. for 30 minutes (1×) and at 140° C. for 15 minutes (4×). After cooling, the reaction mixture was filtered through hyflo (calcined diatomaceous earth) and the solvent removed under reduced pressure. The residue was diluted with ethyl acetate and water, the layers separated, the aqueous phase extracted twice with ethyl acetate and the combined organic layers discarded. The basic water layer was acidified to pH 2-3 with 2N HCl (ice cooling), thoroughly extracted with ethyl acetate (5×), the combined organic phases washed with brine, dried over sodium sulfate and concentrated. Yield: 517 mg of 3-carboxymethyl-4-methyl-benzoic acid methyl ester as a solid, this material was used without further purification in a next step.

¹H-NMR (400 MHz, CDCl₃): δ 2.37 (s, 3H), 3.71 (s, 2H), 3.89 (s, 3H), 7.26 (d, J=7.7 Hz, 1H), 7.84-7.90 (m, 2H), 9.04 (br s, 1H).

LC/MS (ES+): 209 (M+H)⁺

### EXAMPLE 2

### Preparation of 3-(2,4-Dimethyl-6-vinyl-phenyl)-4-hydroxy-8-methoxy-1,8-diaza-spiror[4.5]dec-3-en-2-one (Compound P2.3)

### Step 1: Preparation of 4-[2-(2,4-Dimethyl-6-vinyl-phenyl)-acetylamino]-1-methoxy-piperidine-4-carboxylic acid methyl ester (Compound P3.3)

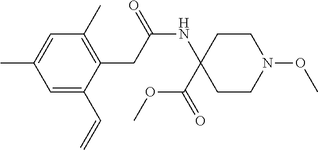

To a solution of 4-amino-1-methoxy-piperidine-4-carboxylic acid methyl ester (600 mg, 3.03 mmol), triethylamine (1.05 ml, 763 mg, 7.54 mmol) and 4-dimethylaminopyridine (DMAP, 4 mg) in tetrahydrofuran (20 ml) at 0-5° C. was added a solution of (2,4-dimethyl-6-vinyl-phenyl)-acetyl chloride (630 mg, 3.03 mmol) in THF (2 ml) dropwise over 1 hour. The reaction mixture was stirred at 0-5° C. for 15 minutes, then at room temperature overnight. The mixture was concentrated in vacuo, diluted with ethyl acetate and water, the layers separated, the aqueous phase extracted once with ethyl acetate, the combined organic phases washed with brine, dried over sodium sulfate and concentrated. The solid residue was suspended in hexane, stirred at room temperature, filtered and dried. Yield: 950 mg of 4-[2-(2,4-dimethyl-6-vinyl-phenyl)-acetylamino]-1-methoxy-piperidine-4-carboxylic acid methyl ester (compound P3.3) as a solid, mp 142-144° C.

¹H-NMR (400 MHz, CD₃OD, selected signals only): δ 2.27 (s, 6H), 3.50 (s, 3H), 3.63 (s, 3H), 3.69 (s, 2H), 5.28 (dd, J=11.0, 1.2 Hz, 1H), 5.57 (dd, J=17.2, 1.2 Hz, 1H), 6.94 (s, 1H), 6.98 (m, 1H), 7.14 (s, 1H).

LC/MS (ES+): 361 (M+H)⁺

### Step 2: Preparation of 3-(2,4-Dimethyl-6-vinyl-phenyl)-4-hydroxy-8-methoxy-1,8-diaza-spiro[4.5]dec-3-en-2-one (Title Compound P2.3)

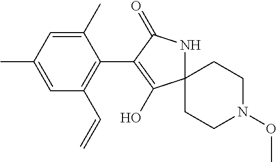

To a solution of 4-[2-(2,4-dimethyl-6-vinyl-phenyl)-acetylamino]-1-methoxy-piperidine-4-carboxylic acid methyl ester (500 mg, 1.39 mmol) in dimethylformamide (20 ml) at 0° C. was added sodium methoxide (225 mg, 4.16 mmol) in one portion and stirring continued at 10° C. for 30 minutes, then at room temperature overnight. The reaction mixture was poured on ice and saturated aqueous ammonium chloride, acidified to pH 5-6 with 6N HCl and thoroughly extracted with ethyl acetate. The combined organic layers were washed with brine, dried over sodium sulfate, concentrated and dried in vacuo. The residue was purified by chromatography on silica gel (ethyl acetate). Yield: 280 mg of 3-(2,4-dimethyl-6-vinyl-phenyl)-4-hydroxy-8-methoxy-1,8-diaza-spiro[4.5]dec-3-en-2-one (title compound P2.3) as a white solid, mp 198-200° C.

¹H-NMR (400 MHz, CD₃OD, selected signals only): δ 2.13 (s, 3H), 2.31 (s, 3H), 3.56 (br s, 3H), 5.14 (dd, J=11.1, 1.4 Hz, 1H), 5.64 (dd, J=17.5, 1.4 Hz, 1H), 6.64 (dd, J=17.5, 11.1 Hz, 1H), 7.02 (s, 1H), 7.29 (s, 1H).

LC/MS (ES+): 329 (M+H)⁺

### EXAMPLE 3

### Preparation of 3-(2,4-Dimethyl-6-vinyl-phenyl)-4-hydroxy-8-methoxy-1-methyl-1,8-diaza-spiro[4.5]dec-3-en-2-one (Compound P2.4)

### Step 1: Preparation of N-(4-Cyano-1-methoxy-piperidin-4-yl)-2-(2,4-dimethyl-6-vinyl-phenyl)-N-methyl-acetamide (Compound P3.4)

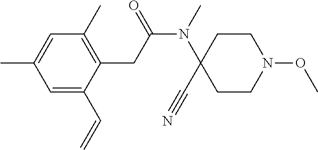

To a solution of 1-methoxy-4-methylamino-piperidine-4-carbonitrile (600 mg, 3.55 mmol) in pyridine (20 ml) was added (2,4-dimethyl-6-vinyl-phenyl)-acetyl chloride (814 mg, 3.90 mmol) dropwise at 0° C. The reaction mixture was stirred at 0° C. for one hour and at room temperature overnight, poured on ice water and acidified to pH 7 with an aqueous 2N HCl solution. The resulting thick precipitate was filtered, washed with cold water, dissolved in ethyl acetate, dried over sodium sulfate and concentrated. The solid residue was suspended in hexane, stirred at room temperature, filtered and dried. Yield: 1.0 g of N-(4-cyano-1-methoxy-piperidin-4-yl)-2-(2,4-dimethyl-6-vinyl-phenyl)-N-methyl-acetamide (compound P3.4) as a pale yellow solid, mp 148-151° C.

¹H-NMR (400 MHz, CD₃OD, selected signals only): δ 2.19 (s, 3H), 2.27 (s, 3H), 3.16 (s, 3H), 3.51 (s, 3H), 3.84 (s, 2H), 5.25 (dd, J=11.0, 1.5 Hz, 1H), 5.53 (dd, J=17.2, 1.5 Hz, 1H), 6.83 (dd, J=17.2, 11.0 Hz, 1H), 6.94 (s, 1H), 7.12 (s, 1H).

LC/MS (ES+): 342 (M+H)⁺

### Step 2: Preparation of 4-{[2-(2,4-Dimethyl-6-vinyl-phenyl)-acetyl]-methyl-amino}-1-methoxy-piperidine-4-carboxylic acid methyl ester (Compound P3.5)

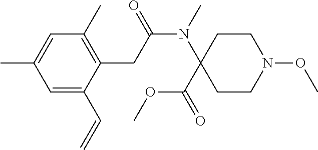

To a suspension of N-(4-cyano-1-methoxy-piperidin-4-yl)-2-(2,4-dimethyl-6-vinyl-phenyl)-N-methyl-acetamide (990 mg, 2.90 mmol) in methanol (2 ml) at 15-20° C. was added concentrated sulfuric acid (0.773 ml, 1.42 g, 14.50 mmol) dropwise over 5 minutes and the reaction mixture was stirred at room temperature overnight. The mixture was poured on ice (20 g), stirred for one hour, then neutralised carefully with 30% aqueous sodium hydroxide to pH 5 (external ice cooling). The thick suspension was filtered, the solid residue washed with cold water, dissolved in dichloromethane, dried over sodium sulfate and concentrated. Yield: 900 mg of 4-{[2-(2,4-dimethyl-6-vinyl-phenyl)-acetyl]-methyl-amino}-1-methoxy-piperidine-4-carboxylic acid methyl ester (compound P3.5) as a pale yellow solid, mp 160-162° C.

¹H-NMR (400 MHz, CD₃OD): δ 1.99 (br m, 2H), 2.16 (s, 3H), 2.26 (s, 3H), 2.34 (br m, 2H), 2.78 (br m, 1H), 2.91 (br m, 1H), 3.18 (br s, 3H), 3.26 (br m, 2H), 3.50 (s, 3H), 3.59 (s, 3H), 3.78 (br s, 2H), 5.24 (dd, J=11.0, 1.5 Hz, 1H), 5.51 (dd, J=17.2, 1.5 Hz, 1H), 6.79 (dd, J=17.2, 11.0 Hz, 1H), 6.91 (s, 1H), 7.10 (s, 1H).

LC/MS (ES+): 375 (M+H)⁺

### Step 3: Preparation of 3-(2,4-Dimethyl-6-vinyl-phenyl)-4-hydroxy-8-methoxy-1-methyl-1,8-diaza-spiro[4.5]dec-3-en-2-one (Title Compound P2.4)

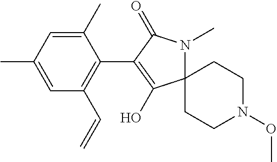

To a solution of 4-{[2-(2,4-dimethyl-6-vinyl-phenyl)-acetyl]-methyl-amino}-1-methoxy-piperidine-4-carboxylic acid methyl ester (900 mg, 2.40 mmol) in dimethylformamide (10 ml) at 0° C. was added sodium methoxide (389 mg, 7.21 mmol) in two portions and stirring continued at 0° C. for 1 hour. The reaction mixture was concentrated in vacuo, poured on cold water (25 ml), acidified to pH 5-6 with 6N HCl, the thick suspension was filtered and washed with cold water (3×20 ml), the solid residue dissolved in ethyl acetate, dried over sodium sulfate and concentrated to yield a first crop of product (400 mg). Further extraction of the aqueous layer, which was saturated with sodium chloride, with ethyl acetate delivered another 390 mg of product after washing with brine, drying over sodium sulfate, and concentration. Yield: 790 mg of 3-(2,4-dimethyl-6-vinyl-phenyl)-4-hydroxy-8-methoxy-1-methyl-1,8-diaza-spiro[4.5]dec-3-en-2-one (title compound P2.4) as a tan-white solid, mp 158-161° C.

¹H-NMR (400 MHz, CD₃OD, selected signals only): δ 2.12 (s, 3H), 2.32 (s, 3H), 2.88 (br signal, 3H), 3.55 (s, 3H), 5.14 (dd, J=11.0, 1.5 Hz, 1H), 5.64 (dd, J=17.2, 1.5 Hz, 1H), 6.62 (dd, J=17.2, 11.0 Hz, 1H), 7.02 (s, 1H), 7.30 (s, 1H).

LC/MS (ES+): 343 (M+H)⁺

### EXAMPLE 4

### Preparation of 4-[2-(5-Acetyl-2-methyl-phenyl)-acetylamino]-1-methoxy-piperidine-4-carboxylic acid methyl ester (Compound P3.1)

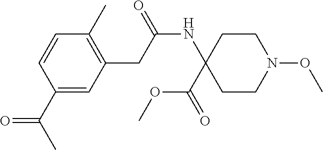 [SMILES: COC(=O)C1(NC(=O)Cc2cc(C(C)=O)ccc2C)CCN(OC)CC1]

A 20-ml microwave vial was charged with 4-[2-(5-bromo-2-methyl-phenyl)-acetylamino]-1-methoxy-piperidine-4-carboxylic acid methyl ester (900 mg, 2.25 mmol), bis(triphenylphosphine)palladium(II) dichloride (50 mg, 0.071 mmol), tributyl(1-ethoxyvinyl)tin (0.92 ml, 984 mg, 2.72 mmol) and dioxane (15 ml) and the mixture stirred vigorously at room temperature for 5 minutes. The vial was then capped and irradiated with microwaves at 150° C. for 30 minutes, at 180° C. for 30 minutes, at 200° C. for 30 minutes and finally at 200° C. for 60 minutes (4×). After cooling, the reaction mixture was filtered through hyflo (calcined diatomaceous earth), the solid inorganic residues washed with dioxane (5 ml), the combined filtrate diluted with 2N HCl (20 ml) and the mixture stirred at room temperature for 2 hours. The mixture was diluted with ethyl acetate, the layers separated, the aqueous phase extracted with ethyl acetate (3×) and the combined organic layers discarded. The acidic water layer was neutralized to pH 7-8 with 4N NaOH (ice cooling), thoroughly extracted with ethyl acetate (4×), the combined organic phases washed with brine, dried over sodium sulfate and concentrated. The residue was purified by chromatography on silica gel (ethyl acetate). Yield: 313 mg of 4-[2-(5-acetyl-2-methyl-phenyl)-acetylamino]-1-methoxy-piperidine-4-carboxylic acid methyl ester (title compound P3.1) as a white solid, mp 138-139° C.

¹H-NMR (400 MHz, CDCl₃, selected signals only): δ 2.38 (br s, 3H), 2.60 (s, 3H), 3.48 (s, 3H), 3.64 (s, 2H), 3.71 (s, 3H), 5.44 (br s, 1H), 7.32 (d, J=7.7 Hz, 1H), 7.79-7.85 (m, 2H).

LC/MS (ES+): 363 (M+H)⁺

### EXAMPLE 5

### Preparation of Carbonic acid 3-(2,4-dimethyl-6-vinyl-phenyl)-8-methoxy-2-oxo-1-(tetrahydro-furan-2-yloxy)-1,8-diaza-spiro[4.5]dec-3-en-4-yl ester ethyl ester (Compound P1.5)

### Step 1: Preparation of 4-{[2-(2,4-Dimethyl-6-vinyl-phenyl)-acetyl]-hydroxy-amino}-1-methoxy-piperidine-4-carboxylic acid methyl ester (Compound P3.6)

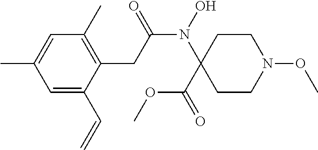

To a solution of 4-hydroxyamino-1-methoxy-piperidine-4-carboxylic acid methyl ester (800 mg, 3.92 mmol) in tetrahydrofuran (25 ml) at 0° C. was added sodium hydrogen carbonate (560 mg, 6.66 mmol), followed by a solution of (2,4-dimethyl-6-vinyl-phenyl)-acetyl chloride (860 mg, 4.12 mmol) in tetrahydrofuran (2 ml) dropwise. The reaction mixture was stirred at 0° C. for one hour and at room temperature overnight. The solvent was evaporated, the residue suspended in water (25 ml), filtered and the solid washed with water (3×20 ml). The solid material was dissolved in ethyl acetate, dried over sodium sulfate, concentrated and the residue suspended in diethyl ether, stirred at room temperature, filtered and dried. Yield: 1.0 g of 4-{[2-(2,4-dimethyl-6-vinyl-phenyl)-acetyl]-hydroxy-amino}-1-methoxy-piperidine-4-carboxylic acid methyl ester (compound P3.6) as a white solid, mp 177-179° C.

¹H-NMR (400 MHz, CD₃OD, selected signals only): δ 2.19 (br s, 3H), 2.26 (s, 3H), 3.52 (br s, 3H), 3.64 (s, 3H), 3.91 (br signal, 2H), 5.24 (dd, J=11.0, 1.5 Hz, 1H), 5.54 (dd, J=17.3, 1.4 Hz, 1H), 6.85 (br m, 1H), 6.92 (s, 1H), 7.12 (s, 1H).

LC/MS (ES+): 377 (M+H)⁺

### Step 2: Preparation of 4-[[2-(2,4-Dimethyl-6-vinyl-phenyl)-acetyl]-(tetrahydro-furan-2-yloxy)-amino]-1-methoxy-piperidine-4-carboxylic acid methyl ester (Compound P3.7)

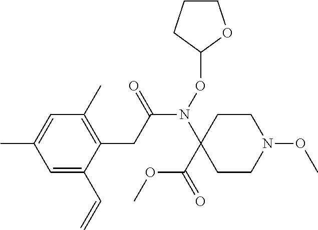

To a solution of 4-{[2-(2,4-dimethyl-6-vinyl-phenyl)-acetyl]-hydroxy-amino}-1-methoxy-piperidine-4-carboxylic acid methyl ester (1.0 g, 2.66 mmol) in dichloromethane (25 ml) was added 2,3-dihydro-furan (0.402 ml, 372 mg, 5.31 mmol) and a catalytic amount of p-toluenesulfonic acid monohydrate (51 mg, 0.27 mmol). The reaction mixture was stirred at reflux overnight and concentrated. The residue was dissolved in hot hexane, filtered, the filtrate concentrated and dried in vacuo. Yield: 1.10 g of 4-[[2-(2,4-dimethyl-6-vinyl-phenyl)-acetyl]-(tetrahydro-furan-2-yloxy)-amino]-1-methoxy-piperidine-4-carboxylic acid methyl ester (compound P3.7) as a gum. This material was used without further purification in the next step.

¹H-NMR (CD₃OD): δ 2.21 (br s, 3H), 2.29 (s, 3H), 3.54 (s, 3H), 3.67 (br s, 3H), 3.86 (br signal, 2H), 4.03 (m, 1H), 4.12 (m, 1H), 5.12 (m, 1H), 5.27 (d, J=11.0 Hz, 1H), 5.58 (dd, J=17.4, 1.6 Hz, 1H), 6.86 (br m, 1H), 6.95 (s, 1H), 7.16 (s, 1H).

LC/MS (ES+): 447 (M+H)⁺

### Step 3: Preparation of 3-(2,4-Dimethyl-6-vinyl-phenyl)-4-hydroxy-8-methoxy-1-(tetrahydro-furan-2-yloxy)-1,8-diaza-spiro[4.5]dec-3-en-2-one (Compound P2.5)

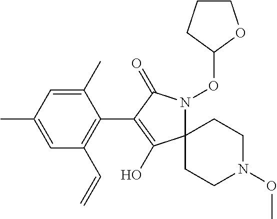

To a solution of 4-[[2-(2,4-dimethyl-6-vinyl-phenyl)-acetyl]-(tetrahydro-furan-2-yloxy)-amino]-1-methoxy-piperidine-4-carboxylic acid methyl ester (1.0 g, 2.24 mmol) in dimethylformamide (30 ml) at 0° C. was added sodium methoxide (363 mg, 6.72 mmol) in four portions and stirring continued at 10° C. for 30 minutes, then at room temperature overnight. The reaction mixture was poured on cold saturated aqueous ammonium chloride, the pH adjusted to 6 with 2N HCl and the aqueous phase thoroughly extracted with ethyl acetate (4×50 ml). The combined organic layers were washed with brine, dried over sodium sulfate and concentrated. The residue was purified by chromatography on silica gel (ethyl acetate/cyclohexane 2:1). Yield: 320 mg of 3-(2,4-dimethyl-6-vinyl-phenyl)-4-hydroxy-8-methoxy-1-(tetrahydro-furan-2-yloxy)-1,8-diaza-spiro[4.5]dec-3-en-2-one (compound P2.5) as a solid, mp 132-135° C.

¹H-NMR (CD₃OD): δ 2.16 (s, 3H), 2.35 (s, 3H), 3.58 (s, 3H), 3.98 (m, 1H), 4.20 (m, 1H), 5.20 (dd, J=11.0, 1.3 Hz, 1H), 5.59 (br m, 1H), 5.69 (m, 1H), 6.65 (dd, J=17.6, 11.0 Hz, 1H), 7.06 (s, 1H), 7.33 (s, 1H).

LC/MS (ES+): 415 (M+H)⁺

### Step 4: Preparation of Carbonic acid 3-(2,4-dimethyl-6-vinyl-phenyl)-8-methoxy-2-oxo-1-(tetrahydro-furan-2-yloxy)-1,8-diaza-spiro[4.5]dec-3-en-4-ylester ethyl ester (Title Compound P1.5)

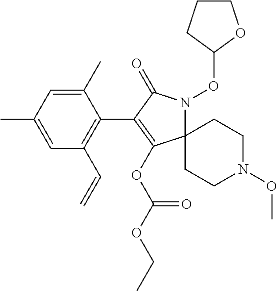

To a solution of 3-(2,4-dimethyl-6-vinyl-phenyl)-4-hydroxy-8-methoxy-1-(tetrahydro-furan-2-yloxy)-1,8-diaza-spiro[4.5]dec-3-en-2-one (220 mg, 0.53 mmol), triethylamine (0.15 ml, 109 mg, 1.06 mmol) and a catalytic amount of 4-dimethylaminopyridine in tetrahydrofuran (15 ml) at 0° C. was added ethyl chloroformate (0.06 ml, 68 mg, 0.58 mmol) dropwise. The suspension was stirred at 0° C. for one hour. The reaction mixture was evaporated, diluted with ethyl acetate and water, the layers separated, the aqueous phase extracted with ethyl acetate (2×50 ml), the combined organic layers washed with water, brine, dried over sodium sulfate and concentrated. The oily residue was purified by chromatography on silica gel (ethyl acetate/cyclohexane 1:1). Yield: 204 mg of carbonic acid 3-(2,4-dimethyl-6-vinyl-phenyl)-8-methoxy-2-oxo-1-(tetrahydro-furan-2-yloxy)-1,8-diaza-spiro[4.5]dec-3-en-4-yl ester ethyl ester (title compound P1.5) as a colorless gum.

¹H-NMR (400 MHz, CD₃OD, selected signals only): δ 1.03 (t, 3H), 2.17 (appar d, 3H), 2.32 (s, 3H), 3.55 (s, 3H), 3.93-4.05 (m, 3H), 4.19 (m, 1H), 5.20 (dd, J=11.0, 1.4 Hz, 1H), 5.65 (m, 2H), 6.58 (m, 1H), 7.01 (s, 1H), 7.26 (s, 1H).

LC/MS (ES+): 487 (M+H)⁺

### EXAMPLE 6

### Preparation of (2-Ethynyl-5-methyl-phenyl)-acetyl chloride

### Step 1: Preparation of (5-Methyl-2-trimethylsilanylethynyl-phenyl)-acetic acid

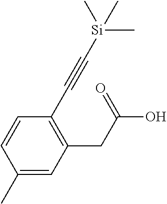 [SMILES: Cc1ccc(C#C[Si](C)(C)C)c(CC(=O)O)c1]

To a solution of (2-iodo-5-methyl-phenyl)-acetic acid (5.5 g, 19.92 mmol) in triethylamine (100 ml) and THF (100 ml) was added copper(I) iodide (152 mg, 0.80 mmol), bis(triphenylphosphine)palladium(II) dichloride (280 mg, 0.40 mmol), followed by ethynyltrimethylsilane (3.31 ml, 2.35 g, 23.89 mmol) dropwise at 15° C. The reaction mixture was stirred at room temperature overnight and concentrated in vacuo. The residue was diluted with dichloromethane (200 ml), filtered over hyflo (calcined diatomaceous earth) and the filtrate washed (4×100 ml) with a 5:1-mixture of water and aqueous 1N HCl. The organic phase was dried over sodium sulfate, concentrated, and the residue purified by chromatography on silica gel (ethyl acetate/cyclohexane 1:3). Yield: 4.13 g of the title compound (5-methyl-2-trimethylsilanylethynyl-phenyl)-acetic acid as a brownish oil.

¹H-NMR (400 MHz, CDCl₃): δ 0.24 (s, 9H), 2.34 (s, 3H), 3.83 (s, 2H), 7.05 (d, J=7.8 Hz, 1H), 7.08 (s, 1H), 7.38 (d, J=7.8 Hz, 1H), 11.40 (br s, 1H).

LC/MS (ES+): 247 (M+H)⁺

### Step 2: Preparation of (2-Ethynyl-5-methyl-phenyl)-acetic acid

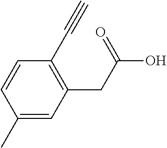

To a solution of (5-methyl-2-trimethylsilanylethynyl-phenyl)-acetic acid (4.0 g, 16.24 mmol) in methanol (140 ml) at room temperature was added potassium carbonate (4.49 g, 32.49 mmol) in 2 portions. The reaction mixture was stirred at room temperature for 2 hours and concentrated under reduced pressure. The residue was taken up in water and acidified to pH 3 under ice cooling. The resulting precipitate was filtered off, washed twice with cold water, the solid dissolved in dichloromethane, the organic phase dried over sodium sulfate and concentrated. Yield: 2.12 g of the title compound (2-ethynyl-5-methyl-phenyl)-acetic acid as a solid, mp 98-101° C.

¹H-NMR (400 MHz, CDCl₃): δ 2.34 (s, 3H), 3.24 (s, 1H), 3.85 (s, 2H), 7.07 (d, J=7.8 Hz, 1H), 7.10 (s, 1H), 7.41 (d, J=7.8 Hz, 1H), 11.10 (br s, 1H).

LC/MS (ES+): 175 (M+H)⁺

### Step 3: Preparation of (2-Ethynyl-5-methyl-phenyl)-acetyl chloride

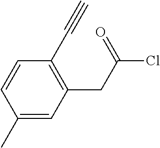

To a solution of (2-ethynyl-5-methyl-phenyl)-acetic acid (2.12 g, 12.17 mmol) in dichloromethane (20 ml) at room temperature were added two drops of dimethylformamide, followed by oxalyl chloride (1.25 ml, 1.85 g, 14.58 mmol) dropwise. The reaction mixture was stirred at room temperature, subsequently at 40° C. to bring gas evolution to completion, then concentrated under reduced pressure. Yield: 2.20 g of the title compound (2-ethynyl-5-methyl-phenyl)-acetyl chloride as a semi-solid, which fully solidified when kept in the fridge.

¹H-NMR (400 MHz, CDCl₃): δ 2.36 (s, 3H), 3.30 (s, 1H), 4.32 (s, 2H), 7.08 (s, 1H), 7.12 (d, J=7.9 Hz, 1H), 7.44 (d, J=7.9 Hz, 1H).

### EXAMPLE 7

### Preparation of 4-{[2-(2-Ethynyl-5-methyl-phenyl)-acetyl]-methoxy-amino}-1-methoxy-piperidine-4-carboxylic acid methyl ester (Compound P3.16)

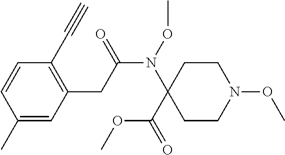 [SMILES: C#Cc1ccc(C)cc1CC(=O)N(OC)C1(C(=O)OC)CCN(OC)CC1]

To a solution of triphenylphosphine (434 mg, 1.65 mmol) in THF (10 ml) at 0° C. was added diisopropyl azodicarboxylate (0.327 ml, 336 mg, 1.66 mmol) dropwise and the resulting precipitate was stirred at 0° C. for 30 minutes. 4-{[2-(2-Ethynyl-5-methyl-phenyl)-acetyl]-hydroxy-amino}-1-methoxy-piperidine-4-carboxylic acid methyl ester (compound P3.15 obtained in analogy to preparation EXAMPLE 5, step 1) (470 mg, 1.30 mmol) in THF (2 ml) was further added dropwise, followed by methanol (0.060 ml, 47.5 mg, 1.48 mmol) in one portion at 0° C. The reaction mixture was stirred at room temperature for two hours and concentrated in vacuo. The residue was purified by chromatography on silica gel (ethyl acetate/cyclohexane 1:2). Yield: 186 mg of 4-{[2-(2-ethynyl-5-methyl-phenyl)-acetyl]-methoxy-amino}-1-methoxy-piperidine-4-carboxylic acid methyl ester (title compound P3.16) as a gum.

¹H-NMR (400 MHz, CDCl₃, selected signals only): δ 2.32 (s, 3H), 3.21 (s, 1H), 3.52 (s, 3H), 3.72 (s, 3H), 3.92 (br signal, total 3H), 7.02 (d, J=7.7 Hz, 1H), 7.08 (br s, 1H), 7.38 (d, J=7.7 Hz, 1H).

LC/MS (ES+): 375 (M+H)⁺

### EXAMPLE 8

### Preparation of 3-(2,6-Dimethyl-4-trimethylsilanylethynyl-phenyl)-4-hydroxy-8-methoxy-1,8-diaza-spiro[4.5]dec-3-en-2-one

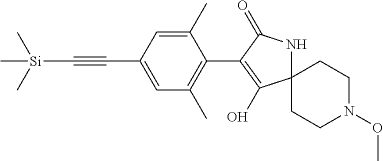

To a solution of 4-hydroxy-3-(4-iodo-2,6-dimethyl-phenyl)-8-methoxy-1,8-diaza-spiro[4.5]dec-3-en-2-one (740 mg, 1.728 mmol) in triethylamine (10 ml) and THF (10 ml) was added copper(I) iodide (13 mg, 0.068 mmol), bis(triphenylphosphine)palladium(II) dichloride (24 mg, 0.034 mmol), followed by ethynyltrimethylsilane (0.287 ml, 203.5 mg, 2.072 mmol) dropwise at 10° C. The reaction mixture was stirred at room temperature overnight and concentrated in vacuo. The residue was diluted with dichloromethane (50 ml), filtered over hyflo (calcined diatomaceous earth) and the filtrate washed (4×) with an aqueous solution of pH 5-6. The organic phase was dried over sodium sulfate, concentrated, and the residue purified by chromatography on silica gel (ethyl acetate). Yield: 243 mg of the title compound 3-(2,6-dimethyl-4-trimethylsilanylethynyl-phenyl)-4-hydroxy-8-methoxy-1,8-diaza-spiro[4.5]dec-3-en-2-one as a tan solid, mp>240° C.

¹H-NMR (400 MHz, CDCl₃, selected signals only): δ 0.24 (s, 9H), 2.10 (s, 6H), 3.56 (br s, 3H), 5.76 (br s, 1H), 7.17 (s, 2H).

LC/MS (ES+): 399 (M+H)⁺

### EXAMPLE 9

### Preparation of 3-(4-Ethynyl-2,6-dimethyl-phenyl)-4-hydroxy-8-methoxy-1-methyl-1,8-diaza-spiro[4.5]dec-3-en-2-one (Compound P2.12)

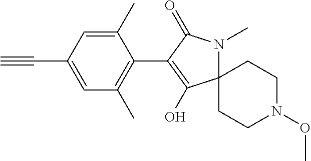 [SMILES: C#Cc1cc(C)c(C2=C(O)C3(CCN(OC)CC3)N(C)C2=O)c(C)c1]

To a solution of 3-(2,6-dimethyl-4-trimethylsilanylethynyl-phenyl)-4-hydroxy-8-methoxy-1-methyl-1,8-diaza-spiro[4.5]dec-3-en-2-one (obtained in analogy to preparation EXAMPLE 8) (167 mg, 0.405 mmol) in methanol (5 ml) at room temperature was added potassium carbonate (112 mg, 0.810 mmol) in 2 portions. The reaction mixture was stirred at room temperature for 2.5 hours and concentrated under reduced pressure. The residue was taken up in water and acidified with a 1 N HCl solution to pH 6 under ice cooling. The resulting precipitate was filtered off, the solid suspended in hexane, filtered and the beige solid dried over phosphorus pentoxide under vacuum at 40° C. overnight. Yield: 107 mg of 3-(4-ethynyl-2,6-dimethyl-phenyl)-4-hydroxy-8-methoxy-1-methyl-1,8-diaza-spiro[4.5]dec-3-en-2-one (title compound P2.12) as a beige solid, mp 258-260° C.

¹H-NMR (400 MHz, CD₃OD, selected signals only): δ 2.14 (s, 6H), 2.89 (br signal, total 3H), 3.41 (s, 1H), 3.55 (s, 3H), 7.20 (s, 2H).

LC/MS (ES+): 341 (M+H)⁺

### EXAMPLE 10

### Preparation of 3-(2,6-Dimethyl-4-prop-1-ynyl-phenyl)-4-hydroxy-8-methoxy-1-methyl-1,8-diaza-spiro[4.5]dec-3-en-2-one (Compound P2.13)

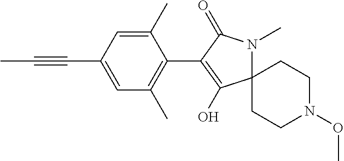 [SMILES: CC#Cc1cc(C)c(C2=C(O)C3(CCN(OC)CC3)N(C)C2=O)c(C)c1]

Propyne was bubbled into a suspension of 4-hydroxy-3-(4-iodo-2,6-dimethyl-phenyl)-8-methoxy-1-methyl-1,8-diaza-spiro[4.5]dec-3-en-2-one (300 mg, 0.678 mmol), copper(I) iodide (13 mg, 0.068 mmol) and bis(triphenylphosphine)palladium(II) dichloride (24 mg, 0.034 mmol) in triethylamine (20 ml) until saturation, leading to brown coloration of the reaction mixture. This mixture was stirred under the remaining propyne atmosphere at room temperature overnight, diluted with saturated aqueous ammonium chloride (20 ml), the layers separated, the aqueous phase extracted with ethyl acetate (3×25 ml), the combined organic layers washed with brine, dried over sodium sulfate and concentrated. The gummy brownish residue was triturated with diethyl ether, filtered, dried, concentrated and the solid redissolved in dichloromethane, treated with active charcoal, filtered and concentrated in vacuo to afford a first crop of product (120 mg). The mother liquor was concentrated and purified by chromatography on silica gel (ethyl acetate) to further afford 55 mg of product. Yield: 175 mg of 3-(2,6-dimethyl-4-prop-1-ynyl-phenyl)-4-hydroxy-8-methoxy-1-methyl-1,8-diaza-spiro[4.5]dec-3-en-2-one (title compound P2.13) as a tan solid, mp>240° C.

¹H-NMR (400 MHz, CD₃OD, selected signals only): δ 2.00 (s, 3H), 2.11 (s, 6H), 2.88 (br signal, total 3H), 3.55 (s, 3H), 7.08 (s, 2H).

LC/MS (ES+): 355 (M+H)⁺

Compounds of the formula I from Table P1, compounds of the formula II from Table P2 and intermediates listed in Table P3 can be prepared by analogous procedures. Either one of the following LC-MS methods was used to characterize the compounds:

### Method A

MS: ZQ Mass Spectrometer from Waters (Single quadrupole mass spectrometer); Ionisation method: Electrospray; Polarity: positive/negative ions; Capillary (kV) 3.00, Cone (V) 30.00, Extractor (V) 2.00, Source Temperature (° C.) 100, Desolvation Temperature (° C.) 250, Cone Gas Flow (L/Hr) 50, Desolvation Gas Flow (L/Hr) 400; Mass range: 150 to 1000 or 100 to 900 Da.
LC: HP 1100 HPLC from Agilent: solvent degasser, quaternary pump (ZCQ)/binary pump (ZDQ), heated column compartment and diode-array detector. Column: Phenomenex Gemini C18, 3 μm particle size, 110 Angström, 30×3 mm, Temp: 60° C.; DAD Wavelength range (nm): 200 to 500; Solvent gradient: A=water+0.05% v/v HCOOH, B=Acetonitril/Methanol (4:1, v/v)+0.04% v/v HCOOH.

| Time (min) | A % | B % | Flow (ml/min) |
|---|---|---|---|
| 0.00 | 95.0 | 5.0 | 1.700 |
| 2.00 | 0.0 | 100.0 | 1.700 |
| 2.80 | 0.0 | 100.0 | 1.700 |
| 2.90 | 95.0 | 5.0 | 1.700 |
| 3.00 | 95.0 | 5.0 | 1.700 |

### Method B

MS: ZMD Mass Spectrometer from Waters (Single quadrupole mass spectrometer); Ionisation method: Electrospray; Polarity: positive/negative ions; Capillary (kV) 3.80, Cone (V) 30.00, Extractor (V) 3.00, Source Temperature (° C.) 150, Desolvation Temperature (° C.) 350, Cone Gas Flow (L/Hr) OFF, Desolvation Gas Flow (L/Hr) 600; Mass range: 150 to 1000 (100 to 1500 for LowMass) or 100 to 900 Da.
LC: HP 1100 HPLC from Agilent: solvent degasser, binary pump, heated column compartment and diode-array detector. Column: Phenomenex Gemini C18, 3 μm particle size, 110 Angström, 30×3 mm, Temp: 60° C.; DAD Wavelength range (nm): 200 to 500; Solvent gradient: A=water+0.05% v/v HCOOH, B=Acetonitril/Methanol (4:1, v:v)+0.04% v/v HCOOH.

| Time (min) | A % | B % | Flow (ml/min) |
|---|---|---|---|
| 0.00 | 95.0 | 5.0 | 1.700 |
| 2.00 | 0.0 | 100.0 | 1.700 |
| 2.80 | 0.0 | 100.0 | 1.700 |
| 2.90 | 95.0 | 5.0 | 1.700 |
| 3.00 | 95.0 | 5.0 | 1.700 |

### Method C

MS: ZQ Mass Spectrometer from Waters (Single quadrupole mass spectrometer); Ionisation method: Electrospray; Polarity: positive/negative ions; Capillary (kV) 3.00, Cone (V) 30.00, Extractor (V) 2.00, Source Temperature (° C.) 100, Desolvation Temperature (° C.) 250, Cone Gas Flow (L/Hr) 50, Desolvation Gas Flow (L/Hr) 400; Mass range: 100 to 900 Da.
LC: HP 1100 HPLC from Agilent: solvent degasser, quaternary pump (ZCQ), heated column compartment and diode-array detector. Column: Phenomenex Gemini C18, 3 μm particle size, 30×3 mm, Temp: 60° C.; DAD Wavelength range (nm): 210 to 500; Solvent gradient: A=water+5% v/v Methanol+0.05% v/v HCOOH, B=Acetonitril+0.05% v/v HCOOH.

| Time (min) | A % | B % | Flow (ml/min) |
|---|---|---|---|
| 0.00 | 100 | 0 | 1.700 |
| 2.00 | 0 | 100 | 1.700 |
| 2.80 | 0 | 100 | 1.700 |
| 2.90 | 100 | 0 | 1.700 |
| 3.00 | 100 | 0 | 1.700 |

The characteristic values obtained for each compound were the retention time (“Rₜ”, recorded in minutes) and the molecular ion as listed in Table P1, Table P2 and in Table P3.

TABLE P1
Physical data of compounds of formula I:

| Compound | | Melting | |
|---|---|---|---|
| No. | Structures | Point | MS/NMR |
| P1.1 | 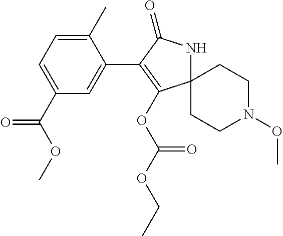 [SMILES: CCOC(=O)OC1=C(c2cc(C(=O)OC)ccc2C)C(=O)NC12CCN(OC)CC2] | gum | LC/MS: 419 (M + H)⁺ Rₜ = 1.64 min |
| P1.2 | 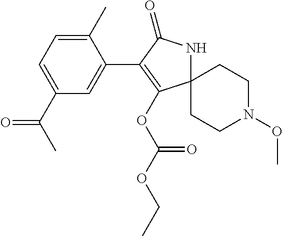 [SMILES: CCOC(=O)OC1=C(c2cc(C(C)=O)ccc2C)C(=O)NC12CCN(OC)CC2] | 185-187° C. | LC/MS: 403 (M + H)⁺ Rₜ = 1.57 min |
| P1.3 | 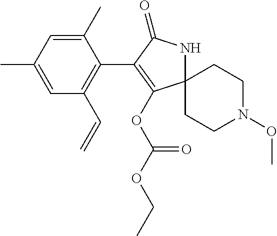 | 152-154° C. | LC/MS: 401 (M + H)⁺ Rₜ = 1.80 min |
| P1.4 | 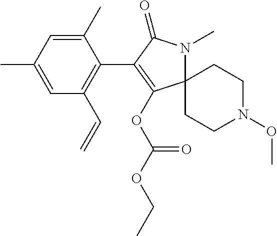 | 131-133° C. | LC/MS: 415 (M + H)⁺ Rₜ = 1.83 min |
| P1.5 | 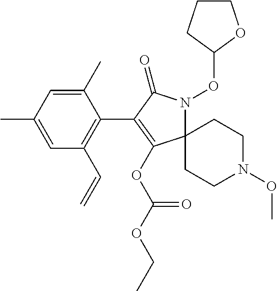 EXAMPLE 5, step 4 | gum | LC/MS: 487 (M + H)⁺ Rₜ = 1.98 min |
| P1.6 | 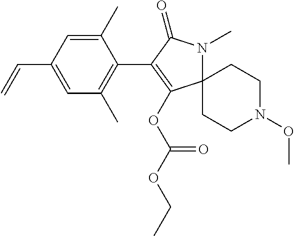 [SMILES: C=Cc1cc(C)c(C2=C(OC(=O)OCC)C3(CCN(OC)CC3)N(C)C2=O)c(C)c1] | 85-88° C. | LC/MS: 415 (M + H)⁺ Rₜ = 1.94 min |
| P1.7 | 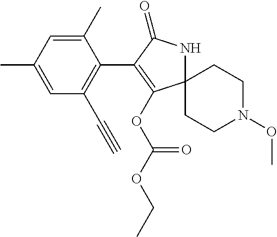 | 210-212° C. | LC/MS: 399 (M + H)⁺ Rₜ = 1.74 min |
| P1.8 | 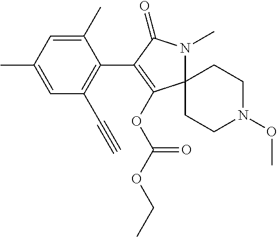 | 135-137° C. | LC/MS: 413 (M + H)⁺ Rₜ = 1.77 min |
| P1.9 | 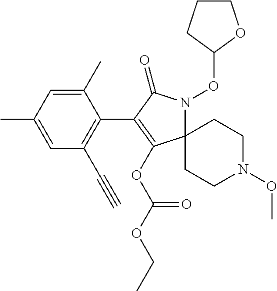 | 68-70° C. | LC/MS: 485 (M + H)⁺ Rₜ = 1.92 min |
| P1.10 | 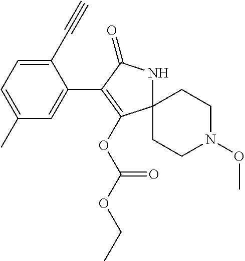 [SMILES: C#Cc1ccc(C)cc1C1=C(OC(=O)OCC)C2(CCN(OC)CC2)NC1=O] | gum | LC/MS: 385 (M + H)⁺ Rₜ = 1.63 min |
| P1.11 | 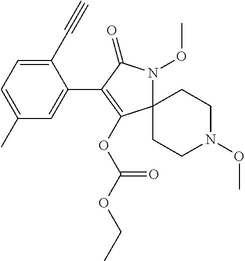 [SMILES: C#Cc1ccc(C)cc1C1=C(OC(=O)OCC)C2(CCN(OC)CC2)N(OC)C1=O] | gum | LC/MS: 415 (M + H)⁺ Rₜ = 1.71 min |
| P1.12 | 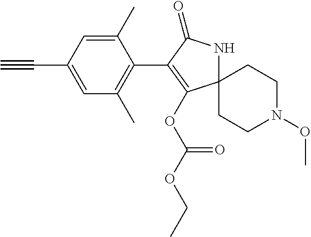 [SMILES: C#Cc1cc(C)c(C2=C(OC(=O)OCC)C3(CCN(OC)CC3)NC2=O)c(C)c1] | 208-210° C. | LC/MS: 399 (M + H)⁺ Rₜ = 1.70 min |
| P1.13 | 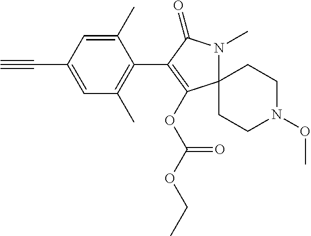 [SMILES: C#Cc1cc(C)c(C2=C(OC(=O)OCC)C3(CCN(OC)CC3)N(C)C2=O)c(C)c1] | gum | LC/MS: 413 (M + H)⁺ Rₜ = 1.76 min |
| P1.14 | 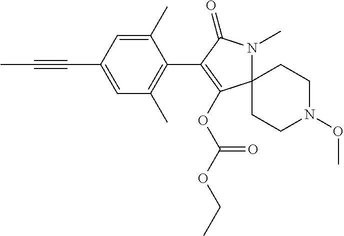 [SMILES: CC#Cc1cc(C)c(C2=C(OC(=O)OCC)C3(CCN(OC)CC3)N(C)C2=O)c(C)c1] | gum | LC/MS: 427 (M + H)⁺ Rₜ = 1.83 min |
| P1.15 | 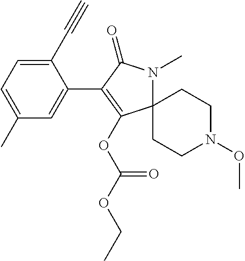 [SMILES: C#Cc1ccc(C)cc1C1=C(OC(=O)OCC)C2(CCN(OC)CC2)N(C)C1=O] | gum | LC/MS: 399 (M + H)⁺ Rₜ = 1.65 min |

TABLE P2
Physical data of compounds of formula II:

| Compound | | Melting | |
|---|---|---|---|
| No. | Structures | Point | MS/NMR |
| P2.1 | 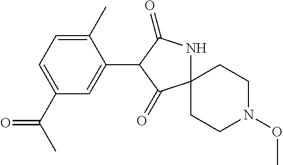 [SMILES: CON1CCC2(CC1)NC(=O)C(c1cc(C(C)=O)ccc1C)C2=O] | 218-220° C. | LC/MS: 331 (M + H)⁺ Rₜ = 1.20 min |
| P2.2 | 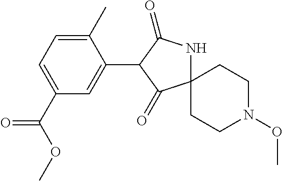 [SMILES: COC(=O)c1ccc(C)c(C2C(=O)NC3(CCN(OC)CC3)C2=O)c1] | 245-247° C. | LC/MS: 347 (M + H)⁺ Rₜ = 1.29 min |
| P2.3 | 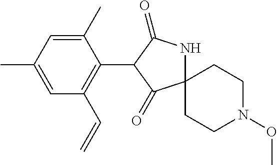 EXAMPLE 2, step 2 | 198-200° C. | LC/MS: 329 (M + H)⁺ Rₜ = 1.42 min |
| P2.4 | 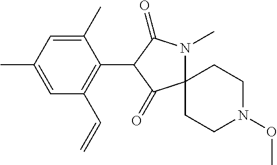 EXAMPLE 3, step 3 | 158-161° C. | LC/MS: 343 (M + H)⁺ Rₜ = 1.49 min |
| P2.5 | 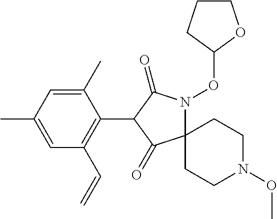 EXAMPLE 5, step 3 | 132-135° C. | LC/MS: 415 (M + H)⁺ Rₜ = 1.70 min |
| P2.6 | 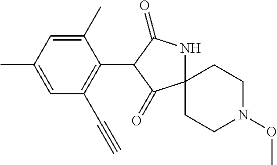 | 246° C. (dec) | LC/MS: 327 (M + H)⁺ Rₜ = 1.42 min |
| P2.7 | 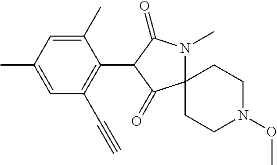 | 198-200° C. | LC/MS: 341 (M + H)⁺ Rₜ = 1.46 min |
| P2.8 | 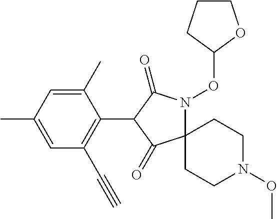 | 94-97° C. | LC/MS: 413 (M + H)⁺ Rₜ = 1.63 min |
| P2.9 | 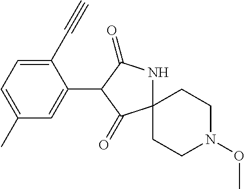 [SMILES: C#Cc1ccc(C)cc1C1C(=O)NC2(CCN(OC)CC2)C1=O] | gum | LC/MS: 313 (M + H)⁺ Rₜ = 1.32 min |
| P2.10 | 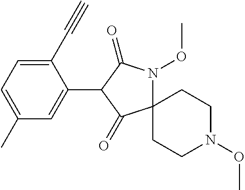 [SMILES: C#Cc1ccc(C)cc1C1C(=O)N(OC)C2(CCN(OC)CC2)C1=O] | 70-72° C. | LC/MS: 343 (M + H)⁺ Rₜ = 1.45 min |
| P2.11 | 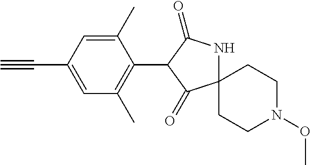 [SMILES: C#Cc1cc(C)c(C2C(=O)NC3(CCN(OC)CC3)C2=O)c(C)c1] | 251-253° C. | LC/MS: 327 (M + H)⁺ Rₜ = 1.37 min |
| P2.12 | 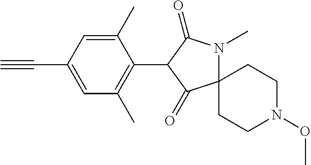 [SMILES: C#Cc1cc(C)c(C2C(=O)N(C)C3(CCN(OC)CC3)C2=O)c(C)c1] EXAMPLE 9 | 258-260° C. | LC/MS: 341 (M + H)⁺ Rₜ = 1.44 min |
| P2.13 | 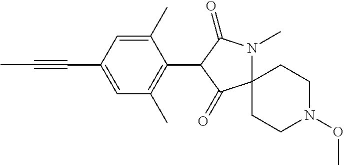 [SMILES: CC#Cc1cc(C)c(C2C(=O)N(C)C3(CCN(OC)CC3)C2=O)c(C)c1] EXAMPLE 10 | 240° C. (dec) | LC/MS: 355 (M + H)⁺ Rₜ = 1.53 min |
| P2.14 | 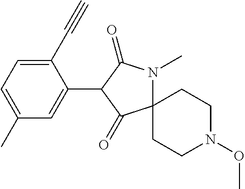 [SMILES: C#Cc1ccc(C)cc1C1C(=O)N(C)C2(CCN(OC)CC2)C1=O] | solid | LC/MS: 327 (M + H)⁺ Rₜ = 1.42 min |

TABLE P3
Physical data of intermediates of formula IV or XI:

| Compound | | | |
|---|---|---|---|
| No. | Structures | Melting Point | MS/NMR |
| P3.1 | 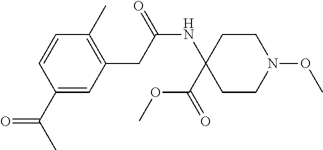 [SMILES: COC(=O)C1(NC(=O)Cc2cc(C(C)=O)ccc2C)CCN(OC)CC1] EXAMPLE 4 | 138-139° C. | LC/MS: 363 (M + H)⁺ Rₜ = 1.39 min |
| P3.2 | 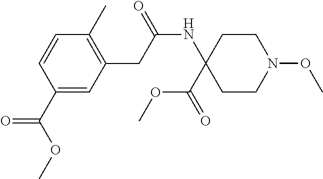 [SMILES: COC(=O)c1ccc(C)c(CC(=O)NC2(C(=O)OC)CCN(OC)CC2)c1] | 138-140° C. | LC/MS: 379 (M + H)⁺ Rₜ = 1.47 min |
| P3.3 | 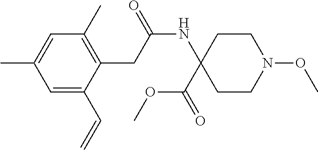 EXAMPLE 2, step 1 | 142-144° C. | LC/MS: 361 (M + H)⁺ Rₜ = 1.74 min |
| P3.4 | 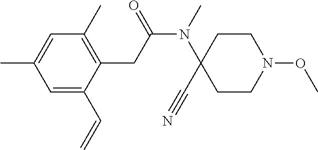 EXAMPLE 3, step 1 | 148-151° C. | LC/MS: 342 (M + H)⁺ Rₜ = 1.78 min |
| P3.5 | 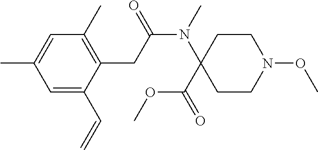 EXAMPLE 3, step 2 | 160-162° C. | LC/MS: 375 (M + H)⁺ Rₜ = 1.83 min |
| P3.6 | 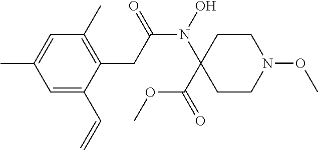 EXAMPLE 5, step 1 | 177-179° C. | LC/MS: 377 (M + H)⁺ Rₜ = 1.72 min |
| P3.7 | 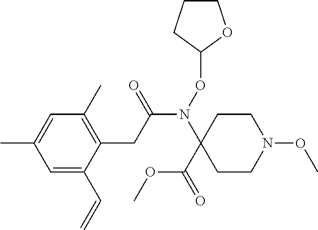 EXAMPLE 5, step 2 | gum | LC/MS: 447 (M + H)⁺ Rₜ = 2.02 min |
| P3.8 | 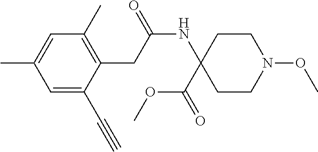 | 138-141° C. | LC/MS: 359 (M + H)⁺ Rₜ = 1.69 min |
| P3.9 | 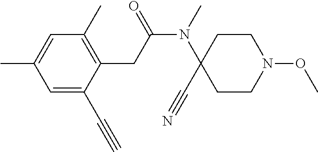 | 154-156° C. | LC/MS: 340 (M + H)⁺ Rₜ = 1.76 min |
| P3.10 | 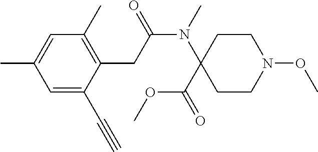 | 123-126° C. | LC/MS: 373 (M + H)⁺ Rₜ = 1.77 min |
| P3.11 | 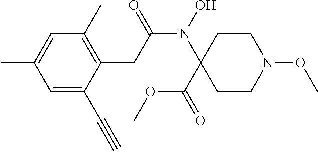 | 186-189° C. | LC/MS: 375 (M + H)⁺ Rₜ = 1.69 min |
| P3.12 | 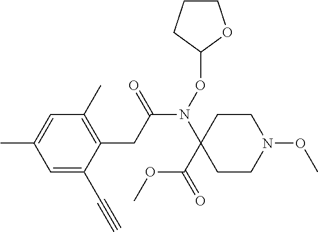 | gum | LC/MS: 445 (M + H)⁺ Rₜ = 1.97 min |
| P3.13 | 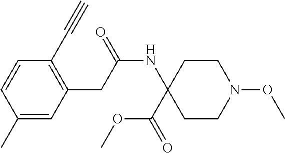 | 110-113° C. | LC/MS: 345 (M + H)⁺ Rₜ = 1.52 min |
| P3.14 | 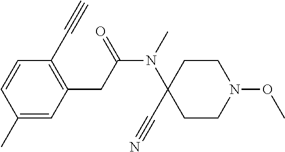 [SMILES: C#Cc1ccc(C)cc1CC(=O)N(C)C1(C#N)CCN(OC)CC1] | solid | LC/MS: 326 (M + H)⁺ Rₜ = 1.61 min |
| P3.15 | 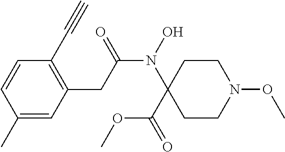 [SMILES: C#Cc1ccc(C)cc1CC(=O)N(O)C1(C(=O)OC)CCN(OC)CC1] | solid | LC/MS: 361 (M + H)⁺ Rₜ = 1.51 min |
| P3.16 | 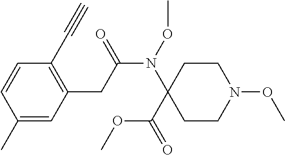 [SMILES: C#Cc1ccc(C)cc1CC(=O)N(OC)C1(C(=O)OC)CCN(OC)CC1] EXAMPLE 7 | gum | LC/MS: 375 (M + H)⁺ Rₜ = 1.75 min |
| P3.17 | 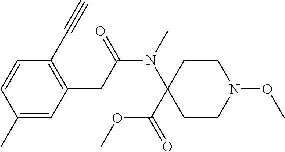 [SMILES: C#Cc1ccc(C)cc1CC(=O)N(C)C1(C(=O)OC)CCN(OC)CC1] | solid | LC/MS: 359 (M + H)⁺ Rₜ = 1.62 min |

### FORMULATION EXAMPLES (%=PERCENT BY WEIGHT)

### EXAMPLE F1

### Emulsion Concentrates

| | a) | b) | c) |
|---|---|---|---|
| Active ingredient | 25% | 40% | 50% |
| Calcium dodecylbenzenesulfonate | 5% | 8% | 6% |
| Castor oil polyethylene glycol ether (36 mol of EO) | 5% | — | — |
| Tributylphenoxypolyethylene glycol ether | — | 12% | 4% |
| (30 mol of EO) | | | |
| Cyclohexanone | — | 15% | 20% |
| Xylene mixture | 65% | 25% | 20% |

Emulsions of any desired concentration can be prepared from such concentrates by dilution with water.

### EXAMPLE F2

### Solutions

| | a) | b) | c) | d) |
|---|---|---|---|---|
| Active ingredient | 80% | 10% | 5% | 95% |
| Ethylene glycol monomethyl ether | 20% | — | — | — |
| Polyethylene glycol MW 400 | — | 70% | — | — |
| N-Methylpyrrolid-2-one | — | 20% | — | — |
| Epoxidized coconut oil | — | — | 1% | 5% |
| Petroleum ether (boiling range: 160-190°) | — | — | 94% | — |

The solutions are suitable for use in the form of microdrops.

### EXAMPLE F3

### Granules

| | a) | b) | c) | d) |
|---|---|---|---|---|
| Active ingredient | 5% | 10% | 8% | 21% |
| Kaolin | 94% | — | 79% | 54% |
| Highly disperse silica | 1% | — | 13% | 7% |
| Attapulgite | — | 90% | — | 18% |

The active ingredient is dissolved in dichloromethane, the solution is sprayed onto the carrier(s), and the solvent is subsequently evaporated in vacuo.

### EXAMPLE F4

### Dusts

| | a) | b) |
|---|---|---|
| Active ingredient | 2% | 5% |
| Highly disperse silica | 1% | 5% |
| Talc | 97% | — |
| Kaolin | — | 90% |

Ready-to-use dusts are obtained by intimately mixing the carriers and the active ingredient.

### EXAMPLE F5

### Wettable Powders

| | a) | b) | c) |
|---|---|---|---|
| Active ingredient | 25% | 50% | 75% |
| Sodium lignosulfonate | 5% | 5% | — |
| Sodium lauryl sulfate | 3% | — | 5% |
| Sodium diisobutylnaphthalenesulfonate | — | 6% | 10% |
| Octylphenoxypolyethylene glycol | — | 2% | — |
| ether (7-8 mol of EO) | | | |
| Highly disperse silica | 5% | 10% | 10% |
| Kaolin | 62% | 27% | — |

The active ingredient is mixed with the additives and the mixture is ground thoroughly in a suitable mill. This gives wettable powders, which can be diluted with water to give suspensions of any desired concentration.

### EXAMPLE F6

### Extruder Granules

| | |
|---|---|
| Active ingredient | 10% |
| Sodium lignosulfonate | 2% |
| Carboxymethylcellulose | 1% |
| Kaolin | 87% |

The active ingredient is mixed with the additives, and the mixture is ground, moistened with water, extruded, granulated and dried in a stream of air.

### EXAMPLE F7

### Coated Granules

| | |
|---|---|
| Active ingredient | 3% |
| Polyethylene glycol (MW 200) | 3% |
| Kaolin | 94% |

In a mixer, the finely ground active ingredient is applied uniformLy to the kaolin, which has been moistened with the polyethylene glycol. This gives dust-free coated granules.

### EXAMPLE F8a

### Suspension Concentrate

| | |
|---|---|
| Active ingredient | 40% |
| Ethylene glycol | 10% |
| Nonylphenoxypolyethylene glycol ether (15 mol of EO) | 6% |
| Sodium lignosulfonate | 10% |
| Carboxymethylcellulose | 1% |
| 37% aqueous formaldehyde solution | 0.2% |
| Silicone oil (75% aqueous emulsion) | 0.8% |
| Water | 32% |

### EXAMPLE F8b

### Suspension Concentrate

| | |
|---|---|
| Active ingredient | 10% |
| Naphthalenesulfonic acid, sodium | 2% |
| salt condensed with formaldehyde | |
| Solution of an acrylic graft copolymer | 8% |
| in water and propyleneglycole | |
| Silicone antifoam emulsion | 0.5% |
| DL-propanediol-(1,2) | 3% |
| Heteropolysaccharide | 0.5% |
| 1,2-Benzisothiazol-3-one | 0.2% |
| Water | 75.8% |

The finely ground active ingredient is mixed intimately with the additives. Suspensions of any desired concentration can be prepared from the thus resulting suspension concentrate by dilution with water.

### EXAMPLE F9

### Powders for Dry Seed Treatment

| | a) | b) | c) |
|---|---|---|---|
| active ingredient | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

### EXAMPLE F10

### Flowable Concentrate for Seed Treatment

| | |
|---|---|
| active ingredient | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% solution in | 0.5% |
| water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### EXAMPLE F11a

### Oil-Based Suspension Concentrate (Based on a Vegetable Oil)

| | |
|---|---|
| Active ingredient | 10% |
| Tristyrylphenole with 16 moles EO | 10% |
| Block copolymer of polyhydroxystearic | 2% |
| acid and polyalkylene glycols | |
| AEROSIL 200 | 1% |
| Rape seed oil methyl ester | 12% |
| Oleic acid | 65% |

### EXAMPLE F11b

### Oil-Based Suspension Concentrate (Based on a Mineral Oil)

| | |
|---|---|
| Active ingredient | 10% |
| Ethoxylated alcohols, C16-18 and C18-unsatd | 5% |
| Dodecyl-benzene sulfonic acid Ca-salt linear | 2.5% |
| 2-Pyrrolidinone, 1-ethenylhexadecyl-, homopolymer | 1% |
| Organophilic clay | 1% |
| Mixture of petroleum | 80.5% |

The finely ground active ingredient is mixed intimately with the additives. Suspensions of any desired concentration can be prepared from the thus resulting suspension concentrate by dilution with water.

Preferably, the term “active ingredient” used above refers to one of the compounds selected from Tables 1 to 333 shown above. It also refers to mixtures of the compound of formula I, in particular a compound selected from said Tables 1 to 333, with other insecticides, fungicides, herbicides, safeners, adjuvants and the like, which mixtures are specifically disclosed above.

### BIOLOGICAL EXAMPLES

These examples illustrate the pesticidal/insecticidal properties of compounds of formula I.

### EXAMPLE B1

### Activity Against Myzus persicae (Green Peach Aphid) (Mixed Population, Feeding/Residual Contact Activity, Preventive)

Sunflower leaf discs are placed on agar in a 24-well microtiter plate and sprayed with test solutions. After drying, the leaf discs are infested with an aphid population of mixed ages. After an incubation period of 6 days, samples are checked for mortality and special effects (e.g. phytotoxicity).

In this test, compounds listed in the tables above show good activity. In particular compounds P1.3, P1.4, P1.5, P1.7, P1.8, P1.9, P1.11, P1.12, P1.13, P2.3, P2.6, P2.7, P2.8, P2.11 and P2.12 show an activity of over 80% at a concentration of 400 ppm.

### EXAMPLE B2

### Activity Against Myzus persicae (Green Peach Aphid) (Mixed Population, Systemic/Feeding Activity, Curative)

Roots of pea seedlings, infested with an aphid population of mixed ages, are placed directly in the test solutions. 6 days after introduction, samples are checked for mortality and special effects on the plant.

In this test, compounds listed in the tables above show good activity. In particular compounds P1.3, P1.7, P1.8, P1.9, P1.12, P1.13, P1.14, P2.6, P2.8, P2.11 and P2.12 show an activity of over 80% at a concentration of 400 ppm.

### EXAMPLE B3

### Activity Against Thrips tabaci (Onion Thrips) (Mixed Population, Feeding/Residual Contact Activity, Preventive)

Sunflower leaf discs are placed on agar in a 24-well microtiter plate and sprayed with test solutions. After drying, the leaf discs are infested with a thrips population of mixed ages. After an incubation period of 6 days, samples are checked for mortality and special effects (e.g. phytotoxicity).

In this test, compounds listed in the tables above show good activity. In particular compounds P1.3, P1.7, P1.8, P1.9, P2.3, P2.6 and P2.8 show an activity of over 80% at a concentration of 400 ppm.

### EXAMPLE B4

### Activity Against Tetranychus urticae (Two-Spotted Spider Mite) (Mixed Population, Feeding/Residual Contact Activity, Preventive)

Bean leaf discs on agar in 24-well microtiter plates are sprayed with test solutions. After drying, the leaf discs are infested with mite populations of mixed ages. 8 days later, discs are checked for egg mortality, larval mortality, and adult mortality.

In this test, compounds listed in the tables above show good activity. In particular compounds P1.3, P1.4, P1.5, P1.7, P1.8, P1.9, P1.11, P1.14, P2.3, P2.6, P2.7 and P2.8 show an activity of over 80% at a concentration of 400 ppm.

### EXAMPLE B5

### Activity Against Myzus persicae (Green Peach Aphid) (Mixed Population, Feeding/Residual Contact Activity, Plant Damage Evaluation)

Cabbage plants infested with a mixed population of Myzus persicae are treated with diluted test solutions of the compounds in a spray chamber. 6 days after treatment, samples are checked for mortality and for plant damage (phytotoxicity), visual assessment being made using a 0-100% rating scale (100%=total damage to plant; 0%=no damage to plant).

In this test, compounds listed in the tables above show good activity against Myzus persicae and acceptable plant compatibility. For example compounds P1.3, P1.4, P1.5, P1.6, P1.7, P1.8, P1.9, P1.10, P1.11, P1.12, P1.13, P1.14, P2.3, P2.4, P2.5, P2.6, P2.7, P2.8, P2.10, P2.11, P2.12 and P2.13 show an activity of greater or equal to 80% against Myzus persicae and damage to cabbage plants less or equal to 10% at a concentration of 200 ppm.

### EXAMPLE B6

### Activity Against Frankliniella occidentalis (Western Flower Thrips)

Bean leaf discs on agar in petri dishes or bean plants in a spray chamber are treated with diluted test solutions. After drying leaf discs are cut and placed in plastic cups on the surface of an agar layer and infested with mixed population. 6 days (leaf discs) or 14 days (plants) after the infestation, samples are checked for reduction of treated population and compared to the non treated population.

In this test, compounds listed in the tables above show good activity. For example compounds P1.9 and P2.7 show an activity of over 80% at a concentration of 200 ppm.

### EXAMPLE B7

### Activity Against Bemisia tabaci (Tobacco White Fly) (Larvicide, Contact/Feeding)

Bean plants are infested with 20-30 adults that were removed after a 4 day egg-laying period. After another 7 days, bean plants with hatched nymphs (N-2) are treated (2 replicates) with the test solutions in a spray chamber. Three weeks later, samples are checked for number of emerged adults. Efficacy was calculeted by comparing number of emerged adults in treated and non treated samples.

In this test, compounds listed in the tables above show good activity. For example compounds P1.3, P1.6, P1.8, P1.9, P2.3, P2.7 and P2.8 show an activity of over 80% at a concentration of 200 ppm.

### EXAMPLE B8

### Activity Against Nilaparvata lugens (Brown Rice Planthopper) (Larvicide, Feeding/Contact)

Rice seedlings are treated with the diluted test solutions in a spray chamber. After drying, they are infested with 20 N₃ nymphs (2 replicates). 6-12 days after the treatment samples are checked for mortality, growth regulation, and effects on the F₁ generation.

In this test, compounds listed in the tables above show good activity. For example compounds P1.3, P1.6, P1.8, P1.9, P2.3, P2.7 and P2.8 show an activity of over 80% at a concentration of 400 ppm.

### EXAMPLE B9

### Activity Against Aphis craccivora (Cowpea Aphid) (Mixed Population, Contact/Feeding)

Pea seedlings, infested with an aphid population of mixed ages, are treated (2 replicates) with diluted test solutions in a spray chamber. 6 days after treatment, samples are checked for mortality.

In this test, compounds listed in the tables above show good activity. For example compound P1.6 shows an activity of over 80% at a concentration of 200 ppm.

### EXAMPLE B10

### Activity Against Aphis craccivora (Cowpea Aphid) (Mixed Population, Systemic/Feeding)

Roots of pea seedlings, infested with an aphid population of mixed ages, are placed (2 replicates) directly in the test solution. 6 days later, samples are checked for mortality.

In this test, compounds listed in the tables above show good activity. For example compounds P1.6, P1.8, P1.9, P2.7 and P2.8 show an activity of over 80% at a concentration of 25 ppm.

### EXAMPLE B11

### Translaminar Activity Against Aphis craccivora (Cowpea Aphid)

French bean leaves (Phaseolus vulgaris) are infested with about 20 mixed age individuals on the lower leaf side using clip cages. 1 day after the infestation, the upper side of the leaves is treated with the test solution by painting. 5 days later, samples are checked for mortality.

In this test, compounds listed in the tables above show good activity. For example compounds P1.3, P1.6, P1.8, P1.9, P2.3 and P2.7 show an activity of over 80% at a concentration of 200 ppm.

### EXAMPLE B12

### Activity Against Aonidiella aurantii (Red Scale)

Treatment of potato tubers by dipping the in the test solution. One day later, tubers are infested with about 50 crawlers. 6-8 weeks after application samples are checked for the number of crawlers of the next generation (compared to the non treated samples).

In this test, compounds listed in the tables above show good activity. For example compounds P1.3, P1.6, P1.9, P2.3, P2.7 and P2.8 show an activity of over 80% at a concentration of 200 ppm.

### EXAMPLE B13

### Drench Activity Against Myzus persicae (Green Peach Aphid)

Pea seedlings cultivated in field soil are treated as drench application and infested with a mixed population of M. persicae. 7 days after infestation, samples are checked for mortality.

In this test, compounds listed in the tables above show good activity. For example compounds P1.8, P1.9, P2.7 and P2.8 show an activity of over 80% at a concentration of 25 ppm.

1. Compounds of the formula I 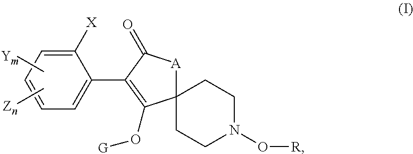
wherein
X, Y and Z independently of each other are C₁₋₄alkyl, C₃₋₆cycloalkyl, C₁₋₄haloalkyl, C₁₋₄alkoxy, halogen, phenyl or phenyl substituted by C₁₋₄alkyl, C₁₋₄haloalkyl, halogen or cyano;
m and n, independently of each other, are 0, 1, 2 or 3 and m+n is 0, 1, 2 or 3;
G is hydrogen, a metal, ammonium, sulfonium or a latentiating group;
A is either NR¹ or NOR¹, wherein R¹ is hydrogen, C₁₋₆alkyl, C₁₋₆haloalkyl, C₃₋₆cycloalkyl, or C₃₋₆cycloalkyl where in the cycloalkyl moiety a methylene group is replaced by O, S or NR₀, where R₀ is C₁₋₆alkyl or C₁₋₆alkoxy, or R¹ is C₃₋₆cycloalkyl(C₁₋₄)alkyl, or C₃₋₆cyclo-alkyl(C₁₋₄)alkyl where in the cycloalkyl moiety a methylene group is replaced by O, S or NR₀, where R₀ is C₁₋₆alkyl or C₁₋₆alkoxy, or R¹ is C₂₋₆alkenyl, C₂₋₆haloalkenyl, C₃₋₆alkynyl, C₁₋₆cyanoalkyl, benzyl, furanyl-(C₁₋₄)alkyl, C₁₋₄alkoxy(C₁₋₄)alkyl, C₁₋₄alkoxy(C₁₋₄)alkoxy(C₁₋₄)alkyl, C₁₋₄alkylthio(C₁₋₄alkyl, C₁₋₄alkylsulfinyl(C₁₋₄)alkyl or C₁₋₄alkylsulfonyl(C₁₋₄)alkyl;
R is hydrogen, C₁₋₆alkyl, C₁₋₆haloalkyl, C₁₋₆cyanoalkyl, C₂₋₆alkenyl, C₂₋₆haloalkenyl, C₃-C₆alkynyl, benzyl, C₁₋₄alkoxy(C₁₋₄)alkyl, or C₁₋₄alkoxy(C₁₋₄)alkoxy(C₁₋₄)alkyl; and
with the proviso that at least one of X, Y or Z is C₂₋₆alkenyl, C₂₋₆alkynyl, —CHO, C₁₋₆alkylcarbonyl or C₁₋₆alkoxycarbonyl;
or an agrochemically acceptable salt or an N-oxide thereof.

2. A process for the preparation of the compounds of the formula I according to claim 1, wherein G is hydrogen, which comprises cyclisation of the compound of formula IV 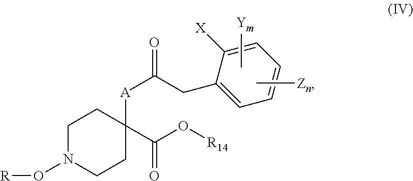
wherein X, Y, Z, m, n, R and A have the meanings assigned to them in claim 1, and R₁₄ is C₁₋₆alkyl, under basic conditions.

3. A pesticidal composition comprising a pesticidal effective amount of at least one compound of formula I according to claim 1.

4. A pesticidal composition according to claim 3, which, in addition to comprising the compound of formula I, comprises formulation adjuvants.

5. A pesticidal composition according to claim 3, which, in addition to comprising the compound of formula I, comprises at least one additional insecticide, acaricide, nemacitide or molluscicide.

6. A pesticidal composition according to claim 3, which, in addition to comprising the compound of formula I, comprises at least one additional fungicide, herbicide, safener or plant growth regulator.

7. A method of combating and controlling pests which comprises applying to a pest, to a locus of a pest, or to a plant susceptible to attack by a pest a pesticidally effective amount of a compound of formula I.

8. A method of combating and controlling pests which comprises applying to a pest, to a locus of a pest, or to a plant susceptible to attack by a pest a pesticidal composition according to claim 3.

9. Compounds of the formula IV 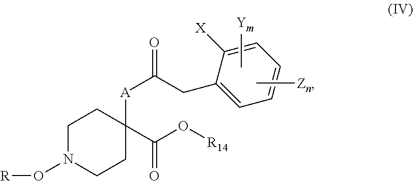
or salts thereof, wherein X, Y, Z, m, n, R and A have the meanings assigned to them in claim 1, and R₁₄ is C₁₋₆alkyl.

10. Compounds of the formula XI 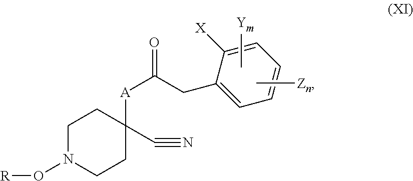
or salts thereof, wherein X, Y, Z, m, n, R and A have the meanings assigned to them in claim 1.
